(19) United States
(12) Patent Application Publication
(10) Pub. No.: US 2007/0219186 A1
(43) Pub. Date: Sep. 20, 2007

(54) Pyrazolo[1,5-A]pyrimidine derivatives and methods of use thereof

(75) Inventors: Ariamala Gopalsamy, Mahwah, NJ (US); Gregory M. Ciszewski, Pearl River, NY (US); Mengxiao Shi, New Rochelle, NY (US); Dan Maarten Berger, New City, NY (US); Nancy Torres, Nyack, NY (US); Jeremy I. Levin, New City, NY (US); Dennis William Powell, Cortland Manor, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/724,689

(22) Filed: Mar. 15, 2007

Related U.S. Application Data
(60) Provisional application No. 60/783,631, filed on Mar. 17, 2006.

(51) Int. Cl. A61K 31/541; A61K 31/5377; A61K 31/519; C07D 487/04

(52) U.S. Cl. 514/227.8; 514/234.2; 514/252.16; 514/259.3; 544/60; 544/114; 544/280

(57) ABSTRACT

The present invention relates to pyrazolo[1,5-a]pyrimidine derivatives, compositions comprising an effective amount of a pyrazolo[1,5-a]pyrimidine derivative and methods for treating or preventing cancer, comprising administering to a subject in need thereof an effective amount of a pyrazolo[1,5-a]pyrimidine derivative.

This application claims priority from copending U.S. provisional application No. 60/783,631, filed Mar. 17, 2006, the entire disclosure of which is hereby incorporated by reference.

### FIELD OF THE INVENTION

The present invention relates to pyrazolo[1,5-a]pyrimidine derivatives, compositions comprising an effective amount of a pyrazolo[1,5-a]pyrimidine derivative and methods for treating or preventing cancer, comprising administering to a subject in need thereof an effective amount of a pyrazolo[1,5-a]pyrimidine derivative.

### BACKGROUND OF THE INVENTION

Cancer is second only to cardiovascular disease as a cause of death in the United States. The American Cancer Society estimated that in 2004, there were 1.6 million new cases of cancer and 655,000 cancer-related deaths. There are currently over 10 million living Americans who have been diagnosed with cancer and the NIH estimates the direct medical costs of cancer as over $100 billion per year with an additional $100 billion in indirect costs due to lost productivity—the largest such costs of any major disease.

Cancer is a process by which the controlling mechanisms that regulate cell growth and differentiation are impaired, resulting in a failure to control cell turnover and growth. This lack of control causes a tumor to grow progressively, enlarging and occupying space in vital areas of the body. If the tumor invades surrounding tissue and is transported to distant sites, death of the individual can result.

The selective killing of cancer cells, while minimizing deleterious effects on normal cells, is a desired goal in cancer therapy. Modalities commonly used in the treatment of cancer include chemotherapy, radiation therapy, surgery and biological therapy (a broad category that includes gene-, protein- or cell-based treatments and immunotherapy). Despite the availability of a variety of anticancer agents, traditional chemotherapy has drawbacks. Many anticancer agents are toxic, and chemotherapy can cause significant, and often dangerous, side effects, including severe nausea, bone marrow depression, liver, heart and kidney damage, and immunosuppression. Since it is difficult to predict the pattern of sensitivity of a neoplastic cell population to anticancer drugs, it is common to use multi-drug regimens.

Despite the significant research efforts and resources which have been directed towards the development of novel anticancer agents and improved methods for treating cancer there remains a need in the art for novel compounds, compositions, and methods that are useful for treating cancer with improved therapeutic indices.

### DETAILED DESCRIPTION OF THE INVENTION

In a first embodiment of this invention, the compounds of Formula I

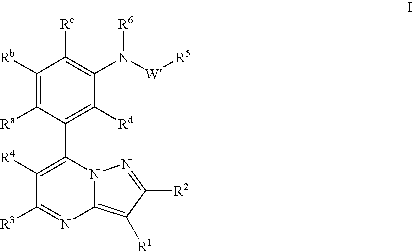

or pharmaceutically acceptable salts or prodrugs thereof include those

wherein
R¹ is selected from the group consisting of R⁷, J, —C(O)OR¹⁶, —C(O)NR⁷R¹⁴, —NR⁶C(O)R¹⁶, nitrile, a 5-7 membered heterocyclic ring or heteroaryl ring containing 1-3 heteroatoms selected from N, O or S, and an aryl ring, wherein the R⁷ group, the R¹⁴ group, the R¹⁶ group, the heterocyclic ring, the heteroaryl ring and the aryl ring can be optionally substituted with one to four substituents selected from the group consisting of -J, —NO₂, —CN, —N₃, —CHO, —CF₃, —OCF₃, —R¹¹, —OR¹¹, —S(O)ₘR¹¹, —NR¹⁵R¹¹, —NR¹²S(O)ₘR¹⁵, —OR⁹OR¹¹, —OR⁹NR¹⁵R¹¹, —N(R¹²)R⁹OR¹⁵, —N(R¹²)R⁹NR¹⁵R¹¹, —NR¹²C(O)R¹⁵, —C(O)R¹¹, —C(O)OR¹¹, —C(O)NR¹²R¹¹, —OC(O)R¹¹, —OC(O)OR¹¹, —OC(O)NR¹⁵R¹¹, —NR¹²C(O)R¹⁵, —NR¹²C(O)OR¹⁵, —NR¹²C(O)NR¹⁵R¹¹, —R⁸OR¹¹, —R⁸NR¹⁵R¹¹, —R⁸S(O)ₘR¹¹, —R⁸C(O)R¹¹, —R⁸C(O)OR¹¹, —R⁸C(O)NR¹⁵R¹¹, —R⁸OC(O)R¹¹, —R⁸OC(O)OR¹¹, —R⁸OC(O)NR¹⁵R¹¹, —R⁸NR¹²C(O)R¹⁵, —R⁸NR¹²C(O)OR¹⁵, —R⁸NR¹²C(O)NR¹⁵R¹¹, R²⁰, —OR⁹R²⁰, —N(R¹²)R⁹R²⁰, —C(O)R²⁰, —OC(O)R²⁰, —NR¹²C(O)R², —R⁸R²⁰, —R⁸C(O)R²¹, —R⁸OC(O)R²⁰, —R⁸NR¹²C(O)R²⁰ and YR¹⁰;

R² is an alkyl of 1-6 carbon atoms, branched alkyl of 3-8 carbon atoms, cis-alkenyl of 2-6 carbon atoms, a trans-alkenyl of 2-6 carbon atoms, an alkynyl of 2-6 carbon atoms, aryl, heteroaryl or heterocyclyl; each of said alkyl of 1-6 carbon atoms, branched alkyl of 3-8 carbon atoms, cis-alkenyl of 2-6 carbon atoms, trans-alkenyl of 2-6 carbon atoms, alkynyl of 2-6 carbon atoms, aryl, heteroaryl or heterocyclyl being optionally substituted with one to four substituents selected from the group consisting of -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹⁷)R⁹OR¹⁷, N(R¹⁷)R⁹NR⁷R¹⁴, —NR¹⁷C(O)R¹¹, —C(O)R¹⁷, —C(O)OR¹⁷, —C(O)NR⁷R¹⁴, —OC(O)R¹⁷, —OC(O)OR¹⁷, —OC(O)NR⁷R¹⁴, NR¹⁷C(O)R¹¹, —NR¹⁷C(O)OR¹¹, —NR¹⁷C(O)NR⁷R¹⁴, —R⁸OR¹⁷, —R⁸NR⁷R¹⁴, —R⁸S(O)ₘR¹⁷, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹⁷C(O)R¹¹, —R⁸NR¹⁷C(O)OR¹¹, —R⁸NR¹⁷C(O)NR⁷R¹⁴ and YR¹⁰;

R^{a}, R^{b}, R^{c}, R^{d}, R³ and R⁴ are independently selected from the group consisting of H, -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷, —OC(O)OR¹⁷, —OC(O)NR⁷R¹⁴, NR¹¹C(O)R¹⁷, —NR¹¹C(O)OR¹⁷, —NR¹¹C(O)NR⁷R¹⁴, —R⁸OR¹⁷, —R⁸NR⁷R¹⁴, —R⁸S(O)ₘR¹⁷, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷, —R⁸NR¹¹C(O)NR⁷R¹⁴ and YR¹⁰;

R⁵ is an alkyl of 1-6 carbon atoms, branched alkyl of 3-8 carbon atoms, cis-alkenyl of 2-6 carbon atoms, a trans-alkenyl of 2-6 carbon atoms, an alkynyl of 2-6 carbon atoms, an aryl ring, a heterocyclic ring or a heteroaryl ring, said heterocylic ring and heteroaryl containing 1-3 heteroatoms selected from N, O or S, wherein the heterocyclic, heteroaryl and aryl rings are optionally substituted with one to four substituents selected from the group consisting of -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷, —OC(O)OR¹⁷, —OC(O)NR⁷R¹⁴NR¹¹C(O)R¹⁷, —NR¹¹C(O)OR¹⁷, —NR¹¹C(O)NR⁷R¹⁴, —R⁸OR¹⁷, —R⁸NR⁷R¹⁴, —R⁸S(O)ₘR¹⁷, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸C(O)NR⁷R¹⁴, —R⁸NR¹¹C(O)R¹⁷, —R⁸NR¹¹C(O)OR¹⁷, —R⁸NR¹¹C(O)NR⁷R¹⁴, R⁸N(R¹²)R⁹OR¹⁵, —NR¹¹C(O)R⁹R¹⁰, —YR⁸R¹⁰, —YR⁸NR⁷R¹⁴ and —YR¹⁰;

R⁶ is H, alkyl of 1-6 carbon atoms or branched alkyl of 3-8 carbon atoms;

R⁷, R¹¹, R¹², R¹⁴, R¹⁵, R¹⁶, and R¹⁷ are independently selected from H, alkyl of 1-6 carbon atoms, branched alkyl of 3-8 carbon atoms, cis-alkenyl of 2-6 carbon atoms, a trans-alkenyl of 2-6 carbon atoms, and an alkynyl of 2-6 carbon atoms; said alkyl, branched alkyl, cis-alkenyl, trans-alkenyl, and alkynyl groups being optionally substituted with 1-3 J atoms; R⁷ and R¹⁴ together with the N to which they are attached may join to form a 3 to 8 membered ring, said 3 to 8 membered ring optionally containing additional heteroatoms N, O, or S(O)ₘ to form a heterocycle which can optionally be substituted with alkyl of 1-6 carbon atoms, carbonyl, hydroxy, alkoxy of 1 to 6 carbon atoms, NH₂, NHR⁶, or N(R⁶)₂;

R⁸ is a divalent group selected from alkyl of 1-6 carbon atoms, alkenyl of 2-6 carbon atoms, and alkynyl of 2-6 carbon atoms;

R⁹ is a divalent alkyl group of 2-6 carbon atoms;

R¹⁰ is selected from the group consisting of a cycloalkyl ring of 3-10 carbons, a bicycloalkyl ring of 3-10 carbons, an aryl, a heterocyclyl ring, a heteroaryl ring, and a heteroaryl fused to 1-3 aryl or heteroaryl rings; any of said heterocyclyl ring and heteroaryl rings containing 1-3 heteroatoms selected from N, O or S; wherein any of the aryl, cycloalkyl, bicycloalkyl, heterocyclic or heteroaryl rings may be optionally substituted with one to four substituents selected from the group consisting of —H, -aryl, —CH₂-aryl, —NH-aryl, —O-aryl, —S(O)ₘ-aryl, -J, —NO₂, —CN, —N₃, —CHO, —CF₃, —OCF₃, —R¹⁷, —OR¹⁷, S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷—, —OC(O)OR¹⁷, —OC(O)NR⁷R¹⁴, —NR¹¹C(O)R¹⁷, —NR¹¹C(O)OR¹⁷, —NR¹¹C(O)NR⁷R¹⁴, —R⁸OR¹⁷, R⁸NR⁷R¹⁴, —R⁸S(O)ₘR¹⁷, —R⁸C(O)R¹⁷, —R⁸C(O)OR¹⁷, —R⁸C(O)NR⁷R¹⁴, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷, and —R⁸NR¹¹C(O)NR⁷R¹⁴;

R²⁰ is a heterocyclic ring containing 3-8 members, at least one member being N which is the point of attachment for the moiety, and optionally said 3 to 8 membered ring containing additional heteroatoms N, O, or S(O)ₘ and said 3-8 membered ring being optionally substituted with 1-4 substituents selected from alkyl of 1-6 carbon atoms, carbonyl, hydroxy, alkoxy of 1 to 6 carbon atoms, NH₂, NHR⁶, or N(R⁶)₂;

J is fluoro, chloro, bromo, or iodo;

m is an integer of 0-2;

W′ is —C(O)— or —C(O)—NR¹⁷—, —SO₂—, or —CO—C(R⁶)₂—; and

Y is selected from the group consisting of a bond, a divalent alkyl group of 1-6 carbon atoms, NH, O, —NR¹⁷, —C≡C—, cis- —CH═CH—, and trans- —CH═CH—.

In one aspect of said first embodiment, R² is pyridyl, furanyl, or thiophenyl.

In another aspect of said first embodiment, R² is pyridyl.

In another aspect of said first embodiment, Y is a bond or a divalent alkyl group.

In another aspect of said first embodiment, R² is substituted phenyl.

In another aspect of said first embodiment, R⁵ is mono- or di-substituted phenyl.

In another aspect of said first embodiment, R⁵ is monosubstituted phenyl.

In another aspect of said first embodiment, R⁵ is disubstituted phenyl.

In another aspect of said first embodiment, R⁵ is mono- or di-substituted phenyl and the substituents are selected from the group consisting of -J, —NO₂, —CN, —N₃, —CHO, —CF₃, —OCF₃, —R¹⁷, and —OR¹⁷.

In another aspect of said first embodiment, the substitutents on R⁵ are selected from -J, —CF₃, and —OR¹⁷.

In another aspect of said first embodiment, J is fluoro or chloro.

In another aspect of said first embodiment, J is fluoro.

In another aspect of said first embodiment, R¹ is selected from the group consisting of H, J, —C(O)OR¹⁶, —C(O)NR⁷R¹⁴, —NR⁶C(O)R¹⁶, a 5-7 membered heterocyclic ring or heteroaryl ring containing 1-3 heteroatoms selected from N, O or S, and an aryl ring, wherein the R⁷ group, the R¹⁴ group, the R¹⁶ group, the heterocyclic ring, the heteroaryl ring and the aryl ring can be optionally substituted.

In another aspect of said first embodiment, R¹ is H or J.

In another aspect of said first embodiment, W′ is C(O).

In another aspect of said first embodiment, R⁵ is phenyl.

In another aspect of said first embodiment R¹ is H.

In another aspect of said first embodiment, R² is an optionally substituted heteroaryl.

In another aspect of said first embodiment R² is substituted aryl.

In another aspect of said first embodiment, W′ is C(O), R⁵ is phenyl, R¹ is H, and R² is substituted aryl or an optionally substituted heteroaryl.

In another aspect of said first embodiment, R² is an optionally substituted aryl, optionally substituted heteroaryl or optionally substituted heterocyclyl.

In a second embodiment of this invention, the compounds of Formula I 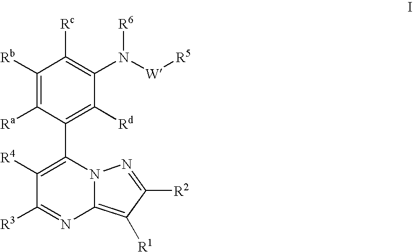
and pharmaceutically acceptable salts and prodrugs thereof include those

wherein
R¹ is —C(O)—NH—R¹³, substituted aryl, substituted heteroaryl, substituted heterocyclyl, —C(O)O-substituted alkyl, —C(O)O-heteroaryl, or substituted alkynyl;
R¹³ is heteroaryl, alkyl of 1 to 6 carbon atoms optionally substituted with heterocyclyl, heteroaryl, alkoxy, optionally substituted aryl, dialkylamino, or alkylamino;
R² is selected from the group consisting of R⁷, J, —C(O)OR¹⁶, —C(O)NR⁷R¹⁴,

—NR⁶C(O)R¹⁶, nitrile, a 5-7 membered heterocyclic ring or heteroaryl ring containing 1-3 heteroatoms selected from N, O or S, and an aryl ring, wherein the R⁷ group, the R¹⁴ group, the R¹⁶ group, the heterocyclic ring, the heteroaryl ring and the aryl ring can be optionally substituted with one to four substituents selected from the group consisting of -J, —NO₂, —CN, —N₃, —CHO, —CF₃, —OCF₃, —R¹¹, —OR¹¹, —S(O)ₘR¹¹, —NR¹⁵R¹¹, —NR¹²S(O)ₘR¹⁵, —OR⁹OR¹¹, —OR⁹NR¹⁵, R¹¹, —N(R¹²)R⁹OR¹⁵, —N(R¹²)R⁹NR¹⁵R¹¹, —NR¹²C(O)R¹⁵, —C(O)R¹¹, —C(O)OR¹¹, —C(O)NR¹²R¹¹, —OC(O)R¹¹, —OC(O)OR¹¹, —OC(O)NR¹⁵R¹¹, NR¹²C(O)R¹⁵, —NR¹²C(O)OR¹⁵, —NR¹²C(O)NR¹⁵R¹¹, —R⁸OR¹¹, —R⁸NR¹⁵R¹¹, —R⁸S(O)ₘR¹¹, —R⁸C(O)R¹¹, —R⁸C(O)OR¹¹, —R⁸C(O)NR¹⁵R¹¹, —R⁸OC(O)R¹¹, R⁸OC(O)OR¹¹, —R⁸OC(O)NR¹⁵R¹¹, —R⁸NR¹²C(O)R¹⁵, —R⁸NR¹²C(O)OR¹⁵, —R⁸NR¹²C(O)NR¹⁵R¹¹, R²⁰, —OR⁹R²⁰, —N(R¹²)R⁹R²⁰, —C(O)R²⁰, —OC(O)R²⁰, —NR¹²C(O)R²⁰, —R⁸R²⁰, —R⁸C(O)R²⁰, —R⁸OC(O)R²⁰, —R⁸NR¹²C(O)R²⁰, and YR¹⁰;

R^{a}, R^{b}, R^{c}, R^{d}, R³ and R⁴ are independently selected from the group consisting of H, -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷, —OC(O)OR¹⁷, —OC(O)NR⁷R¹⁴, NR¹¹C(O)R¹⁷, —NR¹¹C(O)OR¹⁷, —NR¹¹C(O)NR⁷R¹⁴, —R⁸OR¹⁷, —R⁸NR⁷R¹⁴, —R⁸S(O)ₘR¹⁷, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷, —R⁸NR¹¹C(O)NR⁷R¹⁴ and YR¹⁰;

R⁵ is an alkyl of 1-6 carbon atoms, branched alkyl of 3-8 carbon atoms, cis-alkenyl of 2-6 carbon atoms, a trans-alkenyl of 2-6 carbon atoms, an alkynyl of 2-6 carbon atoms, an aryl ring, a heterocyclic ring or a heteroaryl ring, said heterocylic ring and heteroaryl containing 1-3 heteroatoms selected from N, O or S, wherein the heterocyclic, heteroaryl and aryl rings are optionally substituted with one to four substituents selected from the group consisting of -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷, —OC(O)OR¹⁷, —OC(O)NR⁷R¹⁴, NR¹¹C(O)R¹⁷, —NR¹¹C(O)OR¹⁷, —NR¹¹C(O)NR⁷R¹⁴, —R⁸OR¹⁷, —R⁸NR⁷R¹⁴, —R⁸S(O)ₘR¹⁷, —R⁸C(O)R¹⁷, —R^{c}(O)OR¹⁷, —R⁸C(O)NR⁷R¹⁴, —R⁸OC(O)R¹⁷, —ROC(O)OR¹⁷, —R⁸OC(O)NR⁷R¹⁴, —R⁸NR¹¹C(O)R¹⁷, R⁸NR¹¹C(O)OR¹⁷, —R⁸NR¹¹C(O)NR⁷R¹⁴, R⁸N(R¹²)R⁹OR¹⁵, —NR¹¹C(O)R⁹R¹⁰, —YR⁸R¹⁰, —YR⁸NR⁷R¹⁴ and —YR¹⁰;

R⁶ is H, alkyl of 1-6 carbon atoms or branched alkyl of 3-8 carbon atoms;

R⁷, R¹¹, R¹², R¹⁴, R¹⁵, R¹⁶, and R¹⁷ are independently selected from H, alkyl of 1-6 carbon atoms, branched alkyl of 3-8 carbon atoms, cis-alkenyl of 2-6 carbon atoms, a trans-alkenyl of 2-6 carbon atoms, and an alkynyl of 2-6 carbon atoms; said alkyl, branched alkyl, cis-alkenyl, trans-alkenyl, and alkynyl groups being optionally substituted with 1-3 J atoms; R⁷ and R¹⁴ together with the N to which they are attached may join to form a 3 to 8 membered ring, said 3 to 8 membered ring optionally containing additional heteroatoms N, O, or S(O)ₘ to form a heterocycle which can optionally be substituted with alkyl of 1-6 carbon atoms, carbonyl, hydroxy, alkoxy of 1 to 6 carbon atoms;

R⁸ is a divalent group selected from alkyl of 1-6 carbon atoms, alkenyl of 2-6 carbon atoms, and alkynyl of 2-6 carbon atoms;

R⁹ is a divalent alkyl group of 2-6 carbon atoms;

R¹⁰ is selected from the group consisting of a cycloalkyl ring of 3-10 carbons, a bicycloalkyl ring of 3-10 carbons, an aryl, a heterocyclyl ring, a heteroaryl ring, and a heteroaryl fused to 1-3 aryl or heteroaryl rings; any of said heterocyclyl ring and heteroaryl rings containing 1-3 heteroatoms selected from N, O or S; wherein any of the aryl, cycloalkyl, bicycloalkyl, heterocyclic or heteroaryl rings may be optionally substituted with one to four substituents selected from the group consisting of —H, -aryl, —CH₂-aryl, —NH-aryl, —O-aryl, —S(O)ₘ-aryl, -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷—, —OC(O)OR¹⁷, —OC(O)NR⁷R¹⁴, —NR¹¹C(O)R¹⁷, —NR¹¹C(O)OR¹⁷, —NR¹¹C(O)NR⁷R¹⁴, —R⁸OR¹⁷, R⁸NR⁷R¹⁴, —R⁸S(O)ₘR¹⁷, —R⁸C(O)R¹⁷, —R⁸C(O)OR¹⁷, —R⁸C(O)NR⁷R¹⁴, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷, and —R⁸NR¹¹C(O)NR⁷R¹⁴;

R²⁰ is a heterocyclic ring containing 3-8 members, at least one member being N which is the point of attachment for the moiety, and optionally said 3 to 8 membered ring containing additional heteroatoms N, O, or S(O)ₘ and said 3-8 membered ring being optionally substituted with 1-4 substituents selected from alkyl of 1-6 carbon atoms, carbonyl, hydroxy, alkoxy of 1 to 6 carbon atoms, NH₂, NHR⁶, or N(R⁶)₂;

J is fluoro, chloro, bromo, or iodo;

m is an integer of 0-2;

W′ is —C(O)— or —C(O)—NR¹⁷—, —SO₂—, or —CO—C(R⁶)₂—; and

Y is selected from the group consisting of a bond, a divalent alkyl group of 1-6 carbon atoms, NH, O, —NR¹⁷, —C≡C—, cis- —CH═CH—, and trans- —CH═CH—.

In one aspect of the second embodiment, W′ is C(O).

In another aspect of the second embodiment, R⁵ is an optionally substituted aryl.

In another aspect of the second embodiment, R⁵ is an optionally substituted phenyl.

In another aspect of the second embodiment, R⁵ is phenyl.

In another aspect of the second embodiment, R² is H.

In another aspect of the second embodiment, R¹ is substituted aryl.

In another aspect of the second embodiment R¹ is optionally substituted heteroaryl.

In another aspect of the second embodiment, W′ is C(O), R⁵ is phenyl, R² is H, R¹ is substituted aryl or heteroaryl.

In another aspect of the second embodiment, J is fluoro or chloro.

In another aspect of the second embodiment, J is fluoro.

In a third embodiment of this invention, the compounds of Formula I

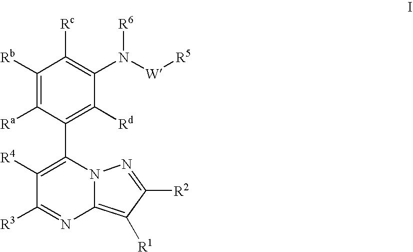

or pharmaceutically acceptable salts or prodrugs thereof include those

wherein
R¹ is selected from the group consisting of H, J, —C(O)OR⁶, —C(O)NR⁷R¹⁴, —NR⁶C(O)R¹⁶, nitrile, a 5-7 membered heterocyclic ring or heteroaryl ring containing 1-3 heteroatoms selected from N, O or S, and an aryl ring, wherein the R⁷ group, the R¹⁴ group, the R¹⁶ group, the heterocyclic ring, the heteroaryl ring and the aryl ring can be optionally substituted with one to four substituents selected from the group consisting of -J, —NO₂, —CN, —N₃, —CHO, —CF₃, —OCF₃, —R¹¹, —OR¹¹, —S(O)ₘR¹¹, —NR¹⁵R¹¹, —NR¹²S(O)ₘR¹⁵, —OR⁹OR¹¹, —OR⁹NR¹⁵R¹¹, —N(R¹²)R⁹OR¹⁵, —N(R¹²)R⁹NR¹⁵R¹¹, —NR¹²C(O)R¹⁵, —C(O)R¹¹, —C(O)OR¹¹, —C(O)NR¹²R¹¹, —OC(O)R¹¹, —OC(O)OR¹¹, —OC(O)NR¹⁵R¹¹, NR¹²C(O)R¹⁵, —NR¹²C(O)OR¹⁵, —NR¹²C(O)NR¹⁵R¹¹, —R⁸OR¹¹, —R⁸NR¹⁵R¹¹, —R⁸S(O)ₘR¹¹, —R⁸C(O)R¹¹, —R⁸C(O)OR¹¹, —R⁸C(O)NR¹⁵R¹¹, —R⁸OC(O)R¹¹, —R⁸OC(O)OR¹¹, —R⁸OC(O)NR¹⁵R¹¹, —R⁸NR¹²C(O)R¹⁵, —R⁸NR¹²C(O)OR¹⁵, —R⁸NR¹²C(O)NR¹⁵R¹¹, R²⁰, —OR⁹R²⁰, —N(R¹²)R⁹R²⁰, —C(O)R²⁰, —OC(O)R²⁰, —NR¹²C(O)R²⁰, —R⁸R²⁰, —R⁸C(O)R²⁰, —R⁸OC(O)R²⁰, —R⁸NR¹²C(O)R²⁰, and YR¹⁰;
R² is selected from the group consisting of H, J, —C(O)OR¹⁶, —C(O)NR⁷R¹⁴, —NR⁶C(O)R¹⁶, nitrile, a 5-7 membered heterocyclic ring or heteroaryl ring containing 1-3 heteroatoms selected from N, O or S, and an aryl ring, wherein the R⁷ group, the R¹⁴ group, the R¹⁶ group, the heterocyclic ring, the heteroaryl ring and the aryl ring can be optionally substituted with one to four substituents selected from the group consisting of -J, —NO₂, —CN, —N₃, —CHO, —CF₃, —OCF₃, —R¹¹, —OR¹¹, —S(O)ₘR¹¹, —NR¹⁵R¹¹, —NR¹²S(O)ₘR¹⁵, —OR⁹OR¹¹, —OR⁹NR¹⁵R¹¹, —N(R¹²)R⁹OR¹⁵, —N(R¹²)R⁹NR¹⁵R¹¹, —NR¹²C(O)R¹⁵, —C(O)R¹¹, —C(O)OR¹¹, —C(O)NR¹²R¹¹, —OC(O)R¹¹, —OC(O)OR¹¹, —OC(O)NR¹⁵R¹¹, NR¹²C(O)R¹⁵, —NR¹²C(O)OR¹⁵, —NR¹²C(O)NR¹⁵R¹¹, —R⁸OR¹¹, —R⁸NR¹⁵R¹¹, —R⁸S(O)ₘR¹¹, —R⁸C(O)R¹¹, —R⁸C(O)OR¹¹, —R⁸C(O)NR¹⁵R¹¹, —R⁸OC(O)R¹¹, —R⁸OC(O)OR¹¹, —R⁸OC(O)NR¹⁵R¹¹, —R⁸NR¹²C(O)R¹⁵, —R⁸NR¹²C(O)OR¹⁵, —R⁸NR¹²C(O)NR¹⁵R¹¹, R²⁰, —OR⁹R²⁰, —N(R¹²)R⁹R²⁰, —C(O)R²⁰, —OC(O)R²⁰, —NR¹²C(O)R²⁰, —R⁸R²⁰, —R⁸C(O)R²⁰, —R⁸OC(O)R²⁰, —R⁸NR¹²C(O)R²⁰, and YR¹⁰;

R^{a}, R^{b}, R^{c}, R^{d}, R³ and R⁴ are independently selected from the group consisting of H, -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷, —OC(O)OR¹⁷, —OC(O)NR⁷R¹⁴, NR¹¹C(O)R¹⁷, —NR¹¹C(O)OR¹⁷, —NR¹¹C(O)NR⁷R¹⁴, —R⁸R¹⁷, —R⁸NR⁷R¹⁴, —R⁸S(O)ₘR¹⁷, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷, —R⁸NR¹¹C(O)NR⁷R¹⁴ and YR¹⁰;

R⁵ is —NH-aryl-heterocyclyl, —NH-aryl-heteroaryl, —CH₂-substituted aryl, —CH₂—R¹⁸, or NH—R¹⁸, said aryl portion, heterocyclyl portion and heteroaryl portion of the —NH-aryl-heterocyclyl and —NH-aryl-heteroaryl groups being optionally substituted;

R⁶ is H, alkyl of 1-6 carbon atoms or branched alkyl of 3-8 carbon atoms;

R⁷, R¹¹, R¹², R¹⁴, R¹⁵, R¹⁶, and R¹⁷ are independently selected from H, alkyl of 1-6 carbon atoms, branched alkyl of 3-8 carbon atoms, cis-alkenyl of 2-6 carbon atoms, a trans-alkenyl of 2-6 carbon atoms, and an alkynyl of 2-6 carbon atoms; said alkyl, branched alkyl, cis-alkenyl, trans-alkenyl, and alkynyl groups being optionally substituted with 1-3 J atoms; R⁷ and R¹⁴ together with the N to which they are attached may join to form a 3 to 8 membered ring;

R⁸ is a divalent group selected from alkyl of 1-6 carbon atoms, alkenyl of 2-6 carbon atoms, and alkynyl of 2-6 carbon atoms;

R⁹ is a divalent alkyl group of 2-6 carbon atoms;

R¹⁰ is selected from the group consisting of a cycloalkyl ring of 3-10 carbons, a bicycloalkyl ring of 3-10 carbons, an aryl, a heterocyclyl ring, a heteroaryl ring, and a heteroaryl fused to 1-3 aryl or heteroaryl rings; any of said heterocyclyl ring and heteroaryl rings containing 1-3 heteroatoms selected from N, O or S; wherein any of the aryl, cycloalkyl, bicycloalkyl, heterocyclic or heteroaryl rings may be optionally substituted with one to four substituents selected from the group consisting of —H, -aryl, —CH₂-aryl, —NH-aryl, —O-aryl, —S(O)ₘ-aryl, -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR⁷, —C(O)NR⁷R¹⁴, —OC(O)R¹⁷—, —OC(O)OR¹⁷, —OC(O)NR⁷R¹⁴, —NR¹¹C(O)R¹⁷, —NR¹¹C(O)OR¹⁷, —NR¹¹C(O)NR⁷R¹⁴, —R⁸OR¹⁷, R⁸NR⁷R¹⁴, —R⁸S(O)ₘR¹⁷, —R⁸C(O)R¹⁷, —R⁸C(O)OR¹⁷, —R⁶C(O)NR⁷R¹⁴, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷, and —R⁸NR¹¹C(O)NR⁷R¹⁴;

R¹⁸ is an aryl ring fused to a heteroaryl ring or heterocyclic ring, such as

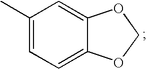

R²⁰ is a heterocyclic ring containing 3-8 members, at least one member being N which is the point of attachment for the moiety, and optionally said 3 to 8 membered ring containing additional heteroatoms N, O, or S(O)ₘ and said 3-8 membered ring being optionally substituted with 1-4 substituents selected from alkyl of 1-6 carbon atoms, carbonyl, hydroxy, alkoxy of 1 to 6 carbon atoms, NH₂, NHR⁶, or N(R⁶)₂;

J is fluoro, chloro, bromo, or iodo;

m is an integer of 0-2;

W′ is —C(O)— or —C(O)—NR¹⁷—, —SO₂—, or —CO—C(R⁶)₂—; and

Y is selected from the group consisting of a bond, a divalent alkyl group of 1-6 carbon atoms, NH, O, —NR¹⁷, —C≡C— cis- —CH═CH—, and trans- —CH═CH—.

In a fourth embodiment of this invention, the compounds of Formula I 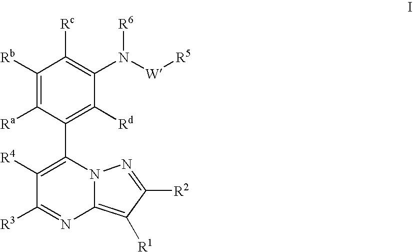
or pharmaceutically acceptable salts or prodrugs thereof include those
wherein

R¹ is selected from the group consisting of H, J, —C(O)OR¹⁶, —NR⁶C(O)R¹⁶, a 5-7 membered heterocyclic ring or heteroaryl ring containing 1-3 heteroatoms selected from N, O or S, and an aryl ring, wherein the R⁷ group, the R¹⁴ group, the R¹⁶ group, can be optionally substituted with one to four substituents selected from the group consisting of -J, —NO₂, —CN, —N₃, —CHO, —CF₃, —OCF₃, —R¹¹, —OR¹¹, —S(O)ₘR¹¹, —NR¹⁵R¹¹, NR¹²S(O)ₘR¹⁵, —OR⁹OR¹¹, —OR⁹NR¹⁵R¹¹, —N(R¹²)R⁹OR¹⁵, —N(R¹²)R⁹NR¹⁵R¹¹, —NR¹²C(O)R¹⁵, —C(O)R¹¹, —C(O)OR¹¹, —C(O)NR¹²R¹¹, —OC(O)R¹¹, —OC(O)OR¹¹, —OC(O)NR¹⁵R¹¹, NR¹²C(O)R¹⁵, —NR¹²C(O)OR¹⁵, —NR¹²C(O)NR¹⁵R¹¹, —R⁸OR¹¹, —R⁸NR¹⁵R¹¹, —R⁸S(O)ₘR¹¹, —R⁸C(O)R¹¹, —R⁸C(O)OR¹¹, —R⁸C(O)NR¹⁵R¹¹, —R⁸OC(O)R¹¹, —R⁸OC(O)OR¹¹, —R⁸OC(O)NR¹⁵R¹¹, —R⁸NR¹²C(O)R¹⁵, —R⁸NR¹²C(O)OR¹⁵, —R⁸NR¹²C(O)NR¹⁵R¹¹ and YR¹⁰;

R² is selected from the group consisting of H, J, —C(O)OR¹⁶, —C(O)NR⁷R¹⁴, —NR⁶C(O)R¹⁶, nitrile;

R^{a}, R^{b}, R^{c}, R^{d}, R³ and R⁴ are independently selected from the group consisting of H, -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷, —OC(O)OR¹⁷, —OC(O)NR⁷R¹⁴, NR¹¹C(O)R¹⁷, —NR¹¹C(O)OR¹⁷, —NR¹¹C(O)NR⁷R¹⁴, —R⁸OR¹⁷, —R⁸NR⁷R¹⁴, —R⁸S(O)ₘR¹⁷, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷, —R⁸NR¹¹C(O)NR⁷R¹⁴ and YR¹⁰;

R⁵ is an alkyl of 1-6 carbon atoms, branched alkyl of 3-8 carbon atoms, cis-alkenyl of 2-6 carbon atoms, a trans-alkenyl of 2-6 carbon atoms, an alkynyl of 2-6 carbon atoms, an aryl ring, a heterocyclic ring or a heteroaryl ring, said heterocylic ring and heteroaryl containing 1-3 heteroatoms selected from N, O or S, wherein the heterocyclic, heteroaryl and aryl rings are optionally substituted with one to four substituents selected from the group consisting of -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R⁷, —OC(O)OR¹⁷, —OC(O)NR⁷R¹⁴, NR¹¹C(O)R¹⁷, —NR⁶C(O)OR¹⁷, —NR¹¹C(O)NR⁷R¹⁴, —R⁸OR¹⁷, —R⁸NR⁷R¹⁴, —R⁸S(O)ₘR¹⁷, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷, —R⁸NR¹¹C(O)NR⁷R¹⁴, R⁸N(R¹²)R⁹OR¹⁵, —NR⁸C(O)R⁹R¹⁰, —YR⁸R¹⁰, —YR⁸NR⁷R¹⁴ and —YR¹⁰;

R⁶ is H, alkyl of 1-6 carbon atoms or branched alkyl of 3-8 carbon atoms;

R⁷, R¹¹, R¹², R¹⁴, R¹⁵, R¹⁶, and R¹⁷ are independently selected from H, alkyl of 1-6 carbon atoms, branched alkyl of 3-8 carbon atoms, cis-alkenyl of 2-6 carbon atoms, a trans-alkenyl of 2-6 carbon atoms, and an alkynyl of 2-6 carbon atoms; or R⁷ and R¹⁴ together with the N to which they are attached may join to form a 3 to 8 membered ring, said 3-8 membered ring optionally containing a heteroatom selected from N, O, and S in addition to said N atom to which R⁷ and R¹⁴ are attached;

R⁸ is a divalent group selected from alkyl of 1-6 carbon atoms, alkenyl of 2-6 carbon atoms, and alkynyl of 2-6 carbon atoms;

R⁹ is a divalent alkyl group of 2-6 carbon atoms;

R¹⁰ is selected from the group consisting of a cycloalkyl ring of 3-10 carbons, a bicycloalkyl ring of 3-10 carbons, an aryl, a heterocyclyl ring, a heteroaryl ring, and a heteroaryl fused to 1-3 aryl or heteroaryl rings; any of said heterocyclyl ring and heteroaryl rings containing 1-3 heteroatoms selected from N, O or S; wherein any of the aryl, cycloalkyl, bicycloalkyl, heterocyclic or heteroaryl rings may be optionally substituted with one to four substituents selected from the group consisting of —H, -aryl, —CH₂-aryl, —NH-aryl, —O-aryl, —S(O)ₘ-aryl, -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷—, —OC(O)OR¹⁷, —OC(O)NR⁷R¹⁴, —NR¹¹C(O)R¹⁷, NR¹¹C(O)OR¹⁷, —NR¹¹C(O)NR⁷R¹⁴, —R⁸OR¹⁷, R⁸NR⁷R¹⁴, —R⁸S(O)ₘR¹⁷, —R⁸C(O)R¹⁷, —R¹¹C(O)OR¹⁷, —R⁸C(O)NR⁷R¹⁴, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷, and —R⁸NR¹¹C(O)NR⁷R¹⁴;

J is fluoro, chloro, bromo, or iodo;

m is an integer of 0-2;

W¹ is —C(O)— or —C(O)—NR¹⁷—, —SO₂—, or —CO—C(R⁶)₂—; and

Y is selected from the group consisting of a bond, a divalent alkyl group of 1-6 carbon atoms, NH, O, —NR¹⁷, —C≡C— cis- —CH═CH—, and trans- —CH═CH—.

In another embodiment of the present invention R^{a}, R^{b}, R^{c}, R^{d}, R³ and R⁴ are independently selected from the group consisting of H, -J, —NO₂, —CN, —N₃, —CHO, —CF₃, —OCF₃, —R¹⁷, and —OR¹⁷.

Reactions are performed in a solvent appropriate to the reagents and materials employed and suitable for the transformation being effected. It is understood by those skilled in the art of organic synthesis that the various functionalities present on the molecule must be consistent with the chemical transformations proposed. This may necessitate judgement as to the order of synthetic steps, protecting groups, if required, and deprotection conditions.

Another embodiment of the invention is a mixture comprising a compound of formula I or a pharmaceutically acceptable salt thereof and an impurity. An impurity is any other chemical or biological entity, such as a different compound, stereoisomer, salt, intermediate, or pollutant of any sort. An impurity may be present in the mixture in an amount greater than the compound itself, but typically is present in an amount less than the desired compound. In another aspect of the invention the impurity may be present in an amount of less than 10% of the amount of the compound. In another aspect of the invention the impurity may be present in an amount of less than 10% of the mixture.

Another embodiment of the present invention comprises a compound selected from the group consisting of:
ethyl 7-(3-{[3-(trifluoromethyl)benzoyl]amino}phenyl)pyrazolo[1,5-a]pyrimidine-3-carboxylate;
ethyl 7-(3-{[4-fluoro-3-(trifluoromethyl)benzoyl]amino}phenyl)pyrazolo[1,5-a]pyrimidine-3-carboxylate;
ethyl 7-[3-(benzoylamino)phenyl]pyrazolo[1,5-a]pyrimidine-3-carboxylate;
ethyl 7-{3-[(3-bromobenzoyl)amino]phenyl}pyrazolo[1,5-a]pyrimidine-3-carboxylate;
ethyl 7-{3-[(1-benzothien-2-ylcarbonyl)amino]phenyl}pyrazolo[1,5-a]pyrimidine-3-carboxylate;
ethyl 7-{3-[(4-chlorobenzoyl)amino]phenyl}pyrazolo[1,5-a]pyrimidine-3-carboxylate;
ethyl 7-(3-{[3-(trifluoromethoxy)benzoyl]amino}phenyl)pyrazolo[1,5-a]pyrimidine-3-carboxylate;
ethyl 7-{3-[(3-methoxybenzoyl)amino]phenyl}pyrazolo[1,5-a]pyrimidine-3-carboxylate;
ethyl 7-(3-{[3-fluoro-4-(trifluoromethyl)benzoyl]amino}phenyl)pyrazolo[1,5-a]pyrimidine-3-carboxylate;
ethyl 7-(3-{[4-(trifluoromethyl)benzoyl]amino}phenyl)pyrazolo[1,5-a]pyrimidine-3-carboxylate;
ethyl 7-{3-[({[4-chloro-3-(trifluoromethyl)phenyl]amino}carbonyl)amino]phenyl}pyrazolo[1,5-a]pyrimidine-3-carboxylate;
ethyl 7-[3-({[3-(trifluoromethyl)phenyl]sulfonyl}amino)phenyl]pyrazolo[1,5-a]pyrimidine-3-carboxylate;
ethyl 7-{3-[(3-cyanobenzoyl)amino]phenyl}pyrazolo[1,5-a]pyrimidine-3-carboxylate;
ethyl 7-[3-({[(2,4-dichlorophenyl)amino]carbonyl}amino)phenyl]pyrazolo[1,5-a]pyrimidine-3-carboxylate;
ethyl 7-{3-[({[4-(ethoxycarbonyl)phenyl]amino}carbonyl)amino]phenyl}pyrazolo[1,5-a]pyrimidine-3-carboxylate;
ethyl 7-{3-[({[3,5-bis(trifluoromethyl)phenyl]amino}carbonyl)amino]phenyl}pyrazolo[1,5-a]pyrimidine-3-carboxylate;
ethyl 7-[3-({[(3,5-dichlorophenyl)amino]carbonyl}amino)phenyl]pyrazolo[1,5-a]pyrimidine-3-carboxylate;
ethyl 7-{3-[({[4-(methylthio)phenyl]amino}carbonyl)amino]phenyl}pyrazolo[1,5-a]pyrimidine-3-carboxylate;
ethyl 7-[3-({[(4-acetylphenyl)amino]carbonyl}amino)phenyl]pyrazolo[1,5-a]pyrimidine-3-carboxylate;
ethyl 7-[3-({[(4-isopropylphenyl)amino]carbonyl}amino)phenyl]pyrazolo[1,5-a]pyrimidine-3-carboxylate;
ethyl 7-(3-{[(2-naphthylamino)carbonyl]amino}phenyl)pyrazolo[1,5-a]pyrimidine-3-carboxylate;
ethyl 7-(3-{[(mesitylamino)carbonyl]amino}phenyl)pyrazolo[1,5-a]pyrimidine-3-carboxylate;
ethyl 7-{3-[({[4-(trifluoromethoxy)phenyl]amino}carbonyl)amino]phenyl}pyrazolo[1,5-a]pyrimidine-3-carboxylate;
ethyl 7-(3-{[({4-[(trifluoromethyl)thio]phenyl}amino)carbonyl]amino}phenyl)pyrazolo[1,5-a]pyrimidine-3-carboxylate;
ethyl 7-[3-({[(3-chloro-4-fluorophenyl)amino]carbonyl}amino)phenyl]pyrazolo[1,5-a]pyrimidine-3-carboxylate;
ethyl 7-(3-{[4-methyl-3-(trifluoromethyl)benzoyl]amino}phenyl)pyrazolo[1,5-a]pyrimidine-3-carboxylate;
ethyl 7-(3-{[2-chloro-5-(trifluoromethyl)benzoyl]amino}phenyl)pyrazolo[1,5-a]pyrimidine-3-carboxylate;
ethyl 7-(3-{[3-chloro-2-fluoro-5-(trifluoromethyl)benzoyl]amino}phenyl)pyrazolo[1,5-a]pyrimidine-3-carboxylate;
ethyl 7-{3-[(4-chloro-2,5-difluorobenzoyl)amino]phenyl}pyrazolo[1,5-a]pyrimidine-3-carboxylate;
ethyl 7-(3-{[4-methoxy-3-(trifluoromethyl)benzoyl]amino}phenyl)pyrazolo[1,5-a]pyrimidine-3-carboxylate;
N-methyl-7-(3-{[3-(trifluoromethyl)benzoyl]amino}phenyl)pyrazolo[1,5-a]pyrimidine-3-carboxamide;
7-(3-{[3-(trifluoromethyl)benzoyl]amino}phenyl)pyrazolo[1,5-a]pyrimidine-3-carboxylic acid;
ethyl 7-{3-[({[3-(trifluoromethyl)phenyl]amino}carbonyl)amino]phenyl}pyrazolo[1,5-a]pyrimidine-3-carboxylate;
ethyl 7-[3-({[(4-chlorophenyl)amino]carbonyl}amino)phenyl]pyrazolo[1,5-a]pyrimidine-3-carboxylate;
ethyl 7-{3-[({[4-(trifluoromethyl)phenyl]amino}carbonyl)amino]phenyl}pyrazolo[1,5-a]pyrimidine-3-carboxylate;
ethyl 7-[3-({[(4-chloro-2-methylphenyl)amino]carbonyl}amino)phenyl]pyrazolo[1,5-a]pyrimidine-3-carboxylate;
ethyl 7-{3-[({[2-chloro-5-(trifluoromethyl)phenyl]amino}carbonyl)amino]phenyl}pyrazolo[1,5-a]pyrimidine-3-carboxylate;
ethyl 7-[3-({[(4-cyanophenyl)amino]carbonyl}amino)phenyl]pyrazolo[1,5-a]pyrimidine-3-carboxylate;
ethyl 7-{3-[({[2-(trifluoromethyl)phenyl]amino}carbonyl)amino]phenyl}pyrazolo[1,5-a]pyrimidine-3-carboxylate;
ethyl 7-[3-({[(3,4-dichlorophenyl)amino]carbonyl}amino)phenyl]pyrazolo[1,5-a]pyrimidine-3-carboxylate;
ethyl 7-[3-({[(4-bromophenyl)amino]carbonyl}amino)phenyl]pyrazolo[1,5-a]pyrimidine-3-carboxylate;
ethyl 7-[3-({[(3,4-dimethylphenyl)amino]carbonyl}amino)phenyl]pyrazolo[1,5-a]pyrimidine-3-carboxylate;
ethyl 7-{3-[({[4-chloro-2-(trifluoromethyl)phenyl]amino}carbonyl)amino]phenyl}pyrazolo[1,5-a]pyrimidine-3-carboxylate;
ethyl 7-{3-[({[4-fluoro-3-(trifluoromethyl)phenyl]amino}carbonyl)amino]phenyl}pyrazolo[1,5-a]pyrimidine-3-carboxylate;
N-(2-methoxyethyl)-7-(3-{[3-(trifluoromethyl)benzoyl]amino}phenyl)pyrazolo[1,5-a]pyrimidine-3-carboxamide;
N-propyl-7-(3-{[3-(trifluoromethyl)benzoyl]amino}phenyl)pyrazolo[1,5-a]pyrimidine-3-carboxamide;
N-pyridin-3-yl-7-(3-{[3-(trifluoromethyl)benzoyl]amino}phenyl)pyrazolo[1,5-a]pyrimidine-3-carboxamide;
N-(2-pyrrolidin-1-ylethyl)-7-(3-{[3-(trifluoromethyl)benzoyl]amino}phenyl)pyrazolo[1,5-a]pyrimidine-3-carboxamide;
N-[2-(dimethylamino)ethyl]-7-(3-{[3-(trifluoromethyl)benzoyl]amino}phenyl)pyrazolo[1,5-a]pyrimidine-3-carboxamide;
N-[3-(4-methylpiperazin-1-yl)propyl]-7-(3-{[3-(trifluoromethyl)benzoyl]amino}phenyl)pyrazolo[1,5-a]pyrimidine-3-carboxamide;
N-ethyl-7-(3-{[3-(trifluoromethyl)benzoyl]amino}phenyl)pyrazolo[1,5-a]pyrimidine-3-carboxamide;
N-(2-morpholin-4-ylethyl)-7-(3-{[3-(trifluoromethyl)benzoyl]amino}phenyl)pyrazolo[1,5-a]pyrimidine-3-carboxamide;
N-(3-morpholin-4-ylpropyl)-7-(3-{[3-(trifluoromethyl)benzoyl]amino}phenyl)pyrazolo[1,5-a]pyrimidine-3-carboxamide;
N-[2-(1-methylpyrrolidin-2-yl)ethyl]-7-(3-{[3-(trifluoromethyl)benzoyl]amino}phenyl)pyrazolo[1,5-a]pyrimidine-3-carboxamide;
N-[3-(1H-imidazol-1-yl)propyl]-7-(3-{[3-(trifluoromethyl)benzoyl]amino}phenyl)pyrazolo[1,5-a]pyrimidine-3-carboxamide;
N-(3-methoxypropyl)-7-(3-{([3-(trifluoromethyl)benzoyl]amino}phenyl)pyrazolo[1,5-a]pyrimidine-3-carboxamide;
N-benzyl-7-(3-{[3-(trifluoromethyl)benzoyl]amino}phenyl)pyrazolo[1,5-a]pyrimidine-3-carboxamide;
ethyl 2-methyl-7-(3-{[3-(trifluoromethyl)benzoyl]amino}phenyl)pyrazolo[1,5-a]pyrimidine-3-carboxylate;
N-[3-(3-pyridin-2-ylpyrazolo[1,5-a]pyrimidin-7-yl)phenyl]-3-(trifluoromethyl)benzamide;
N-{3-[3-(2H-tetrazol-5-yl)pyrazolo[1,5-a]pyrimidin-7-yl]phenyl}-3-(trifluoromethyl)benzamide;
7-(3-{[3-(trifluoromethyl)benzoyl]amino}phenyl)pyrazolo[1,5-a]pyrimidine-3-carboxamide;
N-[3-(3-cyano-2-piperazin-1-ylpyrazolo[1,5-a]pyrimidin-7-yl)phenyl]-3-(trifluoromethyl)benzamide;
N-[3-(3-cyanopyrazolo[1,5-a]pyrimidin-7-yl)phenyl]-3-(trifluoromethyl)benzamide;
ethyl 2-methyl-7-(3-{[4-methyl-3-(trifluoromethyl)benzoyl]amino}phenyl)pyrazolo[1,5-a]pyrimidine-3-carboxylate;
4-methyl-N-[3-(3-pyridin-2-ylpyrazolo[1,5-a]pyrimidin-7-yl)phenyl]-3-(trifluoromethyl)benzamide;
4-methyl-N-{3-[3-(2H-tetrazol-5-yl)pyrazolo[1,5-a]pyrimidin-7-yl]phenyl}-3-(trifluoromethyl)benzamide;
7-(3-{[4-methyl-3-(trifluoromethyl)benzoyl]amino}phenyl)pyrazolo[1,5-a]pyrimidine-3-carboxamide;
N-[3-(3-cyano-2-piperazin-1-ylpyrazolo[1,5-a]pyrimidin-7-yl)phenyl]-4-methyl-3-(trifluoromethyl)benzamide;
N-[3-(3-cyanopyrazolo[1,5-a]pyrimidin-7-yl)phenyl]-4-methyl-3-(trifluoromethyl)benzamide;
ethyl 7-{3-[(3-chlorobenzoyl)amino]phenyl}pyrazolo[1,5-a]pyrimidine-3-carboxylate;
ethyl 7-{3-[(3,4-dichlorobenzoyl)amino]phenyl}pyrazolo[1,5-a]pyrimidine-3-carboxylate;
ethyl 7-{3-[(3,5-dichlorobenzoyl)amino]phenyl}pyrazolo[1,5-a]pyrimidine-3-carboxylate;
ethyl 7-{3-[(3-chloro-4-methoxybenzoyl)amino]phenyl}pyrazolo[1,5-a]pyrimidine-3-carboxylate;
ethyl 7-{3-[(5-chloro-2-methylbenzoyl)amino]phenyl}pyrazolo[1,5-a]pyrimidine-3-carboxylate;
ethyl 7-(3-{[3-fluoro-5-(trifluoromethyl)benzoyl]amino}phenyl)pyrazolo[1,5-a]pyrimidine-3-carboxylate;
ethyl 7-(3-{[4-fluoro-2-(trifluoromethyl)benzoyl]amino}phenyl)pyrazolo[1,5-a]pyrimidine-3-carboxylate;
ethyl 7-{3-[({[3-methoxy-5-(trifluoromethyl)phenyl]amino}carbonyl)amino]phenyl}pyrazolo[1,5-a]pyrimidine-3-carboxylate;
ethyl 7-{3-[({[4-cyano-3-(trifluoromethyl)phenyl]amino}carbonyl)amino]phenyl}pyrazolo[1,5-a]pyrimidine-3-carboxylate;
ethyl 7-{3-[({[4-methyl-3-(trifluoromethyl)phenyl]amino}carbonyl)amino]phenyl}pyrazolo[1,5-a]pyrimidine-3-carboxylate;
ethyl 7-[3-({[(3-chlorophenyl)amino]carbonyl}amino)phenyl]pyrazolo[1,5-a]pyrimidine-3-carboxylate;
ethyl 7-[3-({[(3-chloro-4-methoxyphenyl)amino]carbonyl}amino)phenyl]pyrazolo[1,5-a]pyrimidine-3-carboxylate;
ethyl 7-[3-({[(3-chloro-4-methylphenyl)amino]carbonyl}amino)phenyl]pyrazolo[1,5-a]pyrimidine-3-carboxylate;
ethyl 7-[3-({[(4-bromo-3-chlorophenyl)amino]carbonyl}amino)phenyl]pyrazolo[1,5-a]pyrimidine-3-carboxylate;
ethyl 7-[3-({[(3-chloro-4-morpholin-4-ylphenyl)amino]carbonyl}amino)phenyl]pyrazolo[1,5-a]pyrimidine-3-carboxylate;
ethyl 7-(2-nitro-5-{[3-(trifluoromethyl)benzoyl]amino}phenyl)pyrazolo[1,5-a]pyrimidine-3-carboxylate;
ethyl 7-(4-chloro-3-{[3-(trifluoromethyl)benzoyl]amino}phenyl)pyrazolo[1,5-a]pyrimidine-3-carboxylate;
ethyl 7-(4-(2,6-dimethylmorpholin-4-yl)-3-{[3-(trifluoromethyl)benzoyl]amino}phenyl)pyrazolo[1,5-a]pyrimidine-3-carboxylate;
ethyl 7-(4-methoxy-3-{[3-(trifluoromethyl)benzoyl]amino}phenyl)pyrazolo[1,5-a]pyrimidine-3-carboxylate;
ethyl 7-(4-fluoro-3-{[3-(trifluoromethyl)benzoyl]amino}phenyl)pyrazolo[1,5-a]pyrimidine-3-carboxylate;
ethyl 7-(4-(benzyloxy)-3-{[3-(trifluoromethyl)benzoyl]amino}phenyl)pyrazolo[1,5-a]pyrimidine-3-carboxylate;
ethyl 7-(2-fluoro-5-{[3-(trifluoromethyl)benzoyl]amino}phenyl)pyrazolo[1,5-a]pyrimidine-3-carboxylate;
ethyl 7-(3-{[(3-bromophenyl)acetyl]amino}phenyl)pyrazolo[1,5-a]pyrimidine-3-carboxylate;
ethyl 7-(3-{[(3-fluorophenyl)acetyl]amino}phenyl)pyrazolo[1,5-a]pyrimidine-3-carboxylate;
ethyl 7-(3-{[(3-chlorophenyl)acetyl]amino}phenyl)pyrazolo[1,5-a]pyrimidine-3-carboxylate;
ethyl 7-(3-{[(3,4-dichlorophenyl)acetyl]amino}phenyl)pyrazolo[1,5-a]pyrimidine-3-carboxylate;
ethyl 7-(3-{[(3-methoxyphenyl)acetyl]amino}phenyl)pyrazolo[1,5-a]pyrimidine-3-carboxylate;
ethyl 7-[3-({[3-(trifluoromethyl)phenyl]acetyl}amino)phenyl]pyrazolo[1,5-a]pyrimidine-3-carboxylate;
ethyl 7-(3-{[(3-methylphenyl)acetyl]amino}phenyl)pyrazolo[1,5-a]pyrimidine-3-carboxylate;
ethyl 7-[3-({[3,5-bis(trifluoromethyl)phenyl]acetyl}amino)phenyl]pyrazolo[1,5-a]pyrimidine-3-carboxylate;
ethyl 7-{3-[(1,3-benzodioxol-5-ylacetyl)amino]phenyl}pyrazolo[1,5-a]pyrimidine-3-carboxylate;
ethyl 7-(3-{[(4-methoxy-3-methylphenyl)acetyl]amino}phenyl)pyrazolo[1,5-a]pyrimidine-3-carboxylate;
ethyl 7-(3-{[(2,3,6-trifluorophenyl)acetyl]amino}phenyl)pyrazolo[1,5-a]pyrimidine-3-carboxylate;
ethyl 7-(3-{[4-chloro-3-(trifluoromethyl)benzoyl]amino}phenyl)pyrazolo[1,5-a]pyrimidine-3-carboxylate;
ethyl 7-(3-{[2-methyl-5-(trifluoromethyl)benzoyl]amino}phenyl)pyrazolo[1,5-a]pyrimidine-3-carboxylate;
ethyl 7-(5-{[4-methyl-3-(trifluoromethyl)benzoyl]amino}-2-nitrophenyl)pyrazolo[1,5-a]pyrimidine-3-carboxylate;
ethyl 7-(4-chloro-3-{[4-methyl-3-(trifluoromethyl)benzoyl]amino}phenyl)pyrazolo[1,5-a]pyrimidine-3-carboxylate;
ethyl 7-(4-methoxy-3-{[4-methyl-3-(trifluoromethyl)benzoyl]amino}phenyl)pyrazolo[1,5-a]pyrimidine-3-carboxylate;
ethyl 7-(4-(benzyloxy)-3-{[4-methyl-3-(trifluoromethyl)benzoyl]amino}phenyl)pyrazolo[1,5-a]pyrimidine-3-carboxylate;
N-[3-(2-pyridin-4-ylpyrazolo[1,5-a]pyrimidin-7-yl)phenyl]-3-(trifluoromethyl)benzamide;
N-(3-{2-[3-(dimethylamino)propyl]pyrazolo[1,5-a]pyrimidin-7-yl}phenyl)-3-(trifluoromethyl)benzamide;
N-[3-(2-pyridin-2-ylpyrazolo[1,5-a]pyrimidin-7-yl)phenyl]-3-(trifluoromethyl)benzamide;
N-[3-(2-methylpyrazolo[1,5-a]pyrimidin-7-yl)phenyl]-3-(trifluoromethyl)benzamide;
N-{3-[2-(2-furyl)pyrazolo[1,5-a]pyrimidin-7-yl]phenyl}-3-(trifluoromethyl)benzamide;
N-{3-[2-(2-thienyl)pyrazolo[1,5-a]pyrimidin-7-yl]phenyl}-3-(trifluoromethyl)benzamide;
N-{3-[2-(4-chlorophenyl)pyrazolo[1,5-a]pyrimidin-7-yl]phenyl}-3-(trifluoromethyl)benzamide;
4-methyl-N-[3-(2-methylpyrazolo[1,5-a]pyrimidin-7-yl)phenyl]-3-(trifluoromethyl)benzamide;
4-methyl-N-{3-[2-(2-thienyl)pyrazolo[1,5-a]pyrimidin-7-yl]phenyl}-3-(trifluoromethyl)benzamide;
N-{3-[2-(4-chlorophenyl)pyrazolo[1,5-a]pyrimidin-7-yl]phenyl}-4-methyl-3-(trifluoromethyl)benzamide;
4-methyl-N-[3-(2-phenylpyrazolo[1,5-a]pyrimidin-7-yl)phenyl]-3-(trifluoromethyl)benzamide;
7-{3-[({[4-chloro-3-(trifluoromethyl)phenyl]amino}carbonyl)amino]phenyl}-N-[3-(1H-imidazol-1-yl)propyl]pyrazolo[1,5-a]pyrimidine-3-carboxamide;
7-{3-[({[4-chloro-3-(trifluoromethyl)phenyl]amino}carbonyl)amino]phenyl}-N-(3-methoxypropyl)pyrazolo[1,5-a]pyrimidine-3-carboxamide;
7-{3-[({[4-chloro-3-(trifluoromethyl)phenyl]amino}carbonyl)amino]phenyl}-N-[2-(diethylamino)ethyl]pyrazolo[1,5-a]pyrimidine-3-carboxamide;
7-{3-[({[4-chloro-3-(trifluoromethyl)phenyl]amino}carbonyl)amino]phenyl}-N-(2-morpholin-4-ylethyl)pyrazolo[1,5-a]pyrimidine-3-carboxamide;
methyl 7-(3-{[3-(trifluoromethyl)benzoyl]amino}phenyl)pyrazolo[1,5-a]pyrimidine-3-carboxylate;
2,2,2-trifluoroethyl 7-(3-{[3-(trifluoromethyl)benzoyl]amino}phenyl)pyrazolo[1,5-a]pyrimidine-3-carboxylate;
pyridin-3-yl 7-(3-{[3-(trifluoromethyl)benzoyl]amino}phenyl)pyrazolo[1,5-a]pyrimidine-3-carboxylate;
2-(dimethylamino)ethyl 7-(3-{[3-(trifluoromethyl)benzoyl]amino}phenyl)pyrazolo[1,5-a]pyrimidine-3-carboxylate;
2-methoxyethyl 7-(3-{[3-(trifluoromethyl)benzoyl]amino}phenyl)pyrazolo[1,5-a]pyrimidine-3-carboxylate;
4-chloro-N-[3-(2-pyridin-4-ylpyrazolo[1,5-a]pyrimidin-7-yl)phenyl]-3-(trifluoromethyl)benzamide;
4-methoxy-N-[3-(2-pyridin-4-ylpyrazolo[1,5-a]pyrimidin-7-yl)phenyl]-3-(trifluoromethyl)benzamide;
N-[4-fluoro-3-(trifluoromethyl)phenyl]-N′-[3-(2-pyridin-4-ylpyrazolo[1,5-a]pyrimidin-7-yl)phenyl]urea;
N-[3-(2-pyridin-4-ylpyrazolo[1,5-a]pyrimidin-7-yl)phenyl]-N′-[3-(trifluoromethyl)phenyl]urea;
N-[4-chloro-3-(trifluoromethyl)phenyl]-N′-[3-(2-pyridin-4-ylpyrazolo[1,5-a]pyrimidin-7-yl)phenyl]urea;
4-methyl-N-[3-(2-pyridin-4-ylpyrazolo[1,5-a]pyrimidin-7-yl)phenyl]-3-(trifluoromethyl)benzamide;
N-(3-{2-[4-(hydroxymethyl)phenyl]pyrazolo[1,5-a]pyrimidin-7-yl}phenyl)-3-(trifluoromethyl)benzamide;
N-{3-[2-(4-methoxyphenyl)pyrazolo[1,5-a]pyrimidin-7-yl]phenyl}-3-(trifluoromethyl)benzamide;
3-(trifluoromethyl)-N-(3-{2-[3-(trifluoromethyl)phenyl]pyrazolo[1,5-a]pyrimidin-7-yl}phenyl)benzamide;
N-[3-(2-tert-butylpyrazolo[1,5-a]pyrimidin-7-yl)phenyl]-3-(trifluoromethyl)benzamide;
N-{3-[2-(4-fluorophenyl)pyrazolo[1,5-a]pyrimidin-7-yl]phenyl}-3-(trifluoromethyl)benzamide;
N-[3-(2-{4-[(ethoxymethoxy)methyl]phenyl}pyrazolo[1,5-a]pyrimidin-7-yl)phenyl]-3-(trifluoromethyl)benzamide;
methyl 3-[7-(3-{[3-(trifluoromethyl)benzoyl]amino}phenyl)pyrazolo[1,5-a]pyrimidin-2-yl]benzoate;
4-[7-(3-{[3-(trifluoromethyl)benzoyl]amino}phenyl)pyrazolo[1,5-a]pyrimidin-2-yl]benzyl acetate;
N-[3-(3-bromopyrazolo[1,5-a]pyrimidin-7-yl)phenyl]-N′-[4-chloro-3-(trifluoromethyl)phenyl]urea;
N-[3-(2-pyridin-4-ylpyrazolo[1,5-a]pyrimidin-7-yl)phenyl]benzamide;
2-chloro-N-[3-(2-pyridin-4-ylpyrazolo[1,5-a]pyrimidin-7-yl)phenyl]benzamide;
3-methoxy-N-[3-(2-pyridin-4-ylpyrazolo[1,5-a]pyrimidin-7-yl)phenyl]benzamide;
3-methyl-N-[3-(2-pyridin-4-ylpyrazolo[1,5-a]pyrimidin-7-yl)phenyl]benzamide;
4-methyl-N-[3-(2-pyridin-4-ylpyrazolo[1,5-a]pyrimidin-7-yl)phenyl]benzamide;
3,4-dichloro-N-[3-(2-pyridin-4-ylpyrazolo[1,5-a]pyrimidin-7-yl)phenyl]benzamide;
3-chloro-N-[3-(2-pyridin-4-ylpyrazolo[1,5-a]pyrimidin-7-yl)phenyl]benzamide;
4-chloro-N-[3-(2-pyridin-4-ylpyrazolo[1,5-a]pyrimidin-7-yl)phenyl]benzamide;
N-[3-(2-pyridin-3-ylpyrazolo[1,5-a]pyrimidin-7-yl)phenyl]-3-(trifluoromethyl)benzamide;
N-(3-pyrazolo[1,5-a]pyrimidin-7-ylphenyl)-3-(trifluoromethyl)benzamide;
N-[3-(3-pyridin-4-ylpyrazolo[1,5-a]pyrimidin-7-yl)phenyl]-3-(trifluoromethyl)benzamide;
N-[3-(3-pyridin-3-ylpyrazolo[1,5-a]pyrimidin-7-yl)phenyl]-3-(trifluoromethyl)benzamide;
N-{3-[3-(3-aminophenyl)pyrazolo[1,5-a]pyrimidin-7-yl]phenyl}-3-(trifluoromethyl)benzamide;
N-(3-{3-[4-(dimethylamino)phenyl]pyrazolo[1,5-a]pyrimidin-7-yl}phenyl)-3-(trifluoromethyl)benzamide;
N-{3-[3-(4-chlorophenyl)pyrazolo[1,5-a]pyrimidin-7-yl]phenyl}-3-(trifluoromethyl)benzamide;
N-{3-[3-(4-methoxyphenyl)pyrazolo[1,5-a]pyrimidin-7-yl]phenyl}-3-(trifluoromethyl)benzamide;
N-{3-[3-(4-methylphenyl)pyrazolo[1,5-a]pyrimidin-7-yl]phenyl}-3-(trifluoromethyl)benzamide;
N-[3-(3-bromopyrazolo[1,5-a]pyrimidin-7-yl)phenyl]-4-methyl-3-(trifluoromethyl)benzamide;
N-[3-(3-bromopyrazolo[1,5-a]pyrimidin-7-yl)phenyl]-4-methoxy-3-(trifluoromethyl)benzamide;
N-[3-(3-bromopyrazolo[1,5-a]pyrimidin-7-yl)phenyl]-4-fluoro-3-(trifluoromethyl)benzamide;
N-[3-(3-bromopyrazolo[1,5-a]pyrimidin-7-yl)phenyl]-4-chloro-3-(trifluoromethyl)benzamide;
N-[3-(3-bromopyrazolo[1,5-a]pyrimidin-7-yl)phenyl]-3,4-dichlorobenzamide;
N-[3-(3-bromopyrazolo[1,5-a]pyrimidin-7-yl)phenyl]-N′-[3-(trifluoromethyl)phenyl]urea;
N-[3-(3-bromopyrazolo[1,5-a]pyrimidin-7-yl)phenyl]-N′-[4-fluoro-3-(trifluoromethyl)phenyl]urea;
N-[3-(3-bromopyrazolo[1,5-a]pyrimidin-7-yl)phenyl]-N′-(3,4-dichlorophenyl)urea;
4-methyl-N-[3-(3-pyridin-3-ylpyrazolo[1,5-a]pyrimidin-7-yl)phenyl]-3-(trifluoromethyl)benzamide;
4-methoxy-N-[3-(3-pyridin-3-ylpyrazolo[1,5-a]pyrimidin-7-yl)phenyl]-3-(trifluoromethyl)benzamide;
4-fluoro-N-[3-(3-pyridin-3-ylpyrazolo[1,5-a]pyrimidin-7-yl)phenyl]-3-(trifluoromethyl)benzamide;
4-chloro-N-[3-(3-pyridin-3-ylpyrazolo[1,5-a]pyrimidin-7-yl)phenyl]-3-(trifluoromethyl)benzamide;
N-[3-(3-pyridin-3-ylpyrazolo[1,5-a]pyrimidin-7-yl)phenyl]-N′-[3-(trifluoromethyl)phenyl]urea;
N-[4-chloro-3-(trifluoromethyl)phenyl]-N′-[3-(3-pyridin-3-ylpyrazolo[1,5-a]pyrimidin-7-yl)phenyl]urea;
N-(3,4-dichlorophenyl)-N′-[3-(3-pyridin-3-ylpyrazolo[1,5-a]pyrimidin-7-yl)phenyl]urea;
ethyl 6-methyl-7-(3-{[3-(trifluoromethyl)benzoyl]amino}phenyl)pyrazolo[1,5-a]pyrimidine-3-carboxylate;
N-[3-(6-methyl-2-pyridin-4-ylpyrazolo[1,5-a]pyrimidin-7-yl)phenyl]-3-(trifluoromethyl)benzamide;
N-[3-(3-bromo-6-methylpyrazolo[1,5-a]pyrimidin-7-yl)phenyl]-3-(trifluoromethyl)benzamide;
N-[3-(6-methyl-3-pyridin-3-ylpyrazolo[1,5-a]pyrimidin-7-yl)phenyl]-3-(trifluoromethyl)benzamide;
N-{3-[3-(4-aminophenyl)pyrazolo[1,5-a]pyrimidin-7-yl]phenyl}-4-methyl-3-(trifluoromethyl)benzamide;
N-{3-[3-(3-hydroxyphenyl)pyrazolo[1,5-a]pyrimidin-7-yl]phenyl}-4-methyl-3-(trifluoromethyl)benzamide;
N-{3-[3-(3-cyanophenyl)pyrazolo[1,5-a]pyrimidin-7-yl]phenyl}-4-methyl-3-(trifluoromethyl)benzamide;
N-[3-(3-{3-[(dimethylamino)carbonyl]phenyl}pyrazolo[1,5-a]pyrimidin-7-yl)phenyl]-4-methyl-3-(trifluoromethyl)benzamide;
N-(3-{3-[4-(acetylamino)phenyl]pyrazolo[1,5-a]pyrimidin-7-yl}phenyl)-4-methyl-3-(trifluoromethyl)benzamide;
ethyl 7-(3-{[3-(trifluoromethyl)benzoyl]amino}phenyl)pyrazolo[1,5-a]pyrimidine-2-carboxylate;
N-[3-(2-morpholin-4-ylpyrazolo[1,5-a]pyrimidin-7-yl)phenyl]-3-(trifluoromethyl)benzamide;
N-{3-[2-(dimethylamino)pyrazolo[1,5-a]pyrimidin-7-yl]phenyl}-3-(trifluoromethyl)benzamide;
4-methyl-N-[3-(3-pyridin-4-ylpyrazolo[1,5-a]pyrimidin-7-yl)phenyl]-3-(trifluoromethyl)benzamide;
4-methoxy-N-[3-(3-pyridin-4-ylpyrazolo[1,5-a]pyrimidin-7-yl)phenyl]-3-(trifluoromethyl)benzamide;
4-fluoro-N-[3-(3-pyridin-4-ylpyrazolo[1,5-a]pyrimidin-7-yl)phenyl]-3-(trifluoromethyl)benzamide;
4-chloro-N-[3-(3-pyridin-4-ylpyrazolo[1,5-a]pyrimidin-7-yl)phenyl]-3-(trifluoromethyl)benzamide;
N-[3-(3-pyridin-4-ylpyrazolo[1,5-a]pyrimidin-7-yl)phenyl]-N′-[3-(trifluoromethyl)phenyl]urea;
N-[4-fluoro-3-(trifluoromethyl)phenyl]-N′-[3-(3-pyridin-4-ylpyrazolo[1,5-a]pyrimidin-7-yl)phenyl]urea;
4-methyl-N-[3-(2-morpholin-4-ylpyrazolo[1,5-a]pyrimidin-7-yl)phenyl]-3-(trifluoromethyl)benzamide;
4-methoxy-N-[3-(2-morpholin-4-ylpyrazolo[1,5-a]pyrimidin-7-yl)phenyl]-3-(trifluoromethyl)benzamide;
4-fluoro-N-[3-(2-morpholin-4-ylpyrazolo[1,5-a]pyrimidin-7-yl)phenyl]-3-(trifluoromethyl)benzamide;
4-chloro-N-[3-(2-morpholin-4-ylpyrazolo[1,5-a]pyrimidin-7-yl)phenyl]-3-(trifluoromethyl)benzamide;
3,4-dichloro-N-[3-(2-morpholin-4-ylpyrazolo[1,5-a]pyrimidin-7-yl)phenyl]benzamide;
N-[3-(2-morpholin-4-ylpyrazolo[1,5-a]pyrimidin-7-yl)phenyl]-N′-[3-(trifluoromethyl)phenyl]urea;
N-[4-chloro-3-(trifluoromethyl)phenyl]-N′-[3-(2-morpholin-4-ylpyrazolo[1,5-a]pyrimidin-7-yl)phenyl]urea;
N-(3,4-dichlorophenyl)-N′-[3-(2-morpholin-4-ylpyrazolo[1,5-a]pyrimidin-7-yl)phenyl]urea;
N-[4-fluoro-3-(trifluoromethyl)phenyl]-N′-[3-(2-morpholin-4-ylpyrazolo[1,5-a]pyrimidin-7-yl)phenyl]urea;
N-{3-[2-(4-methylpiperazin-1-yl)pyrazolo[1,5-a]pyrimidin-7-yl]phenyl}-3-(trifluoromethyl)benzamide;
ethyl 7-{3-[(pyridin-3-ylcarbonyl)amino]phenyl}pyrazolo[1,5-a]pyrimidine-3-carboxylate;
N-(3-{3-[3-(dimethylamino)prop-1-yn-1-yl]pyrazolo[1,5-a]pyrimidin-7-yl}phenyl)-3-(trifluoromethyl)benzamide;
tert-butyl 4-[7-(3-{[3-(trifluoromethyl)benzoyl]amino}phenyl)pyrazolo[1,5-a]pyrimidin-2-yl]piperazine-1-carboxylate;
N-{3-[2-(4-benzylpiperazin-1-yl)pyrazolo[1,5-a]pyrimidin-7-yl]phenyl}-3-(trifluoromethyl)benzamide;
N-[3-(2-piperazin-1-ylpyrazolo[1,5-a]pyrimidin-7-yl)phenyl]-3-(trifluoromethyl)benzamide;
N-(3-{2-[3-(dimethylamino)pyrrolidin-1-yl]pyrazolo[1,5-a]pyrimidin-7-yl}phenyl)-3-(trifluoromethyl)benzamide;
N-(3-{2-[(2R)-2-(methoxymethyl)pyrrolidin-1-yl]pyrazolo[1,5-a]pyrimidin-7-yl}phenyl)-3-(trifluoromethyl)benzamide;
N-(3-{2-[(2S)-2-(pyrrolidin-1-ylmethyl)pyrrolidin-1-yl]pyrazolo[1,5-a]pyrimidin-7-yl}phenyl)-3-(trifluoromethyl)benzamide;
tert-butyl {1-[7-(3-{[3-(trifluoromethyl)benzoyl]amino}phenyl)pyrazolo[1,5-a]pyrimidin-2-yl]pyrrolidin-3-yl}carbamate; phenyl}-3-(trifluoromethyl)benzamide;
ethyl 7-{3-[(pyrazin-2-ylcarbonyl)amino]phenyl}pyrazolo[1,5-a]pyrimidine-3-carboxylate;
ethyl 7-(3-{[(1-methyl-1H-pyrrol-2-yl)carbonyl]amino}phenyl)pyrazolo[1,5-a]pyrimidine-3-carboxylate;
ethyl 7-(3-{[(5-methylpyrazin-2-yl)carbonyl]amino}phenyl)pyrazolo[1,5-a]pyrimidine-3-carboxylate;
ethyl 7-(3-{[(4-chloropyridin-2-yl)carbonyl]amino}phenyl)pyrazolo[1,5-a]pyrimidine-3-carboxylate;
ethyl 7-{3-[(isoquinolin-1-ylcarbonyl)amino]phenyl}pyrazolo[1,5-a]pyrimidine-3-carboxylate;
ethyl 7-(3-{[(1-methyl-1H-indol-2-yl)carbonyl]amino}phenyl)pyrazolo[1,5-a]pyrimidine-3-carboxylate;
ethyl 7-(3-{[(5-methyl-2-phenyl-2H-1,2,3-triazol-4-yl)carbonyl]amino}phenyl)pyrazolo[1,5-a]pyrimidine-3-carboxylate;
ethyl 7-(3-{[(5-methyl-2-thienyl)carbonyl]amino}phenyl)pyrazolo[1,5-a]pyrimidine-3-carboxylate;
ethyl 7-(3-{[(5-chloro-2-thienyl)carbonyl]amino}phenyl)pyrazolo[1,5-a]pyrimidine-3-carboxylate;
ethyl 7-(3-{[(5-bromo-2-thienyl)carbonyl]amino}phenyl)pyrazolo[1,5-a]pyrimidine-3-carboxylate;
ethyl 7-{3-[({5-[1-methyl-3-(trifluoromethyl)-1H-pyrazol-5-yl]-2-thienyl}carbonyl)amino]phenyl}pyrazolo[1,5-a]pyrimidine-3-carboxylate;
ethyl 7-{3-[(3,3-dimethylbutanoyl)amino]phenyl}pyrazolo[1,5-a]pyrimidine-3-carboxylate;
ethyl 7-{3-[(3,5,5-trimethylhexanoyl)amino]phenyl}pyrazolo[1,5-a]pyrimidine-3-carboxylate;
ethyl 7-{3-[(3,5-di-tert-butylbenzoyl)amino]phenyl}pyrazolo[1,5-a]pyrimidine-3-carboxylate;
ethyl 7-{3-[(2-bromo-5-chlorobenzoyl)amino]phenyl}pyrazolo[1,5-a]pyrimidine-3-carboxylate;
N-[3-(3-{4-[(methoxyacetyl)amino]phenyl}pyrazolo[1,5-a]pyrimidin-7-yl)phenyl]-3-(trifluoromethyl)benzamide;
N-[3-(3-{4-[(N,N-dimethylglycyl)amino]phenyl}pyrazolo[1,5-a]pyrimidin-7-yl)phenyl]-3-(trifluoromethyl)benzamide;
N-[3-(3-{4-[(3-methoxypropanoyl)amino]phenyl}pyrazolo[1,5-a]pyrimidin-7-yl)phenyl]-3-(trifluoromethyl)benzamide;
N-[3-(3-{4-[(1H-imidazol-4-ylacetyl)amino]phenyl}pyrazolo[1,5-a]pyrimidin-7-yl)phenyl]-3-(trifluoromethyl)benzamide;
N-[3-(3-{4-[(1H-tetrazol-5-ylacetyl)amino]phenyl}pyrazolo[1,5-a]pyrimidin-7-yl)phenyl]-3-(trifluoromethyl)benzamide;
N-{3-[3-(4-{[4-(dimethylamino)butanoyl]amino}phenyl)pyrazolo[1,5-a]pyrimidin-7-yl]phenyl}-3-(trifluoromethyl)benzamide;
N-{3-[3-(4-{[(2-methoxyethoxy)acetyl]amino}phenyl)pyrazolo[1,5-a]pyrimidin-7-yl]phenyl}-3-(trifluoromethyl)benzamide;
1-methyl-N-[3-(2-pyridin-4-ylpyrazolo[1,5-a]pyrimidin-7-yl)phenyl]-1H-pyrrole-2-carboxamide;
N-[3-(2-pyridin-4-ylpyrazolo[1,5-a]pyrimidin-7-yl)phenyl]isoquinoline-1-carboxamide;
1-methyl-N-[3-(2-pyridin-4-ylpyrazolo[1,5-a]pyrimidin-7-yl)phenyl]-1H-indole-2-carboxamide;
5-bromo-N-[3-(2-pyridin-4-ylpyrazolo[1,5-a]pyrimidin-7-yl)phenyl]thiophene-2-carboxamide;
3,3-dimethyl-N-[3-(2-pyridin-4-ylpyrazolo[1,5-a]pyrimidin-7-yl)phenyl]butanamide;
2-bromo-5-chloro-N-[3-(2-pyridin-4-ylpyrazolo[1,5-a]pyrimidin-7-yl)phenyl]benzamide;
ethyl 7-{3-[(3-methylbenzoyl)amino]phenyl}pyrazolo[1,5-a]pyrimidine-3-carboxylate;
ethyl 7-(3-{[(3-tert-butyl-1-methyl-1H-pyrazol-5-yl)carbonyl]amino}phenyl)pyrazolo[1,5-a]pyrimidine-3-carboxylate;
ethyl 7-[3-({[(6-methoxy-1,3-benzothiazol-2-yl)amino]carbonyl}amino)phenyl]pyrazolo[1,5-a]pyrimidine-3-carboxylate;
ethyl 7-(3-{[(1,3-benzodioxol-5-ylamino)carbonyl]amino}phenyl)pyrazolo[1,5-a]pyrimidine-3-carboxylate;
ethyl 7-[3-({[(6-chloro-1,3-benzothiazol-2-yl)amino]carbonyl}amino)phenyl]pyrazolo[1,5-a]pyrimidine-3-carboxylate;
ethyl 7-[3-({[(3-methylisoxazol-5-yl)amino]carbonyl}amino)phenyl]pyrazolo[1,5-a]pyrimidine-3-carboxylate;
ethyl 7-[3-({[(5-methylisoxazol-3-yl)amino]carbonyl}amino)phenyl]pyrazolo[1,5-a]pyrimidine-3-carboxylate;
N-{3-[2-(3-oxopiperazin-1-yl)pyrazolo[1,5-a]pyrimidin-7-yl]phenyl}-3-(trifluoromethyl)benzamide;
N-[3-(2-hydroxypyrazolo[1,5-a]pyrimidin-7-yl)phenyl]-3-(trifluoromethyl)benzamide;
N-{3-[2-(4-oxopiperidin-1-yl)pyrazolo[1,5-a]pyrimidin-7-yl]phenyl}-3-(trifluoromethyl)benzamide;
N-[3-(3-{3-[(methoxyacetyl)amino]phenyl}pyrazolo[1,5-a]pyrimidin-7-yl)phenyl]-3-(trifluoromethyl)benzamide;
N-[3-(3-{3-[(N,N-dimethylglycyl)amino]phenyl}pyrazolo[1,5-a]pyrimidin-7-yl)phenyl]-3-(trifluoromethyl)benzamide;
N-[3-(3-{3-[(3-methoxypropanoyl)amino]phenyl}pyrazolo[1,5-a]pyrimidin-7-yl)phenyl]-3-(trifluoromethyl)benzamide;
N-[3-(3-{3-[(N-acetylglycyl)amino]phenyl}pyrazolo[1,5-a]pyrimidin-7-yl)phenyl]-3-(trifluoromethyl)benzamide;
N-[3-(3-{3-[(1H-tetrazol-5-ylacetyl)amino]phenyl}pyrazolo[1,5-a]pyrimidin-7-yl)phenyl]-3-(trifluoromethyl)benzamide;
N-{3-[2-(2-{[3-(dimethylamino)propyl]amino}pyridin-4-yl)pyrazolo[1,5-a]pyrimidin-7-yl]phenyl}-3-(trifluoromethyl)benzamide;
N-[3-(2-{2-[(3-morpholin-4-ylpropyl)amino]pyridin-4-yl}pyrazolo[1,5-a]pyrimidin-7-yl)phenyl]-3-(trifluoromethyl)benzamide;
N-[3-(2-{2-[(3-piperidin-1-ylpropyl)amino]pyridin-4-yl}pyrazolo[1,5-a]pyrimidin-7-yl)phenyl]-3-(trifluoromethyl)benzamide;
N-[3-(2-{2-[(2-morpholin-4-ylethyl)amino]pyridin-4-yl}pyrazolo[1,5-a]pyrimidin-7-yl)phenyl]-3-(trifluoromethyl)benzamide;
N-{3-[2-(2-{[3-(2-oxopyrrolidin-1-yl)propyl]amino}pyridin-4-yl)pyrazolo[1,5-a]pyrimidin-7-yl]phenyl}-3-(trifluoromethyl)benzamide;
N-{3-[2-(2-{[3-(1H-imidazol-1-yl)propyl]amino}pyridin-4-yl)pyrazolo[1,5-a]pyrimidin-7-yl]phenyl}-3-(trifluoromethyl)benzamide;
N-{3-[2-(2-{[2-(4-hydroxypiperidin-1-yl)ethyl]amino}pyridin-4-yl)pyrazolo[1,5-a]pyrimidin-7-yl]phenyl}-3-(trifluoromethyl)benzamide;
N-[3-(2-{2-[(2-piperidin-1-ylethyl)amino]pyridin-4-yl}pyrazolo[1,5-a]pyrimidin-7-yl)phenyl]-3-(trifluoromethyl)benzamide;
N-[3-(2-{2-[(2-pyrrolidin-1-ylethyl)amino]pyridin-4-yl}pyrazolo[1,5-a]pyrimidin-7-yl)phenyl]-3-(trifluoromethyl)benzamide;
N-{3-[2-(2-{[2-(dimethylamino)ethyl]amino}pyridin-4-yl)pyrazolo[1,5-a]pyrimidin-7-yl]phenyl}-3-(trifluoromethyl)benzamide;
N-[3-(2-{2-[(3-pyrrolidin-1-ylpropyl)amino]pyridin-4-yl}pyrazolo[1,5-a]pyrimidin-7-yl)phenyl]-3-(trifluoromethyl)benzamide;
N-{3-[2-(2-{[2-(2-oxoimidazolidin-1-yl)ethyl]amino}pyridin-4-yl)pyrazolo[1,5-a]pyrimidin-7-yl]phenyl}-3-(trifluoromethyl)benzamide;
N-[3-(2-{2-[(3-aminopropyl)(methyl)amino]pyridin-4-yl}pyrazolo[1,5-a]pyrimidin-7-yl)phenyl]-3-(trifluoromethyl)benzamide;
N-[3-(2-{2-[(2-aminoethyl)(methyl)amino]pyridin-4-yl}pyrazolo[1,5-a]pyrimidin-7-yl)phenyl]-3-(trifluoromethyl)benzamide;
N-(3-{3-[3-(aminocarbonyl)phenyl]pyrazolo[1,5-a]pyrimidin-7-yl}phenyl)-3-(trifluoromethyl)benzamide;
N-[3-(3-{2-[(dimethylamino)methyl]phenyl}pyrazolo[1,5-a]pyrimidin-7-yl)phenyl]-3-(trifluoromethyl)benzamide;
N-[3-(3-{3-[(dimethylamino)methyl]phenyl}pyrazolo[1,5-a]pyrimidin-7-yl)phenyl]-3-(trifluoromethyl)benzamide;
N-[3-(3-{4-[(dimethylamino)methyl]phenyl}pyrazolo[1,5-a]pyrimidin-7-yl)phenyl]-3-(trifluoromethyl)benzamide;
N-{3-[3-(1H-pyrazol-4-yl)pyrazolo[1,5-a]pyrimidin-7-yl]phenyl}-3-(trifluoromethyl)benzamide;
tert-butyl 4-[7-(3-{[3-(trifluoromethyl)benzoyl]amino}phenyl)pyrazolo[1,5-a]pyrimidin-3-yl]-1H-pyrazole-1-carboxylate;
N-{3-[3-(3-furyl)pyrazolo[1,5-a]pyrimidin-7-yl]phenyl}-3-(trifluoromethyl)benzamide;
N-{3-[3-(6-aminopyridin-3-yl)pyrazolo[1,5-a]pyrimidin-7-yl]phenyl}-3-(trifluoromethyl)benzamide;
N-(3-{3-[5-(4-methylpiperazin-1-yl)pent-1-yn-1-yl]pyrazolo[1,5-a]pyrimidin-7-yl}phenyl)-3-(trifluoromethyl)benzamide;
N-[3-(3-{2-[2-(dimethylamino)ethyl]phenyl}pyrazolo[1,5-a]pyrimidin-7-yl)phenyl]-3-(trifluoromethyl)benzamide;
N-[3-(3-{3-[2-(dimethylamino)ethyl]phenyl}pyrazolo[1,5-a]pyrimidin-7-yl)phenyl]-3-(trifluoromethyl)benzamide;
N-{3-[3-(5-morpholin-4-ylpent-1-yn-1-yl)pyrazolo[1,5-a]pyrimidin-7-yl]phenyl}-3-(trifluoromethyl)benzamide;
N-{3-[3-(6-{[2-(dimethylamino)ethyl]amino}pyridin-3-yl)pyrazolo[1,5-a]pyrimidin-7-yl]phenyl}-3-(trifluoromethyl)benzamide;
N-(3-{3-[6-(methylamino)pyridin-3-yl]pyrazolo[1,5-a]pyrimidin-7-yl}phenyl)-3-(trifluoromethyl)benzamide;
N-(3-{3-[4-(hydroxymethyl)phenyl]pyrazolo[1,5-a]pyrimidin-7-yl}phenyl)-3-(trifluoromethyl)benzamide;
N-(3-{3-[3-(hydroxymethyl)phenyl]pyrazolo[1,5-a]pyrimidin-7-yl}phenyl)-3-(trifluoromethyl)benzamide;
N-{3-[2-(4-bromophenyl)pyrazolo[1,5-a]pyrimidin-7-yl]phenyl}-3-(trifluoromethyl)benzamide;
N-[3-(3-{4-[(dimethylamino)sulfonyl]phenyl}pyrazolo[1,5-a]pyrimidin-7-yl)phenyl]-3-(trifluoromethyl)benzamide;
N-[3-(3-{4-[2-(dimethylamino)ethyl]phenyl}pyrazolo[1,5-a]pyrimidin-7-yl)phenyl]-3-(trifluoromethyl)benzamide;
3-(trifluoromethyl)-N-(3-{3-[2-(trifluoromethyl)phenyl]pyrazolo[1,5-a]pyrimidin-7-yl}phenyl)benzamide;
3-(trifluoromethyl)-N-(3-{3-[3-(trifluoromethyl)phenyl]pyrazolo[1,5-a]pyrimidin-7-yl}phenyl)benzamide;
3-(trifluoromethyl)-N-(3-{3-[4-(trifluoromethyl)phenyl]pyrazolo[1,5-a]pyrimidin-7-yl}phenyl)benzamide;
N-{3-[3-(2-cyanophenyl)pyrazolo[1,5-a]pyrimidin-7-yl]phenyl}-3-(trifluoromethyl)benzamide;
N-{3-[3-(3-cyanophenyl)pyrazolo[1,5-a]pyrimidin-7-yl]phenyl}-3-(trifluoromethyl)benzamide;
N-{3-[3-(4-cyanophenyl)pyrazolo[1,5-a]pyrimidin-7-yl]phenyl}-3-(trifluoromethyl)benzamide;
methyl 3-[7-(3-{[3-(trifluoromethyl)benzoyl]amino}phenyl)pyrazolo[1,5-a]pyrimidin-3-yl]benzoate;
methyl 4-[7-(3-{[3-(trifluoromethyl)benzoyl]amino}phenyl)pyrazolo[1,5-a]pyrimidin-3-yl]benzoate;
N-{3-[3-(2-acetylphenyl)pyrazolo[1,5-a]pyrimidin-7-yl]phenyl}-3-(trifluoromethyl)benzamide;
N-{3-[3-(3-acetylphenyl)pyrazolo[1,5-a]pyrimidin-7-yl]phenyl}-3-(trifluoromethyl)benzamide;
N-{3-[3-(4-acetylphenyl)pyrazolo[1,5-a]pyrimidin-7-yl]phenyl}-3-(trifluoromethyl)benzamide;
N-{3-[3-(2-chloropyridin-4-yl)pyrazolo[1,5-a]pyrimidin-7-yl]phenyl}-3-(trifluoromethyl)benzamide;
N-[3-(5-methyl-2-pyridin-4-ylpyrazolo[1,5-a]pyrimidin-7-yl)phenyl]-3-(trifluoromethyl)benzamide;
N-(3-{3-[2-(1-hydroxyethyl)phenyl]pyrazolo[1,5-a]pyrimidin-7-yl}phenyl)-3-(trifluoromethyl)benzamide;
N-(3-{3-[3-(1-hydroxyethyl)phenyl]pyrazolo[1,5-a]pyrimidin-7-yl}phenyl)-3-(trifluoromethyl)benzamide;
N-{3-[3-(2-methylphenyl)pyrazolo[1,5-a]pyrimidin-7-yl]phenyl}-3-(trifluoromethyl)benzamide;
N-{3-[3-(1-methyl-1,2,3,6-tetrahydropyridin-4-yl)pyrazolo[1,5-a]pyrimidin-7-yl]phenyl}-3-(trifluoromethyl)benzamide;
N-(3-{3-[1-(2-pyrrolidin-1-ylethyl)-1H-pyrazol-4-yl]pyrazolo[1,5-a]pyrimidin-7-yl}phenyl)-3-(trifluoromethyl)benzamide;
N-{3-[3-(1-methyl-1H-pyrazol-4-yl)pyrazolo[1,5-a]pyrimidin-7-yl]phenyl}-3-(trifluoromethyl)benzamide;
N-{3-[3-(1-methylpiperidin-4-yl)pyrazolo[1,5-a]pyrimidin-7-yl]phenyl}-3-(trifluoromethyl)benzamide;
N-{3-[3-(3,5-diformylphenyl)pyrazolo[1,5-a]pyrimidin-7-yl]phenyl}-3-(trifluoromethyl)benzamide;
N-{3-[3-(6-fluoropyridin-3-yl)pyrazolo[1,5-a]pyrimidin-7-yl]phenyl}-3-(trifluoromethyl)benzamide;
N-{3-[3-(6-methoxypyridin-3-yl)pyrazolo[1,5-a]pyrimidin-7-yl]phenyl}-3-(trifluoromethyl)benzamide;
N-{3-[3-(5-formyl-2-furyl)pyrazolo[1,5-a]pyrimidin-7-yl]phenyl}-3-(trifluoromethyl)benzamide;
3-[7-(3-{[3-(trifluoromethyl)benzoyl]amino}phenyl)pyrazolo[1,5-a]pyrimidin-3-yl]benzoic acid;
N-(3-{3-[4-(pyrrolidin-1-ylmethyl)phenyl]pyrazolo[1,5-a]pyrimidin-7-yl}phenyl)-3-(trifluoromethyl)benzamide;
N-(3-{3-[5-(pyrrolidin-1-ylmethyl)-2-furyl]pyrazolo[1,5-a]pyrimidin-7-yl}phenyl)-3-(trifluoromethyl)benzamide;
N-[4-fluoro-3-(2-pyridin-4-ylpyrazolo[1,5-a]pyrimidin-7-yl)phenyl]-3-(trifluoromethyl)benzamide;
N-(4-fluoro-3-pyrazolo[1,5-a]pyrimidin-7-ylphenyl)-3-(trifluoromethyl)benzamide; and
N-(3-{3-[5-(pyrrolidin-1-ylmethyl)-3-furyl]pyrazolo[1,5-a]pyrimidin-7-yl}phenyl)-3-(trifluoromethyl)benzamide.

The following definitions are used in connection with the pyrazolo[1,5-a]pyrimidine derivatives of the invention.

Unless otherwise defined, the term “aryl”, as used herein, refers to an aromatic carbocyclic moiety, e.g. having from 6-20 carbon atoms, which may be a single ring or multiple rings fused together or linked covalently, wherein at least one of the rings is aromatic. Any suitable ring position of the aryl moiety may be covalently linked to the defined chemical structure. Examples of aryl include phenyl and napthyl. The aryl group may be optionally substituted. In addition to other optional substituents, the aryl group may be substituted by an oxo substituent meaning one of the ring carbon atoms is part of a carbonyl group.

Carrier shall encompass pharmaceutically acceptable carriers, excipients, and diluents.

Unless otherwise defined, the term “heteroaryl” as used herein means an aromatic heterocyclic ring system, e.g. having from 5-20 ring atoms, which may be a single ring or multiple rings fused together or linked covalently, wherein at least one of the rings is aromatic. The rings may contain one or more hetero atoms, e.g. 1 to 3 heteroatoms, selected from nitrogen, oxygen, or sulfur, wherein the nitrogen or sulfur atom(s) are optionally oxidized, or the nitrogen atom(s) are optionally quaternized. Any suitable ring position of the heteroaryl moiety may be covalently linked to the defined chemical structure. Examples of heteroaryl include 2-pyridyl or indol-1-yl. The heteroaryl group may be optionally substituted. In addition to other optional substituents, the heteroaryl group may be substituted by an oxo substituent meaning one of the ring carbon atoms is part of a carbonyl group.

The term “heterocyclic”, “heterocycle” or “heterocyclyl” as used herein can be used interchangeably to refer to a stable, saturated or partially unsaturated monocyclic or multicyclic heterocyclic ring system e.g. having from 5 to 7 ring members. The heterocyclic ring has in its backbone carbon atoms and one or more heteroatoms, e.g. 1 to 3 heteroatoms, selected from nitrogen, oxygen, and sulfur atoms, wherein the nitrogen or sulfur atom(s) are optionally oxidized, or the nitrogen atom(s) are optionally quaternized. The heterocyclic, heterocycle or heterocyclyl group may be optionally substituted. In addition to other optional substituents, the heterocyclic, heterocycle or heterocyclyl group may be substituted by an oxo substituent meaning one of the ring carbon atoms is part of a carbonyl group. The heterocyclic, heterocycle or heterocyclyl group may contain one of more fused rings.

Unless otherwise defined the optional substituents (as used in the term “optionally substituted”) or the substitutents (as used in the term “substituted”) on the aryl, heteroaryl or heterocycle are selected from the following: -J, —NO₂, —CN, —N₃, —CHO, —CF₃, —OCF₃, —R¹¹, —OR¹¹, S(O)ₘR¹¹, —NR¹⁵R¹¹, —NR¹²S(O)ₘR¹⁵, —OR⁹OR¹¹, —OR⁹NR¹⁵R¹¹, —N(R¹²)R⁹OR¹⁵, —N(R¹²)R⁹NR¹⁵R¹¹, —NR¹²C(O)R¹⁵, —C(O)R¹¹, —C(O)OR¹¹, —C(O)NR¹²R¹¹, —OC(O)R¹¹, —OC(O)OR¹¹, —OC(O)NR¹⁵R¹¹, NR¹²C(O)R¹⁵, —NR¹²C(O)OR¹⁵, —NR¹²C(O)NR¹⁵R¹¹, —R⁸OR¹¹, —R⁸NR¹⁵R¹¹, —R⁸S(O)ₘR¹¹, —R⁸C(O)R¹¹, —R⁸C(O)OR¹¹, —R⁸C(O)NR¹⁵R¹¹, —R⁸OC(O)R¹¹, —R⁸OC(O)OR¹¹, —R⁸OC(O)NR¹⁵R¹¹, —R⁸NR¹²C(O)R¹⁵, —R⁸NR¹²C(O)OR¹⁵, —R⁸NR¹²C(O)NR¹⁵R¹¹, R²⁰, —OR⁹R²⁰, —N(R¹²)R⁹R²⁰, —C(O)R²⁰, —OC(O)R²⁰, —NR¹²C(O)R²⁰, —R⁸R²⁰, —R⁸C(O)R²⁰, —R⁸C(O)R²⁰, —R⁸NR¹²C(O)R²⁰ and YR¹⁰; wherein R⁷, R¹¹, R¹², R¹⁴, R¹⁵ and R¹⁷ are independently selected from H, alkyl of 1-6 carbon atoms, branched alkyl of 3-8 carbon atoms, cis-alkenyl of 2-6 carbon atoms, a trans-alkenyl of 2-6 carbon atoms, and an alkynyl of 2-6 carbon atoms; said alkyl, branched alkyl, cis-alkenyl, trans-alkenyl, and alkynyl groups being optionally substituted with 1-3 J atoms; R⁸ is a divalent group selected from alkyl of 1-6 carbon atoms, alkenyl of 2-6 carbon atoms, and alkynyl of 2-6 carbon atoms; R⁹ is a divalent alkyl group of 2-6 carbon atoms; R¹⁰ is selected from the group consisting of a cycloalkyl ring of 3-10 carbons, a bicycloalkyl ring of 3-10 carbons, an aryl, a heterocyclyl ring, a heteroaryl ring, and a heteroaryl fused to 1-3 aryl or heteroaryl rings; any of said heterocyclyl ring and heteroaryl rings containing 1-3 heteroatoms selected from N, O or S; wherein any of the aryl, cycloalkyl, bicycloalkyl, heterocyclic or heteroaryl rings may be optionally substituted with one to four substituents selected from the group consisting of —H, -aryl, —CH₂-aryl, —NH-aryl, —O-aryl, —S(O)ₘ-aryl, -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷—, —OC(O)OR¹⁷, —OC(O)NR⁷R¹⁴, —NR¹¹C(O)R¹⁷, —NR¹¹C(O)OR¹⁷, —NR¹¹C(O)NR⁷R¹⁴, —R⁸OR¹⁷, R⁸NR⁷R¹⁴, —R⁸S(O)ₘR¹⁷, —R⁸C(O)R¹⁷, —R⁸C(O)OR¹⁷, —R⁸C(O)NR⁷R¹⁴, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷, and —R⁸NR¹¹C(O)NR⁷R¹⁴; R²⁰ is a heterocyclic ring containing 3-8 members, at least one member being N which is the point of attachment for the moiety, and optionally said 3 to 8 membered ring containing additional heteroatoms N, O, or S(O)ₘ and said 3-8 membered ring being optionally substituted with 1-4 substituents selected from alkyl of 1-6 carbon atoms, carbonyl, hydroxy, alkoxy of 1 to 6 carbon atoms, NH₂, NHR⁶, or N(R⁶)₂; J is fluoro, chloro, bromo, or iodo; m is an integer of 0-2; R⁶ is H, alkyl of 1-6 carbon atoms or branched alkyl of 3-8 carbon atoms; and Y is selected from the group consisting of a bond, a divalent alkyl group of 1-6 carbon atoms, NH, O, —NR¹⁷, —C≡C— cis- —CH═CH—, and trans- —CH═CH—. In one embodiment the aryl, heteroaryl, or heterocycle group will have 0-3 substituents. In another embodiment the aryl, heteroaryl or heterocycle group will have 0-4 substituents. In one embodiment the substituted aryl, substituted heteroaryl or substituted heterocycle has one or more independently selected substituents other than H. In another embodiment the substituted aryl, substituted heteroaryl or substituted heterocycle has 1-4 independently selected substituents other than H.

Unless otherwise defined the optional substituents (as used in the term “optionally substituted”) or the substitutents (as used in the term “substituted”) on alkyl may be selected from the following: -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷—, —OC(O)OR¹⁷, —OC(O)NR⁷R¹⁴, —NR¹¹C(O)R¹⁷, —NR¹¹C(O)OR¹⁷, and —NR¹¹C(O)NR⁷R¹⁴; wherein J is fluoro, chloro, bromo, or iodo; R⁷, R¹¹, R¹⁴, and R¹⁷ are independently selected from H, alkyl of 1-6 carbon atoms, branched alkyl of 3-8 carbon atoms, cis-alkenyl of 2-6 carbon atoms, a trans-alkenyl of 2-6 carbon atoms, and an alkynyl of 2-6 carbon atoms; said alkyl, branched alkyl, cis-alkenyl, trans-alkenyl, and alkynyl groups being optionally substituted with 1-3 J atoms; R⁷ and R¹⁴ together with the N to which they are attached may join to form a 3 to 8 membered ring; R⁹ is a divalent alkyl group of 2-6 carbon atoms; and m is an integer of 0-2. In one embodiment the substituted alkyl has one or more independently selected substituents other than H. In another embodiment the substituted alkyl has 1-3 independently selected substituents other than H.

Unless otherwise defined the optional substituents (as used in the term “optionally substituted”) or the substitutents (as used in the term “substituted”) on alkenyl or alkynyl may be selected from the following: CF₃, —R^{9a}OR¹⁷, —R^{9a}NR⁷R¹⁴, —R^{9a}S(O)ₘR¹⁷, —R^{9a}C(O)R¹⁷, —R^{9a}C(O)OR¹⁷, —R^{9a}C(O)NR⁷R¹⁴, R^{9a}C(O)R¹⁷, —R^{9a}C(O)OR¹⁷, —R^{9a}C(O)NR⁷R¹⁴, —R^{9a}OC(O)R¹⁷, —R^{9a}OC(O)OR¹⁷, —R^{9a}OC(O)NR⁷R¹⁴, —R^{9a}NR¹¹C(O)R¹⁷, —R^{9a}NR¹¹C(O)OR¹⁷, and —R^{9a}NR¹¹C(O)NR⁷R¹⁴; wherein R^{9g} is a divalent alkyl of 1-6 carbon atoms; m is an integer of 0-2; R⁷, R¹⁴, and R¹⁷ are independently selected from H, alkyl of 1-6 carbon atoms, branched alkyl of 3-8 carbon atoms, cis-alkenyl of 2-6 carbon atoms, a trans-alkenyl of 2-6 carbon atoms, and an alkynyl of 2-6 carbon atoms; said alkyl, branched alkyl, cis-alkenyl, trans-alkenyl, and alkynyl groups being optionally substituted with 1-3 J atoms; R⁷ and R¹⁴ together with the N to which they are attached may join to form a 3 to 8 membered ring; and J is fluoro, chloro, bromo, or iodo. In one embodiment the optionally substituted alkenyl or optionally substituted alkynyl have 0-3 substituents. In another embodiment the optionally substituted alkenyl or optionally substituted alkynyl have 0-4 substituents. In one embodiment the substituted alkynyl has one or more independently selected substituents other than H. In another embodiment the substituted alkynyl has 1-3 independently selected substituents other than H.

The compounds of this invention may be prepared from: (a) commercially available starting materials (b) known starting materials which may be prepared as described in literature procedures or (c) new intermediates described in the schemes and experimental procedures herein.

Reactions are performed in a solvent appropriate to the reagents and materials employed and suitable for the transformation being effected. It is understood by those skilled in the art of organic synthesis that the various functionalities present on the molecule must be consistent with the chemical transformation proposed. This may necessitate judgement as to the order of synthetic steps.

Compounds of the present invention may be prepared as illustrated in the examples and in following reaction schemes 1 to 4.

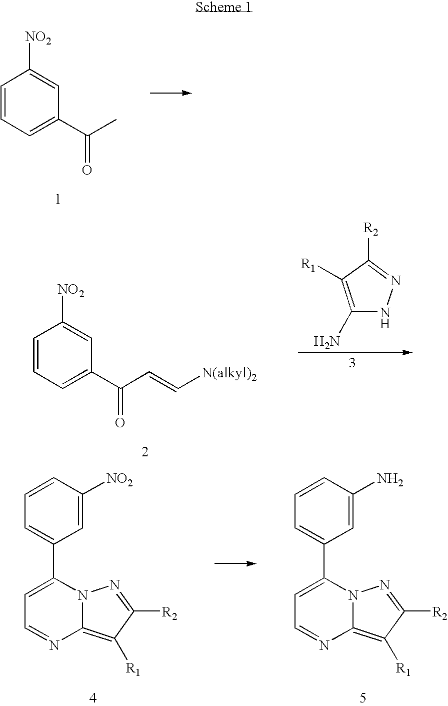

Referring to Scheme 1, reaction of 3-nitroacetophenone 1 with acetals of N,Ndialkylformamides or acetals of N,N-dialkylacetamide optionally in an inert solvent affords an 3-dialkylamino-1-(aryl or heteroaryl)-2-propen-1-one 2. The reaction of substituted 3-aminopyrazole 3 where R₁ and R₂ are herein before defined, and an appropriately substituted 3-dialkylamino-1-(aryl or heteroaryl)-2-propen-1-one 2 in weak acid such as glacial acetic acid or in an inert solvent such as toluene, acetonitrile or dimethoxyethane, at reflux temperature for several hours, produces nitro compound 4. 3-Amino-4-pyrazoles are disclosed in U.S. Pat. Nos. 4,236,005; 4,281,000; 4,521,422; 4,626,538; 4,654,347; and 4,900,836. An appropriately substituted 3-dialkylamino-1-(aryl or heteroaryl)-2-propen-1-one and in particular 3-dialkylamino-1-phenyl-2-propen-1-ones are disclosed in U.S. Pat. Nos. 4,178,449 and 4,236,005. Substituted 3-dimethylamino-1-(3-heteroaryl)-2-propen-1-ones are disclosed in U.S. Pat. Nos. 4,281,000 and 4,521,422. Pyrazolo[1,5-a]pyrimidines prepared by condensation of 3-aminopyrazoles and substituted 3-aminopyrazoles with 1,3-dicarbonyl compounds are described in J. Med. Chem., 18, 645 (1974); J. Med. Chem. 18, 460 (1975); J. Med. Chem., 20, 386 (1977); Synthesis, 673 (1982). The reduction of nitro compound 4 with reducing agents such as Fe, SnC1₂-xH₂O, catalytic hydrogenation and the like, gives aniline 5.

As described in Scheme 2, aniline 5 is reacted with acylating agents such as an acyl chloride 6 or an carboxylic acid anhydride prepared from carboxylic acid 7, in the presence of a organic base such as pyridine, triethylamine, N-methylmorpholine, 4-dimethylaminopyridine and the like, to give amide compounds, 8. Alternatively, amide 8 can be prepared from the reaction of aniline 5 with a carbamate intermediate generated in situ by treating carboxylic acid 7 with an alkyl chloroformate in the presence of a organic base such as pyridine, triethylamine, N—methylmorpholine, 4-dimethylaminopyridine and the like. Amide 8 can also be prepared by reacting carboxylic acid 7 in the presence of coupling reagents like 1-(3-Dimethylaminopropyl)-3-ethylcarbodiimide hydrochloride (EDCl), 1,3-dicyclohexylcarbodiimide (DCC), 1-hydroxybenzotriazole hydrate (HOBT), O-(benzotriazol-1-yl)-N,N, N′,N′-tetramethyluronium hexafluorophosphate (HBTU) and the like, in the presence of base.

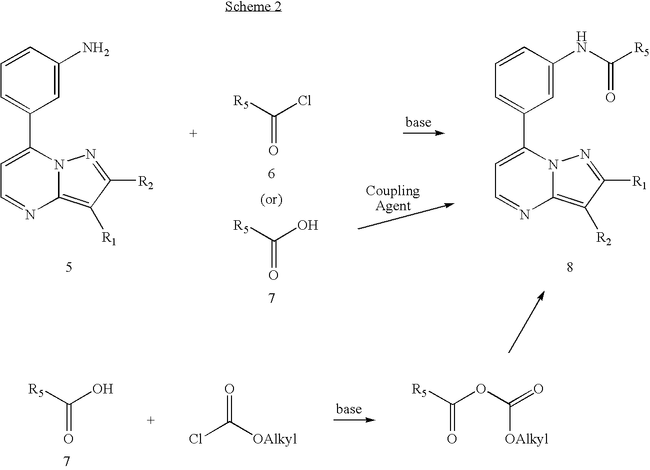

As described in scheme 3, the reaction of aniline 5 with alkyl isocyanate or aryl isocyanate 9 in the presence of an organic base, such as pyridine, triethylamine, N-methylmorpholine, 4-dimethylaminopyridine and the like, gives urea 10. Alternatively, urea 10 can be prepared by treating aniline 5 with phenyl chloroformate or substituted phenyl chloroformate in the presence of base as defined above, to form carbamate 11 followed by reaction with an amine, R⁵NH₂, to give urea 10.

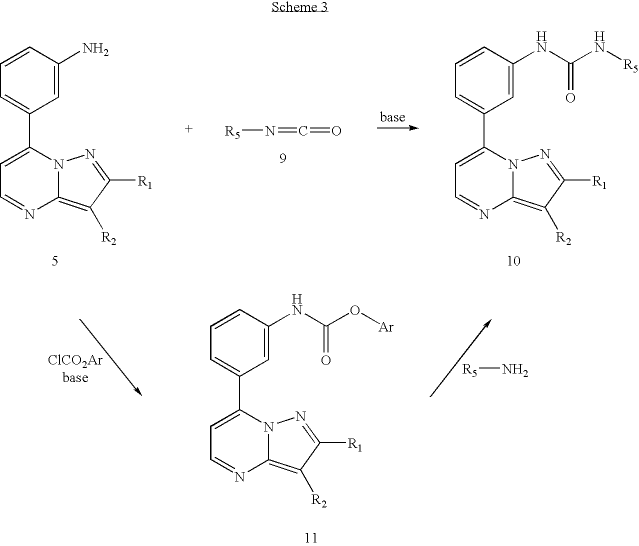

Additionally, certain compounds of Formula (I) can be synthesized by the methodology shown in Scheme 4 below

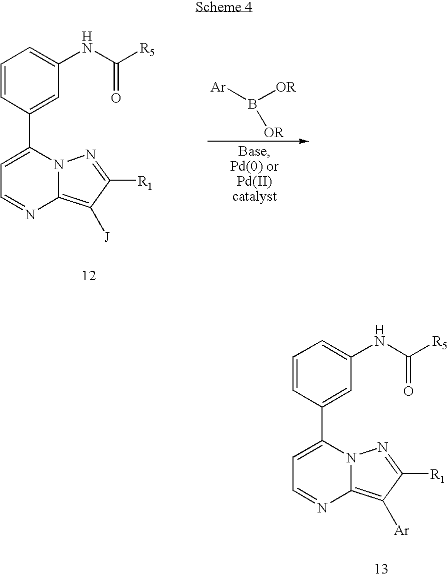

As described in Scheme 4, amide 12, where J is bromo or iodo is reacted with boronic acids or esters in the presence of palladium catalyst, such as palladium tetrakistriphenylphosphine, and a base such as sodium carbonate and the like, to provide pyrazolo[1,5-a]pyrimidine 13.

The compounds of Formula (I) may be obtained as inorganic or organic salts using methods known to those skilled in the art (Richard C. Larock, Comprehensive Organic Transformations, VCH publishers, 411-415, 1989). It is well known to one skilled in the art that an appropriate salt form is chosen based on physical and chemical stability, flowability, hydroscopicity and solubility.

Pharmaceutically acceptable salts of the compounds of Formula (I) with an acidic moiety may be formed from organic and inorganic bases. For example with alkali metals or alkaline earth metals such as sodium, potassium, lithium, calcium, or magnesium or organic bases and N-tetraalkylammonium salts such as N-tetrabutylammonium salts. Similarly, when a compound of this invention contains a basic moiety, salts may be formed from organic and inorganic acids. For example salts may be formed from acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids. The compounds can also be used in the form of esters, carbamates and other conventional prodrug forms, which when administered in such form, convert to the active moiety in vivo.

The present invention accordingly provides a pharmaceutical composition which comprises a compound of this invention in combination or association with a pharmaceutically acceptable carrier. In particular, the present invention provides a pharmaceutical composition which comprises an effective amount of a compound of this invention and a pharmaceutically acceptable carrier.

Standard Pharmacological Test Procedures

Evaluation of representative compounds of this invention in standard pharmacological test procedures indicated that the compounds of this invention possess significant anticancer activity and are in particular inhibitors of B-raf kinase. Based on the activity shown in the standard pharmacological test procedures, the compounds of this invention are therefore useful as antineoplastic agents. In particular, these compounds are useful in treating, inhibiting the growth of, or eradicating neoplasms such as those of the breast, kidney, bladder, mouth, larynx, esophagus, stomach, colon, ovary, lung, pancreas, liver, prostate and skin.

Biological Test Procedure(s)

TITLE: B-Raf Kinase to MEK1 ELISA

PURPOSE: To discover B-Raf Kinase inhibitors that can inhibit growth of tumor cells which contain oncogenic forms of Receptor Tyrosine Kinases or K-Ras, or B-Raf kinase.

Materials and Methods:

Background: Activating B-Raf mutations have been found in 66% of malignant melanomas and in a smaller fraction of other cancers including those of the colorectum (1,2). These findings have made B-Raf an important target in Oncology drug discovery.

Reagents: Flag/GST-tagged recombinant human B-Raf produced in Sf9 insect cells, human non-active Mek-1-GST (recombinant protein produced in E. coli); and a phospho-MEK1 specific poly-clonal Ab from Cell Signaling Technology (cat. #9121).

B-Raf1 Kinase Assay Procedure:

B-Raf-1 is used to phosphorylate GST-MEK1. MEK1 phosphorylation is measured by a phospho-specific antibody (from Cell Signaling Technology, cat. #9121) that detects phosphorylation of two serine residues at positions 217 and 221 on MEK1.

Kinase Assay Protocol

B-Raf Assay Stock Solutions:

1. Assay Dilution Buffer (ADB): 20 mM MOPS, pH 7.2, 25 mM β-glycerol phosphate, 5 mM EGTA, 1 mM sodium orthovanadate, 1 mM dithiothreitol.

2. Magnesium/ATP Cocktail: 200 μM cold ATP and 40 mM magnesium chloride in ADB.

3. Active Kinase: Active B-Raf: used at ˜20 ng per assay point.

4. Non-active GST-MEK1: Use at 100 nM (50 nM final).

5. TBST-Tris (50 mM, pH 7.5), NaCl (150 mM), Tween-20 (0.05%)

6. Anti-GST Ab (Pharmacia)

7. Anti-rabbit Ab/Europium conjugate (Wallac)

Assay Procedure:

1. Add 25 μl of ADB containing B-Raf and Mek per assay (i.e. per well of a 96 well plate)

2. Add 25 μl of 0.2 mM ATP and 40 mM magnesium chloride in ADB.

3. Incubate for 60 minutes at RT in a shaking incubator.

4. Transfer this mixture to an anti-GST Ab coated 96 well plate (Nunc Immunosorb plates coated o/n with a-GST.

5. Incubate for 60 minutes at 30° C. in a shaking incubator

6. Wash 3× with TBST, add Anti-Phospho MEK1 (1:1000)

7. Incubate for 60 minutes at 30° C. in a shaking incubator

8. Wash 3× with TBST, add Anti-rabbit Ab/Europium conjugate (Wallac) (1:500)

9. Incubate for 60 minutes at 30° C. in a shaking incubator

10. Add 100 ul of Wallac Delfia Enhancement Solution and shake for 10 minutes.

11. Read plates in Wallac Victor model Plate Reader.

12. Collect data analyze in Excel for single point and IC50 determinations.

Analysis of Results:

Single point assay—% inhibition at 10 mg/ml (% Inhibition=1−cpd.treated sample/untreated control)

IC50 determinations—done on cpds from single pt assays with >80% inhibition. Typically Raf-1 assay is run at cpd concentrations from 10 μM to 30 nM in half log dilutions. (% inhibition is determined for each cpd concentration)

### REFERENCES

1) Davies H, et al. (2002) Nature 417:906
2) Rajagopalan H, et al. (2002) Nature 418:934

3) Mallon R, et al. (2001) Anal. Biochem. 294:48

| EXAMPLES | % Inhib./10 uM | Median IC50 (μM) |
|---|---|---|
| Example 1 | 66% | 0.72 |
| Example 2 | 84% | 17 |
| Example 3 | 4% | >20.000 |
| Example 4 | 4% | >10.000 |
| Example 5 | 24% | >20.000 |
| Example 6 | 38% | >20.000 |
| Example 7 | 67% | 1.43 |
| Example 8 | 28% | >10.000 |
| Example 9 | 20% | >20.000 |
| Example 10 | 27% | >10.000 |
| Example 11 | 93% | 0.3 |
| Example 12 | 7% | >20.000 |
| Example 13 | 27% | >20.000 |
| Example 14 | 0% | >10.000 |
| Example 15 | 0% | >10.000 |
| Example 16 | 31% | 3.78 |
| Example 17 | 30% | >10.000 |
| Example 18 | 0% | >10.000 |
| Example 19 | 0% | >10.000 |
| Example 20 | 30% | >10.000 |
| Example 21 | 58% | 2.3 |
| Example 22 | 34% | >10.000 |
| Example 23 | 20% | >10.000 |
| Example 24 | 11% | >10.000 |
| Example 25 | 76% | 0.6 |
| Example 26 | 76% | 0.5 |
| Example 27 | 12% | >10.000 |
| Example 28 | 19% | >10.000 |
| Example 29 | 0% | >10.000 |
| Example 30 | 75% | 1.3 |
| Example 31 | 48% | >10.000 |
| Example 32 | 29% | >10.000 |
| Example 33 | 80% | 0.68 |
| Example 34 | 37% | >10.000 |
| Example 35 | 71% | 3.1 |
| Example 36 | 33% | >10.000 |
| Example 37 | 76% | 6.82 |
| Example 38 | 20% | >10.000 |
| Example 39 | 14% | >10.000 |
| Example 40 | 81% | 0.34 |
| Example 41 | 37% | >10.000 |
| Example 42 | 70% | 3.53 |
| Example 43 | 9% | >10.000 |
| Example 44 | 90% | 0.27 |
| Example 45 | 56% | 3 |
| Example 46 | 54% | 3 |
| Example 47 | 35% | >10.000 |
| Example 48 | 43% | 10 |
| Example 49 | 62% | 5.94 |
| Example 50 | 42% | 8 |
| Example 51 | 50% | 3 |
| Example 52 | 60% | 8 |
| Example 53 | 55% | 5 |
| Example 54 | 32% | >10.000 |
| Example 55 | 70% | 2.1 |
| Example 56 | 60% | 1 |
| Example 57 | 51% | 1.64 |
| Example 58 | 4% | >10.000 |
| Example 59 | 37% | >10.000 |
| Example 60 | 1% | >10.000 |
| Example 61 | 16% | >10.000 |
| Example 62 | 54% | 3 |
| Example 63 | 14% | >10.000 |
| Example 64 | 4% | >10.000 |
| Example 65 | 30% | >10.000 |
| Example 66 | 18% | >10.000 |
| Example 67 | 6% | >10.000 |
| Example 68 | 45% | 8.4 |
| Example 69 | 13% | >10.000 |
| Example 70 | 0% | >10.000 |
| Example 71 | 0% | >10.000 |
| Example 72 | 4% | >10.000 |
| Example 73 | 5% | >10.000 |
| Example 74 | 10% | >10.000 |
| Example 75 | 58% | 6.6 |
| Example 76 | 0% | >10.000 |
| Example 77 | 72% | 0.99 |
| Example 78 | 85% | 0.57 |
| Example 79 | 88% | 0.37 |
| Example 80 | 47% | 10 |
| Example 81 | 50% | 6.15 |
| Example 82 | 77% | 0.57 |
| Example 83 | 71% | 0.6 |
| Example 84 | 22% | >10.000 |
| Example 85 | 81% | 0.41 |
| Example 86 | 18% | >10.000 |
| Example 87 | 9% | >10.000 |
| Example 88 | 14% | >10.000 |
| Example 89 | 65% | 2.17 |
| Example 90 | 4% | >10.000 |
| Example 91 | 94% | 0.112 |
| Example 92 | 97% | >10.000 |
| Example 93 | 0% | >10.000 |
| Example 94 | 14% | >10.000 |
| Example 95 | 39% | >10.000 |
| Example 96 | 0% | >10.000 |
| Example 97 | 49% | 8.6 |
| Example 98 | 15% | >10.000 |
| Example 99 | 13% | >10.000 |
| Example 100 | 2% | >10.000 |
| Example 101 | 0% | >10.000 |
| Example 102 | 0% | >10.000 |
| Example 103 | 59% | 2.49 |
| Example 104 | 1% | 10.1 |
| Example 105 | 37% | >10.000 |
| Example 106 | 2% | >10.000 |
| Example 107 | 0% | >10.000 |
| Example 108 | 20% | >10.000 |
| Example 109 | 96% | 0.031 |
| Example 110 | 24% | >10.000 |
| Example 111 | 24% | >3.000 |
| Example 112 | 26% | >10.000 |
| Example 113 | 45% | >10.000 |
| Example 114 | 39% | >10.000 |
| Example 115 | 24% | >10.000 |
| Example 116 | 31% | >10.000 |
| Example 117 | 24% | >10.000 |
| Example 118 | 22% | >10.000 |
| Example 119 | 29% | >10.000 |
| Example 120 | 92% | 0.12 |
| Example 121 | 92% | 0.19 |
| Example 122 | 83% | 0.14 |
| Example 123 | 93% | 0.12 |
| Example 124 | 68% | 0.36 |
| Example 125 | 67% | 0.9 |
| Example 126 | 17% | >10.000 |
| Example 127 | 54% | 7.07 |
| Example 128 | 68% | 0.116 |
| Example 129 | 86% | 0.05 |
| Example 130 | 91% | 0.07 |
| Example 131 | 98% | 0.03 |
| Example 132 | 95% | 0.02 |
| Example 133 | 98% | 0.03 |
| Example 134 | 96% | 0.04 |
| Example 135 | 32% | >10.000 |
| Example 136 | 35% | >10.000 |
| Example 137 | 29% | >10.000 |
| Example 138 | 46% | >10.000 |
| Example 139 | 35% | >10.000 |
| Example 140 | 39% | >10.000 |
| Example 141 | 32% | >10.000 |
| Example 142 | 48% | >10.000 |
| Example 143 | 92% | 0.14 |
| Example 144 | 15% | >10.000 |
| Example 145 | 24% | >10.000 |
| Example 146 | 56% | 4 |
| Example 147 | 66% | 1.4 |
| Example 148 | 20% | >10.000 |
| Example 149 | 72% | 1.25 |
| Example 150 | 66% | 1.1 |
| Example 151 | 33% | >10.000 |
| Example 152 | 9% | >10.000 |
| Example 153 | 31% | >10.000 |
| Example 154 | 89% | 0.15 |
| Example 155 | 103%, | 0.095 |
| Example 156 | 86%, | 0.1 |
| Example 157 | 66%, | 0.3 |
| Example 158 | 39% | >10.000 |
| Example 159 | 5% | >10.000 |
| Example 160 | 51% | |
| Example 161 | 87%, | 0.67 |
| Example 162 | 75% | 2.1 |
| Example 163 | 74% | 4.2 |
| Example 164 | 58% | >10.000 |
| Example 165 | 64% | >10.000 |
| Example 166 | 94% | 1.33 |
| Example 167 | 59% | 4.99 |
| Example 168 | 58% | 2.41 |
| Example 169 | 96% | 0.07 |
| Example 170 | 96% | 0.14 |
| Example 171 | 87% | 0.38 |
| Example 172 | 81% | 1.01 |
| Example 173 | 85% | 0.44 |
| Example 174 | 96% | 0.11 |
| Example 175 | 70% | 0.45 |
| Example 176 | 86% | 0.36 |
| Example 177 | 95% | 0.2 |
| Example 178 | 67% | 3.16 |
| Example 179 | 90% | 0.65 |
| Example 180 | 95% | 0.18 |
| Example 181 | 87% | 0.29 |
| Example 182 | 29% | >10.000 |
| Example 183 | 84% | 0.27 |
| Example 184 | 89% | 0.2 |
| Example 185 | 6% | >10.000 |
| Example 186 | 99% | 0.11 |
| Example 187 | 31% | >10.000 |
| Example 188 | 86% | 0.3 |
| Example 189 | 83% | 0.36 |
| Example 190 | 68% | 0.94 |
| Example 191 | 60% | 1.92 |
| Example 192 | 72% | 1.3 |
| Example 193 | 79% | 0.5 |
| Example 194 | 96% | 0.11 |
| Example 195 | 93% | 0.22 |
| Example 196 | 96% | 0.09 |
| Example 197 | 91% | 0.2 |
| Example 198 | 56% | 1.9 |
| Example 199 | 96% | 0.12 |
| Example 200 | 94% | 0.2 |
| Example 201 | 96% | 0.1 |
| Example 202 | 96% | 0.12 |
| Example 203 | 16% | >10.000 |
| Example 204 | 12% | >10.000 |
| Example 205 | 81% | 1.73 |
| Example 206 | 18% | >10.000 |
| Example 207 | 0% | >10.000 |
| Example 208 | 54% | 6.15 |
| Example 209 | 24% | >10.000 |
| Example 210 | 52% | 2.5 |
| Example 211 | 47% | >10.000 |
| Example 212 | 22% | >10.000 |
| Example 213 | 40% | >10.000 |
| Example 214 | 8% | >10.000 |
| Example 215 | 0% | >10.000 |
| Example 216 | 3% | >10.000 |
| Example 217 | 34% | >10.000 |
| Example 218 | 73% | 4.49 |
| Example 219 | 12% | >10.000 |
| Example 220 | 30% | >10.000 |
| Example 221 | 65% | 9.7 |
| Example 222 | 43% | >10.000 |
| Example 223 | 47% | >10.000 |
| Example 224 | 39% | >10.000 |
| Example 225 | 7% | >10.000 |
| Example 226 | 43% | >10.000 |
| Example 227 | 54% | 1.6 |
| Example 228 | 7% | >10.000 |
| Example 229 | 83% | 0.118 |
| Example 230 | 93% | 0.049 |
| Example 231 | 80% | 0.246 |
| Example 232 | 99% | 0.174 |
| Example 233 | 96% | 1 |
| Example 234 | 96% | 0.019 |
| Example 235 | 84% | 0.169 |
| Example 236 | 50% | 10 |
| Example 237 | 44% | >10.000 |
| Example 238 | 50% | >10.000 |
| Example 239 | 86% | 0.2 |
| Example 240 | 10% | >10.000 |
| Example 241 | 34% | >10.000 |
| Example 242 | 20% | >10.000 |
| Example 243 | 18% | >10.000 |
| Example 244 | 24% | >10.000 |
| Example 245 | 18% | >10.000 |
| Example 246 | 34% | >10.000 |
| Example 247 | 15% | >10.000 |
| Example 248 | 10% | >10.000 |
| Example 249 | 76% | 0.471 |
| Example 250 | 16% | >10.000 |
| Example 251 | 42% | 10 |
| Example 252 | 79% | 0.082 |
| Example 253 | 97% | 0.04 |
| Example 254 | 82% | 0.09 |
| Example 255 | 91% | 0.049 |
| Example 256 | 97% | 0.122 |
| Example 257 | 97% | 0.0745 |
| Example 258 | 95% | 0.1338 |
| Example 259 | 99% | 0.0914 |
| Example 260 | 97% | 0.1558 |
| Example 261 | 94% | 0.1135 |
| Example 262 | 98% | 0.0655 |
| Example 263 | 98% | 0.0658 |
| Example 264 | 97% | 0.0958 |
| Example 265 | 99% | 0.0683 |
| Example 266 | 100% | 0.0533 |
| Example 267 | 99% | 0.1063 |
| Example 268 | 96% | 0.1095 |
| Example 269 | 78% | 2.7858 |
| Example 270 | 80% | 2.0587 |
| Example 271 | 93% | 0.0570 |
| Example 272 | 92% | 0.1167 |
| Example 273 | 97% | 0.0438 |
| Example 274 | 99% | 0.0245 |
| Example 275 | 89% | 0.0840 |
| Example 276 | 58% | 2.0555 |
| Example 277 | 69% | 0.3255 |
| Example 278 | 96% | 0.1115 |
| Example 279 | 91% | 0.8423 |
| Example 280 | 76% | 0.3183 |
| Example 281 | 98% | 0.0407 |
| Example 282 | 79% | 0.5515 |
| Example 283 | 99% | 0.0297 |
| Example 284 | 92% | 0.0780 |
| Example 285 | 93% | 0.0825 |
| Example 286 | 77% | 0.1455 |
| Example 287 | 12% | >10 |
| Example 288 | 35% | >10 |
| Example 289 | 86% | 0.0260 |
| Example 290 | 67% | 0.3670 |
| Example 291 | 33% | >10 |
| Example 292 | 26% | >10 |
| Example 293 | 55% | 7.4750 |
| Example 294 | 42% | >10 |
| Example 295 | 41% | >10 |
| Example 296 | 53% | 1.7720 |
| Example 297 | 75% | 2.1910 |
| Example 298 | 69% | 0.2660 |
| Example 299 | 63% | 0.6187 |
| Example 300 | 71% | 4.4865 |
| Example 301 | 75% | 1.7305 |
| Example 302 | 72% | 0.8000 |
| Example 303 | 80% | 0.2878 |
| Example 304 | 80% | 0.1950 |
| Example 305 | 54% | 0.8927 |
| Example 306 | 68% | 0.8507 |
| Example 307 | 88% | 0.3930 |
| Example 308 | 86% | 0.2810 |
| Example 309 | 63% | 1.3300 |
| Example 310 | 37% | >10 |
| Example 311 | 80% | 10.6570 |
| Example 312 | 77% | 0.5775 |
| Example 313 | 78% | >10 |
| Example 314 | 82% | 0.5145 |
| Example 315 | 102% | 0.0205 |
| Example 316 | 92% | 0.5410 |
| Example 317 | 100% | 0.0423 |
| Example 318 | 59% | 3.5530 |
| Example 319 | 94% | 0.3625 |

The compounds of this invention may be formulated neat or may be combined with one or more pharmaceutically acceptable carriers for administration. For example, solvents, diluents and the like, and may be administered orally in such forms as tablets, capsules, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like, or parenterally in the form of sterile injectable solution or suspension containing from about 0.05 to 5% suspending agent in an isotonic medium. Such pharmaceutical preparations may contain, for example, from about 0.05 up to about 90% of the active ingredient in combination with the carrier, more usually between about 5% and 60% by weight.

The effective dosage of active ingredient employed may vary depending on the particular compound employed, the mode of administration and the severity of the condition being treated. However, in general, satisfactory results are obtained when the compounds of the invention are administered at a daily dosage of from about 0.5 to about 1000 mg/kg of animal body weight, optionally given in divided doses two to four times a day, or in sustained release form. For most large mammals the total daily dosage is from about 1 to 1000 mg, preferably from about 2 to 500 mg. Dosage forms suitable for internal use comprise from about 0.5 to 1000 mg of the active compound in intimate admixture with a solid or liquid pharmaceutically acceptable carrier. This dosage regimen may be adjusted to provide the optimal therapeutic response. For example, several divided doses may be administered daily or the dose may be proportionally reduced as indicated by the exigencies of the therapeutic situation.

The compounds of this invention may be administered orally as well as by intravenous, intramuscular, or subcutaneous routes. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents, and antioxidants, for example, vitamin E, ascorbic acid, BHT and BHA.

The preferred pharmaceutical compositions from the standpoint of ease of preparation and administration are solid compositions, particularly tablets and hard-filled or liquid-filled capsules. Oral administration of the compounds is sometimes desirable.

In some cases it may be desirable to administer the compounds directly to the airways in the form of an aerosol.

The compounds of this invention may also be administered parenterally or intraperitoneally. Solutions or suspensions of these active compounds as a free base or pharmacologically acceptable salt may be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparation contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, polyol (e.g., glycerol, propylene glycol and liquid polyethylene glycol), suitable mixtures thereof, and vegetable oils.

For the treatment of cancer, the compounds of this invention may be administered in combination with other antitumor substances or with radiation therapy. These other substances or radiation treatments may be given at the same or at different times as the compounds of this invention. These combined therapies may effect synergy and result in improved efficacy. For example, the compounds of this invention may be used in combination with mitotic inhibitors such as taxol or vinblastine, alkylating agents such as cisplatin or cyclophosamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, and antiestrogens such as tamoxifen.

As used in accordance with this invention, the term providing an effective amount of a compound means either directly administering such compound, or administering a prodrug, derivative, or analog which will form an effective amount of the compound within the body.

The invention will be more fully described in conjunction with the following specific examples which are not to be construed as limiting the scope of the invention.

### EXAMPLE 1

### Pyrazolo[1,5-a]pyrimidine-3-carboxylic acid, 7-[3-[[3-(trifluoromethyl)-benzoyl]amino]phenyl]-, ethyl ester

MS (electrospray): m/z 455 [M+H]

Step 1: 3-(Dimethylamino)-1-(3-nitrophenyl)-2-propen-1-one 3-Nitroacetophenone (5.0 g, 30.3 mmol) in dimethylformamide-dimethylacetal (10 mL) is heated at reflux overnight. The reaction mixture is cooled to room temperature and evaporated to remove the volatiles. The residue is slurried in ethyl ether and the suspension is filtered and washed with ether to give 10.5 g (79%) of 3-(dimethylamino)-1-(3-nitrophenyl)-2-propen-1-one, 104-105° C.

Step 2: 7-(3-Nitro-phenyl)-pyrazolo[1,5-a]pyrimidine-3-carboxylic acid ethyl ester to a solution of 3-Dimethylamino-1-(3-nitro-phenyl)-propenone (3 mmol) in acetic acid is added 3 amino-4 carbethoxyprazole (3.1 mmol) and heated at 80° C. overnight. The solution is concentrated and the tan solid obtained is taken to the next step without further purification.

Step 3: 7-(3-Amino-phenyl)-pyrazolo[1,5-a]pyrimidine-3-carboxylic acid ethyl ester: A 2.0 L three neck flask equipped with mechanical stirrer is added 7-(3-Nitro-phenyl)-pyrazolo[1,5-a]pyrimidine-3-carboxylic acid ethyl ester (86 mmol), and ammonium chloride (428 mmol) in methanol (200 mL) and water (200 mL). The mixture is stirred for 5 minutes. Iron powder (343 mmol) is added slowly with stirring followed by an additional 200 mL of methanol and 200 mL of water. The reaction mixture is heated gradually to reflux and maintained at reflux overnight, cooled to room temperature and filtered. The red solid cake is washed thoroughly with hot methanol and hot ethyl acetate. The combined filtrates are evaporated to give 7-(3-Amino-phenyl)-pyrazolo[1,5-a]pyrimidine-3-carboxylic acid ethyl ester as a light brown solid. The crude product is used directly for the next step without further purification.

Step 4: Pyrazolo[1,5-a]pyrimidine-3-carboxylic acid, 7-[3-[[3-(trifluoromethyl)-benzoyl]amino]phenyl]-, ethyl ester

To a solution of 7-(3-Amino-phenyl)-pyrazolo[1,5-a]pyrimidine-3-carboxylic acid ethyl ester (0.15 mmol) in pyridine is added 3-(trifluoromethyl)benzoyl chloride (0.17 mmol) and stirred at room temperature for 18 h. The reaction mixture is concentrated and purified by HPLC.

Examples 2-10 are prepared following the above method for Example 1 using the appropriate acid chlorides at the final step.

### EXAMPLE 2

### ethyl 7-(3-{[4-fluoro-3-(trifluoromethyl)benzoyl]amino}phenyl)pyrazolo[1,5-a]pyrimidine-3-carboxylate

MS (electrospray): m/z 473 [M+H]

### EXAMPLE 3

Pyrazolo[1,5-a]pyrimidine-3-carboxylic acid, 7-[3-(benzoylamino)phenyl]-, ethyl ester MS (electrospray): m/z 387 [M+H]

### EXAMPLE 4

ethyl 7-{3-[(3-bromobenzoyl)amino]phenyl}pyrazolo[1,5-a]pyrimidine-3-carboxylate MS (electrospray): m/z 465 [M+H]

### EXAMPLE 5

### ethyl 7-{3-[(1-benzothien-2-ylcarbonyl)amino]phenyl}pyrazolo[1,5-a]pyrimidine-3-carboxylate

MS (electrospray): m/z 443 [M+H]

### EXAMPLE 6

ethyl 7-{3-[(4-chlorobenzoyl)amino]phenyl}pyrazolo[1,5-a]pyrimidine-3-carboxylate MS (electrospray): m/z 421 [M+H]

### EXAMPLE 7

### ethyl 7-(3-{[3-(trifluoromethoxy)benzoyl]amino}phenyl)pyrazolo[1,5-a]pyrimidine-3-carboxylate

MS (electrospray): m/z 471 [M+H]

### EXAMPLE 8

### ethyl 7-{3-[(3-methoxybenzoyl)amino]phenyl}pyrazolo[1,5-a]pyrimidine-3-carboxylate

MS (electrospray): m/z 417 [M+H]

### EXAMPLE 9

### ethyl 7-(3-{[3-fluoro-4-(trifluoromethyl)benzoyl]amino}phenyl)pyrazolo[1,5-a]pyrimidine-3-carboxylate

MS (electrospray): m/z 473 [M+H]

### EXAMPLE 10

### ethyl 7-(3-{[4-(trifluoromethyl)benzoyl]amino}phenyl)pyrazolo[1,5-a]pyrimidine-3-carboxylate

MS (electrospray): m/z 455 [M+H]

Example 11 is prepared following the above method for Example 1 using the appropriate isocyanates at the final step.

### EXAMPLE 11

### ethyl 7-{3-[({[4-chloro-3-(trifluoromethyl)phenyl]amino}carbonyl)amino]phenyl}pyrazolo[1,5-a]pyrimidine-3-carboxylate

MS (electrospray): m/z 504 [M+H]

Example 12 is prepared following the above method for Example 1 using 3-trifluoromethylphenyl sulfonyl chloride at the final step.

### EXAMPLE 12

### ethyl 7-[3-({[3-(trifluoromethyl)phenyl]sulfonyl}amino)phenyl]pyrazolo[1,5-a]pyrimidine-3-carboxylate

MS (electrospray): m/z 491 [M+H]

Example 13 is prepared following the above method for Example 1 using the appropriate acid chlorides at the final step.

### EXAMPLE 13

### ethyl 7-{3-[(3-cyanobenzoyl)amino]phenyl}pyrazolo[1,5-a]pyrimidine-3-carboxylate

MS (electrospray): m/z 412 [M+H]

Examples 14-25 are prepared following the above method for Example 1 using the appropriate isocyanates at the final step.

### EXAMPLE 14

### ethyl 7-[3-({[(2,4-dichlorophenyl)amino]carbonyl}amino)phenyl]pyrazolo[1,5-a]pyrimidine-3-carboxylate

MS (electrospray): m/z 470 [M+H]

### EXAMPLE 15

### ethyl 7-{3-[({[(ethoxycarbonyl)phenyl]amino}carbonyl)amino]phenyl}pyrazolo[1,5-a]pyrimidine-3-carboxylate

MS (electrospray): m/z 474 [M+H]

### EXAMPLE 16

### ethyl 7-{3-[({[3,5-bis(trifluoromethyl)phenyl]amino}carbonyl)amino]phenyl}pyrazolo[1,5-a]pyrimidine-3-carboxylate

MS (electrospray): m/z 538 [M+H]

### EXAMPLE 17

### ethyl 7-[3-({[(3,5-dichlorophenyl)amino]carbonyl}amino)phenyl]pyrazolo[1,5-a]pyrimidine-3-carboxylate

MS (electrospray): m/z 470 [M+H]

### EXAMPLE 18

### ethyl 7-{3-[({[4-(methylthio)phenyl]amino}carbonyl)amino]phenyl}pyrazolo[1,5-a]pyrimidine-3-carboxylate

MS (electrospray): m/z 448 [M+H]

### EXAMPLE 19

### ethyl 7-[3-({[(4-acetylphenyl)amino]carbonyl}amino)phenyl]pyrazolo[1,5-a]pyrimidine-3-carboxylate

MS (electrospray): m/z 444 [M+H]

### EXAMPLE 20

### ethyl 7-[3-({[(4-isopropylphenyl)amino]carbonyl}amino)phenyl]pyrazolo[1,5-a]pyrimidine-3-carboxylate

MS (electrospray): m/z 444 [M+H]

### EXAMPLE 21

### ethyl 7-(3-{[(2-naphthylamino)carbonyl]amino}phenyl)pyrazolo[1,5-a]pyrimidine-3-carboxylate

MS (electrospray): m/z 452 [M+H]

### EXAMPLE 22

### ethyl 7-(3-{[(mesitylamino)carbonyl]amino}phenyl)pyrazolo[1,5-a]pyrimidine-3-carboxylate

MS (electrospray): m/z 444 [M+H]

### EXAMPLE 23

### ethyl 7-{3-[({[4-(trifluoromethoxy)phenyl]amino}carbonyl)amino]phenyl}pyrazolo[1,5-a]pyrimidine-3-carboxylate

MS (electrospray): m/z 486 [M+H]

### EXAMPLE 24

### ethyl 7-(3-{[({4-[(trifluoromethyl)thio]phenyl}amino)carbonyl]amino}phenyl)pyrazolo[1,5-a]pyrimidine-3-carboxylate

MS (electrospray): m/z 502 [M+H]

### EXAMPLE 25

### ethyl 7-[3-({[(3-chloro-4-fluorophenyl)amino]carbonyl}amino)phenyl]pyrazolo[1,5-a]pyrimidine-3-carboxylate

MS (electrospray): m/z 454 [M+H]

Examples 26-30 are prepared following the above method for Example 1 using the appropriate acid chlorides at the final step.

### EXAMPLE 26

### ethyl 7-(3-{[4-methyl-3-(trifluoromethyl)benzoyl]amino}phenyl)pyrazolo[1,5-a]pyrimidine-3-carboxylate

MS (electrospray): m/z 469 [M+H]

### EXAMPLE 27

ethyl 7-(3-{[2-chloro-5-(trifluoromethyl)benzoyl]amino}phenyl)pyrazolo[1,5- MS (electrospray): m/z 489 [M+H]

### EXAMPLE 28

### ethyl 7-(3-{[3-chloro-2-fluoro-5-(trifluoromethyl)benzoyl]amino}phenyl)pyrazolo[1,5-a]pyrimidine-3-carboxylate

MS (electrospray): m/z 507 [M+H]

### EXAMPLE 29

### ethyl 7-{3-[(4-chloro-2,5-difluorobenzoyl)amino]phenyl}pyrazolo[1,5-a]pyrimidine-3-carboxylate

MS (electrospray): m/z 457 [M+H]

### EXAMPLE 30

### ethyl 7-(3-{[4-methoxy-3-(trifluoromethyl)benzoyl]amino}phenyl)pyrazolo[1,5-a]pyrimidine-3-carboxylate

MS (electrospray): m/z 485 [M+H]

### EXAMPLE 31

### N-methyl-7-(3-{[3-(trifluoromethyl)benzoyl]amino}phenyl)-pyrazolo[1,5-a]pyrimidine-3-carboxamide

To a solution of 7-(3-{[3-(trifluoromethyl)benzoyl]amino}phenyl)pyrazolo[1,5-a]pyrimidine-3-carboxylic acid (0.07 mmol) in DMF (1 mL) is added N,N diisopropyl ethylamine (0.16 mmol), pybop (0.16) and let stir for 5 minutes. To this reaction mixture is added a solution of N-methylamine in THF (excess) and stirred overnight. The solution is then concentrated and purified by HPLC.

MS (electrospray): m/z 440 [M+H]

### EXAMPLE 32

### 7-(3-{[3-(trifluoromethyl)benzoyl]amino}phenyl)pyrazolo[1,5-a]pyrimidine-3-carboxylic acid

To a solution of Pyrazolo[1,5-a]pyrimidine-3-carboxylic acid, 7-[3-[[3-(trifluoromethyl)-benzoyl]amino]phenyl]-, ethyl ester (Example 1) (100 mg) in 1:1 methanol-tetrahydrofuran is added 2M solution of lithium hydroxide and stirred at 40 oC for 6 hours. After neutralization with 2N HCl and removing the solvents, the residue is purified by flash column chromatography.

MS (electrospray): m/z 427 [M+H]

Examples 33-44 are prepared following the above method for Example 1 using the appropriate isocyanates at the final step.

### EXAMPLE 33

### ethyl 7-{3-[({[3-(trifluoromethyl)phenyl]amino}carbonyl)amino]phenyl}pyrazolo[1,5-a]pyrimidine-3-carboxylate

MS (electrospray): m/z 470 [M+H]

### EXAMPLE 34

### ethyl 7-[3-({[(4-chlorophenyl)amino]carbonyl}amino)phenyl]pyrazolo[1,5-a]pyrimidine-3-carboxylate

MS (electrospray): m/z 436 [M+H]

### EXAMPLE 35

### ethyl 7-{3-[({[4-(trifluoromethyl)phenyl]amino}carbonyl)amino]phenyl}pyrazolo[1,5-a]pyrimidine-3-carboxylate

MS (electrospray): m/z 470 [M+H]

### EXAMPLE 36

### ethyl 7-[3-({[(4-chloro-2-methylphenyl)amino]carbonyl}amino)phenyl]pyrazolo[1,5-a]pyrimidine-3-carboxylate

MS (electrospray): m/z 450 [M+H]

### EXAMPLE 37

### ethyl 7-{3-[({[2-chloro-5-(trifluoromethyl)phenyl]amino}carbonyl)amino]phenyl}pyrazolo[1,5-a]pyrimidine-3-carboxylate

MS (electrospray): m/z 504 [M+H]

### EXAMPLE 38

### ethyl 7-[3-({[(4-cyanophenyl)amino]carbonyl}amino)phenyl]pyrazolo[1,5-a]pyrimidine-3-carboxylate

MS (electrospray): m/z 427 [M+H]427 M+H 2.17

### EXAMPLE 39

### ethyl 7-{3-[({[2-(trifluoromethyl)phenyl]amino}carbonyl)amino]phenyl}pyrazolo[1,5-a]pyrimidine-3-carboxylate

MS (electrospray): m/z 470 [M+H]

### EXAMPLE 40

### ethyl 7-[3-({[(3,4-dichlorophenyl)amino]carbonyl}amino)phenyl]pyrazolo[1,5-a]pyrimidine-3-carboxylate

MS (electrospray): m/z 470 [M+H]

### EXAMPLE 41

### ethyl 7-[3-({[(4-bromophenyl)amino]carbonyl}amino)phenyl]pyrazolo[1,5-a]pyrimidine-3-carboxylate

MS (electrospray): m/z 480 [M+H]

### EXAMPLE 42

### ethyl 7-[3-({[(3,4-dimethylphenyl)amino]carbonyl}amino)phenyl]pyrazolo[1,5-a]pyrimidine-3-carboxylate

MS (electrospray): m/z 430 [M+H]

### EXAMPLE 43

### ethyl 7-{3-[({[4-chloro-2-(trifluoromethyl)phenyl]amino}carbonyl)amino]phenyl}pyrazolo[1,5-a]pyrimidine-3-carboxylate

MS (electrospray): m/z 504 [M+H]

### EXAMPLE 44

### ethyl 7-{3-[({[4-fluoro-3-(trifluoromethyl)phenyl]amino}carbonyl)amino]phenyl}pyrazolo[1,5-a]pyrimidine-3-carboxylate

MS (electrospray): m/z 488 [M+H]

Examples 45-57 are prepared following the above method for Example 31 using the appropriate amines at the final step.

### EXAMPLE 45

### N-(2-methoxyethyl)-7-(3-{[3-(trifluoromethyl)benzoyl]amino}phenyl)pyrazolo[1,5-a]pyrimidine-3-carboxamide

MS (electrospray): m/z 484 [M+H]

### EXAMPLE 46

### N-propyl-7-(3-{[3-(trifluoromethyl)benzoyl]amino}phenyl)pyrazolo[1,5-a]pyrimidine-3-carboxamide

MS (electrospray): m/z 468 [M+H]

### EXAMPLE 47

### N-pyridin-3-yl-7-(3-{[3-(trifluoromethyl)benzoyl]amino}phenyl)pyrazolo[1,5-a]pyrimidine-3-carboxamide

MS (electrospray): m/z 503 [M+H]

### EXAMPLE 48

### N-(2-pyrrolidin-1-ylethyl)-7-(3-{[3-(trifluoromethyl)benzoyl]amino}phenyl)pyrazolo[1,5-a]pyrimidine-3-carboxamide

MS (electrospray): m/z 523 [M+H]

### EXAMPLE 49

### N-[2-(dimethylamino)ethyl]-7-(3-{[3-(trifluoromethyl)benzoyl]amino}phenyl)pyrazolo[1,5-a]pyrimidine-3-carboxamide

MS (electrospray): m/z 497 [M+H]

### EXAMPLE 50

### N-[3-(4-methylpiperazin-1-yl)propyl]-7-(3-{[3-(trifluoromethyl)benzoyl]amino}phenyl)pyrazolo[1,5-a]pyrimidine-3-carboxamide

MS (electrospray): m/z 566 [M+H]

### EXAMPLE 51

### N-ethyl-7-(3-{[3-(trifluoromethyl)benzoyl]amino}phenyl)pyrazolo[1,5-a]pyrimidine-3-carboxamide

MS (electrospray): m/z 454 [M+H]

### EXAMPLE 52

### N-(2-morpholin-4-ylethyl)-7-(3-{[3-(trifluoromethyl)benzoyl]amino}phenyl)pyrazolo[1,5-a]pyrimidine-3-carboxamide

MS (electrospray): m/z 539 [M+H]

### EXAMPLE 53

### N-(3-morpholin-4-ylpropyl)-7-(3-{[3-(trifluoromethyl)benzoyl]amino}phenyl)pyrazolo[1,5-a]pyrimidine-3-carboxamide

MS (electrospray): m/z 553 [M+H]

### EXAMPLE 54

### N-[2-(1-methylpyrrolidin-2-yl)ethyl]-7-(3-{[3-(trifluoromethyl)benzoyl]amino}phenyl)pyrazolo[1,5-a]pyrimidine-3-carboxamide

MS (electrospray): m/z 537 [M+H]

### EXAMPLE 55

### N-[3-(1H-imidazol-1-yl)propyl]-7-(3-{[3-(trifluoromethyl)benzoyl]amino}phenyl)pyrazolo[1,5-a]pyrimidine-3-carboxamide

MS (electrospray): m/z 534 [M+H]

### EXAMPLE 56

### N-(3-methoxypropyl)-7-(3-{[3-(trifluoromethyl)benzoyl]amino}phenyl)pyrazolo[1,5-a]pyrimidine-3-carboxamide

MS (electrospray): m/z 498 [M+H]

### EXAMPLE 57

N-benzyl-7-(3-{[3-(trifluoromethyl)benzoyl]amino}phenyl)pyrazolo[1,5-a]pyrimidine-3-carboxamide

### EXAMPLE 58

### Ethyl 2-methyl-7-(3-{[3-(trifluoromethyl)benzoyl]amino}phenyl)pyrazolo[1,5-a]pyrimidine-3-carboxylate

Step 1: N-(3-Acetyl-phenyl)-3-trifluoromethyl-benzamide: To a solution of 3 amino acetophenone (3 g, 22 mmol) in pyridine (18 mL) is added 3-trifluoromethyl benzoyl chloride (5 g, 24 mmol) and heated at 50° C. overnight. The solution is then concentrated and the crude product is taken to next step without further purification.

Step 2: N-[3-(3-Dimethylamino-acryloyl)-phenyl]-3-trifluoromethyl-benzamide: The above benzamide is taken up in N,N dimethylformamide dimethyl acetal (5 mL) and heated for 7 hours at 80° C. Resulting solution is then concentrated and used in the next step without any further purification.

Step 3: Ethyl 2-methyl-7-(3-{[3-(trifluoromethyl)benzoyl]amino}phenyl)-pyrazolo[1,5-a]pyrimidine-3-carboxylate: To a solution of N-[3-(3-Dimethylamino-acryloyl)-phenyl]-3-trifluoromethyl-benzamide in acetic acid (1 mL) is added 5-Amino-3-methyl-1H-pyrazole-4-carboxylic acid ethyl ester (47 mg, 0.31 mmol) and heated at 80° C. overnight. The solution is concentrated down and purified by HPLC.

MS (electrospray): m/z 469 [M+H]

Examples 59-63 are prepared following the above method for Example 58 using the appropriate amino pyrazoles at the final step.

### EXAMPLE 59

### N-[3-(3-pyridin-2-ylpyrazolo[1,5-a]pyrimidin-7-yl)phenyl]-3-(trifluoromethyl)benzamide

MS (electrospray): m/z 460 [M+H]

### EXAMPLE 60

### N-{3-[3-(2H-tetrazol-5-yl)pyrazolo[1,5-a]pyrimidin-7-yl]phenyl}-3-(trifluoromethyl)benzamide

MS (electrospray): m/z 451 [M+H]

### EXAMPLE 61

### 7-(3-{[3-(trifluoromethyl)benzoyl]amino}phenyl)pyrazolo[1,5-a]pyrimidine-3-carboxamide

MS (electrospray): m/z 426 [M+H]

### EXAMPLE 62

### N-[3-(3-cyano-2-piperazin-1-ylpyrazolo[1,5-a]pyrimidin-7-yl)phenyl]-3-(trifluoromethyl)benzamide

MS (electrospray): m/z 492 [M+H]

### EXAMPLE 63

### N-[3-(3-cyanopyrazolo[1,5-a]pyrimidin-7-yl)phenyl]-3-(trifluoromethyl)benzamide

MS (electrospray): m/z 408 [M+H]

Examples 64-69 are prepared following the above method for Example 58, but using 4-methyl-3-trifluoromethyl benzoyl chloride in step 1 and appropriate amino pyrazoles in the final step.

### EXAMPLE 64

### ethyl 2-methyl-7-(3-{[4-methyl-3-(trifluoromethyl)benzoyl]amino}phenyl)pyrazolo[1,5-a]pyrimidine-3-carboxylate

MS (electrospray): m/z 483 [M+H]

### EXAMPLE 65

### 4-methyl-N-[3-(3-pyridin-2-ylpyrazolo[1,5-a]pyrimidin-7-yl)phenyl]-3-(trifluoromethyl)benzamide

MS (electrospray): m/z 474 [M+H]

### EXAMPLE 66

### 4-methyl-N-{3-[3-(2H-tetrazol-5-yl)pyrazolo[1,5-a]pyrimidin-7-yl]phenyl}-3-(trifluoromethyl)benzamide

MS (electrospray): m/z 465 [M+H]

### EXAMPLE 67

### 7-(3-{[4-methyl-3-(trifluoromethyl)benzoyl]amino}phenyl)pyrazolo[1,5-a]pyrimidine-3-carboxamide

MS (electrospray): m/z 440 [M+H]

### EXAMPLE 68

### N-[3-(3-cyano-2-piperazin-1-ylpyrazolo[1,5-a]pyrimidin-7-yl)phenyl]-4-methyl-3-(trifluoromethyl)benzamide

MS (electrospray): m/z 506 [M+H]

### EXAMPLE 69

### N-[3-(3-cyanopyrazolo[1,5-a]pyrimidin-7-yl)phenyl]-4-methyl-3-(trifluoromethyl)benzamide

MS (electrospray): m/z 422 [M+H]

### EXAMPLE 70

### Ethyl 7-{3-[(3-chlorobenzoyl)amino]phenyl}pyrazolo[1,5-a]pyrimidine-3-carboxylate

To a solution of 3 chlorobenzoic acid (18.9 mg, 0.12 mmol) in DMF (2 mL) is added N,N diisopropyl ethylamine (31.8 mg 0.11 mmol), pybop (56 mg, 0.11) and stirred for five minutes. To this reaction mixture is added 7-(3-Amino-phenyl)pyrazolo[1,5-a]pyrimidine-3-carboxylic acid ethyl ester (31 mg, 0.11 mmol) and stirred 48 hours. The solution is then concentrated down and purified by HPLC.

MS (electrospray): m/z 421 [M+H]

Examples 71-76 are prepared following the above method for Example 70 using the appropriate benzoic acids.

### EXAMPLE 71

### ethyl 7-{3-[(3,4-dichlorobenzoyl)amino]phenyl}pyrazolo[1,5-a]pyrimidine-3-carboxylate

MS (electrospray): m/z 455 [M+H]

### EXAMPLE 72

### ethyl 7-{3-[(3,5-dichlorobenzoyl)amino]phenyl}pyrazolo[1,5-a]pyrimidine-3-carboxylate

MS (electrospray): m/z 455 [M+H]

### EXAMPLE 73

### ethyl 7-{3-[(3-chloro-4-methoxybenzoyl)amino]phenyl}pyrazolo[1,5-a]pyrimidine-3-carboxylate

MS (electrospray): m/z 451 [M+H]

### EXAMPLE 74

### ethyl 7-{3-[(5-chloro-2-methylbenzoyl)amino]phenyl}pyrazolo[1,5-a]pyrimidine-3-carboxylate

MS (electrospray): m/z 435 [M+H]

### EXAMPLE 75

### ethyl 7-(3-{[3-fluoro-5-(trifluoromethyl)benzoyl]amino}phenyl)pyrazolo[1,5-a]pyrimidine-3-carboxylate

MS (electrospray): m/z 473 [M+H]

### EXAMPLE 76

### ethyl 7-(3-{[4-fluoro-2-(trifluoromethyl)benzoyl]amino}phenyl)pyrazolo[1,5-a]pyrimidine-3-carboxylate

MS (electrospray): m/z 473 [M+H]

Examples 77-79 are prepared following the above method for Example 80 using the appropriate amine.

### EXAMPLE 77

### ethyl 7-{3-[({[3-methoxy-5-(trifluoromethyl)phenyl]amino}carbonyl)amino]phenyl}pyrazolo[1,5-a]pyrimidine-3-carboxylate

MS (electrospray): m/z 500 [M+H]

### EXAMPLE 78

### ethyl 7-{3-[({[4-cyano-3-(trifluoromethyl)phenyl]amino}carbonyl)amino]phenyl}pyrazolo[1,5-a]pyrimidine-3-carboxylate

MS (electrospray): m/z 495.1 [M+H]

### EXAMPLE 79

### ethyl 7-{3-[({[4-methyl-3-(trifluoromethyl)phenyl]amino}carbonyl)amino]phenyl}pyrazolo[1,5-a]pyrimidine-3-carboxylate

MS (electrospray): m/z 484 [M+H]

### EXAMPLE 80

### Ethyl 7-[3-({[(3-chlorophenyl)amino]carbonyl}amino)phenyl]-pyrazolo[1,5-a]pyrimidine-3-carboxylate

To a solution of 7-(3-Amino-phenyl)-pyrazolo[1,5-a]pyrimidine-3-carboxylic acid ethyl ester (50 mg, 0.17 mmol) in pyridine (6 mL) is added 4 nitrophenyl chloroformate (53.4 mg, 0.26 mmol). After stirring for 3 hours at 50° C., 1 amino 3 chlorobenzene (25.5 mg, 0.20 mmol) is added and stirred at 50° C. overnight. The solution is then concentrated and purified by HPLC.

MS (electrospray): m/z 436 [M+H]

Examples 81-84 are prepared following the above method for Example 80 using the appropriate amine.

### EXAMPLE 81

### ethyl 7-[3-({[(3-chloro-4-methoxyphenyl)amino]carbonyl}amino)phenyl]pyrazolo[1,5-a]pyrimidine-3-carboxylate

MS (electrospray): m/z 466 [M+H]

### EXAMPLE 82

### ethyl 7-[3-({[(3-chloro-4-methylphenyl)amino]carbonyl}amino)phenyl]pyrazolo[1,5-a]pyrimidine-3-carboxylate

MS (electrospray): m/z 450.1 [M+H]

### EXAMPLE 83

### ethyl 7-[3-({[(4-bromo-3-chlorophenyl)amino]carbonyl}amino)phenyl]pyrazolo[1,5-a]pyrimidine-3-carboxylate

MS (electrospray): m/z 514 [M+H]

### EXAMPLE 84

### ethyl 7-[3-({[(3-chloro-4-morpholin-4-ylphenyl)amino]carbonyl}amino)phenyl]pyrazolo[1,5-a]pyrimidine-3-carboxylate

MS (electrospray): m/z 521.2 [M+H]

Examples 85-91 are prepared following the method described for Example 1 using the appropriate substituted ketone in the first step.

### EXAMPLE 85

### ethyl 7-(2-nitro-5-{[3-(trifluoromethyl)benzoyl]amino}phenyl)pyrazolo[1,5-a]pyrimidine-3-carboxylate

MS (electrospray): m/z 500.1 [M+H]

### EXAMPLE 86

### ethyl 7-(4-chloro-3-{[3-(trifluoromethyl)benzoyl]amino}phenyl)pyrazolo[1,5-a]pyrimidine-3-carboxylate

MS (electrospray): m/z 489.1 [M+H]

### EXAMPLE 87

### ethyl 7-(4-(2,6-dimethylmorpholin-4-yl)-3-{[3-(trifluoromethyl)benzoyl]amino}phenyl)pyrazolo[1,5-a]pyrimidine-3-carboxylate

MS (electrospray): m/z 568.2 [M+H]

### EXAMPLE 88

### ethyl 7-(4-methoxy-3-{[3-(trifluoromethyl)benzoyl]amino}phenyl)pyrazolo[1,5-a]pyrimidine-3-carboxylate

MS (electrospray): m/z 485.1 [M+H]

### EXAMPLE 89

### ethyl 7-(4-fluoro-3-{[3-(trifluoromethyl)benzoyl]amino}phenyl)pyrazolo[1,5-a]pyrimidine-3-carboxylate

MS (electrospray): m/z 473.1 [M+H]

### EXAMPLE 90

### ethyl 7-(4-(benzyloxy)-3-{[3-(trifluoromethyl)benzoyl]amino}phenyl)pyrazolo[1,5-a]pyrimidine-3-carboxylate

MS (electrospray): m/z 561.2 [M+H]

### EXAMPLE 91

### ethyl 7-(2-fluoro-5-{[3-(trifluoromethyl)benzoyl]amino}phenyl)pyrazolo[1,5-a]pyrimidine-3-carboxylate

MS (electrospray): m/z 473.1 [M+H]

Examples 92-102 are prepared following the method for Example 70 using the appropriate phenyl acetic acids.

### EXAMPLE 92

### ethyl 7-(3-{[(3-bromophenyl)acetyl]amino}phenyl)pyrazolo[1,5-a]pyrimidine-3-carboxylate

MS (electrospray): m/z 479.1 [M+H]

### EXAMPLE 93

### ethyl 7-(3-{[(3-fluorophenyl)acetyl]amino}phenyl)pyrazolo[1,5-a]pyrimidine-3-carboxylate

MS (electrospray): m/z 419.1 [M+H]

### EXAMPLE 94

### ethyl 7-(3-{[(3-chlorophenyl)acetyl]amino}phenyl)pyrazolo[1,5-a]pyrimidine-3-carboxylate

MS (electrospray): m/z 435.1 [M+H]

### EXAMPLE 95

### ethyl 7-(3-{[(3,4-dichlorophenyl)acetyl]amino}phenyl)pyrazolo[1,5-a]pyrimidine-3-carboxylate

MS (electrospray): m/z 469.1 [M+H]

### EXAMPLE 96

### ethyl 7-(3-{[(3-methoxyphenyl)acetyl]amino}phenyl)pyrazolo[1,5-a]pyrimidine-3-carboxylate

MS (electrospray): m/z 431.2 [M+H]

### EXAMPLE 97

### ethyl 7-[3-({[3-(trifluoromethyl)phenyl]acetyl}amino)phenyl]pyrazolo[1,5-a]pyrimidine-3-carboxylate

MS (electrospray): m/z 469.1 [M+H]

### EXAMPLE 98

### ethyl 7-(3-{[(3-methylphenyl)acetyl]amino}phenyl)pyrazolo[1,5-a]pyrimidine-3-carboxylate

MS (electrospray): m/z 415.2 [M+H]

### EXAMPLE 99

### ethyl 7-[3-({[3,5-bis(trifluoromethyl)phenyl]acetyl}amino)phenyl]pyrazolo[1,5-a]pyrimidine-3-carboxylate

MS (electrospray): m/z 537.1 [M+H]

### EXAMPLE 100

### ethyl 7-{3-[(1,3-benzodioxol-5-ylacetyl)amino]phenyl}pyrazolo[1,5-a]pyrimidine-3-carboxylate

MS (electrospray): m/z 445.1 [M+H]

### EXAMPLE 101

### ethyl 7-(3-{[(4-methoxy-3-methylphenyl)acetyl]amino}phenyl)pyrazolo[1,5-a]pyrimidine-3-carboxylate

MS (electrospray): m/z 445.2 [M+H]

### EXAMPLE 102

### ethyl 7-(3-{[(2,3,6-trifluorophenyl)acetyl]amino}phenyl)pyrazolo[1,5-a]pyrimidine-3-carboxylate

MS (electrospray): m/z 455.1 [M+H]

Examples 103-104 are prepared following the above method for Example 1 using the appropriate acid chlorides at the final step.

### EXAMPLE 103

ethyl 7-(3-{[4-chloro-3-(trifluoromethyl)benzoyl]amino}phenyl)pyrazolo[1,5-a]pyrimidine-3-carboxylate

MS (electrospray): m/z 489 [M+H]

### EXAMPLE 104

### ethyl 7-(3-{[2-methyl-5-(trifluoromethyl)benzoyl]amino}phenyl)pyrazolo[1,5-a]pyrimidine-3-carboxylate

MS (electrospray): m/z 469 [M+H]

Examples 105-108 are prepared following the method described for Example 1 using the appropriate substituted ketone in the first step and 4-methyl-3-trifluoromethyl benzoyl chloride as acylating agent in the last step.

### EXAMPLE 105

### ethyl 7-(5-{[4-methyl-3-(trifluoromethyl)benzoyl]amino}-2-nitrophenyl)pyrazolo[1,5-a]pyrimidine-3-carboxylate

MS (electrospray): m/z 514 [M+H]

### EXAMPLE 106

### ethyl 7-(4-chloro-3-{[4-methyl-3-(trifluoromethyl)benzoyl]amino}phenyl)pyrazolo[1,5-a]pyrimidine-3-carboxylate

MS (electrospray): m/z 503 [M+H]

### EXAMPLE 107

### ethyl 7-(4-methoxy-3-{[4-methyl-3-(trifluoromethyl)benzoyl]amino}phenyl)pyrazolo[1,5-a]pyrimidine-3-carboxylate

MS (electrospray): m/z 499 [M+H]

### EXAMPLE 108

ethyl 7-(4-(benzyloxy)-3-{[4-methyl-3-(trifluoromethyl)benzoyl]amino}phenyl)pyrazolo[1,5-a]pyrimidine-3-carboxylate

### EXAMPLE 109

### N-[3-(2-pyridin-4-ylpyrazolo[1,5-a]pyrimidin-7-yl)phenyl]-3-(trifluoromethyl)benzamide

To a solution of N-[3-(3-Dimethylamino-acryloyl)-phenyl]-3-trifluoromethyl-benzamide (50 mg, 0.14) (preparing as described for example 58 in acetic acid (1 mL)) is added 5-pyridin-4-yl-2H-pyrazol-3-ylamine (35 mg, 0.21 mmol) and heated at 80° C. overnight. The solution is concentrated and purified by HPLC.

MS (electrospray): m/z 460 [M+H]

Examples 110-119 are prepared following the above method for Example 109 using the appropriate substituted pyrazole amine.

### EXAMPLE 110

### N-(3-{2-[3-(dimethylamino)propyl]pyrazolo[1,5-a]pyrimidin-7-yl}phenyl)-3-(trifluoromethyl)benzamide

MS (electrospray): m/z 466 [M+H]

### EXAMPLE 111

### N-[3-(2-pyridin-2-ylpyrazolo[1,5-a]pyrimidin-7-yl)phenyl]-3-(trifluoromethyl)benzamide

MS (electrospray): m/z 460 [M+H]

### EXAMPLE 112

### N-[3-(2-methylpyrazolo[1,5-a]pyrimidin-7-yl)phenyl]-3-(trifluoromethyl)benzamide

MS (electrospray): m/z 397 [M+H]

### EXAMPLE 113

### N-{3-[2-(2-furyl)pyrazolo[1,5-a]pyrimidin-7-yl]phenyl}-3-(trifluoromethyl)benzamide

MS (electrospray): m/z 449 [M+H]

### EXAMPLE 114

### N-{3-[2-(2-thienyl)pyrazolo[1,5-a]pyrimidin-7-yl]phenyl}-3-(trifluoromethyl)benzamide

MS (electrospray): m/z 463 [M+H]

### EXAMPLE 115

### N-{3-[2-(4-chlorophenyl)pyrazolo[1,5-a]pyrimidin-7-yl]phenyl}-3-(trifluoromethyl)benzamide

MS (electrospray): m/z 491 [M+H]

### EXAMPLE 116

### 4-methyl-N-[3-(2-methylpyrazolo[1,5-a]pyrimidin-7-yl)phenyl]-3-(trifluoromethyl)benzamide

MS (electrospray): m/z 411 [M+H]

### EXAMPLE 17

### 4-methyl-N-{3-[2-(2-thienyl)pyrazolo[1,5-a]pyrimidin-7-yl]phenyl}-3-(trifluoromethyl)benzamide

MS (electrospray): m/z 479 [M+H]

### EXAMPLE 118

### N-{3-[2-(4-chlorophenyl)pyrazolo[1,5-a]pyrimidin-7-yl]phenyl}-4-methyl-3-(trifluoromethyl)benzamide

MS (electrospray): m/z 507 [M+H]

### EXAMPLE 119

### 4-methyl-N-[3-(2-phenylpyrazolo[1,5-a]pyrimidin-7-yl)phenyl]-3-(trifluoromethyl)benzamide

MS (electrospray): m/z 473 [M+H]

Examples 120-123 are prepared following the above method for Example 31, but using 7-{3-[3-(4-Chloro-3-trifluoromethyl-phenyl)-ureido]-phenyl}-pyrazolo[1,5-a]pyrimidine-3-carboxylic acid (preparing from example 11 as described in example 32) and different amines at the final step.

### EXAMPLE 120

### 7-{3-[({[4-chloro-3-(trifluoromethyl)phenyl]amino}carbonyl)amino]phenyl}-N-[3-(1H-imidazol-1-yl)propyl]pyrazolo[1,5-a]pyrimidine-3-carboxamide

MS (electrospray): m/z 583 [M+H]

### EXAMPLE 121

### 7-{3-[({[4-chloro-3-(trifluoromethyl)phenyl]amino}carbonyl)amino]phenyl}-N-(3-methoxypropyl)pyrazolo[1,5-a]pyrimidine-3-carboxamide

MS (electrospray): m/z 547 [M+H]

### EXAMPLE 122

### 7-{3-[({[4-chloro-3-(trifluoromethyl)phenyl]amino}carbonyl)amino]phenyl}-N-[2-(diethylamino)ethyl]pyrazolo[1,5-a]pyrimidine-3-carboxamide

MS (electrospray): m/z 574 [M+H]

### EXAMPLE 123

### 7-{3-[({[4-chloro-3-(trifluoromethyl)phenyl]amino}carbonyl)amino]phenyl}-N-(2-morpholin-4-ylethyl)pyrazolo[1,5-a]pyrimidine-3-carboxamide

MS (electrospray): m/z 588 [M+H]

Examples 124-128 are prepared following the above method for Example 31 using the appropriate alcohols at the final step.

### EXAMPLE 124

### methyl 7-(3-{[3-(trifluoromethyl)benzoyl]amino}phenyl)pyrazolo[1,5-a]pyrimidine-3-carboxylate

MS (electrospray): m/z 439 [M+H]

### EXAMPLE 125

### 2,2,2-trifluoroethyl 7-(3-{[3-(trifluoromethyl)benzoyl]amino}phenyl)pyrazolo[1,5-a]pyrimidine-3-carboxylate

MS (electrospray): m/z 507 [M+H]

### EXAMPLE 126

### pyridin-3-yl 7-(3-{[3-(trifluoromethyl)benzoyl]amino}phenyl)pyrazolo[1,5-a]pyrimidine-3-carboxylate

MS (electrospray): m/z 504 [M+H]

### EXAMPLE 127

### 2-(dimethylamino)ethyl 7-(3-{[3-(trifluoromethyl)benzoyl]amino}phenyl)pyrazolo[1,5-a]pyrimidine-3-carboxylate

MS (electrospray): m/z 498 [M+H]

### EXAMPLE 128

### 2-methoxyethyl 7-(3-{[3-(trifluoromethyl)benzoyl]amino}phenyl)pyrazolo[1,5-a]pyrimidine-3-carboxylate

MS (electrospray): m/z 483 [M+H]

Examples 129-134 are following the method for Example 1 using 5-pyridin-4-yl-2H-pyrazol-3-ylamine in Step 2 and reacting with different acid chlorides or isocyanates in Step 4.

### EXAMPLE 129

### 4-chloro-N-[3-(2-pyridin-4-ylpyrazolo[1,5-a]pyrimidin-7-yl)phenyl]-3-(trifluoromethyl)benzamide

MS (electrospray): m/z 494 [M+H]

### EXAMPLE 130

### 4-methoxy-N-[3-(2-pyridin-4-ylpyrazolo[1,5-a]pyrimidin-7-yl)phenyl]-3-(trifluoromethyl)benzamide

MS (electrospray): m/z 490 [M+H]

### EXAMPLE 131

### N-[4-fluoro-3-(trifluoromethyl)phenyl]-N′-[3-(2-pyridin-4-ylpyrazolo[1,5-a]pyrimidin-7-yl)phenyl]urea

MS (electrospray): m/z 493 [M+H]

### EXAMPLE 132

### N-[3-(2-pyridin-4-ylpyrazolo[1,5-a]pyrimidin-7-yl)phenyl]-N′-[3-(trifluoromethyl)phenyl]urea

MS (electrospray): m/z 475 [M+H]

### EXAMPLE 133

### N-[4-chloro-3-(trifluoromethyl)phenyl]-N′-[3-(2-pyridin-4-ylpyrazolo[1,5-a]pyrimidin-7-yl)phenyl]urea

MS (electrospray): m/z 509 [M+H]

### EXAMPLE 134

### 4-methyl-N-[3-(2-pyridin-4-ylpyrazolo[1,5-a]pyrimidin-7-yl)phenyl]-3-(trifluoromethyl)benzamide

MS (electrospray): m/z 474 [M+H]

Examples 135-142 are prepared following the above method for Example 58 using the appropriate amino pyrazoles at the final step.

### EXAMPLE 135

### N-(3-{2-[4-(hydroxymethyl)phenyl]pyrazolo[1,5-a]pyrimidin-7-yl}phenyl)-3-(trifluoromethyl)benzamide

MS (electrospray): m/z 487 [M+H]

### EXAMPLE 136

### N-{3-[2-(4-methoxyphenyl)pyrazolo[1,5-a]pyrimidin-7-yl]phenyl}-3-(trifluoromethyl)benzamide

MS (electrospray): m/z 487 [M+H]

### EXAMPLE 137

### 3-(trifluoromethyl)-N-(3-{2-[3-(trifluoromethyl)phenyl]pyrazolo[1,5-a]pyrimidin-7-yl}phenyl)benzamide

MS (electrospray): m/z 525 [M+H]

### EXAMPLE 138

### N-[3-(2-tert-butylpyrazolo[1,5-a]pyrimidin-7-yl)phenyl]-3-(trifluoromethyl)benzamide

MS (electrospray): m/z 437 [M+H]

### EXAMPLE 139

### N-{3-[2-(4-fluorophenyl)pyrazolo[1,5-a]pyrimidin-7-yl]phenyl}-3-(trifluoromethyl)benzamide

MS (electrospray): m/z 475 [M+H]

### EXAMPLE 140

### N-[3-(2-{4-[(ethoxymethoxy)methyl]phenyl}pyrazolo[1,5-a]pyrimidin-7-yl)phenyl]-3-(trifluoromethyl)benzamide

MS (electrospray): m/z 545 [M+H]

### EXAMPLE 141

### methyl 3-[7-(3-{[3-(trifluoromethyl)benzoyl]amino}phenyl)pyrazolo[1,5-a]pyrimidin-2-yl]benzoate

MS (electrospray): m/z 515 [M+H]

### EXAMPLE 142

### 4-[7-(3-{[3-(trifluoromethyl)benzoyl]amino}phenyl)pyrazolo[1,5-a]pyrimidin-2-yl]benzyl acetate

MS (electrospray): m/z 504.5 pos mode 2.71 [M+H}

Example 143 is made following the method for Example 1 using 4-Bromo-2H-pyrazol-3-ylamine in Step 2 and reacting with different acid chlorides or isocyantes in Step 4.

### EXAMPLE 143

### N-[3-(3-bromopyrazolo[1,5-a]pyrimidin-7-yl)phenyl]-N′-[4-chloro-3-(trifluoromethyl)phenyl]urea

MS (electrospray): m/z 508 [M+H]

Examples 144-152 are prepared following the method for Example 1 using 5-pyridin-4-yl-2H-pyrazol-3-ylamine in Step 2 and reacting with different acid chlorides or isocyanates in Step 4.

### EXAMPLE 144

### N-[3-(2-pyridin-4-ylpyrazolo[1,5-a]pyrimidin-7-yl)phenyl]benzamide

MS (electrospray): m/z 390 [M+H]

### EXAMPLE 145

### 2-chloro-N-[3-(2-pyridin-4-ylpyrazolo[1,5-a]pyrimidin-7-yl)phenyl]benzamide

MS (electrospray): m/z 424.1 [M+H]

### EXAMPLE 146

### 3-methoxy-N-[3-(2-pyridin-4-ylpyrazolo[1,5-a]pyrimidin-7-yl)phenyl]benzamide

MS (electrospray): m/z 420.2 [M+H]

### EXAMPLE 147

3-methyl-N-[3-(2-pyridin-4-ylpyrazolo[1,5-a]pyrimidin-7-yl)phenyl]benzamide

### EXAMPLE 148

### 4-methyl-N-[3-(2-pyridin-4-ylpyrazolo[1,5-a]pyrimidin-7-yl)phenyl]benzamide

MS (electrospray): m/z 404.2 [M+H]

### EXAMPLE 149

### 3,4-dichloro-N-[3-(2-pyridin-4-ylpyrazolo[1,5-a]pyrimidin-7-yl)phenyl]benzamide

MS (electrospray): m/z 458.1 [M+H]

### EXAMPLE 150

### 3-chloro-N-[3-(2-pyridin-4-ylpyrazolo[1,5-a]pyrimidin-7-yl)phenyl]benzamide

MS (electrospray): m/z 424.1 [M+H]

### EXAMPLE 151

### 4-chloro-N-[3-(2-pyridin-4-ylpyrazolo[1,5-a]pyrimidin-7-yl)phenyl]benzamide

MS (electrospray): m/z 424.1 [M+H]

### EXAMPLE 152

### N-[3-(2-pyridin-3-ylpyrazolo[1,5-a]pyrimidin-7-yl)phenyl]-3-(trifluoromethyl)benzamide

MS (electrospray): m/z 458.1 [M+H]

Example 153 is prepared following the above method for Example 58 using the appropriate amino pyrazoles at the final step.

### EXAMPLE 153

### N-(3-pyrazolo[1,5-a]pyrimidin-7-ylphenyl)-3-(trifluoromethyl)benzamide

MS (electrospray): m/z 381.1 [M+H]

### EXAMPLE 154

### N-[3-(3-pyridin-4-ylpyrazolo[1,5-a]pyrimidin-7-yl)phenyl]-3-(trifluoromethyl)benzamide

To a solution of N-[3-(3-Bromo-pyrazolo[1,5-a]pyrimidin-7-yl)-phenyl]-3-trifluoromethyl-benzamide (50 mg, 0.11 mmol) in DME is added pyridine-3-boronic acid (26 mg, 0.21 mmol), tetrakis (triphenylphosphine) palladium (0) (20 mg, 0.017 mmol), sodium carbonate (0.5 mL 2M). After microwaving at 175° C. for 1000 seconds, the solution is concentrated down and purified by flash chromatography (Hexane:Etoac)

MS (electrospray): m/z 460.1 [M+H]

Examples 155-160 are prepared following the above method for Example 154 using the appropriate substituted boronic acids.

### EXAMPLE 155

### N-[3-(3-pyridin-3-ylpyrazolo[1,5-a]pyrimidin-7-yl)phenyl]-3-(trifluoromethyl)benzamide

MS (electrospray): m/z 458.1 [M+H]

### EXAMPLE 156

### N-{3-[3-(3-aminophenyl)pyrazolo[1,5-a]pyrimidin-7-yl]phenyl}-3-(trifluoromethyl)benzamide

MS (electrospray): m/z 472.1 [M+H]

### EXAMPLE 157

### N-(3-{3-[4-(dimethylamino)phenyl]pyrazolo[1,5-a]pyrimidin-7-yl}phenyl)-3-(trifluoromethyl)benzamide

MS (electrospray): m/z 500.2 [M+H]

### EXAMPLE 158

### N-{3-[3-(4-chlorophenyl)pyrazolo[1,5-a]pyrimidin-7-yl]phenyl}-3-(trifluoromethyl)benzamide

MS (electrospray): m/z 493.5 [M+H]

### EXAMPLE 159

### N-{3-[3-(4-methoxyphenyl)pyrazolo[1,5-a]pyrimidin-7-yl]phenyl}-3-(trifluoromethyl)benzamide

MS (electrospray): m/z 317.5 [M+H]

### EXAMPLE 160

### N-{3-[3-(4-methylphenyl)pyrazolo[1,5-a]pyrimidin-7-yl]phenyl}-3-(trifluoromethyl)benzamide

MS (electrospray): m/z 471.2 [M+H]

Examples 161-168 are made following the method for Example 1 using 4-Bromo-2H-pyrazol-3-ylamine in Step 2 and reacting with different acid chlorides or isocyantes in Step 4.

### EXAMPLE 161

### N-[3-(3-bromopyrazolo[1,5-a]pyrimidin-7-yl)phenyl]-4-methyl-3-(trifluoromethyl)benzamide

MS (electrospray): m/z 473 [M+H]

### EXAMPLE 162

### N-[3-(3-bromopyrazolo[1,5-a]pyrimidin-7-yl)phenyl]-4-methoxy-3-(trifluoromethyl)benzamide

MS (electrospray): m/z 489 [M+H]

### EXAMPLE 163

### N-[3-(3-bromopyrazolo[1,5-a]pyrimidin-7-yl)phenyl]-4-fluoro-3-(trifluoromethyl)benzamide

MS (electrospray): m/z 477 [M+H]

### EXAMPLE 164

### N-[3-(3-bromopyrazolo[1,5-a]pyrimidin-7-yl)phenyl]-4-chloro-3-(trifluoromethyl)benzamide

MS (electrospray): m/z 493 [M+H]

### EXAMPLE 165

### N-[3-(3-bromopyrazolo[1,5-a]pyrimidin-7-yl)phenyl]-3,4-dichlorobenzamide

MS (electrospray): m/z 458.9 [M+H]

### EXAMPLE 166

### N-[3-(3-bromopyrazolo[1,5-a]pyrimidin-7-yl)phenyl]-N′-[3-(trifluoromethyl)phenyl]urea

MS (electrospray): m/z 474 [M+H]

### EXAMPLE 167

### N-[3-(3-bromopyrazolo[1,5-a]pyrimidin-7-yl)phenyl]-N′-[4-fluoro-3-(trifluoromethyl)phenyl]urea

MS (electrospray): m/z 492 [M+H]

### EXAMPLE 168

### N-[3-(3-bromopyrazolo[1,5-a]pyrimidin-7-yl)phenyl]-N′-(3,4-dichlorophenyl)urea

MS (electrospray): m/z 474 [M+H]

Examples 169-175 are prepared from 3-(3-Pyridin-3-yl-pyrazolo[1,5-a]pyrimidin-7-yl)-phenylamine using different acid chlorides and isocyanates.

### EXAMPLE 169

### 4-methyl-N-[3-(3-pyridin-3-ylpyrazolo[1,5-a]pyrimidin-7-yl)phenyl]-3-(trifluoromethyl)benzamide

MS (electrospray): m/z 474.1 [M+H]

### EXAMPLE 170

### 4-methoxy-N-[3-(3-pyridin-3-ylpyrazolo[1,5-a]pyrimidin-7-yl)phenyl]-3-(trifluoromethyl)benzamide

MS (electrospray): m/z 488.1 [M+H]

### EXAMPLE 171

### 4-fluoro-N-[3-(3-pyridin-3-ylpyrazolo[1,5-a]pyrimidin-7-yl)phenyl]-3-(trifluoromethyl)benzamide

MS (electrospray): m/z 476.1 [M+H]

### EXAMPLE 172

### 4-chloro-N-[3-(3-pyridin-3-ylpyrazolo[1,5-a]pyrimidin-7-yl)phenyl]-3-(trifluoromethyl)benzamide

MS (electrospray): m/z 492.1 [M+H]

### EXAMPLE 173

### N-[3-(3-pyridin-3-ylpyrazolo[1,5-a]pyrimidin-7-yl)phenyl]-N′-[3-(trifluoromethyl)phenyl]urea

MS (electrospray): m/z 475.1 [M+H]

### EXAMPLE 174

### N-[4-chloro-3-(trifluoromethyl)phenyl]-N′-[3-(3-pyridin-3-ylpyrazolo[1,5-a]pyrimidin-7-yl)phenyl]urea

MS (electrospray): m/z 507.1 [M+H]

### EXAMPLE 175

### N-(3,4-dichlorophenyl)-N′-[3-(3-pyridin-3-ylpyrazolo[1,5-a]pyrimidin-7-yl)phenyl]urea

MS (electrospray): m/z 473.1 [M+H]

Examples 176-179 are prepared following the procedure for example 1 using 1-(3-Nitro-phenyl)-propan-1-one in Step 1.

### EXAMPLE 176

### ethyl 6-methyl-7-(3-{[3-(trifluoromethyl)benzoyl]amino}phenyl)pyrazolo[1,5-a]pyrimidine-3-carboxylate

MS (electrospray): m/z 467.1 [M+H]

### EXAMPLE 177

### N-[3-(6-methyl-2-pyridin-4-ylpyrazolo[1,5-a]pyrimidin-7-yl)phenyl]-3-(trifluoromethyl)benzamide

MS (electrospray): m/z 472.1 [M+H]

### EXAMPLE 178

### N-[3-(3-bromo-6-methylpyrazolo[1,5-a]pyrimidin-7-yl)phenyl]-3-(trifluoromethyl)benzamide

MS (electrospray): m/z 473 [M+H]

### EXAMPLE 179

### N-[3-(6-methyl-3-pyridin-3-ylpyrazolo[1,5-a]pyrimidin-7-yl)phenyl]-3-(trifluoromethyl)benzamide

MS (electrospray): m/z 472.1 [M+H]

Examples 180-184 are prepared following the above method for Example 154 using the appropriate substituted boronic acids.

### EXAMPLE 180

### N-{3-[3-(4-aminophenyl)pyrazolo[1,5-a]pyrimidin-7-yl]phenyl}-4-methyl-3-(trifluoromethyl)benzamide

MS (electrospray): m/z 486.2 [M+H]

### EXAMPLE 181

### N-{3-[3-(3-hydroxyphenyl)pyrazolo[1,5-a]pyrimidin-7-yl]phenyl}-4-methyl-3-(trifluoromethyl)benzamide

MS (electrospray): m/z 487.1 [M+H]

### EXAMPLE 182

### N-{3-[3-(3-cyanophenyl)pyrazolo[1,5-a]pyrimidin-7-yl]phenyl}-4-methyl-3-(trifluoromethyl)benzamide

MS (electrospray): m/z 496.1 [M+H]

### EXAMPLE 183

### N-[3-(3-{3-[(dimethylamino)carbonyl]phenyl}pyrazolo[1,5-a]pyrimidin-7-yl)phenyl]-4-methyl-3-(trifluoromethyl)benzamide

MS (electrospray): m/z 542.2 [M+H]

### EXAMPLE 184

### N-(3-{3-[4-(acetylamino)phenyl]pyrazolo[1,5-a]pyrimidin-7-yl}phenyl)-4-methyl-3-(trifluoromethyl)benzamide

MS (electrospray): m/z 528.2 [M+H]

Examples 185-187 are prepared following the procedure for example 58 using various substituted amino pyrazoles.

### EXAMPLE 185

ethyl 7-(3-{[3-(trifluoromethyl)benzoyl]amino}phenyl)pyrazolo[1,5-a]pyrimidine-2-carboxylate

MS (electrospray): m/z 453.1 [M+H]

### EXAMPLE 186

### N-[3-(2-morpholin-4-ylpyrazolo[1,5-a]pyrimidin-7-yl)phenyl]-3-(trifluoromethyl)benzamide

MS (electrospray): m/z 468.2 [M+H]

### EXAMPLE 187

### N-{3-[2-(dimethylamino)pyrazolo[1,5-a]pyrimidin-7-yl]phenyl}-3-(trifluoromethyl)benzamide

MS (electrospray): m/z 426.1 [M+H]

Examples 188 to 193 are prepared following the method for Example 1 using 3-(3-(pyridin-4-yl)pyrazolo[1,5-a]pyrimidin-7-yl)aniline and reacting with different acid chlorides or isocyanates in Step 4.

### EXAMPLE 188

### 4-methyl-N-[3-(3-pyridin-4-ylpyrazolo[1,5-a]pyrimidin-7-yl)phenyl]-3-(trifluoromethyl)benzamide

MS (electrospray): m/z 474.1 [M+H]

### EXAMPLE 189

### 4-methoxy-N-[3-(3-pyridin-4-ylpyrazolo[1,5-a]pyrimidin-7-yl)phenyl]-3-(trifluoromethyl)benzamide

MS (electrospray): m/z 490.1 [M+H]

### EXAMPLE 190

### 4-fluoro-N-[3-(3-pyridin-4-ylpyrazolo[1,5-a]pyrimidin-7-yl)phenyl]-3-(trifluoromethyl)benzamide

MS (electrospray): m/z 478.1 [M+H]

### EXAMPLE 191

### 4-chloro-N-[3-(3-pyridin-4-ylpyrazolo[1,5-a]pyrimidin-7-yl)phenyl]-3-(trifluoromethyl)benzamide

MS (electrospray): m/z 494.1 [M+H]

### EXAMPLE 192

N-[3-(3-pyridin-4-ylpyrazolo[1,5-a]pyrimidin-7-yl)phenyl]-N′-[3-(trifluoromethyl)phenyl]urea

### EXAMPLE 193

N-[4-fluoro-3-(trifluoromethyl)phenyl]-N′-[3-(3-pyridin-4-ylpyrazolo[1,5-a]pyrimidin-7-yl)phenyl]urea

### EXAMPLES 194-202

Step 1: 3-morpholino-1H-pyrazol-5-amine is prepared following the procedure for example 203 using morpholine in the place of N-methyl-piperazine in step 3.

Step 2: The above pyrazole is further condensed with 3-Dimethylamino-1-(3-nitro-phenyl)-propenone as detailed in example 1, reduced and reacted with the required acid chlorides or isocyanates in the final step.

### EXAMPLE 194

### 4-methyl-N-[3-(2-morpholin-4-ylpyrazolo[1,5-a]pyrimidin-7-yl)phenyl]-3-(trifluoromethyl)benzamide

MS (electrospray): m/z 482.2 [M+H]

### EXAMPLE 195

### 4-methoxy-N-[3-(2-morpholin-4-ylpyrazolo[1,5-a]pyrimidin-7-yl)phenyl]-3-(trifluoromethyl)benzamide

MS (electrospray): m/z 498.2 [M+H]

### EXAMPLE 196

### 4-fluoro-N-[3-(2-morpholin-4-ylpyrazolo[1,5-a]pyrimidin-7-yl)phenyl]-3-(trifluoromethyl)benzamide

MS (electrospray): m/z 486.1 [M+H]

### EXAMPLE 197

### 4-chloro-N-[3-(2-morpholin-4-ylpyrazolo[1,5-a]pyrimidin-7-yl)phenyl]-3-(trifluoromethyl)benzamide

MS (electrospray): m/z 502.1 [M+H]

### EXAMPLE 198

### 3,4-dichloro-N-[3-(2-morpholin-4-ylpyrazolo[1,5-a]pyrimidin-7-yl)phenyl]benzamide

MS (electrospray): m/z 466.1 [M+H]

### EXAMPLE 199

### N-[3-(2-morpholin-4-ylpyrazolo[1,5-a]pyrimidin-7-yl)phenyl]-N′-[3-(trifluoromethyl)phenyl]urea

MS (electrospray): m/z 483.2 [M+H]

### EXAMPLE 200

### N-[4-chloro-3-(trifluoromethyl)phenyl]-N′-[3-(2-morpholin-4-ylpyrazolo[1,5-a]pyrimidin-7-yl)phenyl]urea

MS (electrospray): m/z 517.1 [M+H]

### EXAMPLE 201

### N-(3,4-dichlorophenyl)-N′-[3-(2-morpholin-4-ylpyrazolo[1,5-a]pyrimidin-7-yl)phenyl]urea

MS (electrospray): m/z 483.1 [M+H]

### EXAMPLE 202

### N-[4-fluoro-3-(trifluoromethyl)phenyl]-N′-[3-(2-morpholin-4-ylpyrazolo[1,5-a]pyrimidin-7-yl)phenyl]urea

MS (electrospray): m/z 501.2 [M+H]

### EXAMPLE 203

### N-{3-[2-(4-methylpiperazin-1-yl)pyrazolo[1,5-a]pyrimidin-7-yl]phenyl}-3-(trifluoromethyl)benzamide

Step 1: A 250 mL three-necked round-bottom flask equipped with magnetic stirrer, condenser and septum is charged with a solution of cyano-acetic acid ethyl ester (11.3 g, 100 mmol) in 100 mL of DMF. Dried potassium carbonate (13.8 g, 100 mmol) is added and the mixture is stirred at room temperature for 2 hours. Carbon disulfide (18.0 mL, 300 mL) is added and the mixture is stirred at room temperature for another 2 hours. Methyl iodide (12.5 mL, 200 mmol) is then added and the mixture is stirred for another 4 hours. The reaction mixture is poured into 400 mL of water. The precipitate is collected by filtration. 2-Cyano-3,3-bis-methylsulfanyl-acrylic acid ethyl ester (18.5 g, 85% yield) is isolated by crystallization from EtOH/H₂O (3:1).

¹H NMR (CDCl₃) δ 4.2 (q, 2H), 2.7 (s, 3H), 2.6 (s, 3H), 1.3 (t, 3H)

Step 2: To 2-cyano-3,3-bis-methylsulfanyl-acrylic acid ethyl ester (10.2 g, 47 mmol) in 25 mL of THF is slowly added 1N sodium hydroxide (50 mL, 50 mmol). The reaction mixture is stirred at room temperature for 2 hours and then is concentrated to remove most of THF. The resulting aqueous solution is washed with 100 mL of EtOAc. Then the aqueous layer is collected and cooled to 0° C. Then 2N HCl is slowly added and a precipitate is formed. 2-Cyano-3,3-bis-methylsulfanyl-acrylic acid (2.3 g, 26% yield) is isolated by filtration.

Step 3: To 2-Cyano-3,3-bis-methylsulfanyl-acrylic acid (1.7 g, 9.0 mmol) in 13 mL of methanol is added N-methyl-piperazine (1.6 g, 16 mmol) and triethylamine (1.3 mL, 9.0 mmol). The reaction mixture is stirred at 25° C. over night. The reaction mixture is concentrated and purified by flash chromatography (eluting with 0-20% MeOH/CH₂Cl₂) to give 3-(4-Methyl-piperazin-1-yl)-3-methylsulfanyl-acrylonitrile (1.3 g, 73% yield).

HPLC: Rt=0.16 min; MS 198 [M+H]

¹H NMR (CDCl₃) δ 4.28 (s, 1H), 3.36 (m, 4H), 2.46 (m, 4H), 2.35 (s, 3H), 2.30 (s, 3H).

Step 4: A mixture of 3-(4-Methyl-piperazin-1-yl)-3-methylsulfanyl-acrylonitrile (0.33 g, 1.7 mmol) and 2 mL of hydrazine in 5 mL of ethanol is refluxed for 24 hours. Then the reaction mixture is concentrated to give 5-(4-Methyl-piperazin-1-yl)-2H-pyrazol-3-ylamine (0.30 g), which is directly used in the next step without further purification.

HPLC: Rt=0.16 min; MS 182 [M+H]

Step 5: A mixture of N-[3-(3-dimethylamino-acryloyl)-phenyl]-3-trifluoromethyl-benzamide (40 mg, 0.22 mmol) and 5-(4-Methyl-piperazin-1-yl)-2H-pyrazol-3-ylamine (37 mg, 0.20 mmol) in 2 mL of acetic acid is heated at 80° C. over night. Then the reaction mixture is concentrated and diluted with ethyl acetate. N-{3-[2-(4-methylpiperazin-1-yl)pyrazolo[1,5-a]pyrimidin-7-yl]phenyl}-3-(trifluoromethyl)benzamide (67.3 mg, 70% yield) is obtained by reverse phase chromatography purification.

HPLC: Rt=2.0 min; MS 481 [M+H]

MS (electrospray): m/z 481.2 [M+H]

Example 204 is prepared following the method for Example 1 using the appropriate acid chloride at the final step.

### EXAMPLE 204

### ethyl 7-{3-[(pyridin-3-ylcarbonyl)amino]phenyl}pyrazolo[1,5-a]pyrimidine-3-carboxylate

MS (electrospray): m/z 499.5 pos 2.32

### EXAMPLE 205

### N-(3-{3-[3-(dimethylamino)prop-1-yn-1-yl]pyrazolo[1,5-a]pyrimidin-7-yl}phenyl)-3-(trifluoromethyl)benzamide

Step 1: A mixture of 3-aminoacetophenone (3.0 g, 22 mmol), 3-trifluoromethyl-benzoyl chloride (4.5 g, 22 mmol) and pyridine (3.5 mL, 43 mmol) in 25 mL of methylene chloride is stirred at room temperature for 2 hours. The reaction mixture is diluted with 200 mL of methylene and washed with 50 mL of 2N HCl and 100 mL of brine. Then collected organic layer is dried over sodium sulfate and concentrated to give N-(3-ccetyl-phenyl)-3-trifluoromethyl-benzamide (6.7 g, 100% yield), which is used in the next step without further purification.

HPLC: Rt=2.6 min; MS 308 [M+H]

Step 2: A mixture of N-(3-ccetyl-phenyl)-3-trifluoromethyl-benzamide (6.7 g, 22 mmol) in 15 mL of DFM-DMA was heated at 60° C. for 20 hours. LC/MS shows that the reaction is completed. The reaction mixture is concentrated to give N-[3-(3-dimethylamino-acryloyl)-phenyl]-3-trifluoromethyl-benzamide (7.9 g) as a yellow solid.

The product is used in the next step without further purification.

Step 3: A mixture of N-[3-(3-dimethylamino-acryloyl)-phenyl]-3-trifluoromethyl-benzamide (3.9 g, 11 mmol) and 5-bromo-2H-pyrazol-3-ylamine (1.9 g, 12 mmol) in 30 mL of acetic acid is heated at 80° C. over night. The reaction mixture is concentrated and diluted with ethyl acetate. The organic solution is washed with saturated sodium bicarbonate and brine. The collected organic layer is concentrated and purified by flash chromatography (120 g silica gel column, eluting with 0-30% EtOAc/Hexane) to give N-[3-(3-bromo-pyrazolo[1,5-a]pyrimidin-7-yl)-phenyl]-3-trifluoromethyl-benzamide (3.1 g, 60% yield)

HPLC: Rt=2.9 min; MS 461, 463 [M+H]

Step 4: A mixture of N-[3-(3-bromo-pyrazolo[1,5-a]pyrimidin-7-yl)-phenyl]-3-trifluoromethyl-benzamide (46 mg, 0.10 mmol), dimethyl-prop-2-ynyl-amine (8.3 mg, 0.1 mmol), tetrakis (triphenylphosphine) palladium (6 mg, 0.005 mmol) and copper (I) iodide (2 mg, 0.010 mmol) in 2 mL of triethylamine is stirred at 80° C. for 16 hours.

Then the reaction mixture is concentrated and purified by reverse phase chromatography to give N-(3-{3-[3-(dimethylamino)prop-1-yn-1-yl]pyrazolo[1,5-a]pyrimidin-7-yl}phenyl)-3-(trifluoromethyl)benzamide (12 mg, 27% yield).

MS (electrospray): m/z 464 [M+H]

Examples 206-213, are prepared following the procedure described for example 203 by using corresponding starting materials

### EXAMPLE 206

### tert-butyl 4-[7-(3-{[3-(trifluoromethyl)benzoyl]amino}phenyl)pyrazolo[1,5-a]pyrimidin-2-yl]piperazine-1-carboxylate

MS (electrospray): m/z 567.2 [M+H]

### EXAMPLE 207

### N-{3-[2-(4-benzylpiperazin-1-yl)pyrazolo[1,5-a]pyrimidin-7-yl]phenyl}-3-(trifluoromethyl)benzamide

MS (electrospray): m/z 557.2 [M+H]

### EXAMPLE 208

### N-[3-(2-piperazin-1-ylpyrazolo[1,5-a]pyrimidin-7-yl)phenyl]-3-(trifluoromethyl)benzamide

MS (electrospray): m/z 467.2 [M+H]

### EXAMPLE 209

### N-(3-{2-[3-(dimethylamino)pyrrolidin-1-yl]pyrazolo[1,5-a]pyrimidin-7-yl}phenyl)-3-(trifluoromethyl)benzamide

MS (electrospray): m/z 495.2 [M+H]

### EXAMPLE 210

### N-(3-{2-[(2R)-2-(methoxymethyl)pyrrolidin-1-yl]pyrazolo[1,5-a]pyrimidin-7-yl}phenyl)-3-(trifluoromethyl)benzamide

MS (electrospray): m/z 496.2 [M+H]

### EXAMPLE 211

### N-(3-{2-[(2S)-2-(pyrrolidin-1-ylmethyl)pyrrolidin-1-yl]pyrazolo[1,5-a]pyrimidin-7-yl}phenyl)-3-(trifluoromethyl)benzamide

MS (electrospray): m/z 535.2 [M+H]

### EXAMPLE 212

### tert-butyl {1-[7-(3-{[3-(trifluoromethyl)benzoyl]amino}phenyl)pyrazolo[1,5-a]pyrimidin-2-yl]pyrrolidin-3-yl}carbamate

MS (electrospray): m/z 567.2 [M+H]

### EXAMPLE 213

### N-{3-[2-(3-aminopyrrolidin-1-yl)pyrazolo[1,5-a]pyrimidin-7-yl]phenyl}-3-(trifluoromethyl)benzamide

MS (electrospray): m/z 467.2 [M+H]

Examples 214-228 are prepared following the procedure described for example 1 by using corresponding acids or acid chlorides in the final step.

### EXAMPLE 214

### ethyl7-{3-[(pyrazin-2-ylcarbonyl)amino]phenyl}pyrazolo[1,5-a]pyrimidine-3-carboxylate

MS (electrospray): m/z 389.1 [M+H]

### EXAMPLE 215

### ethyl7-(3-{[(1-methyl-1H-pyrrol-2-yl)carbonyl]amino}phenyl)pyrazolo[1,5-a]pyrimidine-3-carboxylate

MS (electrospray): m/z 390.1 [M+H]

### EXAMPLE 216

### Ethyl7-(3-{[(5-methylpyrazin-2-yl)carbonyl]amino}phenyl)pyrazolo[1,5-a]pyrimidine-3-carboxylate

MS (electrospray): m/z 403.1 [M+H]

### EXAMPLE 217

### ethyl7-(3-{[(4-chloropyridin-2-yl)carbonyl]amino}phenyl)pyrazolo[1,5-a]pyrimidine-3-carboxylate

MS (electrospray): m/z 422.1 [M+H]

### EXAMPLE 218

### ethyl7-{3-[(isoquinolin-1-ylcarbonyl)amino]phenyl}pyrazolo[1,5-a]pyrimidine-3-carboxylate

MS (electrospray): m/z 438.1 [M+H]

### EXAMPLE 219

### ethyl 7-(3-{[(1-methyl-1H-indol-2-yl)carbonyl]amino}phenyl)pyrazolo[1,5-a]pyrimidine-3-carboxylate

MS (electrospray): m/z 440.2 [M+H]

### EXAMPLE 220

ethyl7-(3-{[(5-methyl-2-phenyl-2H-1,2,3-triazol-4-yl)carbonyl]amino}phenyl)pyrazolo[1,5-a]pyrimidine-3-carboxylate

### EXAMPLE 221

### ethyl7-(3-{[(5-methyl-2-thienyl)carbonyl]amino}phenyl)pyrazolo[1,5-a]pyrimidine-3-carboxylate

MS (electrospray): m/z 407.1 [M+H]

### EXAMPLE 222

### ethyl 7-(3-{[(5-chloro-2-thienyl)carbonyl]amino}phenyl)pyrazolo[1,5-a]pyrimidine-3-carboxylate

MS (electrospray): m/z 427.1 [M+H]

### EXAMPLE 223

ethyl7-(3-{[(5-bromo-2-thienyl)carbonyl]amino}phenyl)pyrazolo[1,5-a]pyrimidine-3-carboxylate

### EXAMPLE 224

### ethyl7-{3-[({5-[1-methyl-3-(trifluoromethyl)-1H-pyrazol-5-yl]-2-thienyl}carbonyl)amino]phenyl}pyrazolo[1,5-a]pyrimidine-3-carboxylate

MS (electrospray): m/z 541.1 [M+H]

### EXAMPLE 225

### ethyl7-{3-[(3,3-dimethylbutanoyl)amino]phenyl}pyrazolo[1,5-a]pyrimidine-3-carboxylate

MS (electrospray): m/z 381.2 [M+H]

### EXAMPLE 226

### ethyl7-{3-[(3,5,5-trimethylhexanoyl)amino]phenyl}pyrazolo[1,5-a]pyrimidine-3-carboxylate

MS (electrospray): m/z 423.2 [M+H]

### EXAMPLE 227

### ethyl7-{3-[(3,5-di-tert-butylbenzoyl)amino]phenyl}pyrazolo[1,5-a]pyrimidine-3-carboxylate

MS (electrospray): m/z 499.3 [M+H]

### EXAMPLE 228

### ethyl7-{3-[(2-bromo-5-chlorobenzoyl)amino]phenyl}pyrazolo[1,5-a]pyrimidine-3-carboxylate

MS (electrospray): m/z 499 [M+H]

Examples 229-235 are prepared from N-{3-[3-(4-Amino-phenyl)-pyrazolo[1,5-a]pyrimidin-7-yl]-phenyl}-3-trifluoromethyl-benzamide (prepared following the procedure for example 154) and different acid chlorides or acids.

### EXAMPLE 229

### N-[3-(3-{4-[(methoxyacetyl)amino]phenyl}pyrazolo[1,5-a]pyrimidin-7-yl)phenyl]-3-(trifluoromethyl)benzamide

MS (electrospray) m/z 544.2 [M+H]

### EXAMPLE 230

### N-[3-(3-{4-[(N,N-dimethylglycyl)amino]phenyl}pyrazolo[1,5-a]pyrimidin-7-yl)phenyl]-3-(trifluoromethyl)benzamide

MS (electrospray): m/z 557.2 [M+H]

### EXAMPLE 231

### N-[3-(3-{4-[(3-methoxypropanoyl)amino]phenyl}pyrazolo[1,5-a]pyrimidin-7-yl)phenyl]-3-(trifluoromethyl)benzamide

MS (electrospray): m/z 558.2 [M+H]

### EXAMPLE 232

### N-[3-(3-{4-[(1H-imidazol-4-ylacetyl)amino]phenyl}pyrazolo[1,5-a]pyrimidin-7-yl)phenyl]-3-(trifluoromethyl)benzamide

MS (electrospray): m/z 582.2 [M+H]

### EXAMPLE 233

### N-[3-(3-{4-[(1H-tetrazol-5-ylacetyl)amino]phenyl}pyrazolo[1,5-a]pyrimidin-7-yl)phenyl]-3-(trifluoromethyl)benzamide

MS (electrospray): m/z 582.2 [M+H]

### EXAMPLE 234

### N-{3-[3-(4-{[4-(dimethylamino)butanoyl]amino}phenyl)pyrazolo[1,5-a]pyrimidin-7-yl]phenyl}-3-(trifluoromethyl)benzamide

MS (electrospray): m/z 587.2 [M+H]

### EXAMPLE 235

### N-{3-[3-(4-{[(2-methoxyethoxy)acetyl]amino}phenyl)pyrazolo[1,5-a]pyrimidin-7-yl]phenyl}-3-(trifluoromethyl)benzamide

MS (electrospray): m/z 588.2 [M+H]

Examples 236-248 are prepared following the procedure described for example 1 by using corresponding acids or acid chlorides in the final step.

### EXAMPLE 236

### 1-methyl-N-[3-(2-pyridin-4-ylpyrazolo[1,5-a]pyrimidin-7-yl)phenyl]-1H-pyrrole-2-carboxamide

MS (electrospray): m/z 395.2 [M+H]

### EXAMPLE 237

### N-[3-(2-pyridin-4-ylpyrazolo[1,5-a]pyrimidin-7-yl)phenyl]isoquinoline-1-carboxamide

MS (electrospray): m/z 443.2 [M+H]

### EXAMPLE 238

### 1-methyl-N-[3-(2-pyridin-4-ylpyrazolo[1,5-a]pyrimidin-7-yl)phenyl]-1H-indole-2-carboxamide

MS (electrospray): m/z 445.2 [M+H]

### EXAMPLE 239

### 5-bromo-N-[3-(2-pyridin-4-ylpyrazolo[1,5-a]pyrimidin-7-yl)phenyl]thiophene-2-carboxamide

MS (electrospray): m/z 476 [M+H]

### EXAMPLE 240

### 3,3-dimethyl-N-[3-(2-pyridin-4-ylpyrazolo[1,5-a]pyrimidin-7-yl)phenyl]butanamide

MS (electrospray): m/z 386.2 [M+H]

### EXAMPLE 241

### 2-bromo-5-chloro-N-[3-(2-pyridin-4-ylpyrazolo[1,5-a]pyrimidin-7-yl)phenyl]benzamide

MS (electrospray): m/z 504 [M+H]

### EXAMPLE 242

### ethyl 7-{3-[(3-methylbenzoyl)amino]phenyl}pyrazolo[1,5-a]pyrimidine-3-carboxylate

MS (electrospray): m/z 401.2 [M+H]

### EXAMPLE 243

### ethyl7-(3-{[(3-tert-butyl-1-methyl-1H-pyrazol-5-yl)carbonyl]amino}phenyl)pyrazolo[1,5-a]pyrimidine-3-carboxylate

MS (electrospray): m/z 447.2 [M+H]

### EXAMPLE 244

ethyl7-[3-({[(6-methoxy-1,3-benzothiazol-2-yl)amino]carbonyl}amino)phenyl]pyrazolo[1,5-a]pyrimidine-3-carboxylate

### EXAMPLE 245

### ethyl7-(3-{[(1,3-benzodioxol-5-ylamino)carbonyl]amino}phenyl)pyrazolo[1,5-a]pyrimidine-3-carboxylate

MS (electrospray): m/z 446.1 [M+H]

### EXAMPLE 246

ethyl7-[3-({[(6-chloro-1,3-benzothiazol-2-yl)amino]carbonyl}amino)phenyl]pyrazolo[1,5-a]pyrimidine-3-carboxylate

### EXAMPLE 247

### ethyl7-[3-({[(3-methylisoxazol-5-yl)amino]carbonyl}amino)phenyl]pyrazolo[1,5-a]pyrimidine-3-carboxylate

MS (electrospray): m/z 407.1 [M+H]

### EXAMPLE 248

### ethyl7-[3-({[(5-methylisoxazol-3-yl)amino]carbonyl}amino)phenyl]pyrazolo[1,5-a]pyrimidine-3-carboxylate

MS (electrospray): m/z 407.1 [M+H]

Example 249, are prepared following the procedure described for example 203 by using corresponding starting materials

### EXAMPLE 249

### N-{3-[2-(3-oxopiperazin-1-yl)pyrazolo[1,5-a]pyrimidin-7-yl]phenyl}-3-(trifluoromethyl)benzamide

MS (electrospray): m/z 481.2 [M+H]

Example 250 is prepared following the procedure described for example 203 by using 5-Amino-1H-pyrazol-3-ol instead of 5-(4-Methyl-piperazin-1-yl)-2H-pyrazol-3-ylamine in step 5.

### EXAMPLE 250

### N-[3-(2-hydroxypyrazolo[1,5-a]pyrimidin-7-yl)phenyl]-3-(trifluoromethyl)benzamide

MS (electrospray): m/z 399.1 [M+H]

### EXAMPLE 251

### N-{3-[2-(4-oxopiperidin-1-yl)pyrazolo[1,5-a]pyrimidin-7-yl]phenyl}-3-(trifluoromethyl)benzamide

To N-{3-[2-(1,4-Dioxa-8-aza-spiro[4,5]dec-8-yl)-pyrazolo[1,5-a]pyrimidin-7-yl]-phenyl}-3-trifluoromethyl-benzamide [[prepared by using the procedure described in Example 203 substituting 1,4-Dioxa-8-aza-spiro[4,5]decane for N-methyl-piperazine] (25 mg, 0.05 mmol) is added pyridinium p-toluenesulfonate (25 mg, 0.10 mmol) and 1 mL of acetone and 1 mL of water. The reaction mixture is stirred at room temperature for 5 hours and then concentrated. N-{3-[2-(4-oxopiperidin-1-yl)pyrazolo[1,5-a]pyrimidin-7-yl]phenyl}-3-(trifluoromethyl)benzamide (9.8 mg, 41% yield) is obtained by reverse phase chromatography purification.

HPLC: Rt=2.35 min; MS 480 [M+H]

MS (electrospray): m/z 480.2 [M+H]

Examples 252-256 are prepared from N-{3-[3-(3-Amino-phenyl)-pyrazolo[1,5-a]pyrimidin-7-yl]-phenyl}-3-trifluoromethyl-benzamide (prepared following the procedure for example 154) and different acid chlorides or acids.

### EXAMPLE 252

### N-[3-(3-{3-[(methoxyacetyl)amino]phenyl}pyrazolo[1,5-a]pyrimidin-7-yl)phenyl]-3-(trifluoromethyl)benzamide

MS (electrospray): m/z 546.2 [M+H]

### EXAMPLE 253

### N-[3-(3-{3-[(N,N-dimethylglycyl)amino]phenyl}pyrazolo[1,5-a]pyrimidin-7-yl)phenyl]-3-(trifluoromethyl)benzamide

MS (electrospray): m/z 557.2 [M+H]

### EXAMPLE 254

### N-[3-(3-{3-[(3-methoxypropanoyl)amino]phenyl}pyrazolo[1,5-a]pyrimidin-7-yl)phenyl]-3-(trifluoromethyl)benzamide

MS (electrospray): m/z 560.2 [M+H]

### EXAMPLE 255

### N-[3-(3-{3-[(N-acetylglycyl)amino]phenyl}pyrazolo[1,5-a]pyrimidin-7-yl)phenyl]-3-(trifluoromethyl)benzamide

MS (electrospray): m/z 573.2 [M+H]

### EXAMPLE 256

### N-[3-(3-{3-[(1H-tetrazol-5-ylacetyl)amino]phenyl}pyrazolo[1,5-a]pyrimidin-7-yl)phenyl]-3-(trifluoromethyl)benzamide

MS (electrospray): m/z 584.2 [M+H]

### EXAMPLE 257

### N-{3-[2-(2-{[3-(dimethylamino)propyl]amino}pyridin-4-yl)pyrazolo[1,5-a]pyrimidin-7-yl]phenyl}-3-(trifluoromethyl)benzamide

Step 1: A mixture of 2-chloro-isonicotinic acid (4.0 g, 25.4 mmol), sodium bicarbonate (5.33 g, 63.48 mmol) and iodomethane (9.7 mL, 156.0 mmol) in N,N dimethyl formamide (60 mL) is stirred at room temperature for 20 hours. The mixture is poured into water and extracted with ether. The organic layer is washed with brine, dried over anhydrous sodium sulfate and filtered. Evaporation of the filtrate provides an oil which solidifies on standing to yield 3.8 g (87%) of 2-chloro-isonicotinic acid methyl ester as a white solid. MS 172.0 [M+H].

Step 2: A solution of dry THF (100 ml) CH₃CN (2.1 ml, 29.2 mmol) and tBuOK (5.4 g, 43.8 mmol) is stirred at 0° C. for 5 minutes; then 2-chloro-isonicotinic acid methyl ester is added. The reaction is then stirred at room temperature for 10 minutes: TLC indicated the reaction is complete. Toluene is added and the solvent evaporated to give a mixture of 3-(2-chloro-pyridin-4-yl)-3-oxo-propionitrile and only one side product (the corresponding 2-Chloro-isonicotinic acid).

This mixture is utilized without any purification in the next step.

Step 3: To a solution of crude 3-(2-chloro-pyridin-4-yl)-3-oxo-propionitrile in Ethanol (200 ml), NH₂NH₂.H₂O (13 ml) and conc. HCl (11 ml) are added. After stirring the mixture over night at 70° C., the reaction is complete. Ethanol is evaporated, the mixture diluted with water and the product extracted with EtOAc. Side-products are water-soluble and the organic phase contained only the 5-(2-chloro-pyridin-4-yl)-2H-pyrazol-3-ylamine (3.7 g), which is recovered pure as a pale yellow solid without any further purification. (61% of yield over 3 steps)

¹H (300 MHz, DMSO-d₆): 11.93(s br, 1H); 8.34(d, 1H); 7.70(d, 1H); 7.64(dd, 1H); 5.92(s, 1H); 5.08(s br, 2H).

Step 4: A solution of N-(3-(3-(dimethylamino)acryloyl)phenyl)-3-(trifluoromethyl)benzamide (4 g, 11 mmol) in 100 ml of AcOH is stirred at room temperature for 15 minutes; 5-(2-chloro-pyridin-4-yl)-2H-pyrazol-3-ylamine (3.7 g, 11 mmol) is added and the mixture is heated on reflux for 3 h. A 2M solution of K₂CO₃ is added until pH=6, then the mixture is diluted with water, EtOAc and MeOH and the product filtered. N-{3-[2-(2-Chloro-pyridin-4-yl)-pyrazolo[1,5-a]pyrimidin-7-yl]-phenyl}-3-trifluoromethyl-benzamide is recovered pure as a white solid in 75% yield without any further purification.

¹H (300 MHz, DMSO-d₆): 10.81(s br, 1H); 8.97(dd, 1H); 8.69(d, 1H); 8.54(d, 1H); 8.40(s, 1H); 8.36(d, 1H); 8.29(s, 1H); 8.09(dd, 1H); 7.94(m, 3H); 7.81(dd, 1H); 7.64(dd, 1H); 7.64(s, 1H); 7.39(d, 1H).

Step 5: N-{3-[2-(2-Chloro-pyridin-4-yl)-pyrazolo[1,5-a]pyrimidin-7-yl]-phenyl}-3-trifluoromethyl-benzamide (200 mg, 0.41 mmol) is suspended in anhydrous pyridine (3 mL) and 3-dimethylaminopropylamine (3 mL) is added. The reaction mixture is heated at 170° C. in a microwave oven for 40 minutes, then an additional amount of amine (2 mL) is added and the reaction mixture is heated under microwave irradiation. 4/5 cycles are required to force the reaction to completion (including the addition of amine).

The solvent is evaporated, EtOAc and water are added and the organic layer is separated and dried over Na₂SO₄. After evaporation of the solvent, the crude is firstly purified on silica gel (EtOAc and EtOAc:MeOH=1:1) and then by preparative HPLC leading to the pure N-{3-[2-(2-{[3-(dimethylamino)propyl]amino}pyridin-4-yl)pyrazolo[1,5-a]pyrimidin-7-yl]phenyl}-3-(trifluoromethyl)benzamide (61 mg, 27% yield) as a yellow solid.

¹H (300 MHz, DMSO-d₆): 10.77(s, 1H); 8.78 (s br,1H); 8.71(d, 1H); 8.67(s, 1H); 8.34(s, 1H); 8.31(d, 1H); 8.09(d, 1H); 8.05-7.89(m, 3H); 7.81(dd, 1H); 7.66(dd, 1H); 7.58(s, 1H); 7.54(s,1H); 7.47(d, 1H); 7.34(d,1H); 3.45(dd, 2H); 3.14(m, 2H); 2.78(s,6H); 1.95(m,2H).

MS (electrospray): m/z 560.2 [M+H]

Examples 258-270 are prepared following the method for Example 257, by reacting N-{3-[2-(2-Chloro-pyridin-4-yl)-pyrazolo[1,5-a]pyrimidin-7-yl]-phenyl}-3-trifluoromethyl-benzamide with the corresponding amines in the final step in pyridine or NMP.

### EXAMPLE 258

### N-[3-(2-{2-[(3-morpholin-4-ylpropyl)amino]pyridin-4-yl}pyrazolo[1,5-a]pyrimidin-7-yl)phenyl]-3-(trifluoromethyl)benzamide

MS (electrospray): m/z 602.3 [M+H]

### EXAMPLE 259

### N-[3-(2-{2-[(3-piperidin-1-ylpropyl)amino]pyridin-4-yl}pyrazolo[1,5-a]pyrimidin-7-yl)phenyl]-3-(trifluoromethyl)benzamide

MS (electrospray): m/z 600.3 [M+H]

### EXAMPLE 260

### N-[3-(2-{2-[(2-morpholin-4-ylethyl)amino]pyridin-4-yl}pyrazolo[1,5-a]pyrimidin-7-yl)phenyl]-3-(trifluoromethyl)benzamide

MS (electrospray): m/z 588.3 [M+H]

### EXAMPLE 261

### N-{3-[2-(2-{[3-(2-oxopyrrolidin-1-yl)propyl]amino}pyridin-4-yl)pyrazolo[1,5-a]pyrimidin-7-yl]phenyl}-3-(trifluoromethyl)benzamide

MS (electrospray): m/z 600.3 [M+H]

### EXAMPLE 262

### N-{3-[2-(2-{[3-(1H-imidazol-1-yl)propyl]amino}pyridin-4-yl)pyrazolo[1,5-a]pyrimidin-7-yl]phenyl}-3-(trifluoromethyl)benzamide

MS (electrospray): m/z 583.3 [M+H]

### EXAMPLE 263

### N-{3-[2-(2-{[2-(4-hydroxypiperidin-1-yl)ethyl]amino}pyridin-4-yl)pyrazolo[1,5-a]pyrimidin-7-yl]phenyl}-3-(trifluoromethyl)benzamide

MS (electrospray): m/z 602.4 [M+H]

### EXAMPLE 264

### N-[3-(2-{2-[(2-piperidin-1-ylethyl)amino]pyridin-4-yl}pyrazolo[1,5-a]pyrimidin-7-yl)phenyl]-3-(trifluoromethyl)benzamide

MS (electrospray): m/z 586.3 [M+H]

### EXAMPLE 265

### N-[3-(2-{2-[(2-pyrrolidin-1-ylethyl)amino]pyridin-4-yl}pyrazolo[1,5-a]pyrimidin-7-yl)phenyl]-3-(trifluoromethyl)benzamide

MS (electrospray): m/z 572.4 [M+H]

### EXAMPLE 266

### N-{3-[2-(2-{[2-(dimethylamino)ethyl]amino}pyridin-4-yl)pyrazolo[1,5-a]pyrimidin-7-yl]phenyl}-3-(trifluoromethyl)benzamide

MS (electrospray): m/z 546.3 [M+H]

### EXAMPLE 267

### N-[3-(2-{2-[(3-pyrrolidin-1-ylpropyl)amino]pyridin-4-yl}pyrazolo[1,5-a]pyrimidin-7-yl)phenyl]-3-(trifluoromethyl)benzamide

MS (electrospray): m/z 586.4 [M+H]

### EXAMPLE 268

### N-{3-[2-(2-{[2-(2-oxoimidazolidin-1-yl)ethyl]amino}pyridin-4-yl)pyrazolo[1,5-a]pyrimidin-7-yl]phenyl}-3-(trifluoromethyl)benzamide

MS (electrospray): m/z 587.3 [M+H]

### EXAMPLE 269

### N-[3-(2-{2-[(3-aminopropyl)(methyl)amino]pyridin-4-yl}pyrazolo[1,5-a]pyrimidin-7-yl)phenyl]-3-(trifluoromethyl)benzamide

MS (electrospray): m/z 546.4 [M+H]

### EXAMPLE 270

### N-[3-(2-{2-[(2-aminoethyl)(methyl)amino]pyridin-4-yl}pyrazolo[1,5-a]pyrimidin-7-yl)phenyl]-3-(trifluoromethyl)benzamide

MS (electrospray): m/z 532.4 [M+H]

### EXAMPLE 271

### N-(3-{3-[3-(aminocarbonyl)phenyl]pyrazolo[1,5-a]pyrimidin-7-yl}phenyl)-3-(trifluoromethyl)benzamide

Step 1: To N-[3-(3-bromo-pyrazolo[1,5-a]pyrimidin-7-yl)-phenyl]-3-trifluoromethyl-benzamide (500 mg, 1.08 mmol), Pd(PPh₃)₄ (63 mg, 0.054 mmol), K₂CO₃ (2M solution, 1.6 ml, 3.2 mmol) and DME (5 ml) are added. The mixture is stirred at room temperature for 30 minutes, and then 3-ethoxycarbonylphenylboronic acid (316 mg, 1.63 mmol) is added. The reaction is heated at 85° C. overnight, then DME is evaporated. The residue is diluted with water and extracted with CH₂Cl₂+5% MeOH. The obtained crude is purified by a silica gel column chromatography (eluent: gradient from CH₂Cl₂ to CH₂Cl₂-MeOH 96:4). 3-{7-[3-(3-Trifluoromethyl-benzoylamino)-phenyl]-pyrazolo[1,5-a]pyrimidin-3-yl}-benzoic acid ethyl ester is used in next step without further purification.

Step 2: To 3-{7-[3-(3-trifluoromethyl-benzoylamino)-phenyl]-pyrazolo[1,5-a]pyrimidin-3-yl}-benzoic acid ethyl ester (1.08 mmol), NaOH (excess) and EtOH are added. The resulting mixture is stirred at room temperature until disappearance of the starting material; then the solvent is evaporated. The residue is suspended in Et₂O saturated with HCl and stirred at room temperature for 15 minutes. The solvent is evaporated affording a crude product that is purified by a silica gel column chromatography (eluent: gradient from AcOEt to AcOEt-MeOH 10:1). 3-{7-[3-(3-Trifluoromethyl-benzoylamino)-phenyl]-pyrazolo[1,5-a]pyrimidin-3-yl}-benzoic acid is obtained as a yellow solid in 64% yield (350 mg) in two steps from N-[3-(3-bromo-pyrazolo[1,5-a]pyrimidin-7-yl)-phenyl]-3-trifluoromethyl-benzamide.

(1H DMSO): 12.89(s br, 1H); 10.73(s, 1H); 8.88(s, 1H); 8.83(dd, 1H); 8.80(d, 1H); 8.55(dd, 1H); 8.40(d, 1H); 8.34(s, 1H); 8.31(d, 1H); 8.06(d, 1H); 7.99(d, 1H); 7.90-7.77(m, 2H); 7.69-7.51(m, 3H); 7.40-7.24(m, 1H).

Step 3: To a solution of 3-{7-[3-(3-trifluoromethyl-benzoylamino)-phenyl]-pyrazolo[1,5-a]pyrimidin-3-yl}-benzoic acid (345 mg, 0.687 mmol) in DMF (10 ml), DIPEA (275 μl, 1.58 mmol) and pybop (822 mg, 1.58 mmol) are added. The mixture is stirred at room temperature for 5 minutes, and then NH₃ in THF is added (excess). After stirring at room temperature overnight, the solvent is evaporated. The crude material is firstly purified by a silica gel column chromatography (eluent: gradient from AcOEt to AcOEt-MeOH 25:1) and then by preparative HPLC leading to the pure 3-{7-[3-(3-trifluoromethyl-benzoylamino)-phenyl]-pyrazolo[1,5-a]pyrimidin-3-yl}-benzamide (61.3 mg, 18% yield) as a yellow solid.

(1H DMSO): 10.73(s, 1H); 8.85(s, 1H); 8.79(d, 1H); 8.60(dd, 1H); 8.55(dd, 1H); 8.39(d, 1H); 8.35(s, 1H); 8.31(d, 1H); 8.06(d, 1H); 7.99(d, 1H); 7.98(s br, 1H); 7.88(d, 1H); 7.81(dd, 1H); 7.76(d, 1H); 7.65(dd, 1H); 7.54(dd, 1H).

MS (electrospray): m/z 502.2 [M+H]

Examples 272-274 are prepared following the method for example 154 by reaction of N-[3-(3-bromo-pyrazolo[1,5-a]pyrimidin-7-yl)-phenyl]-3-trifluoromethyl-benzamide with the corresponding substituted boronic acids.

### EXAMPLE 272

### N-[3-(3-{2-[(dimethylamino)methyl]phenyl}pyrazolo[1,5-a]pyrimidin-7-yl)phenyl]-3-(trifluoromethyl)benzamide

MS (electrospray): m/z 516.3 [M+H]

### EXAMPLE 273

### N-[3-(3-{3-[(dimethylamino)methyl]phenyl}pyrazolo[1,5-a]pyrimidin-7-yl)phenyl]-3-(trifluoromethyl)benzamide

MS (electrospray): m/z 516.3 [M+H]

### EXAMPLE 274

### N-[3-(3-{4-[(dimethylamino)methyl]phenyl}pyrazolo[1,5-a]pyrimidin-7-yl)phenyl]-3-(trifluoromethyl)benzamide

MS (electrospray): m/z 516.2 [M+H]

Examples 275 and 276 are prepared following a modified procedure described for example 290, using 4-(4,4,5,5-tetramethyl-1,3,2-dioxaborolan-2-yl)-pyrazole-1-carboxylic acid tert-butyl ester (amounts) and N-[3-(3-bromo-pyrazolo[1,5-a]pyrimidin-7-yl)-phenyl]-3-trifluoromethyl-benzamide at 80° C. for 5 hrs. The crude mixture is purified by flash chromatography (Hexanes:EtOAc) to give N-{3-[3-(1H-pyrazol-4-yl)pyrazolo[1,5-a]pyrimidin-7-yl]phenyl}-3-(trifluoromethyl)benzamide (32.8 mg, 17% yield) and tert-butyl 4-[7-(3-{[3-(trifluoromethyl)benzoyl]amino}phenyl)pyrazolo[1,5-a]pyrimidin-3-yl]-1H-pyrazole-1-carboxylate (29.8 mg, 13% yield).

### EXAMPLE 275

### N-{3-[3-(1H-pyrazol-4-yl)pyrazolo[1,5-a]pyrimidin-7-yl]phenyl}-3-(trifluoromethyl)benzamide

MS (electrospray): m/z 4491 [M+H]

### EXAMPLE 276

### tert-butyl 4-[7-(3-{[3-(trifluoromethyl)benzoyl]amino}phenyl)pyrazolo[1,5-a]pyrimidin-3-yl]-1H-pyrazole-1-carboxylate

MS (electrospray): m/z 549.2 [M+H]

Example 277 is prepared following the procedure described for example 290 using the appropriate substituted boronic acids or boronic esters.

### EXAMPLE 277

### N-{3-[3-(3-furyl)pyrazolo[1,5-a]pyrimidin-7-yl]phenyl}-3-(trifluoromethyl)benzamide

MS (electrospray): m/z 447.1 [M+H]

Example 278 is prepared following the method for example 154 by reaction of N-[3-(3-bromo-pyrazolo[1,5-a]pyrimidin-7-yl)-phenyl]-3-trifluoromethyl-benzamide with the corresponding substituted boronic acids.

### EXAMPLE 278

### N-{3-[3-(6-aminopyridin-3-yl)pyrazolo[1,5-a]pyrimidin-7-yl]phenyl}-3-(trifluoromethyl)benzamide

MS (electrospray): m/z 475.2 [M+H]

Example 279 is prepared following the method for Example 205 by reaction of N-[3-(3-Bromo-pyrazolo[1,5-a]pyrimidin-7-yl)-phenyl]-3-trifluoromethyl-benzamide with the corresponding substituted alkynes.

### EXAMPLE 279

### N-(3-{3-[5-(4-methylpiperazin-1-yl)pent-1-yn-1-yl]pyrazolo[1,5-a]pyrimidin-7-yl}phenyl)-3-(trifluoromethyl)benzamide

MS (electrospray): m/z 547.2 [M+H]

Examples 280 and 281 are prepared following the method for example 154 by reaction of N-[3-(3-bromo-pyrazolo[1,5-a]pyrimidin-7-yl)-phenyl]-3-trifluoromethyl-benzamide with the corresponding substituted boronic acids.

### EXAMPLE 280

### N-[3-(3-{2-[2-(dimethylamino)ethyl]phenyl}pyrazolo[1,5-a]pyrimidin-7-yl)phenyl]-3-(trifluoromethyl)benzamide

MS (electrospray): m/z 530.2 [M+H]

### EXAMPLE 281

### N-[3-(3-{3-[2-(dimethylamino)ethyl]phenyl}pyrazolo[1,5-a]pyrimidin-7-yl)phenyl]-3-(trifluoromethyl)benzamide

MS (electrospray): m/z 530.1 [M+H]

Example 282 is prepared following the method for Example 205 by reaction of N-[3-(3-Bromo-pyrazolo[1,5-a]pyrimidin-7-yl)-phenyl]-3-trifluoromethyl-benzamide with the corresponding substituted alkynes.

### EXAMPLE 282

### N-{3-[3-(5-morpholin-4-ylpent-1-yn-1-yl)pyrazolo[1,5-a]pyrimidin-7-yl]phenyl}-3-(trifluoromethyl)benzamide

MS (electrospray): m/z 534.1 [M+H]

### EXAMPLE 283

### N-{3-[3-(6-{[2-(Dimethylamino)ethyl]amino}pyridin-3-yl)pyrazolo[1,5-a]pyrimidin-7-yl]phenyl}-3-(trifluoromethyl)benzamide

Step 1: To a solution of N-[3-(3-bromo-pyrazolo[1,5-a]pyrimidin-7-yl)-phenyl]-3-trifluoromethyl-benzamide (300 mg, 0.65 mmol) in DME (3 ml), Pd(PPh₃)₄ (112 mg, 0.15 eq), K₂CO₃ (2M solution, 3 ml, 10 eq), and 2-chloropyridine-5-boronic acid (200 mg, 1.3 mmol) are added. The reaction mixture is heated at 175° C. in a microwave oven for 20 minutes, then diluted with a saturated solution of NaHCO₃ and extracted with AcOEt.

The organic phase is dried and evaporated under vacuum; the crude N-{3-[3-(6-chloro-pyridin-3-yl)-pyrazolo[1,5-a]pyrimidin-7-yl]-phenyl}-3-trifluoromethyl-benzamide is used in next step without further purification.

Step 2: N-{3-[3-(6-Chloro-pyridin-3-yl)-pyrazolo[1,5-a]pyrimidin-7-yl]-phenyl}-3-trifluoromethyl-benzamide is reacted with N,N-dimethylaminoethylamine following the method for Example 257 with pyridine as the solvent to provide N-{3-[3-(6-{[2-(dimethylamino)ethyl]amino}pyridin-3-yl)pyrazolo[1,5-a]pyrimidin-7-yl]phenyl}-3-(trifluoromethyl)benzamide (13.9 mg, 3.9% yield for the 2 steps).

(1H CD3OD): 8.82(m, 1H); 8.65(d, 1H); 8.56(m, 1H); 8.51(s, 1H); 8.31-8.21(m, 3H); 7.96-7.86(m, 3H); 7.74(dd, 1H); 7.62(dd, 1H); 7.20(d, 1H); 6.81(dd, 1H); 3.72(m, 2H); 3.37(m, 2H); 2.99(s, 6H).

MS (electrospray): m/z 546.2 [M+H]

Example 284 is prepared by reaction of N-{3-[3-(6-Chloro-pyridin-3-yl)-pyrazolo[1,5-a]pyrimidin-7-yl]-phenyl}-3-trifluoromethyl-benzamide with methylamine following the method for Example 257 with pyridine as the solvent to provide N-(3-{3-[6-(methylamino)pyridin-3-yl]pyrazolo[1,5-a]pyrimidin-7-yl}phenyl)-3-(trifluoromethyl)benzamide (13.7 mg, 4.1% yield for the 2 steps).

(1H CD3OD): 8.73(m, 1H); 8.69(d, 1H); 8.59(s, 1H); 8.56(dd, 1H); 8.49(dd, 1H); 8.29(m, 1H); 8.24(m, 1H); 7.97-7.87(m, 3H); 7.74(m, 1H); 7.62(dd, 1H); 7.24(d, 1H); 6.99(d, 1H); 3.04(s, 3H).

### EXAMPLE 284

### N-(3-{3-[6-(methylamino)pyridin-3-yl]pyrazolo[1,5-a]pyrimidin-7-yl}phenyl)-3-(trifluoromethyl)benzamide

MS (electrospray): m/z 489.1 [M+H]

Examples 285 and 286 are prepared following the procedure described for example 290 using the appropriate substituted boronic acids or boronic esters.

### EXAMPLE 285

### N-(3-{3-[4-(hydroxymethyl)phenyl]pyrazolo[1,5-a]pyrimidin-7-yl}phenyl)-3-(trifluoromethyl)benzamide

MS (electrospray): m/z 487.3 [M+H]

### EXAMPLE 286

### N-(3-{3-[3-(hydroxymethyl)phenyl]pyrazolo[1,5-a]pyrimidin-7-yl}phenyl)-3-(trifluoromethyl)benzamide

MS (electrospray): m/z 487.4 [M+H]

Example 287 is prepared by reacting 3-(4-bromophenyl)-1H-pyrazol-5-amine with N-[3-(3-dimethylamino-acryloyl)-phenyl]-3-trifluoromethyl-benzamide, using the method of example 109.

### EXAMPLE 287

### N-{3-[2-(4-bromophenyl)pyrazolo[1,5-a]pyrimidin-7-yl]phenyl}-3-(trifluoromethyl)benzamide

MS (electrospray): m/z 537.2 [M+H]

Examples 288-289 are prepared following the method for example 154 by reaction of N-[3-(3-bromo-pyrazolo[1,5-a]pyrimidin-7-yl)-phenyl]-3-trifluoromethyl-benzamide with the corresponding substituted boronic acids.

### EXAMPLE 288

### N-[3-(3-{4-[(dimethylamino)sulfonyl]phenyl}pyrazolo[1,5-a]pyrimidin-7-yl)phenyl]-3-(trifluoromethyl)benzamide

MS (electrospray): m/z 566.5 [M+H]

### EXAMPLE 289

### N-[3-(3-{4-[2-(dimethylamino)ethyl]phenyl}pyrazolo[1,5-a]pyrimidin-7-yl)phenyl]-3-(trifluoromethyl)benzamide

MS (electrospray): m/z 530.1 [M+H]

### EXAMPLE 290

### 3-(trifluoromethyl)-N-(3-{3-[2-(trifluoromethyl)phenyl]pyrazolo[1,5-a]pyrimidin-7-yl}phenyl)benzamide

To a solution of N-[3-(3-bromo-pyrazolo[1,5-a]pyrimidin-7-yl)-phenyl]-3-trifluoromethyl-benzamide, (100 mg, 0.216 mmol) in ethylene glycol dimethyl ether (3 mL) is added 2-(trifluoromethyl)phenylboronic acid (82 mg, 0.433 mmol), [1,1′-bis(diphenylphosphino)ferrocene]dichloropalladium(II), complex with dichloromethane (35 mg, 0.043 mmol), sodium carbonate (2M aqueous solution, 0.43 mL, 0.864 mmol). After microwaving at 100° C. for 1000 seconds, the solution is diluted with ethyl acetate, filtered with celite, concentrated, and purified by HPLC to provide 3-(trifluoromethyl)-N-(3-{3-[2-(trifluoromethyl)phenyl]pyrazolo[1,5-a]pyrimidin-7-yl}phenyl)benzamide (11.8 mg, 10% yield).

HPLC: Rt=2.75 min; MS 525.1 [M−H]

Examples 291-300 are prepared following the procedure described for example 290 using the appropriate substituted boronic acids or boronic esters.

N-(3-{3-[3-(hydroxymethyl)phenyl]pyrazolo[1,5-a]pyrimidin-7-yl}phenyl)-3-(trifluoromethyl)benzamide

MS (electrospray): m/z 487.4 [M+H]

### EXAMPLE 291

### 3-(trifluoromethyl)-N-(3-{3-[3-(trifluoromethyl)phenyl]pyrazolo[1,5-a]pyrimidin-7-yl}phenyl)benzamide

MS (electrospray): m/z 525.1 [M+H]

### EXAMPLE 292

### 3-(trifluoromethyl)-N-(3-{3-[4-(trifluoromethyl)phenyl]pyrazolo[1,5-a]pyrimidin-7-yl}phenyl)benzamide

MS (electrospray): m/z 525.1 [M+H]

### EXAMPLE 293

### N-{3-[3-(2-cyanophenyl)pyrazolo[1,5-a]pyrimidin-7-yl]phenyl}-3-(trifluoromethyl)benzamide

MS (electrospray): m/z 484.1 [M+H]

### EXAMPLE 294

### N-{3-[3-(3-cyanophenyl)pyrazolo[1,5-a]pyrimidin-7-yl]phenyl}-3-(trifluoromethyl)benzamide

MS (electrospray): m/z 484.1 [M+H]

### EXAMPLE 295

### N-{3-[3-(4-cyanophenyl)pyrazolo[1,5-a]pyrimidin-7-yl]phenyl}-3-(trifluoromethyl)benzamide

MS (electrospray): m/z 484.1 [M+H]

### EXAMPLE 296

### methyl 3-[7-(3-{[3-(trifluoromethyl)benzoyl]amino}phenyl)pyrazolo[1,5-a]pyrimidin-3-yl]benzoate

MS (electrospray): m/z 517.1 [M+H]

### EXAMPLE 297

### methyl 4-[7-(3-{[3-(trifluoromethyl)benzoyl]amino}phenyl)pyrazolo[1,5-a]pyrimidin-3-yl]benzoate

MS (electrospray): m/z 517.1 [M+H]

### EXAMPLE 298

### N-{3-[3-(2-acetylphenyl)pyrazolo[1,5-a]pyrimidin-7-yl]phenyl}-3-(trifluoromethyl)benzamide

MS (electrospray): m/z 501.1 [M+H]

### EXAMPLE 299

### N-{3-[3-(3-acetylphenyl)pyrazolo[1,5-a]pyrimidin-7-yl]phenyl}-3-(trifluoromethyl)benzamide

MS (electrospray): m/z 501.1 [M+H]

### EXAMPLE 300

### N-{3-[3-(4-acetylphenyl)pyrazolo[1,5-a]pyrimidin-7-yl]phenyl}-3-(trifluoromethyl)benzamide

MS (electrospray): m/z 501.1 [M+H]

Example 301 is prepared following the method for Example 154 by reaction of N-[3-(3-bromo-pyrazolo[1,5-a]pyrimidin-7-yl)-phenyl]-3-trifluoromethyl-benzamide with the corresponding substituted boronic acids.

### EXAMPLE 301

### N-{3-[3-(2-chloropyridin-4-yl)pyrazolo[1,5-a]pyrimidin-7-yl]phenyl}-3-(trifluoromethyl)benzamide

MS (electrospray): m/z 494.3 [M+H]

### EXAMPLE 302

### N-[3-(5-methyl-2-pyridin-4-ylpyrazolo[1,5-a]pyrimidin-7-yl)phenyl]-3-(trifluoromethyl)benzamide

Step 1: A mixture of 3-dimethylamino-1-(3-nitro-phenyl)-but-2-en-1-one (0.205 g, 1.28 mmol) and 5-pyridin-4-yl-2H-pyrazol-3-ylamine (0.3 g, 1.28 mmol) in 3 mL of acetic acid is heated at 80° C. over night. After cooling to room temperature, a solid precipitates out that is filtered, washed with cold acetic acid and dried in vacuo to provide pure 5-methyl-7-(3-nitro-phenyl)-2-pyridin-4-yl-pyrazolo[1,5-a]pyrimidine (0.131 g, 31% yield) as a yellow solid.

MS 332.2 [M+H]

Step 2: To a suspension of 5-methyl-7-(3-nitro-phenyl)-2-pyridin-4-yl-pyrazolo[1,5-a]pyrimidine (0.10 g, 0.30 mmol) in a mixture of dimethylformamide, tetrahydrofuran and methanol (3 mL/2 mL/2 mL) is added 0.015 g of 10% palladium on carbon under nitrogen. The nitrogen atmosphere is replaced with hydrogen, and the reaction mixture is stirred at room temperature under a hydrogen balloon. Following the replacement of hydrogen with nitrogen, the palladium on carbon is removed by filtration, and further washed with 1:1 methylene chloride/methanol. After removing the solvents in vacuo, the product 3-(5-methyl-2-pyridin-4-yl-pyrazolo[1,5-a]pyrimidin-7-yl)-phenylamine is precipitated from ether and used directly in the next step.

MS 302.0 [M+H].

Step 3: A mixture of 3-(5-methyl-2-pyridin-4-yl-pyrazolo[1,5-a]pyrimidin-7-yl)-phenylamine (91 mg, 0.3 mmol), 3-trifluoromethyl-benzoyl chloride (0.045 mL, 0.3 mmol) and 1 mL of pyridine is stirred at room temperature for 3 days. The solvent is removed in vacuo, and the crude product is dissolved in DMSO. After removal of insoluble material by filtration, the product is purified by HPLC (reverse phase) to provide N-[3-(5-Methyl-2-pyridin-4-yl-pyrazolo[1,5-a]pyrimidin-7-yl)-phenyl]-3-trifluoromethyl-benzamide (23 mg, 16% yield) as an off-white solid.

MS 474.3 [M+H]

MS (electrospray): m/z 474.3 [M+H]

### EXAMPLE 303

### N-(3-{3-[2-(1-hydroxyethyl)phenyl]pyrazolo[1,5-a]pyrimidin-7-yl}phenyl)-3-(trifluoromethyl)benzamide

To a solution of N-{3-[3-(2-acetylphenyl)pyrazolo[1,5-a]pyrimidin-7-yl]phenyl}-3-(trifluoromethyl)benzamide, N-{3-[3-(2-acetylphenyl)pyrazolo[1,5-a]pyrimidin-7-yl]phenyl}-3-(trifluoromethyl)benzamide (20 mg, 0.039 mmol) in methanol (3 mL) is added sodium borohydride (4 mg, 0.12 mmol). The mixture is stirred at room temperature for 4 hrs and solvent is removed under vacuum. The residue is dissolved to ethyl acetate, washed with water, dried over anhydrous sodium sulfate, concentrated, purified by flash chromatography to provide N-(3-{3-[2-(1-hydroxyethyl)phenyl]pyrazolo[1,5-a]pyrimidin-7-yl}phenyl)-3-(trifluoromethyl)benzamide (8 mg, 40% yield).

MS (electrospray): m/z 503.2 [M+H]

Example 304 is prepared following the procedure described for example 303 from N-{3-[3-(3-acetylphenyl)pyrazolo[1,5-a]pyrimidin-7-yl]phenyl}-3-(trifluoromethyl)benzamide (14. 7 mg, 69% yield).

MS (electrospray): m/z 503.2 [M+H]

Example 305 is prepared following the procedure described for example 290 using the appropriate substituted boronic acids or boronic esters.

### EXAMPLE 305

### N-{3-[3-(2-methylphenyl)pyrazolo[1,5-a]pyrimidin-7-yl]phenyl}-3-(trifluoromethyl)benzamide

MS (electrospray): m/z 473.1 [M+H]

### EXAMPLE 306

### N-{3-[3-(1-methyl-1,2,3,6-tetrahydropyridin-4-yl)pyrazolo[1,5-a]pyrimidin-7-yl]phenyl}-3-(trifluoromethyl)benzamide

Step 1: A mixture of 3-amino pyrazole (44 mg, 0.5 mmol) and 1-methyl-4-piperidone (0.12 mL, 1.0 mmol) in glacial acetic acid (1 mL) is stirred at room temperature for 3 hours. The solvent is evaporated to dryness to yield 4-(1-methyl-1,2,3,6-tetrahydro-pyridin-4-yl)-2H-pyrazo-3-ylamine as a yellow foam which is used in the next step without further purification.

Step 2: N-{3-[3-(1-methyl-1,2,3,6-tetrahydropyridin-4-yl)pyrazolo[1,5-a]pyrimidin-7-yl]phenyl}-3-(trifluoromethyl)benzamide is prepared following the method used for example 205 (step 3) from crude 4-(1-methyl-1,2,3,6-tetrahydro-pyridin-4-yl)-2H-pyrazo-3-ylamine and N-[3-(3-dimethylamino-acryloyl)-phenyl]-3-trifluoromethyl-benzamide. Mp: 163°-165° C., MS (electrospray): m/z 478.3 [M+H].

### EXAMPLE 307

### N-(3-{3-[1-(2-pyrrolidin-1-ylethyl)-1H-pyrazol-4-yl]pyrazolo[1,5-a]pyrimidin-7-yl}phenyl)-3-(trifluoromethyl)benzamide

To a solution of N-{3-[3-(1H-pyrazol-4-yl)pyrazolo[1,5-a]pyrimidin-7-yl]phenyl}-3-(trifluoromethyl)benzamide, N-{3-[3-(1H-pyrazol-4-yl)pyrazolo[1,5-a]pyrimidin-7-yl]phenyl}-3-(trifluoromethyl)benzamide (33.8 mg, 0.075 mmol) in N—N-dimethylformamide (3 mL) is added 1-(2-chloroethyl)pyrrolidine hydrochloride (15.3 mg, 0.090 mmol), and cesium carbonate (5.8 mg, 0.18 mmol), and catalytic amount of tetrabutylammonium iodide (5.50 mg, 0.015 mmol). The mixture is heated at 65° C. for 24 hrs. The solution is diluted to ethyl acetate, washed with water, dried over sodium sulfate, filtered, concentrated, and purified by flash chromatography (EtOAc: MeOH) to give N-(3-{3-[1-(2-pyrrolidin-1-ylethyl)-1H-pyrazol-4-yl]pyrazolo[1,5-a]pyrimidin-7-yl}phenyl)-3-(trifluoromethyl)benzamide as a yellow solid (17.6 mg, 43% yield).

MS (electrospray): m/z 546.4 [M+H]

Example 308 is prepared following the procedure described for example 290 using the appropriate substituted boronic acids or boronic esters.

### EXAMPLE 308

### N-{3-[3-(1-methyl-1H-pyrazol-4-yl)pyrazolo[1,5-a]pyrimidin-7-yl]phenyl}-3-(trifluoromethyl)benzamide

MS (electrospray): m/z 463.3 [M+H]

### EXAMPLE 309

### N-{3-[3-(1-methylpiperidin-4-yl)pyrazolo[1,5-a]pyrimidin-7-yl]phenyl}-3-(trifluoromethyl)benzamide

A mixture of N-{3-[3-(1-methyl-1,2,3,6-tetrahydropyridin-4-yl)pyrazolo[1,5-a]pyrimidin-7-yl]phenyl}-3-(trifluoromethyl)benzamide (0.06 g, 0.13 mmol) and 10% palladium on carbon (20 mg) in ethanol (15 mL) is shaken on Parr shaker with H₂ at 45 psi for 20 hours. The mixture is filtered through a pad of Celite, washed with ethanol and filtrate is evaporated to dryness. The residue is purified by silica gel flash chromatography (methanol/methylene chloride) to yield 0.042 g (68%) of N-{3-[3-(1-methylpiperidin-4-yl)pyrazolo[1,5-a]pyrimidin-7-yl]phenyl}-3-(trifluoromethyl)benzamide as a white solid. Mp: 183°-185° C., MS 480.3 [M+H].

Examples 310-312 are prepared following the procedure described for example 290 using the appropriate substituted boronic acids or boronic esters.

### EXAMPLE 310

### N-{3-[3-(3,5-diformylphenyl)pyrazolo[1,5-a]pyrimidin-7-yl]phenyl}-3-(trifluoromethyl)benzamide

MS (electrospray): m/z 513.3 [M+H]

### EXAMPLE 311

### N-{3-[3-(6-fluoropyridin-3-yl)pyrazolo[1,5-a]pyrimidin-7-yl]phenyl}-3-(trifluoromethyl)benzamide

MS (electrospray): m/z 478.1 [M+H]

### EXAMPLE 312

### N-{3-[3-(6-methoxypyridin-3-yl)pyrazolo[1,5-a]pyrimidin-7-yl]phenyl}-3-(trifluoromethyl)benzamide

MS (electrospray): m/z 490.1 [M+H]

Example 313 is prepared following the method for Example 154 by reaction of N-[3-(3-bromo-pyrazolo[1,5-a]pyrimidin-7-yl)-phenyl]-3-trifluoromethyl-benzamide with the corresponding substituted boronic acids.

### EXAMPLE 313

### N-{3-[3-(5-formyl-2-furyl)pyrazolo[1,5-a]pyrimidin-7-yl]phenyl}-3-(trifluoromethyl)benzamide

MS (electrospray): m/z 477.2 [M+H]

### EXAMPLE 314

### 3-[7-(3-{[3-(trifluoromethyl)benzoyl]amino}phenyl)pyrazolo[1,5-a]pyrimidin-3-yl]benzoic acid

MS (electrospray): m/z 503.2 [M+H]

To a solution of methyl 3-[7-(3-{[3-(trifluoromethyl)benzoyl]amino}phenyl)pyrazolo[1,5-a]pyrimidin-3-yl]benzoate (16 mg, 0.031 mmol) in methanol-THF-water (4 mL/2 mL/1 mL) is added LiOH solution (2N, 0.14 mL, 0.27 mmol). The mixture is stirred at room temperature for 24 hrs. The solution is acidified with 10% citric acid. The whole solution is extracted with ethyl acetate. The organic layer is washed with water, dried over sodium sulfate, and concentrated to give 3-[7-(3-{[3-(trifluoromethyl)benzoyl]amino}phenyl)pyrazolo[1,5-a]pyrimidin-3-yl]benzoic acid as a yellow solid (14 mg, 90% yield).

MS 503.2 [M+H]

### EXAMPLE 315

### N-(3-{3-[4-(pyrrolidin-1-ylmethyl)phenyl]pyrazolo[1,5-a]pyrimidin-7-yl}phenyl)-3-(trifluoromethyl)benzamide

Step 1: A mixture of (4-bromomethylphenyl) boronic acid (0.118 g, 0.51 mmol) and pyrrolidine (0.082 mL, 1.0 mmol) is stirred 1 mL tetrahydrofuran at room temperature for 2 hours. The reaction mixture is concentrated under reduced pressure and the crude 4-pyrrolidin-1-ylmethylphenylboronic acid is used in the next step without further purification.

Step 2: N-(3-{3-[4-(Pyrrolidin-1-ylmethyl)phenyl]pyrazolo[1,5-a]pyrimidin-7-yl}phenyl)-3-(trifluoromethyl)benzamide is prepared following the method for Example 154 by reaction of N-[3-(3-bromo-pyrazolo[1,5-a]pyrimidin-7-yl)-phenyl]-3-trifluoromethyl-benzamide with 4-pyrrolidin-1-ylmethylphenylboronic acid (0.058 g, 42% yield).

MS: [M+H]⁺ 542.4, MS-HPLC: [M+H]⁺ 542.2 at t_{R} 11.6 min.

MS (electrospray): m/z 542.4 [M+H]

### EXAMPLE 316

### N-(3-{3-[5-(pyrrolidin-1-ylmethyl)-2-furyl]pyrazolo[1,5-a]pyrimidin-7-yl}phenyl)-3-(trifluoromethyl)benzamide

MS (electrospray): m/z 530.4 [M+H]

A solution of N-{3-[3-(5-formyl-2-furyl)pyrazolo[1,5-a]pyrimidin-7-yl]phenyl}-3-(trifluoromethyl)benzamide (0.074 g, 0.155 mmol) and pyrrolidine (0.062 mL, 0.775 mmol) in 1.6 mL of 1-methyl-2-pyrrolidinone and 3.2 mL of dichloromethane is cooled to 5° C. To this is added sodium triacetoxyborohydride (0.13 g, 0.620 mmol). The mixture is stirred five minutes, then three drops of glacial acetic acid are added. Stir overnight at room temperature. Saturated sodium bicarbonate is added and the resultant aqueous mixture is partitioned with dichloromethane and washed with brine. The collected organic layer is dried over sodium sulfate, concentrated under reduced pressure, then purified by flash chromatography on silica gel in 0-15% MeOH/CH₂Cl₂ to give N-(3-{3-[5-(pyrrolidin-1-ylmethyl)-2-furyl]pyrazolo[1,5-a]pyrimidin-7-yl}phenyl)-3-(trifluoromethyl)benzamide (0.010 g, 12% yield).

MS: [M−H] 530.4, HRMS [M+H]⁺ 532.19542.

Examples 317 and 318 are prepared following the procedure described for example 109 by using corresponding starting materials.

### EXAMPLE 317

### N-[4-fluoro-3-(2-pyridin-4-ylpyrazolo[1,5-a]pyrimidin-7-yl)phenyl]-3-(trifluoromethyl)benzamide

MS (electrospray): m/z 478.3 [M+H]

### EXAMPLE 318

### N-(4-fluoro-3-pyrazolo[1,5-a]pyrimidin-7-ylphenyl)-3-(trifluoromethyl)benzamide

MS (electrospray): m/z 401.2 [M+H]

Example 319 is prepared following the procedure described for example 316 by using corresponding starting materials.

### EXAMPLE 319

### N-(3-{3-[5-(pyrrolidin-1-ylmethyl)-3-furyl]pyrazolo[1,5-a]pyrimidin-7-yl}phenyl)-3-(trifluoromethyl)benzamide

MS (electrospray): m/z 532.4 [M+H]

MS: [M+H] 532.4, MS-HPLC: [M+H]⁺ 532 at t_{R} 11.4 min.

### EXAMPLE 320

### N-[3-(3-iodopyrazolo[1,5-a]pyrimidin-7-yl)phenyl]-3-(trifluoromethyl)benzamide

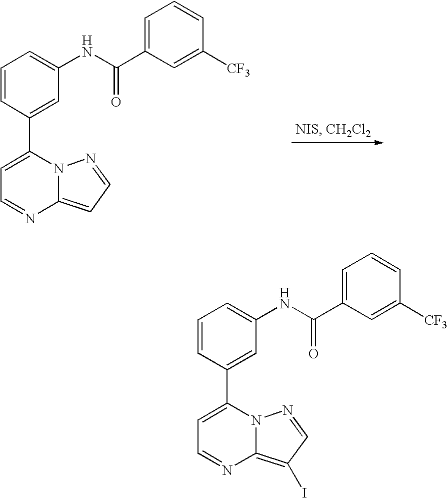

To a solution of N-(3-pyrazolo[1,5-a]pyrimidin-7-ylphenyl)-3-(trifluoromethyl)benzamide, (example 153) (100 mg, 0.261 mmol) in dichloromethane (5 mL) was added N-iodosuccinimide (120 mg, 0.533 mmol). The reaction mixture was stirred at room temperature overnight. After removing the solid by filtration, the filtrate was concentrated to give yellow residue and purified by HPLC (130 mg, 98% yield).

MS 509.2 [M+H]

Example 321 was prepared following the procedure for Example 283, step 1, by reacting N-[3-(3-bromo-pyrazolo[1,5-a]pyrimidin-7-yl)-phenyl]-3-trifluoromethylbenzamide with 5-formylfuran-3-boronic acid pinacol ester. Following purification by silica gel chromatography, N-{3-[3-(5-formyl-3-furyl)pyrazolo[1,5-a]pyrimidin-7-yl]phenyl}-3-(trifluoromethyl)benzamide was obtained as a yellow solid in 33% yield.

### EXAMPLE 321

### N-{3-[3-(5-formyl-3-furyl)pyrazolo[1,5-a]pyrimidin-7-yl]phenyl}-3-(trifluoromethyl)benzamide

MS: [M+H]⁺ 477.1

Example 322 was prepared following the procedure for Example 316, by reacting N-{3-[3-(5-formyl-3-furyl)pyrazolo[1,5-a]pyrimidin-7-yl]phenyl}-3-(trifluoromethyl)benzamide with N-ethylpiperazine. Following purification by silica gel chromatography, N-[3-(3-{5-[(4-ethylpiperazin-1-yl)methyl]-3-furyl}pyrazolo[1,5-a]pyrimidin-7-yl)phenyl]-3-(trifluoromethyl)benzamide was obtained in 89% yield.

### EXAMPLE 322

### N-[3-(3-{5-[(4-ethylpiperazin-1-yl)methyl]-3-furyl}pyrazolo[1,5-a]pyrimidin-7-yl)phenyl]-3-(trifluoromethyl)benzamide

MS: [M+H]⁺ 575.5

### EXAMPLE 323

### 3-(trifluoromethyl)-N-(3-{3-[(trimethylsilyl)ethynyl]pyrazolo[1,5-a]pyrimidin-7-yl}phenyl)benzamide

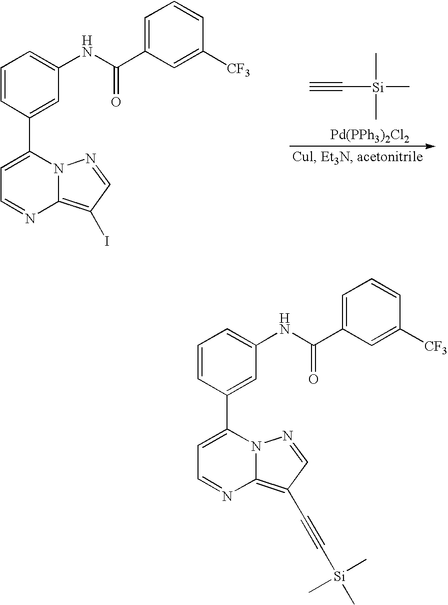

To a solution of N-[3-(3-iodopyrazolo[1,5-a]pyrimidin-7-yl)phenyl]-3-(trifluoromethyl)benzamide, (example 320) (240 mg, 0.473 mmol) and trimethylsilylacetylene (193 mg, 0.27 mL, 1.96 mmol) in triethylamine (2 mL) and acetonitrile (2 mL) were added dichlorobis(triphenylphosphine)palladium (11)(92 mg, 131 mmol) and copper iodide (49 mg, 0.262 mmol) under nitrogen atmosphere. The mixture was stirred at room temperature overnight. The mixture was concentrated and dissolved into ethylacetate. This organic solution washed with saturated sodium chloride solution, dried over sodium sulfate, filtered, and concentrated to give black residue. This residue was purified by column chromatography (hexanes:ethylacetate) to give yellow solid (31 mg, 14% yield).

MS 479.3 [M+H]

### EXAMPLE 324

### N-{3-[2-(imidazol-5-yl)pyrazolo[1,5-a]pyrimidin-7-yl]phenyl}-3-(trifluoromethyl)benzamide

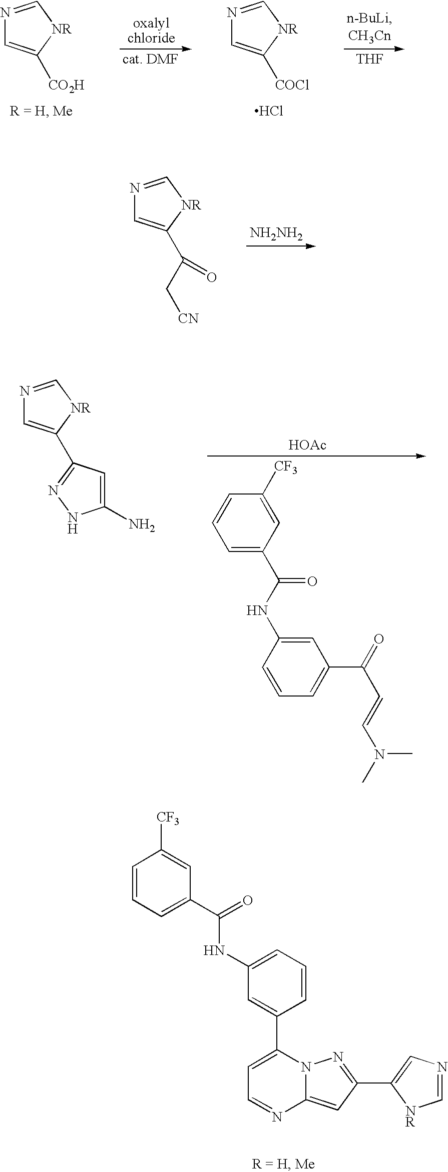

(R=H)

Step 1: To a 10 mL solution of oxalyl chloride was added 4-imidazolecarboxylic acid (1.0 g, 8.92 mmol) at room temperature, followed by 2 drops of dimethylformamide. The mixture was heated to reflux for 90 minutes, then cooled to room temperature. Following removal of the solvent by evaporation, toluene was added and evaporated to dryness twice to provide 1.49 g of imidazole-4-carbonyl chloride hydrochloride in quantitative yield as a yellow solid. This was used in the subsequent step without purification.

Step 2: A 50 mL solution of tetrahydrofuran was cooled to −78° C. using a dry ice/acetone bath. To this was added n-butyllithium (18.8 mL of a 2.5 M hexane solution, 44.6 mmol). After 10 minutes, acetonitrile (2.8 mL, 53.5 mmol) was slowly added with a syringe over an additional 10 minutes. The resulting solution was stirred for an additional 30 minutes, during which time a white precipitate formed. To this was added 1.49 g of crude imidazole-4-carbonyl chloride hydrochloride in two portions. The mixture was further stirred for 45 minutes, by which time a solid precipitated from the red solution. The solution was warmed to 0° C., and the solid filtered off. Purification of the solid was carried out by silica gel chromatography (eluting with a gradient of 7:3 to 85:15 methylene chloride/methanol) to provide 0.60 g (50% yield) of 3-(imidazol-5-yl)-3-oxopropanenitrile as a dark, waxy solid.

Step 3: To a solution of 3-(imidazol-5-yl)-3-oxopropanenitrile (0.3 g, 2.22 mmol) in ethanol (8 ml), NH₂NH₂.H₂O (0.2 ml, 4.19 mmol) and one drop of concentrated HCl were added. After stirring the mixture at reflux for 3 hours, the reaction was complete. Ethanol was evaporated under aspirator pressure, then under high vacuum to provide crude 5-(imidazol-4-yl)-2H-pyrazol-3-ylamine as a thick brown oil, which was used in the next step without purification.

Step 4: By the procedure of example 58, step 3, crude 5-(imidazol-4-yl)-2H-pyrazol-3-ylamine (0.11 g, 0.74 mmol) was reacted with N-[3-(3-dimethylamino-acryloyl)-phenyl]-3-trifluoromethyl-benzamide in acetic acid (2.5 mL) to provide 0.056 g (17% yield) of N-{3-[2-(imidazol-5-yl)pyrazolo[1,5-a]pyrimidin-7-yl]phenyl}-3-(trifluoromethyl)benzamide as a pale yellow solid.

MS (electrospray): m/z 449.3 [M+H]

### EXAMPLE 325

### 3,5-difluoro-N-[3-(2-pyridin-4-ylpyrazolo[1,5-a]pyrimidin-7-yl)phenyl]-benzamide

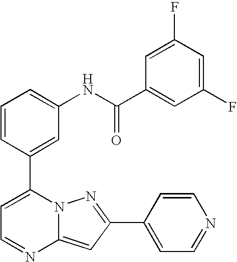

### Step 1: 3-(2-pyridin-4-ylpyrazolo[1,5-a]pyrimidin-7-yl)aniline, dihydrochloride salt

A solution of 8.726 g (27.5 mmoles) of 7-(3-nitrophenyl)-2-pyridin-4-ylpyrazolo[1,5-a]pyrimidine (prepared following the method of Example 1, Steps 1 & 2, using 5-pyridin-4-yl-2H-pyrazol-3-ylamine in Step 2) in 150 mL of acetic acid was warmed to 80° C. and 7.679 g (137.5 mmoles) of iron powder was added in several portions over 15 minutes. The mixture was stirred at 80° C. for 3 hours and allowed to cool to room temperature. The precipitated solid was filtered and the filtrate was concentrated and filtered 4 additional times. The collected solid inorganic salts were discarded and the final mother liquor was concentrated to dryness to give a mixture of the desired aniline and its corresponding acetanilide. This crude mixture was refluxed for 3 hours in a 5:2 mixture of ethanol and ˜10N hydrochloric acid. After cooling and stirring overnight at room temp the product was filtered, washed with ethanol and dried to give the product as a tan solid, used without additional purification.

MS (electrospray): m/z 288 [M+H]

### Step 2: 3,5-difluoro-N-[3-(2-pyridin-4-ylpyrazolo[1,5-a]pyrimidin-7-yl)phenyl]benzamide

To a mixture of 0.097 g (0.30 mmoles) of 3-(2-pyridin-4-ylpyrazolo[1,5-a]pyrimidin-7-yl)aniline, dihydrochloride salt and 0.005 g (0.04 mmoles) of 4-(dimethylamino)pyridine was added 0.106 g (0.60 mmoles) of 3,5-difluorobenzoyl chloride. Filtration of the reaction mixture, addition of 10 mL of water to the filtrate and filtering of the resulting precipitate provided 0.148 g of crude product. Purification of a 0.060 g portion by HPLC gave 0.047 g of pure 3,5-difluoro-N-[3-(2-pyridin-4-ylpyrazolo[1,5-a]pyrimidin-7-yl)phenyl]benzamide

MS (electrospray): m/z 428 [M+H]

### EXAMPLE 326

### Ethyl 7-(2-methoxy-5-{[3-(trifluoromethyl)benzoyl]amino}phenyl)pyrazolo[1,5-a]pyrimidine-3-carboxylate

### Step 1: (2E)-3-(dimethylamino)-1-(2-methoxy-5-nitrophenyl)prop-2-en-1-one

To a solution of 0.800 g (4.37 mmol) of 5′-amino-2′-fluoroacetophenone was added 3 mL of MDF-dimethyl acetal and the resulting mixture was heated at reflux overnight and then concentrated in vacuo. The residue was triturated with ether and filtered to provide (2E)-3-(dimethylamino)-1-(2-methoxy-5-nitrophenyl)prop-2-en-1-one.

### Step 2: Ethyl 7-(2-methoxy-5-nitrophenyl)pyrazolo[1,5-a]pyrimidine-3-carboxylate

According to the method of Example 58, Step 3, (2E)-3-(dimethylamino)-1-(2-methoxy-5-nitrophenyl)prop-2-en-1-one and 3-amino-4-carbethoxypyrazole gave ethyl 7-(2-methoxy-5-nitrophenyl)pyrazolo[1,5-a]pyrimidine-3-carboxylate.

### Step 3: Ethyl 7-(5-amino-2-methoxyphenyl)pyrazolo[1,5-a]pyrimidine-3-carboxylate

To a solution of 0.074 g (0.216 mmol) of ethyl 7-(2-methoxy-5-nitrophenyl)pyrazolo[1,5-a]pyrimidine-3-carboxylate in 3 mL of ethyl acetate was added 0.010 g of 10% Pd/C and the resulting mixture was placed under 1 atm hydrogen using a balloon. The reaction was stirred at room temperature for 12 hours and then filtered through Celite. The filtrate was concentrated in vacuo to provide ethyl 7-(5-amino-2-methoxyphenyl)pyrazolo[1,5-a]pyrimidine-3-carboxylate which was used without purification for the next step.

Step 4: According to the procedure of Example 1, Step 4, ethyl 7-(5-amino-2-methoxyphenyl)pyrazolo[1,5-a]pyrimidine-3-carboxylate was converted into ethyl 7-(2-methoxy-5-{[3-(trifluoromethyl)benzoyl]amino}phenyl)pyrazolo[1,5-a]pyrimidine-3-carboxylate, isolated as a yellow solid after column chromatography eluting with ethyl acetate/hexanes (3:2).

MS (electrospray): m/z 485 [M+H]

### EXAMPLE 327

### N-(4-methoxy-3-pyrazolo[1,5-a]pyrimidin-7-ylphenyl)-3-(trifluoromethyl)benzamide

According to the procedure of used for the preparation of ethyl 7-(2-methoxy-5-{[3-(trifluoromethyl)benzoyl]amino}phenyl)pyrazolo[1,5-a]pyrimidine-3-carboxylate, but using 3-aminopyrazole in Step 2 instead of 3-amino-4-carbethoxypyrazole, N-(4-methoxy-3-pyrazolo[1,5-a]pyrimidin-7-ylphenyl)-3-(trifluoromethyl)benzamide was obtained as an off-white solid. MS (electrospray): m/z 413 [M+H]

### EXAMPLE 328

### N-[3-(3-ethynylpyrazolo[1,5-a]pyrimidin-7-yl)phenyl]-3-(trifluoromethyl)benzamide

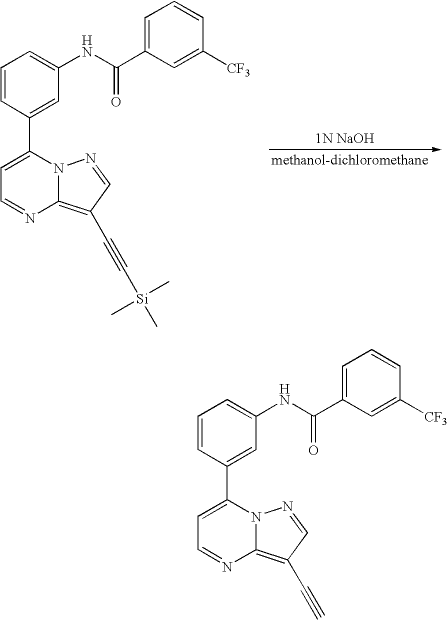

To a solution of 3-(trifluoromethyl)-N-(3-{3-[(trimethylsilyl)ethynyl]pyrazolo[1,5-a]pyrimidin-7-yl}phenyl)benzamide, (25 mg, 0.052 mmol) in methanol-dichloromethane (5 mL-1 mL) was added 1 mL of 1 N sodium hydroxide solution. The mixture was stirred at room temperature for 1 h and neutralized with 1 N hydrochloride solution. The mixture was concentrated to remove the organic solvent and extracted into ethylacetate. The organic layers washed with water, dried over sodium sulfate, filtered and concentrated to give yellow residue. The residue was purified by column chromatography (hexanes:ethylacetate) to give yellow solid (9 mg, 43% yield).

MS 406.9 [M+H]

Example 329 was prepared following the procedure for Example 283, step 1, by reacting N-[3-(3-bromo-pyrazolo[1,5-a]pyrimidin-7-yl)-phenyl]-3-trifluoromethylbenzamide with 1-methyl-4-[4-(4,4,5,5-tetramethyl-[1,3,2]dioxaborolan-2-yl)-phenyl]-piperazine. Following purification by silica gel chromatography, N-[3-(3-{4-[(4-methylpiperazin-1-yl)methyl]phenyl}pyrazolo[1,5-a]pyrimidin-7-yl)phenyl]-3-(trifluoromethyl)benzamide was obtained as a yellow solid in 38% yield.

### EXAMPLE 329

### N-[3-(3-{4-[(4-methylpiperazin-1-yl)methyl]phenyl}pyrazolo[1,5-a]pyrimidin-7-yl)phenyl]-3-(trifluoromethyl)benzamide

MS: [M+H]⁺ 571.4

### EXAMPLE 330

### N-{3-[3-(2-methoxypyrimidin-5-yl)pyrazolo[1,5-a]pyrimidin-7-yl]phenyl}-3-(trifluoromethyl)benzamide

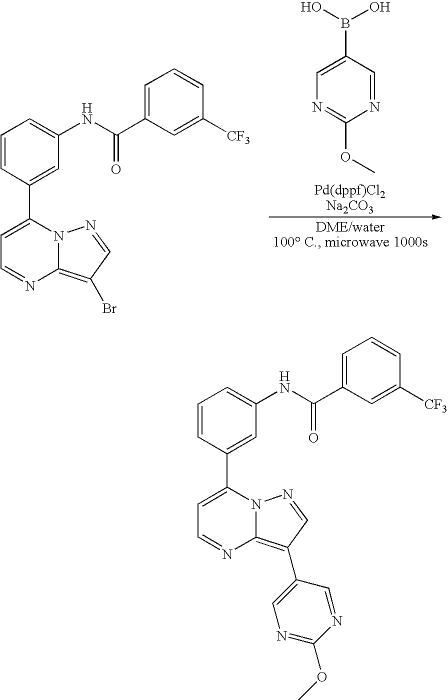

To a solution of N-[3-(3-bromo-pyrazolo[1,5-a]pyrimidin-7-yl)-phenyl]-3-trifluoromethyl-benzamide, (100 mg, 0.216 mmol) in ethylene glycol dimethyl ether (3 mL) was added 2-methoxypyrimidine-5-boronic acid (66 mg, 0.433 mmol), [1,1′-bis(diphenylphosphino)ferrocene]dichloropalladium(II), complex with dichloromethane (35 mg, 0.043 mmol), sodium carbonate (2M aqueous solution, 0.43 mL, 0.864 mmol). After microwaving at 100° C. for 1000 seconds, the solution was diluted with ethyl acetate, filtered with celite, concentrated, and purified by HPLC (10 mg, 10% yield).

MS 489.2 [M−H]

### EXAMPLE 331

### N-[3-(3-{3,5-bis[(dimethylamino)methyl]phenyl}pyrazolo[1,5-a]pyrimidin-7-yl)phenyl]-3-(trifluoromethyl)benzamide

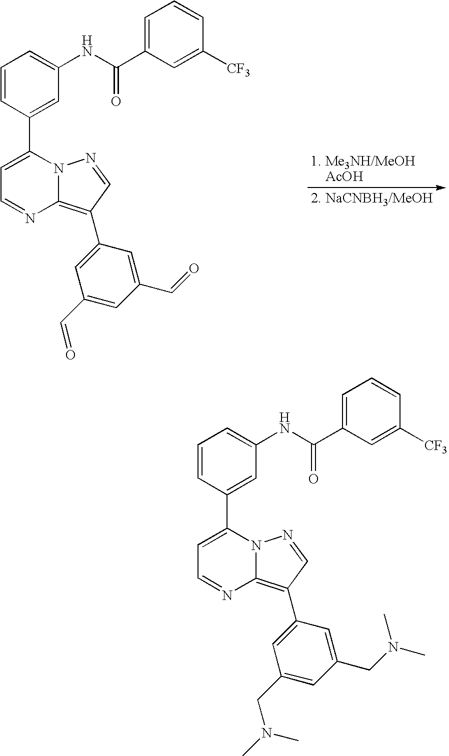

To a solution of N-{3-[3-(3,5-diformylphenyl)pyrazolo[1,5-a]pyrimidin-7-yl]phenyl}-3-(trifluoromethyl)benzamide, (42 mg, 0.08 mmol) in methanol (10 mL) was added dimethylamine (2M solution in methanol, 0.16 mL, 0.32 mmol). After 1 h of stirring acetic acid (5 mg, 0.08 mmol) and sodium cyanoborohydride (20 mg, 0.32 mmol) were added to the solution. The reaction mixture was stirred at room temperature overnight, concentrated to give yellow residue. This residue was dissolved into methanol and filtered to remove solid residue. The filtrate was concentrated and purified by HPLC (9.7 mg, 22% yield).

MS 573.4 [M+H]

### EXAMPLE 332

### N-[3-(3-iodopyrazolo[1,5-a]pyrimidin-7-yl)-4-methoxyphenyl]-3-(trifluoromethyl)benzamide

To 0.204 g (0.495 mmol) of N-(4-methoxy-3-pyrazolo[1,5-a]pyrimidin-7-ylphenyl)-3-(trifluoromethyl)benzamide in 10 mL of dichloromethane was added 0.134 g (0.594 mmol) of N-iodosuccinimide and the reaction was stirred at room temperature for 24 hours. The resulting mixture washed with saturated sodium bisulfite and water, dried over magnesium sulfate, filtered and concentrated in vacuo to provide 0.266 g of N-[3-(3-iodopyrazolo[1,5-a]pyrimidin-7-yl)-4-methoxyphenyl]-3-(trifluoromethyl)benzamide. MS (electrospray): m/z 539 [M+H]

### EXAMPLE 333

### N-[4-fluoro-3-(3-iodopyrazolo[1,5-a]pyrimidin-7-yl)phenyl]-3-(trifluoromethyl)benzamide

To 0.036 g (0.09 mmol) of the product of N-(4-fluoro-3-pyrazolo[1,5-a]pyrimidin-7-ylphenyl)-3-(trifluoromethyl)benzamide (Example 318) in 5 mL of chloroform was added 0.024 g (0.108 mmol) of N-iodosuccinimide and the reaction was stirred at room temperature for 24 hours. The resulting mixture washed with saturated sodium bisulfite and water, dried over magnesium sulfate, filtered and concentrated in vacuo. The residue washed with ethyl acetate to provide N-[4-fluoro-3-(3-iodopyrazolo[1,5-a]pyrimidin-7-yl)phenyl]-3-(trifluoromethyl)benzamide.

MS (electrospray): m/z 527 [M+H]

### EXAMPLE 334

### 3-(difluoromethyl)-N-[3-(2-pyridin-4-ylpyrazolo[1,5-a]pyrimidin-7-yl)phenyl]benzamide

### Step 1: 3-(difluoromethyl)benzoyl chloride

A mixture of 0.10 g (0.61 mmol) of 3-(difluoromethyl)benzoic acid (WO 2002050019), oxalyl chloride (0.16 mL, 1.83 mmol) and DMF (3 drops) in dioxane (1 mL) was stirred at room temperature for 2 hours. The solvents were then removed under reduced pressure to provide crude 3-(difluoromethyl)benzoyl chloride.

Step 2: A mixture of 3-(2-(pyridin-4-yl)pyrazolo[1,5-a]pyrimidin-7-yl)aniline (0.219 mg, 0.61 mmol), 3-(difluoromethyl)benzoyl chloride (0.116 mg, 0.61 mmol), triethylamine 0.85 mL, 6.1 mmol) in NMP was stirred at room temperature for 3 h. The reaction was filtered and the crude mixture was first purified on HPLC (acetonitrile/water/trifluoroacetic acid), followed by further purification on silica gel using 5% methanol in dichloromethane to give 3-(difluoromethyl)-N-[3-(2-pyridin-4-ylpyrazolo[1,5-a]pyrimidin-7-yl)phenyl]benzamide as a yellow solid (13.3 mg, 10% yield).

MS 442.3 [M+H].

Example 335 was prepared following the procedure for Example 283, step 1, by reacting N-[3-(3-bromo-pyrazolo[1,5-a]pyrimidin-7-yl)-phenyl]-3-trifluoromethylbenzamide with 1-methyl-4-[4-(4,4,5,5-tetramethyl-1,3,2-dioxoborolan-2-yl)pyridin-2-yl]piperazine. Following purification by silica gel chromatography, N-(3-{3-[6-(4-methylpiperazin-1-yl)pyridin-3-yl]pyrazolo[1,5-a]pyrimidin-7-yl}phenyl)-3-(trifluoromethyl)benzamide was obtained as a yellow solid in 38% yield.

### EXAMPLE 335

### N-(3-{3-[6-(4-methylpiperazin-1-yl)pyridin-3-yl]pyrazolo[1,5-a]pyrimidin-7-yl}phenyl)-3-(trifluoromethyl)benzamide

MS: [M+H]⁺ 558.4

Examples 336, 337, 338 and 339 are prepared following the procedure described for example 330 by using corresponding starting materials.

### EXAMPLE 336

### N-{3-[3-(3,5-dimethylisoxazol-4-yl)pyrazolo[1,5-a]pyrimidin-7-yl]phenyl}-3-(trifluoromethyl)benzamide

MS (electrospray): m/z 478.3 [M+H]

### EXAMPLE 337

### N-[3-(3-pyrimidin-5-ylpyrazolo[1,5-a]pyrimidin-7-yl)phenyl]-3-(trifluoromethyl)benzamide

MS (electrospray): m/z 461.1 [M+H]

### EXAMPLE 338

### N-(3-{3-[2-(dimethylamino)pyrimidin-5-yl]pyrazolo[1,5-a]pyrimidin-7-yl}phenyl)-3-(trifluoromethyl)benzamide

MS (electrospray): m/z 504.1 [M+H]

### EXAMPLE 339

### N-{3-[3-(2-morpholin-4-ylpyrimidin-5-yl)pyrazolo[1,5-a]pyrimidin-7-yl]phenyl}-3-(trifluoromethyl)benzamide

MS (electrospray): m/z 546.3 [M+H]

### EXAMPLE 340

### 7-[3-(3-Trifluoromethyl-benzoylamino)-phenyl]-pyrazolo[1,5-a]pyrimidine-2-carboxylic acid

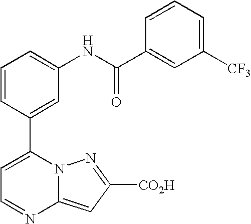

### Step 1: N-(3-Acetyl-phenyl)-3-trifluoromethyl-benzamide

To a solution of 1-(3-Amino-phenyl)-ethanone (5.0 g, 36.99 mmol) in CH₂Cl₂ (100 mL) and pyridine (4.48 mL, 55.48 mmol) was added 3-trifluoromethyl-benzoyl chloride (6 mL, 40.68 mmol) dropwise at 0° C. The reaction was allowed to warm to 25° C. over 19 h. The reaction was diluted in CH₂Cl₂ (100 mL) and the organic washed with 1N HCl (25 mL) and brine (100 mL). The organic was dried over MgSO₄, filtered concentrated to obtain a crude solid. The crude was further purified by Biotage chromatography (cartridge 40L), eluent 1:4 EtOAc-Hex to obtain N-(3-acetyl-phenyl)-3-trifluoromethyl-benzamide as an amorphous solid (10.9 g, 96%).

Mass Spectrum (+ESI): 308 (M+H)⁺.

### Step 2: N-[3-(3-Dimethylamino-acryloyl)-phenyl]-3-trifluoromethyl-benzamide

N-(3-Acetyl-phenyl)-3-trifluoromethyl-benzamide (11.0 g, 35.73 mmol) was dissolved in DMF-DMA (50 mL) and heated to 100° C. After 5 h solvent was removed under reduced pressure to give an orange viscous oil. The crude mixture was further purified by Biotage chromatography (cartridge 40L), eluent 1:4 EtOAc-Hex, then 100% EtOAc to obtain N-[3-(3-dimethylamino-acryloyl)-phenyl]-3-trifluoromethyl-benzamide as an amorphous solid (9.8 g, 75.7%). Mass Spectrum (+ESI): 363 (M+H)⁺.

### Step 3. Isoxazole-5-carboxylic acid methyl ester

A mixture of isoxazole-5-carboxylic acid (2.5 g 22.1 mmol), sodium bicarbonate (5.57 g, 66.32 mmol) and iodomethane (8.26 mL, 132.65 mmol) in DMF (30 mL) was stirred at 25° C. over 19 h. The mixture was diluted in H₂O (30 mL) and extracted with ether (2×50 mL). The ether layer washed with brine, dried over MgSO₄, filtered, concentrated in vacuo to obtain a crude oil. The crude was further purified by Biotage chromatography (cartridge 40m), eluent EtOAc-Hexanes (1:2), then 100% EtOAc to obtain isoxazole-5-carboxylic acid methyl ester as an amorphous solid (1.2 g, 42.7%).

Mass Spectrum (+ESI): 128 (M+H)⁺.

### Step 4. 3-Isoxazol-5-yl-3-oxo-propionitrile

To a suspension of potassium t-butoxide (1M THF, 14.2 mL, 14.16 mmol) was added a premixed solution of isoxazole-5-carboxylic acid methyl ester (1.2 g, 9.44 mmol) and CH₃CN (0.464 g, 11.33 mmol) in toluene. The solid immediately precipitated out and became difficult to stir. The reaction was heated to 80° C. over 19 h. The potassium salts were collected by filtration, washed with toluene, ether and dried to obtain 3-isoxazol-5-yl-3-oxo-propionitrile as a brown solid (1.68 g).

Mass Spectrum (+ESI): 137 (M+H)⁺.

### Step 5. 5-Isoxazol-5-yl-2H-pyrazol-3-ylamine

A mixture of 3-isoxazol-5-yl-3-oxo-propionitrile (1.2 g, 9.44 mmol), hydrazine monohydrate (0.916 mL, 18.84 mmol), HCl (0.717 mL, 23.6 mmol) in ethanol (25 mL) was heated to reflux for 19 h. Thin layer chromatography revealed that the reaction was largely complete. The mixture was cooled, filtered (inorganic solids were discarded), then basified with saturated aqueous NaHCO₃ (2 mL) and the mixture was evaporated to dryness to obtain 5-isoxazol-5-yl-2H-pyrazol-3-ylamine (0.5 g, 35.1%).

Mass Spectrum (+ESI): 151 (M+H)⁺.

### Step 6. 7-[3-(3-Trifluoromethyl-benzoylamino)-phenyl]-pyrazolo[1,5-a]pyrimidine-2-carboxylic acid

A solution of N-[3-(3-dimethylamino-acryloyl)-phenyl]-3-trifluoromethyl-benzamide (0.965, 2.66 mmol) and 5-Isoxazol-5-yl-2H-pyrazol-3-ylamine (0.5 g, 3.33 mmol) in acetic acid was heated at 10° C. over 19 h. Solvent was removed in vacuo. The crude oil (1.2 g) was diluted in ethyl acetate (200 mL) and the organic was washed with saturated aqueous NaHCO₃ (2×50 mL) and brine (50 mL). The organic was dried over MgSO₄, filtered, and concentrated in vacuo to obtain a crude oil. The crude was further purified by Biotage chromatography (cartridge 40m), eluent 100% EtOAc, then 5% methanol in EtOAc to obtain 7-[3-(3-trifluoromethyl-benzoylamino)-phenyl]-pyrazolo[1,5-a]pyrimidine-2-carboxylic acid as an amorphous solid (0.2 g, 16.8%).

Mass Spectrum (+ESI): 450 (M+H)⁺.

Examples 341 and 342 were prepared following a modified procedure described for example 330, by reacting N-[4-fluoro-3-(3-iodopyrazolo[1,5-a]pyrimidin-7-yl)phenyl]-3-(trifluoromethyl)benzamide and 4-(4,4,5,5-tetramethyl-1,3,2-dioxaborolan-2-yl)-pyrazole-1-carboxylic acid tert-butyl ester under thermal conditions (80° C. for 16 hours in 1,2-dimethoxyethane/aqueous potassium carbonate). The crude compounds were purified by flash chromatography (hexanes:ethyl acetate) to give N-{4-fluoro-3-[3-(1H-pyrazol-4-yl)pyrazolo[1,5-a]pyrimidin-7-yl]phenyl}-3-(trifluoromethyl)benzamide (11.6 mg, 13% yield) and tert-butyl 4-[7-(2-fluoro-5-{[3-(trifluoromethyl)benzoyl]amino}phenyl)pyrazolo[1,5-a]pyrimidin-3-yl]-1H-pyrazole-1-carboxylate (11.7 mg, 11% yield).

### EXAMPLE 341

### N-{4-fluoro-3-[3-(1H-pyrazol-4-yl)pyrazolo[1,5-a]pyrimidin-7-yl]phenyl}-3-(trifluoromethyl)benzamide

MS 467.3 [M+H]

### EXAMPLE 342

### tert-butyl 4-[7-(2-fluoro-5-{[3-(trifluoromethyl)benzoyl]amino}phenyl)pyrazolo[1,5-a]pyrimidin-3-yl]-1H-pyrazole-1-carboxylate

MS 567.3 [M+H]

Example 343 was prepared following the procedure for Example 257, step 5, by reacting N-{3-[3-(2-chloropyridin-4-yl)pyrazolo[1,5-a]pyrimidin-7-yl]phenyl}-3-(trifluoromethyl)benzamide with N-methylpiperazine in NMP. Following purification by HPLC (acetonitrile/water/trifluoroacetic acid), 14 mg of N-(3-{3-[2-(4-methylpiperazin-1-yl)pyridin-4-yl]pyrazolo[1,5-a]pyrimidin-7-yl}phenyl)-3-(trifluoromethyl)benzamide was obtained as a beige solid (9% yield).

### EXAMPLE 343

### N-(3-{3-[2-(4-methylpiperazin-1-yl)pyridin-4-yl]pyrazolo[1,5-a]pyrimidin-7-yl}phenyl)-3-(trifluoromethyl)benzamide

MS: [M+H]⁺ 558.4

### EXAMPLE 344

### N-(4-fluoro-3-{3-[6-(4-methylpiperazin-1-yl)pyridin-3-yl]pyrazolo[1,5-a]pyrimidin-7-yl}phenyl)-3-(trifluoromethyl)benzamide

A mixture of N-[4-fluoro-3-(3-iodopyrazolo[1,5-a]pyrimidin-7-yl)phenyl]-3-(trifluoromethyl)benzamide (80 mg, 0.15 mmol), 1-methyl-4-[4-(4,4,5,5-tetramethyl-1,3,2-dioxoborolan-2-yl)pyridin-2-yl]piperazine (55 mg, 0.18 mmol), 1,1′-Bis(diphenylphosphino)ferrocene]dichloropalladium(II), complex with CH₂Cl₂ (6 mg, 0.007 mmol) were combined in 0.8 mL of ethylene glycol dimethyl ether and 0.4 mL of 2M aqueous sodium carbonate and heated by microwave at 170° C. for 1200 s. The reaction mixture was evaporated, re-dissolved in DMSO and purified by reverse phase HPLC to give N-(4-fluoro-3-{3-[6-(4-methylpiperazin-1-yl)pyridin-3-yl]pyrazolo[1,5-a]pyrimidin-7-yl}phenyl)-3-(trifluoromethyl)benzamide as a yellow solid, 43 mg, 42% yield.

MS: [M+H]⁺ 576.3

### EXAMPLE 345

### N-[4-chloro-3-(2-pyridin-4-ylpyrazolo[1,5-a]pyrimidin-7-yl)phenyl]-3-(trifluoromethyl)benzamide

According to the procedure of N-[3-(2-pyridin-4-ylpyrazolo[1,5-a]pyrimidin-7-yl)phenyl]-3-(trifluoromethyl)benzamide (Example 109), N-{4-chloro-3-[(2E)-3-(dimethylamino)prop-2-enoyl]phenyl}-3-(trifluoromethyl)benzamide (prepared according to the procedure of Example 58, Step 1, from 5′-amino-2′-chloroacetophenone) and 5-pyridin-4-yl-2H-pyrazol-3-ylamine provided N-[4-chloro-3-(2-pyridin-4-ylpyrazolo[1,5-a]pyrimidin-7-yl)phenyl]-3-(trifluoromethyl)benzamide as a tan solid.

MS (electrospray): m/z 494 [M+H]

### EXAMPLE 346

### Ethyl 7-(2-chloro-5-{[3-(trifluoromethyl)benzoyl]amino}phenyl)pyrazolo[1,5-a]pyrimidine-3-carboxylate

According to the method of ethyl 2-methyl-7-(3-{[3-(trifluoromethyl)benzoyl]amino}phenyl)-pyrazolo[1,5-a]pyrimidine-3-carboxylate (Example 58), starting from 5′-amino-2′-chloroacetophenone and using 3-amino-4-carbethoxypyrazole in Step 3, ethyl 7-(2-chloro-5-{[3-(trifluoromethyl)benzoyl]amino}phenyl)pyrazolo[1,5-a]pyrimidine-3-carboxylate was obtained as a white solid after trituration with ether.

MS (electrospray): m/z 489 [M+H]

### EXAMPLE 347

### N-(4-chloro-3-pyrazolo[1,5-a]pyrimidin-7-ylphenyl)-3-(trifluoromethyl)benzamide

According to the procedure of N-[3-(2-pyridin-4-ylpyrazolo[1,5-a]pyrimidin-7-yl)phenyl]-3-(trifluoromethyl)benzamide (Example 109), N-{4-chloro-3-[(2E)-3-(dimethylamino)prop-2-enoyl]phenyl}-3-(trifluoromethyl)benzamide (prepared according to the procedure of Example 58, Step 1, from 5′-amino-2′-chloroacetophenone) and 3-aminopyrazole provided N-(4-chloro-3-pyrazolo[1,5-a]pyrimidin-7-ylphenyl)-3-(trifluoromethyl)benzamide.

MS (electrospray): m/z 417 [M+H]

Example 348 was prepared following the procedure for Example 344, by reacting N-[3-(3-bromo-pyrazolo[1,5-a]pyrimidin-7-yl)-phenyl]-3-trifluoromethyl-benzamide with 1-[4-(4,4,5,5-tetramethyl-[1,3,2]dioxaborolan-2-yl)-phenyl]-piperazine. Following purification by HPLC (acetonitrile/water/trifluoroacetic acid), 15 mg of N-{3-[3-(4-piperazin-1-ylphenyl)pyrazolo[1,5-a]pyrimidin-7-yl]phenyl}-3-(trifluoromethyl)benzamide was obtained as a beige solid, 11% yield.

### EXAMPLE 348

### N-{3-[3-(4-piperazin-1-ylphenyl)pyrazolo[1,5-a]pyrimidin-7-yl]phenyl}-3-(trifluoromethyl)benzamide

MS: [M+H]⁺ 543.3

### EXAMPLE 349

### N-{3-[2-(1-Methyl-1H-imidazol-5-yl)pyrazolo[1,5-a]pyrimidin-7-yl]phenyl}-3-(trifluoromethyl)benzamide

N-{3-[2-(1-Methyl-1H-imidazol-5-yl)pyrazolo[1,5-a]pyrimidin-7-yl]phenyl}-3-(trifluoromethyl)benzamide was prepared following the method of N-{3-[2-(imidazol-5-yl)pyrazolo[1,5-a]pyrimidin-7-yl]phenyl}-3-(trifluoromethyl)benzamide (Example 324), using 3-methyl-3H-imidazole-4-carboxylic acid as the starting material.

MS (electrospray): m/z 463.3 [M+H]

### EXAMPLE 350

### tert-Butyl 2-[7-(3-{[3-(trifluoromethyl)benzoyl]amino}phenyl)pyrazolo[1,5-a]pyrimidin-3-yl]-1H-pyrrole-1-carboxylate was prepared by the method of N-{3-[3-(2-methoxypyrimidin-5-yl)pyrazolo[1,5-a]pyrimidin-7-yl]phenyl}-3-(trifluoromethyl)benzamide (Example 330)

MS (electrospray): m/z 548.3 [M+H]

### EXAMPLE 351

### N-[4-chloro-3-(3-iodopyrazolo[1,5-a]pyrimidin-7-yl)phenyl]-3-(trifluoromethyl)benzamide

To 0.085 g (0.0.204 mmol) of N-(4-chloro-3-pyrazolo[1,5-a]pyrimidin-7-ylphenyl)-3-(trifluoromethyl)benzamide in 10 mL of chloroform was added 0.055 g (0.245 mmol) of N-iodosuccinimide and the reaction was stirred at room temperature for 24 hours. The resulting mixture washed with saturated sodium bisulfite and water, dried over magnesium sulfate, filtered and concentrated in vacuo. The residue was triturated with ether to provide N-[4-chloro-3-(3-iodopyrazolo[1,5-a]pyrimidin-7-yl)phenyl]-3-(trifluoromethyl)benzamide.

MS (electrospray): m/z 541 [M−H]

### EXAMPLE 352

### Methyl 7-(2-fluoro-5-{[3-(trifluoromethyl)benzoyl]amino}phenyl)pyrazolo[1,5-a]pyrimidine-3-carboxylate

To 0.075 g (0.159 mmol) of ethyl 7-(2-fluoro-5-{[3-(trifluoromethyl)benzoyl]amino}phenyl)pyrazolo[1,5-a]pyrimidine-3-carboxylate (Example 91) dissolved in 1 mL of THF and 1 mL of methanol was added 0.6 mL of 1N sodium hydroxide and the reaction was stirred at room temperature for 12 hours. The resulting mixture was acidified with 1N HCl and extracted with ethyl acetate. The organics were washed with water, dried over magnesium sulfate, filtered and concentrated in vacuo to provide methyl 7-(2-fluoro-5-{[3-(trifluoromethyl)benzoyl]amino}phenyl)pyrazolo[1,5-a]pyrimidine-3-carboxylate as a white solid.

MS (electrospray): m/z 459 [M+H]

Example 353 was prepared following the procedure for Example 344, by reacting N-[3-(3-bromo-pyrazolo[1,5-a]pyrimidin-7-yl)-phenyl]-3-trifluoromethyl-benzamide with 1-methyl-4-[4-(4,4,5,5-tetramethyl-[1,3,2]dioxaborolan-2-yl)-phenyl]-piperazine. Following purification by HPLC (acetonitrile/water/trifluoroacetic acid), 11 mg of N-(3-{3-[4-(4-methylpiperazin-1-yl)phenyl]pyrazolo[1,5-a]pyrimidin-7-yl}phenyl)-3-(trifluoromethyl)benzamide was obtained as a beige solid, 8% yield.

### EXAMPLE 353

### N-(3-{3-[4-(4-methylpiperazin-1-yl)phenyl]pyrazolo[1,5-a]pyrimidin-7-yl}phenyl)-3-(trifluoromethyl)benzamide

MS: [M+H]⁺ 557.4

Example 354 was prepared following the procedure for Example 344, by reacting N-[3-(3-bromo-pyrazolo[1,5-a]pyrimidin-7-yl)-phenyl]-3-trifluoromethyl-benzamide with N-[5-(4,4,5,5-tetramethyl-[1,3,2]dioxaborolan-2-yl)-pyridin-2-yl]-acetamide. Following purification by HPLC (acetonitrile/water/trifluoroacetic acid), 12 mg of N-{3-[3-(6-acetamidopyridin-3-yl)pyrazolo[1,5-a]pyrimidin-7-yl]phenyl}-3-(trifluoromethyl)benzamide was obtained as a yellow-beige solid, 10% yield.

### EXAMPLE 354

### N-{3-[3-(6-acetamidopyridin-3-yl)pyrazolo[1,5-a]pyrimidin-7-yl]phenyl}-3-(trifluoromethyl)benzamide

MS: [M+H]⁺ 517.3

### EXAMPLE 355

### N-[3-(3-bromopyrazolo[1,5-a]pyrimidin-7-yl)-4-chlorophenyl]-3-(trifluoromethyl)benzamide

To 0.085 g (0.0.204 mmol) of N-(4-chloro-3-pyrazolo[1,5-a]pyrimidin-7-ylphenyl)-3-(trifluoromethyl)benzamide in 10 mL of chloroform was added 0.044 g (0.245 mmol) of N-bromosuccinimide and the reaction was stirred at room temperature for 12 hours. The resulting mixture washed with saturated sodium bisulfite and water, dried over magnesium sulfate, filtered and concentrated in vacuo. The residue was triturated with ether to provide 0.056 g of N-[3-(3-bromopyrazolo[1,5-a]pyrimidin-7-yl)-4-chlorophenyl]-3-(trifluoromethyl)benzamide.

MS (electrospray): m/z 493 [M−H]

### EXAMPLE 356

### Ethyl 7-(3-{[4-{[(tert-butoxycarbonyl)(methyl)amino]methyl}-3-(trifluoromethyl)benzoyl]amino}phenyl)pyrazolo[1,5-a]pyrimidine-3-carboxylate

### Step 1: 4-Bromomethyl-3-trifluoromethylbenzoic acid methyl ester

To a solution of 3.526 g (16.17 mmol) of 4-methyl-3-trifluoromethylbenzoic acid methyl ester in 100 mL of carbon tetrachloride was added 3.17 g (17.79 mmol) of N-bromosuccinimide and the reaction was heated at reflux under a high intensity lamp for 3 hours. After cooling to room temperature the reaction washed with water, dried over magnesium sulfate, filtered and concentrated in vacuo. The residue was used as is for the next step.

### Step 2: Methyl 4-((methylamino)methyl)-3-(trifluoromethyl)benzoate

To 0.500 g (1.684 mmol) of the crude 4-bromomethyl-3-trifluoromethylbenzoic acid methyl ester from Step 1 in 15 mL of THF was added 8.4 mL of 2M methylamine (16.8 mmol) solution in THF and 0.050 g of tetrabutylammonium iodide. The resulting mixture was stirred overnight at room temperature and concentrated in vacuo. The residue was diluted with ethyl acetate and washed with water, and the organics were then dried over sodium sulfate, filtered and concentrated in vacuo to provide methyl 4-((methylamino)methyl)-3-(trifluoromethyl)benzoate which was used without purification in the next step.

### Step 3: Methyl 4-((tert-butoxycarbonyl(methyl)amino)methyl)-3-(trifluoromethyl)benzoate

To a solution of 0.192 g (0.777 mmol) of methyl 4-((methylamino)methyl)-3-(trifluoromethyl)benzoate in 3 mL of dichloromethane was added 0.187 g (0.855 mmol) of di-tert-butyl-dicarbonate and 0.22 mL (1.555 mmol) of triethylamine and the resulting mixture was stirred overnight at room temperature and then concentrated in vacuo. The residue was chromatographed on silica gel eluting with ethyl acetate/hexanes (1:5) to provide 0.232 g of methyl 4-((tert-butoxycarbonyl(methyl)amino)methyl)-3-(trifluoromethyl)benzoate as a yellow oil.

MS (electrospray): m/z 348 [M+H]

### Step 4: 4-{[(tert-butoxycarbonyl)(methyl)amino]methyl}-3-(trifluoromethyl)benzoic acid

To a solution of 0.187 g (0.539 mmol) of methyl 4-((tert-butoxycarbonyl(methyl)amino)methyl)-3-(trifluoromethyl)benzoate in 3 mL of THF and 3 mL of methanol was added 2.7 mL of 1N sodium hydroxide and the reaction was stirred at room temperature for 12 hours. The resulting mixture was neutralized with 0.5 N HCl and then extracted with ethyl acetate. The combined organics were washed with water and brine, dried over magnesium sulfate, filtered and concentrated in vacuo to give 0.179 g of 4-{[(tert-butoxycarbonyl)(methyl)amino]methyl}-3-(trifluoromethyl)benzoic acid. MS (electrospray): m/z 332 [M−H]

### Step 5: Ethyl 7-(3-{[4-{[(tert-butoxycarbonyl)(methyl)amino]methyl}-3-(trifluoromethyl)benzoyl]amino}phenyl)pyrazolo[1,5-a]pyrimidine-3-carboxylate

To a solution of 0.069 g (0.245 mmol) of 4-{[(tert-butoxycarbonyl)(methyl)amino]methyl}-3-(trifluoromethyl)benzoic acid in 4 mL of DMF was added 0.042 mL of Hunig's base followed by 0.127 g (0.245 mmol) of PyBop and 0.057 g (0.204 mmol) of 7-(3-amino-phenyl)-pyrazolo[1,5-a]pyrimidine-3-carboxylic acid ethyl ester from Example 1, Step 3. The resulting mixture was stirred at room temperature for 48 hours and then concentrated in vacuo. The residue was diluted with ethyl acetate, washed with water and saturated sodium bicarbonate solution, dried over magnesium sulfate, filtered and concentrated in vacuo. The residue was chromatographed on silica gel eluting with ethyl acetate/hexanes (2:1) to provide 0.043 g of ethyl 7-(3-{[4-{[(tert-butoxycarbonyl)(methyl)amino]methyl}-3-(trifluoromethyl)benzoyl]amino}phenyl)pyrazolo[1,5-a]pyrimidine-3-carboxylate as a pale yellow solid. MS (electrospray): m/z 598 [M+H]

### EXAMPLE 357

### Ethyl 7-[3-({4-[(methylamino)methyl]-3-(trifluoromethyl)benzoyl}amino)phenyl]pyrazolo[1,5-a]pyrimidine-3-carboxylate

Hydrogen chloride gas was bubbled into a solution of 0.046 g (0.077 mmol) of ethyl 7-(3-{[4-{[(tert-butoxycarbonyl)(methyl)amino]methyl}-3-(trifluoromethyl)benzoyl]amino}phenyl)pyrazolo[1,5-a]pyrimidine-3-carboxylate in 5 mL of dichloromethane for 10 minutes. The reaction vessel was then sealed and let sit overnight at room temperature. The reaction mixture was then diluted with ether and the resulting precipitate was filtered and dried in vacuo to give ethyl 7-[3-({4-[(methylamino)methyl]-3-(trifluoromethyl)benzoyl}amino)phenyl]pyrazolo[1,5-a]pyrimidine-3-carboxylate hydrochloride as a yellow solid.

MS (electrospray): m/z 498 [M+H]

### EXAMPLE 358

### N-[4-chloro-3-(3-chloropyrazolo[1,5-a]pyrimidin-7-yl)phenyl]-3-(trifluoromethyl)benzamide

To 0.085 g (0.0.204 mmol) of N-(4-chloro-3-pyrazolo[1,5-a]pyrimidin-7-ylphenyl)-3-(trifluoromethyl)benzamide in 10 mL of chloroform was added 0.033 g (0.245 mmol) of N-chlorosuccinimide and the reaction was stirred at room temperature for 24 hours. An additional 0.033 g of N-chlorosuccinimide was then added to the reaction and it was stirred at room temperature for 48 hours. The resulting mixture washed with saturated sodium bisulfite and water, dried over magnesium sulfate, filtered and concentrated in vacuo. The residue was chromatographed on silica gel eluting with ethylacetate:hexanes (1:2) to provide a yellow oil which was triturated with ethyl acetate to give N-[4-chloro-3-(3-chloropyrazolo[1,5-a]pyrimidin-7-yl)phenyl]-3-(trifluoromethyl)benzamide as a white solid.

MS (electrospray): m/z 449 [M−H]

### EXAMPLE 359

### Ethyl 7-(3-{[3-nitro-5-(trifluoromethyl)benzoyl]amino}phenyl)pyrazolo[1,5-a]pyrimidine-3-carboxylate

To a stirred solution of 246.9 mg (1.05 mmol) of 3-nitro-5-trifluoromethyl benzoic acid in 5 ml of DMF was added 676.5 mg (1.3 mmol) of (benzotriazol-1-yloxy)tripyrrolidino-phosphonium hexafluorophosphate (PyBOP) at 0° C., followed by adding 0.43 ml of diisopropylethylamine, 282.3 mg (1 mmol) of 7-(3-amino-phenyl)-pyrazolo[1,5-a]pyrimidine-3-carboxylic acid ethyl ester. The solution was stirred at room temperature overnight. After removal of the volatile material, 100 ml of ethyl acetate was added. The organic phase washed with water and a saturated sodium chloride solution, then dried over magnesium sulfate. The product was obtained by flash column chromatography eluting with ethyl acetate/hexane with quantitative yield (500.1 mg).

MS (ESI) m/z: 500.4 (M+H)⁺

### EXAMPLE 360

### N-[3-(2-pyridin-4-ylpyrazolo[1,5-a]pyrimidin-7-yl)phenyl]-2-[3-(trifluoromethyl)phenyl]acetamide

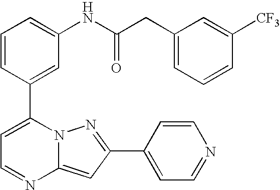

Prepared following the method used for 3,5-difluoro-N-[3-(2-pyridin-4-ylpyrazolo[1,5-a]pyrimidin-7-yl)phenyl]benzamide (Example 325), using (3-trifluoromethylphenyl)acetyl chloride in Step 2. The final purification was by silica gel column chromatography.

MS (electrospray): m/z 474 [M+H]

### EXAMPLE 361

### Methyl 7-(2-chloro-5-{[3-(trifluoromethyl)benzoyl]amino}phenyl)pyrazolo[1,5-a]pyrimidine-3-carboxylate

To 0.065 g (0.133 mmol) of the ethyl 7-(2-chloro-5-{[3-(trifluoromethyl)benzoyl]amino}phenyl)pyrazolo[1,5-a]pyrimidine-3-carboxylate dissolved in 1 mL of THF and 1 mL of methanol was added 0.7 mL of 1N sodium hydroxide and the reaction was stirred at room temperature for 24 hours. The resulting mixture was acidified with 1N HCl and extracted with dichloromethane. The organics were washed with water, dried over magnesium sulfate, filtered and concentrated in vacuo. The resulting residue was triturated with ether, filtered and dried in vacuo to provide methyl 7-(2-chloro-5-{[3-(trifluoromethyl)benzoyl]amino}phenyl)pyrazolo[1,5-a]pyrimidine-3-carboxylate.

MS (electrospray): m/z 475 [M+H]

### EXAMPLE 362

### Ethyl 7-(3-{[3-amino-5-(trifluoromethyl)benzoyl]amino}phenyl)pyrazolo[1,5-a]pyrimidine-3-carboxylate

To a stirred solution of 3.00 g (6.0 mmol) of ethyl 7-(3-{[3-nitro-5-(trifluoromethyl)benzoyl]amino}phenyl)pyrazolo[1,5-a]pyrimidine-3-carboxylate in 80 ml of ethanol and 30 ml of water was added 2.89 g (54 mmol) of ammonium chloride and 1.01 g (18 mmol) of iron powder. The reaction mixture was refluxed for 3 hr. After filtration, the solution was concentrated. To this was added 300 ml of ethyl acetate. The organic phase washed with water and a saturated sodium chloride solution, then dried over magnesium sulfate. Pure ethyl 7-(3-{[3-amino-5-(trifluoromethyl)benzoyl]amino}phenyl)pyrazolo[1,5-a]pyrimidine-3-carboxylate (2.75 g, 98% yield) was obtained following evaporation of the solvent.

MS (ESI) m/z 470.5 (M+H)⁺

Examples 363 and 364 were prepared following the procedure described for example 330, by N-[3-(3-iodopyrazolo[1,5-a]pyrimidin-7-yl)-4-methoxyphenyl]-3-(trifluoromethyl)benzamide and 4-(4,4,5,5-tetramethyl-1,3,2-dioxaborolan-2-yl)pyrazole-1-carboxylic acid tert-butyl ester. The crude compounds were purified by flash chromatography to give N-{4-methoxy-3-[3-(1H-pyrazol-4-yl)pyrazolo[1,5-a]pyrimidin-7-yl]phenyl}-3-(trifluoromethyl)benzamide (19 mg, 20% yield) and tert-butyl 4-[7-(2-methoxy-5-{[3-(trifluoromethyl)benzoyl]amino}phenyl)pyrazolo[1,5-a]pyrimidin-3-yl]-1H-pyrazole-1-carboxylate (28.5 mg, 22% yield).

### EXAMPLE 363

### N-{4-methoxy-3-[3-(1H-pyrazol-4-yl)pyrazolo[1,5-a]pyrimidin-7-yl]phenyl}-3-(trifluoromethyl)benzamide

MS (ESI) m/z 479.3 [M+H]

### EXAMPLE 364

### tert-butyl 4-[7-(2-methoxy-5-{[3-(trifluoromethyl)benzoyl]amino}phenyl)pyrazolo[1,5-a]pyrimidin-3-yl]-1H-pyrazole-1-carboxylate

MS (ESI) m/z 579.4 [M+H]

Examples 365 to 375 are prepared following the method for 3,5-difluoro-N-[3-(2-pyridin-4-ylpyrazolo[1,5-a]pyrimidin-7-yl)phenyl]benzamide (Example 325) using the appropriate acid chloride in Step 2. At the end of the Step 2 reaction the solvent is evaporated under a stream of nitrogen and the residue is purified by HPLC.

### EXAMPLE 365

### 3-bromo-N-[3-(2-pyridin-4-ylpyrazolo[1,5-a]pyrimidin-7-yl)phenyl]-benzamide, trifluoroacetic acid salt

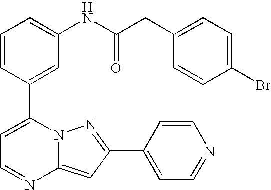

MS (electrospray): m/z 470 [M+H]

### EXAMPLE 366

### 3-fluoro-N-[3-(2-pyridin-4-ylpyrazolo[1,5-a]pyrimidin-7-yl)phenyl]benzamide, trifiroroacetic acid salt

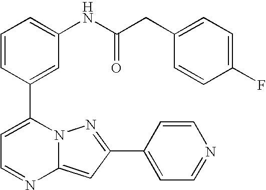

MS (electrospray): m/z 410 [M+H]

### EXAMPLE 367

### 3-nitro-N-[3-(2-pyridin-4-ylpyrazolo[1,5-a]pyrimidin-7-yl)phenyl]benzamide, trifluoroacetic acid salt

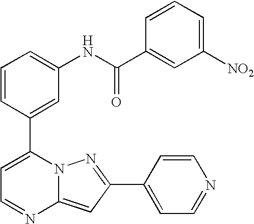

MS (electrospray): m/z 437 [M+H]

### EXAMPLE 368

### 3-cyano-N-[3-(2-pyridin-4-ylpyrazolo[1,5-a]pyrimidin-7-yl)phenyl]benzamide, trifluoroacetic acid salt

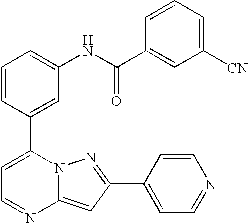

MS (electrospray): m/z 417 [M+H]

### EXAMPLE 369

### N-[3-(2-pyridin-4-ylpyrazolo[1,5-a]pyrimidin-7-yl)phenyl]-3-(trifluoromethoxy)benzamide, trifluoroacetic acid salt

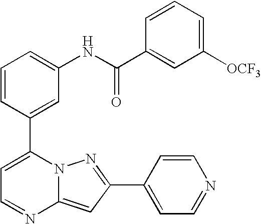

MS (electorspray): m/z 476 [M+H]

### EXAMPLE 370

### 3-(dimethylamino)-N-[3-(2-pyridin-4-ylpyrazolo[1,5-a]pyrimidin-7-yl)phenyl]benzamide, trifluoroacetic acid salt

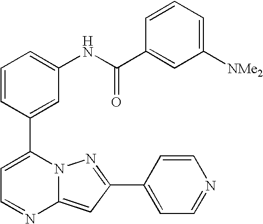

MS (electrospray): m/z 435 [M+H]

### EXAMPLE 371

### 3,4-difluoro-N-[3-(2-pyridin-4-ylpyrazolo[1,5-a]pyrimidin-7-yl)phenyl]-benzamide, trifluoroacetic acid salt

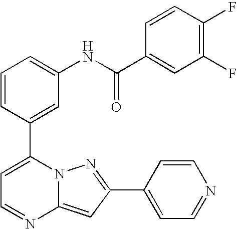

MS (electrospray): m/z 428 [M+H]

### EXAMPLE 372

### N-[3-(2-pyridin-4-ylpyrazolo[1,5-a]pyrimidin-7-yl)phenyl]-1,3-benzodioxole-5-carboxamide, trifluoroacetic acid salt

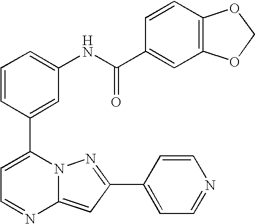

MS (electrospray): m/z 436 [M+H]

### EXAMPLE 373

### 4-fluoro-N-[3-(2-pyridin-4-ylpyrazolo[1,5-a]pyrimidin-7-yl)phenyl]-3-(trifluoromethyl)benzamide, trifluoroacetic acid salt

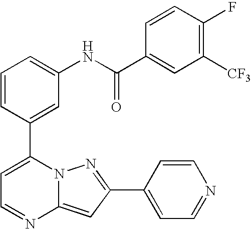

MS (electrospray): m/z 478 [M+H]

### EXAMPLE 374

### 4-bromo-3-methyl-N-[3-(2-pyridin-4-ylpyrazolo[1,5-a]pyrimidin-7-yl)phenyl]benzamide, trifluoroacetic acid salt

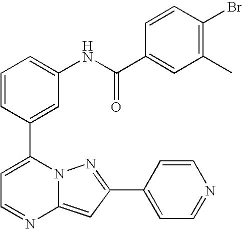

MS (electrospray): m/z 484 [M+H]

### EXAMPLE 375

### 3-fluoro-N-[3-(2-pyridin-4-ylpyrazolo[1,5-a]pyrimidin-7-yl)phenyl]-4-(trifluoromethyl)benzamide

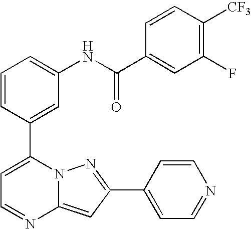

MS (electrospray): m/z 478 [M+H]

### EXAMPLE 376

### Ethyl 7-[3-({3-[(chloroacetyl)amino]-5-(trifluoromethyl)benzoyl}amino)phenyl]pyrazolo[1,5-a]pyrimidine-3-carboxylate

To the stirred solution of 1.41 g (3.0 mmol) of ethyl 7-(3-{[3-amino-5-(trifluoromethyl)benzoyl]amino}phenyl)pyrazolo[1,5-a]pyrimidine-3-carboxylate

in 20 ml of CH₂Cl₂ was added 360 mg (3 mmol) of chloroacetyl chloride at 0° C., followed by adding 356 mg (4.5 mmol) of pyridine. After addition was complete, the reaction mixture was warmed to room temperature and stirred at room temperature overnight. After removal of the volatile material, 400 ml of ethyl acetate was added. The organic phase washed with 0.1 N HCl, water and a saturated sodium chloride solution, dried over magnesium sulfate. Ethyl 7-[3-({3-[(chloroacetyl)amino]-5-(trifluoromethyl)benzoyl}amino)phenyl]pyrazolo[1,5-a]pyrimidine-3-carboxylate (1.22 g, 74% yield) was obtained on purification by flash column chromatography.

MS (ESI) m/z 546.5 (M+H)⁺

### EXAMPLE 377

### 1-(3-{[3-(2-Pyridin-4-ylpyrazolo[1,5-a]pyrimidin-7-yl)phenyl]carbamoyl}-benzyl)pyridinium chloride

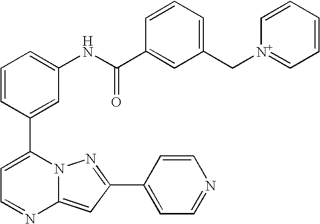

1-(3-{[3-(2-Pyridin-4-ylpyrazolo[1,5-a]pyrimidin-7-yl)phenyl]carbamoyl}-benzyl)pyridinium chloride was prepared following the method used for 3,5-difluoro-N-[3-(2-pyridin-4-ylpyrazolo[1,5-a]pyrimidin-7-yl)phenyl]benzamide (Example 325), using 3-(chloromethyl)benzoyl chloride in Step 2. At the end of step 2 the product is isolated by filtration from the reaction mixture.

MS (electrospray): m/z 483 [M]

Example 378 was prepared following the procedure described for example 330, using the corresponding starting materials.

### EXAMPLE 378

### N-{3-[3-(3,5-dimethyl-1H-pyrazol-4-yl)pyrazolo[1,5-a]pyrimidin-7-yl]phenyl}-3-(trifluoromethyl)benzamide

MS (ESI) m/z 477.5

### EXAMPLE 379

### N-{3-[3-(5-Oxo-4,5-dihydro-[1,3,4]oxadiazol-2-yl)-pyrazolo[1,5-a]pyrimidin-7-yl]-phenyl}-3-trifluoromethyl-benzamide

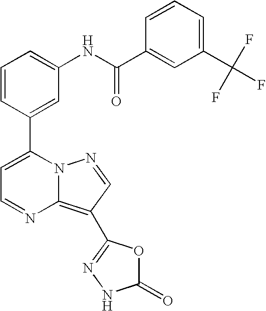

### Step 1. 7-(3-Nitro-phenyl)-pyrazolo[1,5-a]pyrimidine-3-carboxylic acid ethyl ester

A solution of 3-dimethylamino-1-(3-nitro-phenyl)-propenone (4.0 g, 18.16 mmol) and 5-amino-1H-pyrazole-4-carboxylic acid ethyl ester (3.4 g, 21.79) in acetic acid was heated to 110° C. over 7 h. The mixture was allowed to cool to 25° C. over 19 h. A precipitate formed. The precipitate was filtered and washed with 20% EtOAc in hexane to obtain 7-(3-nitro-phenyl)-pyrazolo[1,5-a]pyrimidine-3-carboxylic acid ethyl ester as a white amorphous solid (4.2 g, 74.1%).

Mass Spectrum (+ESI): 313 (M+H)⁺.

### Step 2. 7-(3-Amino-phenyl)-pyrazolo[1,5-a]pyrimidine-3-carboxylic acid ethyl ester

To a slurry of 7-(3-nitro-phenyl)-pyrazolo[1,5-a]pyrimidine-3-carboxylic acid ethyl ester (4.0 g, 12.8 mmol) in EtOH:H₂O) (50:30 mL) was added Iron (2.15 g, 38.42 mmol) and ammonium chloride (6.2 g, 11.53 mmol). The resulting mixture was heated to reflux, then cooled to 25° C. over 19 h. The crude mixture was filtered through a pad of celite, and the resulting solution was extracted with EtOAc. Following separation of the layers, the organic layer was dried over MgSO₄, filtered and concentrated to obtain a crude solid. The crude product was further purified by Biotage chromatography (cartridge 40m), eluent 100% EtOAc to obtain 7-(3-amino-phenyl)-pyrazolo[1,5-a]pyrimidine-3-carboxylic acid ethyl ester as an amorphous solid (2.6 g, 72.2%).

Mass Spectrum (+ESI): 283 (M+N₂)⁺.

### Step 3. 7-[3-(3-Trifluoromethyl-benzoylamino)-phenyl]-pyrazolo[1,5-a]pyrimidine-3-carboxylic acid ethyl ester

To a solution of 7-(3-amino-phenyl)-pyrazolo[1,5-a]pyrimidine-3-carboxylic acid ethyl ester (2.6 g, 9.22 mmol) in CH₂Cl₂ (40 mL) and pyridine (1.5 mL, 19.2 mmol) was added 3-trifluoromethyl-benzoyl chloride (2.1 mL, 14.08 mmol) dropwise at 0° C. The reaction was allowed to warm to 25° C. over 19 h. The reaction was diluted in CH₂Cl₂ (100 mL) and subsequently washed with 1N HCl (25 mL) and brine (100 mL). Following separation of the layers, the organic layer was dried over MgSO₄, filtered and concentrated to obtain a crude solid. The crude was further purified by Biotage chromatography (cartridge 40L), eluent 2:1 EtOAc-hexane to obtain 7-[3-(3-trifluoromethyl-benzoylamino)-phenyl]-pyrazolo[1,5-a]pyrimidine-3-carboxylic acid ethyl ester as an amorphous solid (1.9 g, 45.35%).

Mass Spectrum (+ESI): 455 (M+H)⁺.

### Step 4. N-[3-(3-Hydrazinocarbonyl-pyrazolo[1,5-a]pyrimidin-7-yl)-phenyl]-3-trifluoromethyl-benzamide

A solution of 7-[3-(3-trifluoromethyl-benzoylamino)-phenyl]-pyrazolo[1,5-a]pyrimidine-3-carboxylic acid ethyl ester (1.5 g, 3.3 mmol) in ethanol (40 mL) and hydrazine hydrate (21 mL) was heated to reflux under continuous N₂ flow for 1 hour. The solvents were removed in vacuo. The residue was triturated in water (40 mL) and the precipitate collected by filtration and dried to obtain N-[3-(3-hydrazinocarbonyl-pyrazolo[1,5-a]pyrimidin-7-yl)-phenyl]-3-trifluoromethyl-benzamide as a yellow solid (2.1 g). This material was used as is in the next step.

Mass Spectrum (+ESI): 441 (M+H)⁺.

### Step 5. N-{3-[3-(5-Oxo-4,5-dihydro-[1,3,4]oxadiazol-2-yl)-pyrazolo[1,5-a]pyrimidin-7-yl]-phenyl}-3-trifluoromethyl-benzamide

To a stirred solution of triphosgene (0.337 g, 1.13 mmol) in dioxane (5 mL) was added N-[3-(3-hydrazinocarbonyl-pyrazolo[1,5-a]pyrimidin-7-yl)-phenyl]-3-trifluoromethyl-benzamide (0.5 g, 1.13 mmol) in dioxane (10 mL) at 0° C. The reaction was stirred for 19 h at 25° C., then heated to 50° C. for 19 h. Following removal of solvent in vacuo, the residue was diluted in EtOAc (100 mL) and washed with brine (2×20 mL). The organic layer was dried over MgSO₄, filtered, and concentrated to obtain a crude solid. The crude material was further purified by Biotage chromatography (cartridge 40s), eluent 2:1 EtOAc-hexane, then 100% EtOAc to obtain N-{3-[3-(5-oxo-4,5-dihydro-[1,3,4]oxadiazol-2-yl)-pyrazolo[1,5-a]pyrimidin-7-yl]-phenyl}-3-trifluoromethyl-benzamide as a neon yellow-green solid (0.195 g, 36.2%).

Mass Spectrum (+ESI): 467 (M+H)⁺.

Examples 380 and 381 were prepared following the procedure described for example 330 by using corresponding starting materials.

### EXAMPLE 380

### N-{4-chloro-3-[3-(1H-pyrazol-4-yl)pyrazolo[1,5-a]pyrimidin-7-yl]phenyl}-3-(trifluoromethyl)benzamide

MS (ESI) m/z 483.4 [M+H]

### EXAMPLE 381

### N-{3-[3-(3,5-dimethyl-1H-pyrazol-4-yl)pyrazolo[1,5-a]pyrimidin-7-yl]-4-fluorophenyl}-3-(trifluoromethyl)benzamide

MS (ESI) m/z 495.3 [M+H]

### EXAMPLE 382

### 4-[(Methylamino)methyl]-N-[3-(2-pyridin-4-ylpyrazolo[1,5-a]pyrimidin-7-yl)phenyl]-3-(trifluoromethyl)benzamide

To a solution of 0.075 (0.225 mmol) of 4-{[(tert-butoxycarbonyl)(methyl)amino]methyl}-3-(trifluoromethyl)benzoic acid in 4 mL of DMF was added 0.039 mL of diisopropylethylamine (Hunig's base) followed by 0.17 g (0.225 mmol) of PyBop and 0.059 g (0.205 mmol) of 3-(2-pyridin-4-ylpyrazolo[1,5-a]pyrimidin-7-yl)aniline. The resulting mixture was stirred at room temperature for 24 hours and then diluted with ethyl acetate, washed with water, dried over sodium sulfate, filtered and concentrated in vacuo. The residue was chromatographed on silica gel eluting with chloroform/methanol (98:2) to provide 0.058 g of the desired benzamide. This benzamide was dissolved in 5 mL of dichloromethane/methanol (95:5) and hydrogen chloride gas was bubbled in for 10 minutes. The reaction vessel was then sealed and let sit overnight at room temperature. The reaction mixture was then diluted with ether and the resulting precipitate was filtered and dried in vacuo to give 4-[(methylamino)methyl]-N-[3-(2-pyridin-4-ylpyrazolo[1,5-a]pyrimidin-7-yl)phenyl]-3-(trifluoromethyl)benzamide hydrochloride as a yellow solid.

MS (electrospray): m/z 503 [M+H]

### EXAMPLE 383

### 4-{[(2-Methoxyethyl)amino]methyl}-N-[3-(2-pyridin-4-ylpyrazolo[1,5-a]pyrimidin-7-yl)phenyl]-3-(trifluoromethyl)benzamide

### Step 1: Methyl 4-{[(2-methoxyethyl)amino]methyl}-3-(trifluoromethyl)benzoate

To 0.500 g (1.684 mmol) of crude 4-bromomethyl-3-trifluoromethylbenzoic acid methyl ester in 15 mL of THF was added 0.73 mL of 2-methoxyethylamine (8.42 mmol) solution in THF and 0.050 g of tetrabutylammonium iodide. The resulting mixture was stirred overnight at room temperature and concentrated in vacuo. The residue was diluted with ethyl acetate and washed with water, and the organics were then dried over sodium sulfate, filtered and concentrated in vacuo. The residue was chromatographed on silica gel eluting with a gradient of ethyl acetate/hexanes (1:1) to 100% ethyl acetate to provide methyl 4-{[(2-methoxyethyl)amino]methyl}-3-(trifluoromethyl)benzoate which was used in the next step.

MS (electrospray): m/z 292 [M+H]

### Step 2: Methyl 4-{[(tert-butoxycarbonyl)(2-methoxyethyl)amino]methyl}-3-(trifluoromethyl)benzoate

To a solution of 0.217 g (0.746 mmol) of methyl 4-{[(2-methoxyethyl)amino]methyl}-3-(trifluoromethyl)benzoate in 3 mL of dichloromethane was added 0.179 g (0.820 mmol) of di-tert-butyl-dicarbonate and 0.21 mL (1.491 mmol) of triethylamine and the resulting mixture was stirred 48 hours at room temperature and then concentrated in vacuo. The residue was chromatographed on silica gel eluting with ethyl acetate/hexanes (1:3) to provide 0.276 g of methyl 4-{[(tert-butoxycarbonyl)(2-methoxyethyl)amino]methyl}-3-(trifluoromethyl)benzoate as a colorless oil.

MS (electrospray): m/z 392 [M+H]

### Step 3: 4-{[(tert-butoxycarbonyl)(2-methoxyethyl)amino]methyl}-3-(trifluoromethyl)benzoic acid

To a solution of 0.252 g (0.645 mmol) of methyl 4-{[(tert-butoxycarbonyl)(2-methoxyethyl)amino]methyl}-3-(trifluoromethyl)benzoate in 4 mL of THF and 4 mL of methanol was added 3.2 mL of 1N sodium hydroxide and the reaction was stirred at room temperature for 13 hours. The resulting mixture was neutralized with 1 N HCl and then extracted with ethyl acetate. The combined organics were washed with water and brine, dried over magnesium sulfate, filtered and concentrated in vacuo to give 0.230 g of 4-{[(tert-butoxycarbonyl)(2-methoxyethyl)amino]methyl}-3-(trifluoromethyl)benzoic acid. MS (electrospray): m/z 376 [M−H]

### Step 4: 4-{[(2-methoxyethyl)amino]methyl}-N-[3-(2-pyridin-4-ylpyrazolo[1,5-a]pyrimidin-7-yl)phenyl]-3-(trifluoromethyl)benzamide

To a solution of 0.093 (0.238 mmol) of 4-{[(tert-butoxycarbonyl)(2-methoxyethyl)amino]methyl}-3-(trifluoromethyl)benzoic acid in 4 mL of DMF was added 0.041 mL of Hunig's base followed by 0.124 g (0.238 mmol) of PyBop and 0.078 g (0.216 mmol) of 3-(2-pyridin-4-ylpyrazolo[1,5-a]pyrimidin-7-yl)aniline. The resulting mixture was stirred at room temperature for 24 hours and then diluted with ethyl acetate, washed with water, dried over sodium sulfate, filtered and concentrated in vacuo. The residue was chromatographed on a preparative TLC plate eluting with chloroform/methanol (95:5) to provide 0.061 g of the desired benzamide. This benzamide was dissolved in 5 mL of chloroform/methanol (95:5) and hydrogen chloride gas was bubbled in for 10 minutes. The reaction vessel was then sealed and let sit overnight at room temperature. The reaction mixture was then diluted with ether and the resulting precipitate was filtered and dried in vacuo to give 4-{[(2-methoxyethyl)amino]methyl}-N-[3-(2-pyridin-4-ylpyrazolo[1,5-a]pyrimidin-7-yl)phenyl]-3-(trifluoromethyl)benzamide hydrochloride as a yellow solid.

MS (electrospray): m/z 547 [M+H]

Example 384 was prepared following the procedure described for example 330 by using corresponding starting materials.

### EXAMPLE 384

### 3-(trifluoromethyl)-N-(3-{3-[1-(triisopropylsilyl)-1H-pyrrol-3-yl]pyrazolo[1,5-a]pyrimidin-7-yl}phenyl)benzamide

MS (electrospray): m/z 604.5 [M+H]

### EXAMPLE 385

### Ethyl 7-(3-{[3-{[(4-methylpiperazin-1-yl)acetyl]amino}-5-(trifluoromethyl)benzoyl]amino}phenyl)pyrazolo[1,5-a]pyrimidine-3-carboxylate

To a stirred solution of 65.5 mg (0.12 mmol) of ethyl 7-[3-({3-[(chloroacetyl)amino]-5-(trifluoromethyl)benzoyl}amino)phenyl]pyrazolo[1,5-a]pyrimidine-3-carboxylate in 2 ml of DMF was added 36 mg (0.36 mmol) of 1-methylpiperizine and 24.3 mg (0.24 mmol) of triethylamine. The reaction mixture was heated at 60° C. overnight. The reaction mixture was cooled to room temperature and water was added. The resulting precipitate was filtered off and washed with water to give 50.1 mg of ethyl 7-(3-{[3-{[(4-methylpiperazin-1-yl)acetyl]amino}-5-(trifluoromethyl)benzoyl]amino}phenyl)pyrazolo[1,5-a]pyrimidine-3-carboxylate (68% yield).

MS (ESI) m/z 610.2 (M+H)

### EXAMPLE 386

### Ethyl 7-[3-({3-[(pyrrolidin-1-ylacetyl)amino]-5-(trifluoromethyl)benzoyl}amino)phenyl]pyrazolo[1,5-a]pyrimidine-3-carboxylate

According to the procedure of ethyl 7-(3-{[3-{[(4-methylpiperazin-1-yl)acetyl]amino}-5-(trifluoromethyl)benzoyl]amino}phenyl)pyrazolo[1,5-a]pyrimidine-3-carboxylate (example 385), 65.5 mg of ethyl 7-[3-({3-[(chloroacetyl)amino]-5-(trifluoromethyl)benzoyl}amino)phenyl]pyrazolo[1,5-a]pyrimidine-3-carboxylate was reacted with 26.0 mg of pyrrolidine to provide 61.2 mg of ethyl 7-[3-({3-[(pyrrolidin-1-ylacetyl)amino]-5-(trifluoromethyl)benzoyl}amino)phenyl]pyrazolo[1,5-a]pyrimidine-3-carboxylate (88% yield).

MS (ESI) m/z 581.2 (M+H)⁺

### EXAMPLE 387

### Ethyl 7-[3-({3-[(morpholin-4-ylacetyl)amino]-5-(trifluoromethyl)benzoyl}amino)phenyl]pyrazolo[1,5-a]pyrimidine-3-carboxylate

According to the procedure of ethyl 7-(3-{[3-{[(4-methylpiperazin-1-yl)acetyl]amino}-5-(trifluoromethyl)benzoyl]amino}phenyl)pyrazolo[1,5-a]pyrimidine-3-carboxylate (example 385), 65.5 mg of ethyl 7-[3-({3-[(chloroacetyl)amino]-5-(trifluoromethyl)benzoyl}amino)phenyl]pyrazolo[1,5-a]pyrimidine-3-carboxylate was reacted with 31.4 mg of morpholine provided 63.5 mg of ethyl 7-[3-({3-[(morpholin-4-ylacetyl)amino]-5-(trifluoromethyl)benzoyl}amino)phenyl]pyrazolo[1,5-a]pyrimidine-3-carboxylate (89% yield).

MS (ESI) m/z 597.2 (M+H)⁺

### EXAMPLE 388

### N-{3-[3-(1H-pyrrol-3-yl)pyrazolo[1,5-a]pyrimidin-7-yl]phenyl}-3-(trifluoromethyl)benzamide

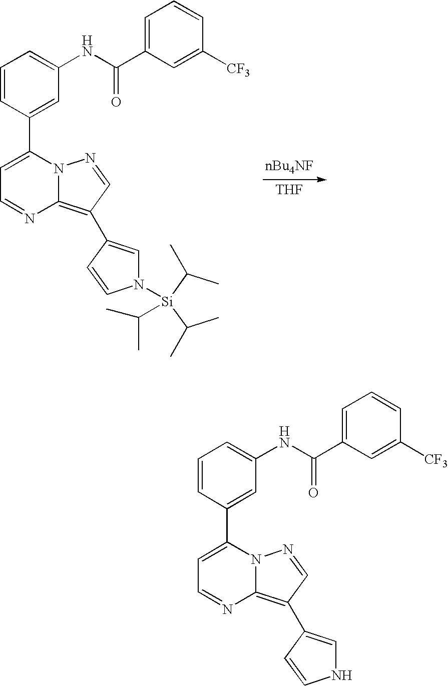

To a solution of 3-(trifluoromethyl)-N-(3-{3-[1-(triisopropylsilyl)-1H-pyrrol-3-yl]pyrazolo[1,5-a]pyrimidin-7-yl}phenyl)benzamide (259 mg, 0.429 mmol) in THF (3 mL) was added tetra-n-butyl ammonium fluoride (1.0 M solution in THF, 0.42 mL, 0.429 mmol). The mixture was stirred at room temperature for 10 min. and solvent was removed. The residue was purified by chromatography to give a yellow solid (36 mg, 20% yield).

MS 448.3 [M+H].

### EXAMPLE 389

### N-(3-{2-[2-(2,2-Dimethyl-propionylamino)-pyridin-4-yl]-pyrazolo[1,5-a]pyrimidin-7-yl}-phenyl)-3-trifluoro methyl-benzamide

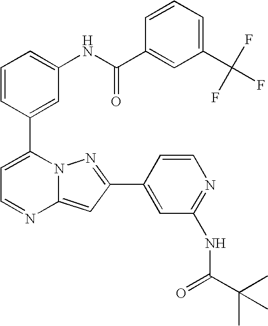

### Step 1. N-[4-(2-Cyano-acetyl)-pyridin-2-yl]-2,2-dimethyl-propionamide

To a suspension of t-butoxide in toluene was added premixed solution of 2-(2,2-dimethyl-propionylamino)-isonicotinic acid methyl ester (1 g, 4.23 mmol) and CH₃CN (0.208 g, 5.07 mmol). The solid immediately precipitated out and became difficult to stir. The reaction was heated to 80° C. over 19 h. The potassium salt was collected by filtration, washed with toluene, ether and dried to obtain N-[4-(2-cyano-acetyl)-pyridin-2-yl]-2,2-dimethyl-propionamide as a yellow solid (0.9 g, 94.7%).

Mass Spectrum (+ESI): 246 (M+H)⁺.

### Step 2. N-[4-(5-Amino-1H-pyrazol-3-yl)-pyridin-2-yl]-2,2-dimethyl-propionamide

To a solution of N-[4-(2-cyano-acetyl)-pyridin-2-yl]-2,2-dimethyl-propionamide (0.9 g, 3.99 mmol) in ethanol (20 mL), hydrazine hydrate (0.4 mL, 8 mmol) and HCl (con. 0.30 mL, 10 mmol) was heated to reflux under continuous N₂ flow for 19 h. A precipitate formed after cooling to 25° C. The solid filtered and the filtrate was concentrated in vacuo to obtain viscous oil. The crude was purified by Biotage (40s), eluent 100% EtOAc to obtain N-[4-(5-amino-1H-pyrazol-3-yl)-pyridin-2-yl]-2,2-dimethyl-propionamide as an amorphous solid (0.190 g, 20%).

Mass Spectrum (+ESI): 260 (M+H)⁺.

### Step 3. N-(3-{2-[2-(2,2-Dimethyl-propionylamino)-pyridin-4-yl]-pyrazolo[1,5-a]pyrimidin-7-yl}-phenyl)-3-trifluoromethyl-benzamide

To a solution of N-[3-(3-dimethylamino-acryloyl)-phenyl]-3-trifluoromethyl-benzamide (0.239 g, 0.662 mmol) and N-[4-(5-amino-1H-pyrazol-3-yl)-pyridin-2-yl]-2,2-dimethyl-propionamide (0.190 g, 0.0732 mmol) in THF (20 mL) was heated to 110° C. over 7 h. The mixture was allowed to cool to 25° C. over 19 h. Solvent was removed in vacuo to obtain a light brown residue. The crude was purified by Biotage (40s), eluent 4:1 EtOAc-Hex to obtain N-(3-{2-[2-(2,2-dimethyl-propionylamino)-pyridin-4-yl]-pyrazolo[1,5-a]pyrimidin-7-yl}-phenyl)-3-trifluoromethyl-benzamide as an amorphous solid (0.138 g, 35%).

Mass Spectrum (+ESI): 539 (M+H)⁺.

### EXAMPLE 390

### 3-Nitro-N-[3-(2-pyridin-4-ylpyrazolo[1,5-a]pyrimidin-7-yl)phenyl]-5-(trifluoromethyl)benzamide

To a stirred solution of 298.7 mg (1.3 mmol) of 3-nitro-5-trifluoromethyl benzoic acid in 7 ml of DMF was added 781.2 mg (1.5 mmol) of PyBOP at 0° C., followed by adding 0.91 ml of Hunig's base, 416 mg (1.1 mmol) of pyrazolo[1,5-a]pyrimidine-3-carboxylic acid, 7-(3-aminophenyl)-, ethyl ester. The solution was stirred at room temperature overnight. After removal of the volatile material, 90 ml of ethyl acetate was added. The organic phase washed with water and a saturated sodium chloride solution, dried over magnesium sulfate. Purification by flash column chromatography, eluting with ethyl acetate/hexane, provided 480.7 mg of 3-nitro-N-[3-(2-pyridin-4-ylpyrazolo[1,5-a]pyrimidin-7-yl)phenyl]-5-(trifluoromethyl)benzamide (83% yield).

MS (ESI) m/z: 505.4 (M+H)⁺

### EXAMPLE 391

### 3-Amino-N-[3-(2-pyridin-4-ylpyrazolo[1,5-a]pyrimidin-7-yl)phenyl]-5-(trifluoromethyl)benzamide

To a stirred solution of 470 mg (0.93 mmol) of 3-nitro-N-[3-(2-pyridin-4-ylpyrazolo[1,5-a]pyrimidin-7-yl)phenyl]-5-(trifluoromethyl)benzamide in 15 ml of ethanol and 5 ml of water was added 449 mg (8.4 mmol) of ammonium chloride and 156 mg (2.8 mmol) of iron powder. The reaction mixture was refluxed for 3 hr. After filtration, the solution was concentrated. Ethyl acetate (100 ml) was added. The organic phase washed with water and a saturated sodium chloride solution, then dried over magnesium sulfate. Following removal of solvent, 146 mg of 3-amino-N-[3-(2-pyridin-4-ylpyrazolo[1,5-a]pyrimidin-7-yl)phenyl]-5-(trifluoromethyl)benzamide was obtained (33% yield).

MS (ESI) m/z 475.2 (M+H)⁺

### EXAMPLE 392

### 3-[(Chloroacetyl)amino]-N-[3-(2-pyridin-4-ylpyrazolo[1,5-a]pyrimidin-7-yl)phenyl]-5-(trifluoromethyl)benzamide

To a stirred solution of 135 mg (0.28 mmol) of 3-amino-N-[3-(2-pyridin-4-ylpyrazolo[1,5-a]pyrimidin-7-yl)phenyl]-5-(trifluoromethyl)benzamide in 4 ml of methylene chloride (CH₂Cl₂) and 2.5 ml of DMF was added 35.4 mg (0.13 mmol) of chloroacetyl chloride at 0° C., followed by adding 33.2 mg (0.42 mmol) of pyridine. After addition was complete, the reaction mixture was warmed to room temperature and stirred at room temperature overnight. After removal of the volatile material, 40 ml of ethyl acetate was added. The precipitate was filtered off and washed with ethyl acetate/hexane. After drying in vacuo, 130 mg of 3-[(chloroacetyl)amino]-N-[3-(2-pyridin-4-ylpyrazolo[1,5-a]pyrimidin-7-yl)phenyl]-5-(trifluoromethyl)benzamide was obtained (83% yield).

MS (ESI) m/z 551.2 (M+H)⁺

### EXAMPLE 393

### N-[3-(2-Pyridin-4-ylpyrazolo[1,5-a]pyrimidin-7-yl)phenyl]-3-[(pyrrolidin-1-ylacetyl)amino]-5-(trifluoromethyl)benzamide

To a stirred solution of 61 mg (0.11 mmol) of 3-[(chloroacetyl)amino]-N-[3-(2-pyridin-4-ylpyrazolo[1,5-a]pyrimidin-7-yl)phenyl]-5-(trifluoromethyl)benzamide in 2 ml of DMF was added 24 mg (0.33 mmol) of pyrrolidine and 22 mg (0.22 mmol) of triethylamine. The reaction mixture was heated at 50° C. overnight. After removal of the volatile material, the resulting solid washed with ethyl acetate/hexane and water, then dried. This provided 29 mg of N-[3-(2-pyridin-4-ylpyrazolo[1,5-a]pyrimidin-7-yl)phenyl]-3-[(pyrrolidin-1-ylacetyl)amino]-5-(trifluoromethyl)benzamide was obtained (46% yield).

MS (ESI) m/z 586.3 (M+H).

### EXAMPLE 394

### N-(4-chloro-3-pyrazolo[1,5-a]pyrimidin-7-ylphenyl)-4-(morpholin-4-ylmethyl)-3-(trifluoromethyl)benzamide

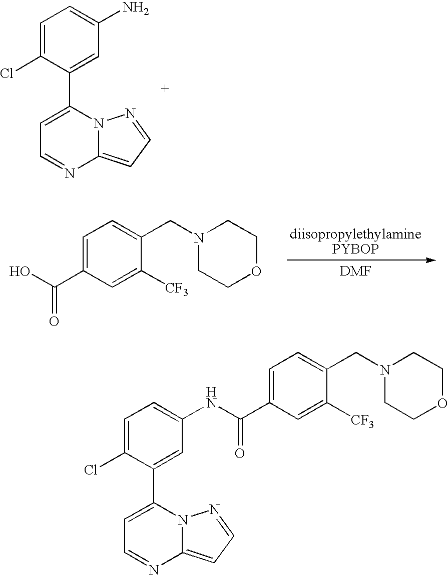

A mixture of 4-chloro-3-pyrazolo[1,5-a]pyrimidin-7-ylaniline (100 mg, 0.408 mmol), 4-(morpholin-4-ylmethyl)-3-(trifluoromethyl)benzoic acid (159 mg, 0.490 mmol), N,N-diisopropylethylamine (126 mg, 0.17 mL, 0.980 mmol), and benzotriazol-1-yloxytripyrrolidinophosphonium hexafluorophosphate (PYBOP, 254 mg, 0.490 mmol) were dissolved in DMF (5 mL). The mixture was stirred overnight at room temperature, diluted into ethyl acetate, washed with saturated sodium chloride solution, dried over sodium sulfate, filtered and concentrated to give a brown residue. This brown residue was purified by chromatography to give N-(4-chloro-3-pyrazolo[1,5-a]pyrimidin-7-ylphenyl)-4-(morpholin-4-ylmethyl)-3-(trifluoromethyl)benzamide as a yellow solid (74 mg, 35% yield).

MS 516.2 [M+H]

Example 395 was prepared following the procedure described for example 394, using the corresponding starting materials.

### EXAMPLE 395

### 4-(morpholin-4-ylmethyl)-N-[3-(2-pyridin-4-ylpyrazolo[1,5-a]pyrimidin-7-yl)phenyl]-3-(trifluoromethyl)benzamide

MS 559.4 [M+H]

What is claimed is:

1. A compound of Formula I 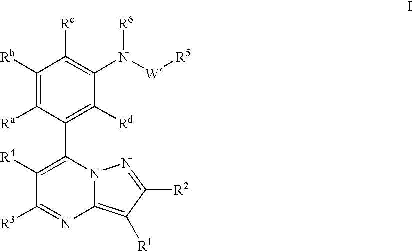
or a pharmaceutically acceptable salt or a prodrug thereof
wherein
R¹ is selected from the group consisting of R⁷, J, —C(O)OR¹⁶, —C(O)NR⁷R¹⁴, —NR⁶C(O)R¹⁶, nitrile, a 5-7 membered heterocyclic ring or heteroaryl ring containing 1-3 heteroatoms selected from N, O or S, and an aryl ring, wherein the R⁷ group, the R¹⁴ group, the R¹⁶ group, the heterocyclic ring, the heteroaryl ring and the aryl ring can be optionally substituted with one to four substituents selected from the group consisting of -J, —NO₂, —CN, —N₃, —CHO, —CF₃, —OCF₃, —R¹¹, —OR¹¹, —S(O)ₘR¹¹, —NR¹⁵R¹¹, —NR¹²S(O)ₘR¹⁵, —OR⁹OR¹¹, —OR⁹NR¹⁵R¹¹, —N(R¹²)R⁹OR¹⁵, —N(R¹²)R⁹NR¹⁵R¹¹, —NR¹²C(O)R¹⁵, —C(O)R¹¹, —C(O)OR¹¹, —C(O)NR¹²R¹¹, —OC(O)R¹¹, —OC(O)OR¹¹, —OC(O)NR¹⁵R¹¹, NR¹²C(O)R¹⁵, —NR¹²C(O)OR¹⁵, —NR¹²C(O)NR¹⁵R¹¹, —R⁸OR¹¹, —R⁸NR¹⁵R¹¹, —R¹¹S(O)ₘR¹¹, —R⁸C(O)R¹¹, —R⁸C(O)OR¹¹, —R⁸C(O)NR¹⁵R¹¹, —R⁸OC(O)R¹¹, —R⁸OC(O)OR¹¹, —R⁸OC(O)NR¹⁵R¹¹, —R⁸NR¹²C(O)R¹⁵, —R⁸NR¹²C(O)OR¹⁵, —R⁸NR¹²C(O)NR¹⁵R¹¹, R²⁰, —OR⁹R²⁰, —N(R¹²)R⁹R²⁰, —C(O)R²⁰, —OC(O)R²⁰, —NR¹²C(O)R²⁰, —R⁸R²⁰, —R⁸C(O)R²⁰, —R⁸OC(O)R²⁰, —R⁸NR¹²C(O)R²⁰ and YR¹⁰;
R² is an alkyl of 1-6 carbon atoms, branched alkyl of 3-8 carbon atoms, cis-alkenyl of 2-6 carbon atoms, a trans-alkenyl of 2-6 carbon atoms, an alkynyl of 2-6 carbon atoms, aryl, heteroaryl or heterocyclyl; each of said alkyl of 1-6 carbon atoms, branched alkyl of 3-8 carbon atoms, cis-alkenyl of 2-6 carbon atoms, trans-alkenyl of 2-6 carbon atoms, alkynyl of 2-6 carbon atoms, aryl, heteroaryl or heterocyclyl being optionally substituted with one to four substituents selected from the group consisting of -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹⁷)R⁹OR¹⁷, —N(R¹¹)R⁹NR⁷R¹⁴, —NR¹⁷C(O)R¹¹, —C(O)R¹⁷, —C(O)OR¹⁷, —C(O)NR⁷R¹⁴, —OC(O)R¹⁷, —OC(O)OR¹⁷, —OC(O)NR⁷R¹⁴, NR¹⁷C(O)R¹¹, —NR¹⁷C(O)OR¹¹, —NR¹⁷C(O)NR⁷R¹⁴, —R⁸OR¹⁷, —R⁸NR⁷R¹⁴, —R⁸S(O)ₘR¹⁷, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹⁷C(O)R¹¹, —R⁸NR¹⁷C(O)OR¹¹, —R⁸NR¹⁷C(O)NR⁷R¹⁴ and YR¹⁰;
R^{a}, R^{b}, R^{c}, R^{d}, R³ and R⁴ are independently selected from the group consisting of H, -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷, —OC(O)OR¹¹, —OC(O)NR⁷R¹⁴, NR¹¹C(O)R¹⁷, —NR¹¹C(O)OR¹⁷, —NR¹¹C(O)NR⁷R¹⁴, —R⁸OR¹⁷, —R⁸NR⁷R¹⁴, —R⁸S(O)ₘR¹⁷, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷, —R⁸NR¹¹C(O)NR⁷R¹⁴ and YR¹⁰;
R⁵ is an alkyl of 1-6 carbon atoms, branched alkyl of 3-8 carbon atoms, cis-alkenyl of 2-6 carbon atoms, a trans-alkenyl of 2-6 carbon atoms, an alkynyl of 2-6 carbon atoms, an aryl ring, a heterocyclic ring or a heteroaryl ring, said heterocylic ring and heteroaryl containing 1-3 heteroatoms selected from N, O or S, wherein the heterocyclic, heteroaryl and aryl rings are optionally substituted with one to four substituents selected from the group consisting of -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R⁷, —OC(O)OR¹⁷, —OC(O)NR⁷R¹⁴, NR¹¹C(O)R¹⁷, —NR¹¹C(O)OR¹⁷, —NR¹¹C(O)NR⁷R¹⁴, —R⁸OR¹⁷, —R⁸NR⁷R¹⁴, —R⁸S(O)ₘR¹⁷, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷, —R⁸NR¹¹C(O)NR⁷R¹⁴, —YR⁸R¹⁰, —YR⁸NR⁷R¹⁴ and —YR¹⁰;
R⁶ is H, alkyl of 1-6 carbon atoms or branched alkyl of 3-8 carbon atoms;
R⁷, R¹¹, R¹², R¹⁴, R¹⁵, R¹⁶, and R¹⁷ are independently selected from H, alkyl of 1-6 carbon atoms, branched alkyl of 3-8 carbon atoms, cis-alkenyl of 2-6 carbon atoms, a trans-alkenyl of 2-6 carbon atoms, and an alkynyl of 2-6 carbon atoms; said alkyl, branched alkyl, cis-alkenyl, trans-alkenyl, and alkynyl groups being optionally substituted with 1-3 J atoms; R⁷ and R¹⁴ together with the N to which they are attached may join to form a 3 to 8 membered ring, said 3 to 8 membered ring optionally containing additional heteroatoms N, O, or S(O)ₘ to form a heterocycle which can optionally be substituted with alkyl of 1-6 carbon atoms, carbonyl, hydroxy, alkoxy of 1 to 6 carbon atoms, NH₂, NHR⁶, or N(R⁶)₂;
R⁸ is a divalent group selected from alkyl of 1-6 carbon atoms, alkenyl of 2-6 carbon atoms, and alkynyl of 2-6 carbon atoms;
R⁹ is a divalent alkyl group of 2-6 carbon atoms;
R¹⁰ is selected from the group consisting of a cycloalkyl ring of 3-10 carbons, a bicycloalkyl ring of 3-10 carbons, an aryl, a heterocyclyl ring, a heteroaryl ring, and a heteroaryl fused to 1-3 aryl or heteroaryl rings; any of said heterocyclyl ring and heteroaryl rings containing 1-3 heteroatoms selected from N, O or S; wherein any of the aryl, cycloalkyl, bicycloalkyl, heterocyclic or heteroaryl rings may be optionally substituted with one to four substituents selected from the group consisting of —H, -aryl, —CH₂-aryl, —NH-aryl, —O-aryl, —S(O)ₘ-aryl, -J, —NO₂, —CN, —N₃, —CHO, —CF₃, —OCF₃, —R¹⁷,
—OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷—, —OC(O)OR¹⁷, —OC(O)NR⁷R¹⁴, —NR¹¹C(O)R¹⁷, —NR¹¹C(O)OR¹⁷, —NR¹¹C(O)NR⁷R¹⁴, —R⁸OR¹⁷, R⁸NR⁷R¹⁴, —R⁸S(O)ₘR¹⁷, —R⁸C(O)R¹⁷, —R⁸C(O)OR¹⁷, —R⁸C(O)NR⁷R¹⁴, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷, and —R⁸NR¹¹C(O)NR⁷R¹⁴;
R²⁰ is a heterocyclic ring containing 3-8 members, at least one member being N which is the point of attachment for the moiety, and optionally said 3 to 8 membered ring containing additional heteroatoms N, O, or S(O)ₘ and said 3-8 membered ring being optionally substituted with 1-4 substituents selected from alkyl of 1-6 carbon atoms, carbonyl, hydroxy, alkoxy of 1 to 6 carbon atoms, NH₂, NHR⁶, or N(R⁶)₂;
J is fluoro, chloro, bromo, or iodo;
m is an integer of 0-2;
W′ is —C(O)— or —C(O)—NR¹⁷—, —SO₂—, or —CO—C(R⁶)₂—; and
Y is selected from the group consisting of a bond, a divalent alkyl group of 1-6 carbon atoms, NH, O, —NR¹⁷, —C≡C—, cis- —CH═CH—, and trans- —CH═CH—.

2. A compound of Formula I 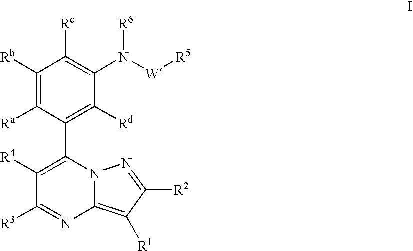
or a pharmaceutically acceptable salt or prodrug thereof
wherein
R¹ is —C(O)—NH—R¹³, substituted aryl, substituted heteroaryl, substituted heterocyclyl, —C(O)O-substituted alkyl, —C(O)O-heteroaryl, or substituted alkynyl;
R¹³ is heteroaryl, alkyl of 1 to 6 carbon atoms optionally substituted with heterocyclyl, heteroaryl, alkoxy, optionally substituted aryl, dialkylamino, or alkylamino;
R² is selected from the group consisting of R⁷, J, —C(O)OR¹⁶, —C(O)NR⁷R¹⁴, —NR⁶C(O)R¹⁶, nitrile, a 5-7 membered heterocyclic ring or heteroaryl ring containing 1-3 heteroatoms selected from N, O or S, and an aryl ring, wherein the R⁷ group, the R¹⁴ group, the R¹⁶ group, the heterocyclic ring, the heteroaryl ring and the aryl ring can be optionally substituted with one to four substituents selected from the group consisting of -J, —NO₂, —CN, —N₃, —CHO, —CF₃, —OCF₃, —R¹¹, —OR¹¹, —S(O)ₘR¹¹, —NR¹⁵R¹¹, —NR¹²S(O)ₘR¹⁵, —OR⁹OR¹¹, —OR⁹NR¹⁵R¹¹, —N(R¹²)R⁹OR¹⁵, —N(R¹²)R⁹NR¹⁵R¹¹, —NR²C(O)R¹⁵, —C(O)R¹¹, —C(O)OR¹¹, —C(O)NR¹²R¹¹, —OC(O)R¹¹, —OC(O)OR¹¹, —OC(O)NR¹⁵R¹¹, NR¹²C(O)R¹⁵, —NR¹²C(O)OR¹⁵, —NR¹²C(O)NR¹⁵R¹¹, —R⁸OR¹¹, —R⁸NR¹⁵R¹¹, —R⁸S(O)ₘR¹¹, —R⁶C(O)R¹¹, —R⁸C(O)OR¹¹, —R⁸C(O)NR¹⁵R¹¹, —R⁸OC(O)R¹¹, —R⁸OC(O)OR¹¹, —R⁸OC(O)NR¹⁵R¹¹, —R⁸NR¹²C(O)R¹⁵, —R⁸NR¹²C(O)OR¹⁵, —R⁸NR¹²C(O)NR¹⁵R¹¹, R²⁰, —OR⁹R²⁰, —N(R¹²)R⁹R²⁰, —C(O)R²⁰, —OC(O)R²⁰, —NR¹²C(O)R²⁰, —R⁸R²⁰, —R⁸C(O)R²⁰, —R⁸OC(O)R²⁰, —R⁸NR¹²C(O)R²⁰, and YR¹⁰;
R^{a}, R^{b}, R^{c}, R^{d}, R³ and R⁴ are independently selected from the group consisting of H, -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷, —OC(O)OR¹⁷, —OC(O)NR⁷R¹⁴, NR¹¹C(O)R¹⁷, —NR¹¹C(O)OR¹⁷, —NR¹¹C(O)NR⁷R¹⁴, —R⁸OR¹⁷, —R⁸NR⁷R¹⁴, R⁸S(O)ₘR¹⁷, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷, —R⁸NR¹¹C(O)NR⁷R¹⁴ and YR¹⁰;
R⁵ is an alkyl of 1-6 carbon atoms, branched alkyl of 3-8 carbon atoms, cis-alkenyl of 2-6 carbon atoms, a trans-alkenyl of 2-6 carbon atoms, an alkynyl of 2-6 carbon atoms, an aryl ring, a heterocyclic ring or a heteroaryl ring, said heterocylic ring and heteroaryl containing 1-3 heteroatoms selected from N, O or S, wherein the heterocyclic, heteroaryl and aryl rings are optionally substituted with one to four substituents selected from the group consisting of -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷, —OC(O)OR¹⁷, —OC(O)NR⁷R¹⁴, NR¹¹C(O)R¹⁷, —NR¹¹C(O)OR¹⁷, —NR¹¹C(O)NR⁷R¹⁴, —R⁸OR¹⁷, —R⁸NR⁷R¹⁴, —R⁸S(O)ₘR¹⁷, —R⁸C(O)R¹⁷, —R⁶C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷, —R⁸NR¹¹C(O)NR⁷R¹⁴, —YR⁸R¹⁰, —YR⁸NR⁷R¹⁴ and —YR¹⁰;
R⁶ is H, alkyl of 1-6 carbon atoms or branched alkyl of 3-8 carbon atoms;
R⁷, R¹¹, R¹², R¹⁴, R¹⁵, R¹⁶, and R¹⁷ are independently selected from H, alkyl of 1-6 carbon atoms, branched alkyl of 3-8 carbon atoms, cis-alkenyl of 2-6 carbon atoms, a trans-alkenyl of 2-6 carbon atoms, and an alkynyl of 2-6 carbon atoms; said alkyl, branched alkyl, cis-alkenyl, trans-alkenyl, and alkynyl groups being optionally substituted with 1-3 J atoms; R⁷ and R¹⁴ together with the N to which they are attached may join to form a 3 to 8 membered ring, said 3 to 8 membered ring optionally containing additional heteroatoms N, O, or S(O)ₘ to form a heterocycle which can optionally be substituted with alkyl of 1-6 carbon atoms, carbonyl, hydroxy, alkoxy of 1 to 6 carbon atoms;
R⁸ is a divalent group selected from alkyl of 1-6 carbon atoms, alkenyl of 2-6 carbon atoms, and alkynyl of 2-6 carbon atoms;
R⁹ is a divalent alkyl group of 2-6 carbon atoms;
R¹⁰ is selected from the group consisting of a cycloalkyl ring of 3-10 carbons, a bicycloalkyl ring of 3-10 carbons, an aryl, a heterocyclyl ring, a heteroaryl ring, and a heteroaryl fused to 1-3 aryl or heteroaryl rings; any of said heterocyclyl ring and heteroaryl rings containing 1-3 heteroatoms selected from N, O or S; wherein any of the aryl, cycloalkyl, bicycloalkyl, heterocyclic or heteroaryl rings may be optionally substituted with one to four substituents selected from the group consisting of —H, -aryl, —CH₂-aryl, —NH-aryl, —O-aryl, —S(O)ₘ-aryl, -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷—, —OC(O)OR¹⁷, —OC(O)NR⁷R¹⁴, —NR¹¹C(O)R¹⁷, —NR¹¹C(O)OR¹⁷, —NR¹¹C(O)NR⁷R¹⁴, —R⁸OR¹⁷, R⁸NR⁷R¹⁴, —R⁸S(O)ₘR¹⁷, —R⁸C(O)R¹⁷, —R⁸C(O)OR¹⁷, —R⁸C(O)NR⁷R¹⁴, —R⁸C(O)R¹⁷, —R⁸C(O)OR¹⁷, —R⁸C(O)NR⁷R¹⁴, —R⁸OC(O)R⁷, R⁸OC(O)OR¹⁷, —R⁸OC(O)NR⁷R¹⁴, —R⁸NR¹¹C(O)R¹⁷, —R⁸NR¹¹C(O)OR¹⁷ and —R⁸NR¹¹C(O)NR⁷R¹⁴;
R²⁰ is a heterocyclic ring containing 3-8 members, at least one member being N which is the point of attachment for the moiety, and optionally said 3 to 8 membered ring containing additional heteroatoms N, O, or S(O)ₘ and said 3-8 membered ring being optionally substituted with 1-4 substituents selected from alkyl of 1-6 carbon atoms, carbonyl, hydroxy, alkoxy of 1 to 6 carbon atoms, NH₂, NHR⁶, or N(R⁶)₂;
J is fluoro, chloro, bromo, or iodo;
m is an integer of 0-2;
W′ is —C(O)— or —C(O)—NR¹⁷—, —SO₂—, or —CO—C(R⁶)₂—; and
Y is selected from the group consisting of a bond, a divalent alkyl group of 1-6 carbon atoms, NH, O, —NR¹⁷, —C≡C—, cis- —CH═CH—, and trans- —CH═CH—.

3. A compound of Formula I 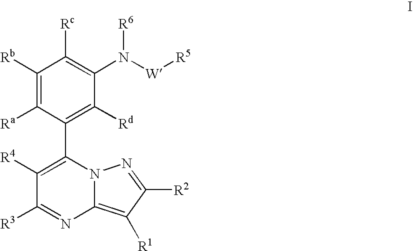
or a pharmaceutically acceptable salt or prodrug thereof
wherein
R¹ is selected from the group consisting of H, J, —C(O)OR¹⁶, —C(O)NR⁷R¹⁴, —NR⁶C(O)R¹⁶, nitrile, a 5-7 membered heterocyclic ring or heteroaryl ring containing 1-3 heteroatoms selected from N, O or S, and an aryl ring, wherein the R⁷ group, the R¹⁴ group, the R¹⁶ group, the heterocyclic ring, the heteroaryl ring and the aryl ring can be optionally substituted with one to four substituents selected from the group consisting of -J, —NO₂, —CN, —N₃, —CHO, —CF₃, —OCF₃, —R¹¹, —OR¹¹, —S(O)ₘR¹¹, —NR¹⁵R¹¹, —NR¹²S(O)ₘR¹⁵, —OR⁹OR¹¹, —OR⁹NR¹⁵R¹¹, —N(R¹²)R⁹OR¹⁵, —N(R¹²)R⁹NR¹⁵R¹¹, —NR¹²C(O)R¹⁵, —C(O)R¹¹, —C(O)OR¹¹, —C(O)NR¹²R¹¹, —OC(O)R¹¹, —OC(O)OR¹¹, —OC(O)NR¹⁵R¹¹, NR¹²C(O)R¹⁵, —NR¹²C(O)OR¹⁵, —NR¹²C(O)NR¹⁵R¹¹, —R⁸OR¹¹, —R⁸NR¹⁵R¹¹, R⁸S(O)ₘR¹¹, —R⁸C(O)R¹¹, —R⁶C(O)OR¹¹, —R⁸C(O)NR¹⁵R¹¹, —R⁸OC(O)R¹¹, —R⁸OC(O)OR¹¹, —R⁸OC(O)NR¹⁵R¹¹, —R⁸NR¹²C(O)R¹⁵, —R⁸NR¹²C(O)OR¹⁵, —R⁸NR¹²C(O)NR¹⁵R¹¹, R²⁰, —OR⁹R²⁰, —N(R¹²)R⁹R²⁰, —C(O)R²⁰, —OC(O)R²⁰, —NR¹²C(O)R²⁰, —R⁸R²⁰, —R⁸C(O)R²⁰, —R⁸OC(O)R²⁰, —R⁸NR¹²C(O)R²⁰, and YR¹⁰;
R² is selected from the group consisting of H, J, —C(O)OR⁶, —C(O)NR⁷R¹⁴, —NR⁶C(O)R¹⁶, nitrile, a 5-7 membered heterocyclic ring or heteroaryl ring containing 1-3 heteroatoms selected from N, O or S, and an aryl ring, wherein the R⁷ group, the R¹⁴ group, the R¹⁶ group, the heterocyclic ring, the heteroaryl ring and the aryl ring can be optionally substituted with one to four substituents selected from the group consisting of -J, —NO₂, —CN, —N₃, —CHO, —CF₃, —OCF₃, —R¹¹, —OR¹¹, —S(O)ₘR¹¹, —NR¹⁵R¹¹, —NR¹²S(O)ₘR¹⁵, —OR⁹OR¹¹, —OR⁹NR¹⁵R¹¹, —N(R¹²)R⁹OR¹⁵, —N(R¹²)R⁹NR¹⁵R¹¹, —NR¹²C(O)R¹⁵, —C(O)R¹¹, —C(O)OR¹¹, —C(O)NR¹²R¹¹, —OC(O)R¹¹, —OC(O)OR¹¹, —OC(O)NR¹⁵R¹¹, NR¹²C(O)R¹⁵, —NR¹²C(O)OR¹⁵, —NR¹²C(O)NR¹⁵R¹¹, —R⁸OR¹¹, —R⁸NR¹⁵R¹¹, —R⁸S(O)ₘR¹¹, —R⁸C(O)R¹¹, —R⁸C(O)OR¹¹, —R⁸C(O)NR¹⁵R¹¹, —R⁸OC(O)R¹¹, —R⁸OC(O)OR¹¹—R⁸OC(O)NR¹⁵R¹¹, —R⁸NR¹²C(O)R¹⁵, —R⁸NR¹²C(O)OR¹⁵, —R⁸NR¹²C(O)NR¹⁵R¹¹, R²⁰, —OR⁹R²⁰, —N(R¹²)R⁹R²⁰, —C(O)R²⁰, —OC(O)R²⁰, —NR¹²C(O)R²⁰, —R⁸R²⁰, —R⁸C(O)R²⁰, —R⁸OC(O)R²⁰, —R⁸NR¹²C(O)R²⁰, and YR¹⁰;
R^{a}, R^{b}, R^{c}, R^{d}, R³ and R⁴ are independently selected from the group consisting of H, -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷, —OC(O)OR¹⁷, —C(O)NR⁷R¹⁴, NR¹¹C(O)R¹⁷, —NR¹¹C(O)OR¹⁷, —NR¹¹C(O)NR⁷R¹⁴, —R⁸OR¹⁷, —R⁸NR⁷R¹⁴, —R⁸S(O)ₘR¹⁷, —R⁸C(O)R¹⁷, —R⁸C(O)OR¹⁷, —R⁶C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷, —R⁸NR¹¹C(O)NR⁷R¹⁴ and YR¹⁰;
R⁵ is —NH-aryl-heterocyclyl, —NH-aryl-heteroaryl, —CH₂-substituted aryl, —CH₂—R¹⁸, or NH—R¹⁸, said aryl portion, heterocyclyl portion and heteroaryl portion of the —NH-aryl-heterocyclyl and —NH-aryl-heteroaryl groups being optionally substituted;
R⁶ is H, alkyl of 1-6 carbon atoms or branched alkyl of 3-8 carbon atoms;
R⁷, R¹¹, R¹², R¹⁴, R¹⁵, R¹⁶, and R¹⁷ are independently selected from H, alkyl of 1-6 carbon atoms, branched alkyl of 3-8 carbon atoms, cis-alkenyl of 2-6 carbon atoms, a trans-alkenyl of 2-6 carbon atoms, and an alkynyl of 2-6 carbon atoms; said alkyl, branched alkyl, cis-alkenyl, trans-alkenyl, and alkynyl groups being optionally substituted with 1-3 J atoms; R⁷ and R¹⁴ together with the N to which they are attached may join to form a 3 to 8 membered ring;
R⁸ is a divalent group selected from alkyl of 1-6 carbon atoms, alkenyl of 2-6 carbon atoms, and alkynyl of 2-6 carbon atoms;
R⁹ is a divalent alkyl group of 2-6 carbon atoms;
R¹⁰ is selected from the group consisting of a cycloalkyl ring of 3-10 carbons, a bicycloalkyl ring of 3-10 carbons, an aryl, a heterocyclyl ring, a heteroaryl ring, and a heteroaryl fused to 1-3 aryl or heteroaryl rings; any of said heterocyclyl ring and heteroaryl rings containing 1-3 heteroatoms selected from N, O or S; wherein any of the aryl, cycloalkyl, bicycloalkyl, heterocyclic or heteroaryl rings may be optionally substituted with one to four substituents selected from the group consisting of —H, -aryl, —CH₂-aryl, —NH-aryl, —O-aryl, —S(O)ₘ-aryl, -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷—, —OC(O)OR¹⁷, —OC(O)NR⁷R¹⁴, —NR¹¹C(O)R¹⁷, —NR¹¹C(O)OR¹⁷, —NR¹¹C(O)NR⁷R¹⁴, —R⁸OR¹⁷, R⁸NR⁷R¹⁴, —R⁸S(O)ₘR¹⁷, —R⁸C(O)R¹⁷, —R⁸C(O)OR¹⁷, —R⁸C(O)NR⁷R¹⁴, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷ and R⁸NR¹¹C(O)NR⁷R¹⁴;
R¹⁸ is an aryl ring fused to a heteroaryl ring or heterocyclic ring, such as 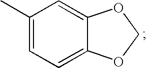
R²⁰ is a heterocyclic ring containing 3-8 members, at least one member being N which is the point of attachment for the moiety, and optionally said 3 to 8 membered ring containing additional heteroatoms N, O, or S(O)ₘ and said 3-8 membered ring being optionally substituted with 1-4 substituents selected from alkyl of 1-6 carbon atoms, carbonyl, hydroxy, alkoxy of 1 to 6 carbon atoms, NH₂, NHR⁶, or N(R⁶)₂;
J is fluoro, chloro, bromo, or iodo;
m is an integer of 0-2;
W′ is —C(O)— or —C(O)—NR¹⁷—, —SO₂—, or —CO—C(R⁶)₂—; and
Y is selected from the group consisting of a bond, a divalent alkyl group of 1-6 carbon atoms, NH, O, —NR¹⁷, —C≡C—, cis- —CH═CH—, and trans- —CH═CH—.

4. A compound of Formula I 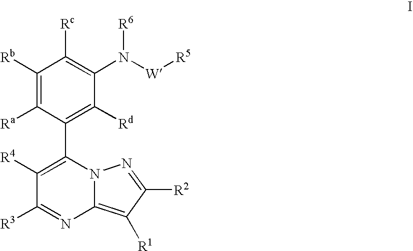
or a pharmaceutically acceptable salt or prodrug thereof
wherein
R¹ is selected from the group consisting of H, J, —C(O)OR¹⁶, —NR⁶C(O)R¹⁶, a 5-7 membered heterocyclic ring or heteroaryl ring containing 1-3 heteroatoms selected from N, O or S, and an aryl ring, wherein the R⁷ group, the R¹⁴ group, the R¹⁶ group, can be optionally substituted with one to four substituents selected from the group consisting of -J, —NO₂, —CN, —N₃, —CHO, —CF₃, —OCF₃, —R¹¹, —OR¹¹, —S(O)ₘR¹¹, —NR¹⁵R¹¹, —NR¹²S(O)ₘR¹⁵, —OR⁹OR¹¹, —OR⁹NR¹⁵R¹¹, —N(R¹²)R⁹OR¹⁵, —N(R¹²)R⁹NR¹⁵R¹¹, —NR¹²C(O)R¹⁵, —C(O)R¹¹, —C(O)OR¹¹, —C(O)NR¹²R¹¹, —OC(O)R¹¹, —OC(O)OR¹¹, —OC(O)NR¹⁵R¹¹, NR¹²C(O)R¹⁵, —NR¹²C(O)OR¹⁵, —NR¹²C(O)NR¹⁵R¹¹, —R⁸OR¹¹, —R⁸NR¹⁵R¹¹, —R⁸S(O)ₘR¹¹, —R⁸C(O)R¹¹, —R⁸C(O)OR¹¹, —R⁸C(O)NR¹⁵R¹¹, —R⁸OC(O)R¹¹, —R⁸OC(O)OR¹¹, —R⁸OC(O)NR¹⁵R¹¹, —R⁸NR¹²C(O)R¹⁵, —R⁸NR¹²C(O)OR¹⁵, —R⁸NR¹²C(O)NR¹⁵R¹¹ and YR¹⁰;
R² is selected from the group consisting of H, J, —C(O)OR¹⁶, —C(O)NR⁷R¹⁴, —NR⁶C(O)R¹⁶, nitrile;
R^{a}, R^{b}, R^{c}, R^{d}, R³ and R⁴ are independently selected from the group consisting of H, -J, —NO₂, —CN, —N₃, —CHO, —CF₃, —OCF₃, —R⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷, —OC(O)OR¹⁷, —OC(O)NR⁷R¹⁴NR¹¹C(O)R¹⁷, —NR¹¹C(O)OR¹⁷, —NR¹¹C(O)NR⁷R¹⁴, —R⁸OR¹⁷, —R⁸NR⁷R¹⁴, —R⁸S(O)ₘR¹⁷, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷, —R⁸NR¹¹C(O)NR⁷R¹⁴ and YR¹⁰;
R⁵ is an alkyl of 1-6 carbon atoms, branched alkyl of 3-8 carbon atoms, cis-alkenyl of 2-6 carbon atoms, a trans-alkenyl of 2-6 carbon atoms, an alkynyl of 2-6 carbon atoms, an aryl ring, a heterocyclic ring or a heteroaryl ring, said heterocylic ring and heteroaryl containing 1-3 heteroatoms selected from N, O or S, wherein the heterocyclic, heteroaryl and aryl rings are optionally substituted with one to four substituents selected from the group consisting of -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷, —OC(O)OR¹⁷, —OC(O)NR⁷R¹⁴NR¹¹C(O)R¹⁷, —NR¹¹C(O)OR¹⁷, —NR¹¹C(O)NR⁷R¹⁴, —R⁸OR¹⁷, —R⁸NR⁷R¹⁴, —R⁸S(O)ₘR¹⁷, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷, —R⁸NR¹¹C(O)NR⁷R¹⁴, —YR⁸R¹⁰, —YR⁸NR⁷R¹⁴ and —YR¹⁰;
R⁶ is H, alkyl of 1-6 carbon atoms or branched alkyl of 3-8 carbon atoms;
R⁷, R¹¹, R¹², R¹⁴, R¹⁵, R¹⁶, and R¹⁷ are independently selected from H, alkyl of 1-6 carbon atoms, branched alkyl of 3-8 carbon atoms, cis-alkenyl of 2-6 carbon atoms, a trans-alkenyl of 2-6 carbon atoms, and an alkynyl of 2-6 carbon atoms; or R⁷ and R¹⁴ together with the N to which they are attached may join to form a 3 to 8 membered ring, said 3-8 membered ring optionally containing a heteroatom selected from N, O, and S in addition to said N atom to which R⁷ and R¹⁴ are attached;
R⁸ is a divalent group selected from alkyl of 1-6 carbon atoms, alkenyl of 2-6 carbon atoms, and alkynyl of 2-6 carbon atoms;
R⁹ is a divalent alkyl group of 2-6 carbon atoms;
R¹⁰ is selected from the group consisting of a cycloalkyl ring of 3-10 carbons, a bicycloalkyl ring of 3-10 carbons, an aryl, a heterocyclyl ring, a heteroaryl ring, and a heteroaryl fused to 1-3 aryl or heteroaryl rings; any of said heterocyclyl ring and heteroaryl rings containing 1-3 heteroatoms selected from N, O or S; wherein any of the aryl, cycloalkyl, bicycloalkyl, heterocyclic or heteroaryl rings may be optionally substituted with one to four substituents selected from the group consisting of —H, -aryl, —CH₂-aryl, —NH-aryl, —O-aryl, —S(O)ₘ-aryl, -J, —NO₂, —CN, —N₃, —CHO, —CF₃, —OCF₃, —R¹⁷, —OR¹⁷, —S(O)ₘR¹⁷, —NR⁷R¹⁴, —NR¹¹S(O)ₘR¹⁷, —OR⁹OR¹⁷, —OR⁹NR⁷R¹⁴, —N(R¹¹)R⁹OR¹⁷, —N(R¹¹)R⁹NR⁷R¹⁴, —NR¹¹C(O)R¹⁷, —C(O)R¹⁷, —C(O)OR¹⁷, —C(O)NR⁷R¹⁴, —OC(O)R¹⁷, —OC(O)OR¹⁷, —OC(O)NR⁷R¹⁴, —NR⁸C(O)R¹¹, —NR¹¹C(O)OR¹⁷, —NR¹¹C(O)NR⁷R¹⁴, —R⁸OR¹⁷, R⁸NR⁷R¹⁴, —R⁸S(O)ₘR¹⁷, —R⁸C(O)R¹⁷, —R⁸C(O)OR¹⁷, —R⁸C(O)NR⁷R¹⁴, —R⁸C(O)R¹⁷, —R⁸C(O)OR¹⁷, —R⁸C(O)NR⁷R¹⁴, —R⁸OC(O)R¹⁷, —R⁸OC(O)OR¹⁷, —R⁸OC(O)NR⁷R¹⁴, —R⁸NR¹¹C(O)R¹⁷, —R⁸NR¹¹C(O)OR¹⁷, and —R⁸NR¹¹C(O)NR⁷R¹⁴;
J is fluoro, chloro, bromo, or iodo;
m is an integer of 0-2;
W¹ is —C(O)— or —C(O)—NR¹⁷, —SO₂—, or —CO—C(R⁶)₂—; and
Y is selected from the group consisting of a bond, a divalent alkyl group of 1-6 carbon atoms, NH, O, —NR¹⁷, —C≡C—, cis- —CH═CH—, and trans- —CH═CH—.

5. The compound according to claim 1 wherein R² is pyridyl, furanyl, or thiophenyl.

6. The compound according to claim 1 wherein Y is a bond or a divalent alkyl group.

7. The compound according to claim 1 wherein R² is substituted phenyl.

8. The compound according to claim 1 wherein R⁵ is mono- or di-substituted phenyl.

9. The compound according to claim 1 wherein R⁵ is mono- or di-substituted phenyl and the substituents on R⁵ are selected from the group consisting of -J, —NO₂, —CN, —N₃, —CHO, —CF₃, —OCF₃, —R⁷, and —OR¹⁷.

10. The compound according to claim 9 wherein the substitutents on R⁵ are selected from -J, —CF₃, and —OR⁷.

11. The compound according to claim 1 wherein R¹ is selected from the group consisting of H, J, —C(O)OR¹⁶, —C(O)NR⁷R¹⁴, —NR⁶C(O)R¹⁶, a 5-7 membered heterocyclic ring or heteroaryl ring containing 1-3 heteroatoms selected from N, O or S, and an aryl ring, wherein the R⁷ group, the R¹⁴ group, the R¹⁶ group, the heterocyclic ring, the heteroaryl ring and the aryl ring can be optionally substituted.

12. The compound according to claim 11 wherein R¹ is H or J.

13. The compound according to claim 1 wherein W′ is C(O).

14. The compound according to claim 1 wherein W′ is C(O), R⁵ is phenyl, R¹ is H, and R² is substituted aryl or an optionally substituted heteroaryl.

15. The compound according to claim 1 wherein R^{a}, R^{b}, R^{c}, R^{d}, R³ and R⁴ are independently selected from the group consisting of H, -J, —NO₂, —CN, —N₃, —CHO, —CF₃, —OCF₃, —R⁷, and —OR⁷.

16. The compound according to claim 2 wherein R^{a}, R^{b}, R^{c}, R^{d}, R³ and R⁴ are independently selected from the group consisting of H, -J, —NO₂, —CN, —N₃, —CHO, —CF₃, —OCF₃, —R¹⁷, and —OR¹⁷.

17. The compound according to claim 2 wherein W′ is C(O).

18. The compound according to claim 2 wherein R⁵ is an optionally substituted aryl.

19. The compound according to claim 2 wherein R² is H.

20. The compound according to claim 2 wherein R¹ is substituted aryl or optionally substituted heteroaryl.

21. The compound according to claim 2 wherein W′ is C(O), R⁵ is phenyl, R² is H, R¹ is substituted aryl or heteroaryl.

22. The compound according to claim 3 wherein R^{a}, R^{b}, R^{c}, R^{d}, R³ and R⁴ are independently selected from the group consisting of H, -J, —NO₂, —CN, —N₃, —CHO, —CF₃, —OCF₃, —R¹⁷, and —OR¹⁷.

23. The compound according to claim 4 wherein R^{a}, R^{b}, R^{c}, R^{d}, R³ and R⁴ are independently selected from the group consisting of H, -J, —NO₂, —CN, —N₃, —CHO, —CF₃, —OCF₃, —R¹⁷, and —OR¹⁷.

24. A composition comprising an effective amount of a compound of claim 1 and a physiologically acceptable vehicle.

25. A composition comprising an effective amount of a compound of claim 2 and a physiologically acceptable vehicle.

26. A composition comprising an effective amount of a compound of claim 3 and a physiologically acceptable vehicle.

27. A composition comprising an effective amount of a compound of claim 4 and a physiologically acceptable vehicle.

28. A method of treating, inhibiting the growth of, or eradicating neoplasms in a mammal in need thereof which comprises administering to said mammal an effective amount of a compound of claim 1.

29. A method of treating, inhibiting the growth of, or eradicating neoplasms in a mammal in need thereof which comprises administering to said mammal an effective amount of a compound of claim 2.

30. A method of treating, inhibiting the growth of, or eradicating neoplasms in a mammal in need thereof which comprises administering to said mammal an effective amount of a compound of claim 3.

31. A method of treating, inhibiting the growth of, or eradicating neoplasms in a mammal in need thereof which comprises administering to said mammal an effective amount of a compound of claim 4.

32. A method of treating cancer in a mammal in need thereof which comprises administering to said mammal an effective amount of a compound of claim 1.

33. The method according to claim 32 wherein the cancer is selected from the group consisting of breast, kidney, bladder, mouth, larynx, esophagus, stomach, colon, ovary, lung, pancreas, skin, liver, prostate and brain cancer.

34. The method according to claim 33 wherein the mammal is a human.

35. A method of treating cancer in a mammal in need thereof which comprises administering to said mammal an effective amount of a compound of claim 2.

36. The method according to claim 35 wherein the cancer is selected from the group consisting of breast, kidney, bladder, mouth, larynx, esophagus, stomach, colon, ovary, lung, pancreas, skin, liver, prostate and brain cancer.

37. The method according to claim 36 wherein the mammal is a human.

38. A method of treating cancer in a mammal in need thereof which comprises administering to said mammal an effective amount of a compound of claim 3.

39. The method according to claim 38 wherein the cancer is selected from the group consisting of breast, kidney, bladder, mouth, larynx, esophagus, stomach, colon, ovary, lung, pancreas, skin, liver, prostate and brain cancer.

40. The method according to claim 39 wherein the mammal is a human.

41. A method of treating cancer in a mammal in need thereof which comprises administering to said mammal an effective amount of a compound of claim 4.

42. The method according to claim 41 wherein the cancer is selected from the group consisting of breast, kidney, bladder, mouth, larynx, esophagus, stomach, colon, ovary, lung, pancreas, skin, liver, prostate and brain cancer.

43. The method according to claim 42 wherein the mammal is a human.

44. A compound selected from the group consisting of:
ethyl 7-(3-{[3-(trifluoromethyl)benzoyl]amino}phenyl)pyrazolo[1,5-a]pyrimidine-3-carboxylate;
ethyl 7-(3-{[4-fluoro-3-(trifluoromethyl)benzoyl]amino}phenyl)pyrazolo[1,5-a]pyrimidine-3-carboxylate;
ethyl 7-[3-(benzoylamino)phenyl]pyrazolo[1,5-a]pyrimidine-3-carboxylate;
ethyl 7-{3-[(3-bromobenzoyl)amino]phenyl}pyrazolo[1,5-a]pyrimidine-3-carboxylate;
ethyl 7-{3-[(1-benzothien-2-ylcarbonyl)amino]phenyl}pyrazolo[1,5-a]pyrimidine-3-carboxylate;
ethyl 7-{3-[(4-chlorobenzoyl)amino]phenyl}pyrazolo[1,5-a]pyrimidine-3-carboxylate;
ethyl 7-(3-{[3-(trifluoromethoxy)benzoyl]amino}phenyl)pyrazolo[1,5-a]pyrimidine-3-carboxylate;
ethyl 7-{3-[(3-methoxybenzoyl)amino]phenyl}pyrazolo[1,5-a]pyrimidine-3-carboxylate;
ethyl 7-(3-{[3-fluoro-4-(trifluoromethyl)benzoyl]amino}phenyl)pyrazolo[1,5-a]pyrimidine-3-carboxylate;
ethyl 7-(3-{[4-(trifluoromethyl)benzoyl]amino}phenyl)pyrazolo[1,5-a]pyrimidine-3-carboxylate;
ethyl 7-{3-[({[4-chloro-3-(trifluoromethyl)phenyl]amino}carbonyl)amino]phenyl}pyrazolo[1,5-a]pyrimidine-3-carboxylate;
ethyl 7-[3-({[3-(trifluoromethyl)phenyl]sulfonyl}amino)phenyl]pyrazolo[1,5-a]pyrimidine-3-carboxylate;
ethyl 7-{3-[(3-cyanobenzoyl)amino]phenyl}pyrazolo[1,5-a]pyrimidine-3-carboxylate;
ethyl 7-[3-({[(2,4-dichlorophenyl)amino]carbonyl}amino)phenyl]pyrazolo[1,5-a]pyrimidine-3-carboxylate;
ethyl 7-{3-[({[4-(ethoxycarbonyl)phenyl]amino}carbonyl)amino]phenyl}pyrazolo[1,5-a]pyrimidine-3-carboxylate;
ethyl 7-{3-[({[3,5-bis(trifluoromethyl)phenyl]amino}carbonyl)amino]phenyl}pyrazolo[1,5-a]pyrimidine-3-carboxylate;
ethyl 7-[3-({([(3,5-dichlorophenyl)amino]carbonyl}amino)phenyl]pyrazolo[1,5-a]pyrimidine-3-carboxylate;
ethyl 7-{3-[({[4-(methylthio)phenyl]amino}carbonyl)amino]phenyl}pyrazolo[1,5-a]pyrimidine-3-carboxylate;
ethyl 7-[3-({[(4-acetylphenyl)amino]carbonyl}amino)phenyl]pyrazolo[1,5-a]pyrimidine-3-carboxylate;
ethyl 7-[3-({[(4-isopropylphenyl)amino]carbonyl}amino)phenyl]pyrazolo[1,5-a]pyrimidine-3-carboxylate;
ethyl 7-(3-{[(2-naphthylamino)carbonyl]amino}phenyl)pyrazolo[1,5-a]pyrimidine-3-carboxylate;
ethyl 7-(3-{[(mesitylamino)carbonyl]amino}phenyl)pyrazolo[1,5-a]pyrimidine-3-carboxylate;
ethyl 7-{3-[({[4-(trifluoromethoxy)phenyl]amino}carbonyl)amino]phenyl}pyrazolo[1,5-a]pyrimidine-3-carboxylate;
ethyl 7-(3-{[({4-[(trifluoromethyl)thio]phenyl}amino)carbonyl]amino}phenyl)pyrazolo[1,5-a]pyrimidine-3-carboxylate;
ethyl 7-[3-({[(3-chloro-4-fluorophenyl)amino]carbonyl}amino)phenyl]pyrazolo[1,5-a]pyrimidine-3-carboxylate;
ethyl 7-(3-{[4-methyl-3-(trifluoromethyl)benzoyl]amino}phenyl)pyrazolo[1,5-a]pyrimidine-3-carboxylate;
ethyl 7-(3-{[2-chloro-5-(trifluoromethyl)benzoyl]amino}phenyl)pyrazolo[1,5-a]pyrimidine-3-carboxylate;
ethyl 7-(3-{[3-chloro-2-fluoro-5-(trifluoromethyl)benzoyl]amino}phenyl)pyrazolo[1,5-a]pyrimidine-3-carboxylate;
ethyl 7-{3-[(4-chloro-2,5-difluorobenzoyl)amino]phenyl}pyrazolo[1,5-a]pyrimidine-3-carboxylate;
ethyl 7-(3-{[4-methoxy-3-(trifluoromethyl)benzoyl]amino}phenyl)pyrazolo[1,5-a]pyrimidine-3-carboxylate;
N-methyl-7-(3-{[3-(trifluoromethyl)benzoyl]amino}phenyl)pyrazolo[1,5-a]pyrimidine-3-carboxamide;
7-(3-{[3-(trifluoromethyl)benzoyl]amino}phenyl)pyrazolo[1,5-a]pyrimidine-3-carboxylic acid;
ethyl 7-{3-[({[3-(trifluoromethyl)phenyl]amino}carbonyl)amino]phenyl}pyrazolo[1,5-a]pyrimidine-3-carboxylate;
ethyl 7-[3-({[(4-chlorophenyl)amino]carbonyl}amino)phenyl]pyrazolo[1,5-a]pyrimidine-3-carboxylate;
ethyl 7-{3-[({[4-(trifluoromethyl)phenyl]amino}carbonyl)amino]phenyl}pyrazolo[1,5-a]pyrimidine-3-carboxylate;
ethyl 7-[3-({[(4-chloro-2-methylphenyl)amino]carbonyl}amino)phenyl]pyrazolo[1,5-a]pyrimidine-3-carboxylate;
ethyl 7-{3-[({[2-chloro-5-(trifluoromethyl)phenyl]amino}carbonyl)amino]phenyl}pyrazolo[1,5-a]pyrimidine-3-carboxylate;
ethyl 7-[3-({[(4-cyanophenyl)amino]carbonyl}amino)phenyl]pyrazolo[1,5-a]pyrimidine-3-carboxylate;
ethyl 7-{3-[({[2-(trifluoromethyl)phenyl]amino}carbonyl)amino]phenyl}pyrazolo[1,5-a]pyrimidine-3-carboxylate;
ethyl 7-[3-({[(3,4-dichlorophenyl)amino]carbonyl}amino)phenyl]pyrazolo[1,5-a]pyrimidine-3-carboxylate;
ethyl 7-[3-({[(4-bromophenyl)amino]carbonyl}amino)phenyl]pyrazolo[1,5-a]pyrimidine-3-carboxylate;
ethyl 7-[3-({[(3,4-dimethylphenyl)amino]carbonyl}amino)phenyl]pyrazolo[1,5-a]pyrimidine-3-carboxylate;
ethyl 7-{3-[({[4-chloro-2-(trifluoromethyl)phenyl]amino}carbonyl)amino]phenyl}pyrazolo[1,5-a]pyrimidine-3-carboxylate;
ethyl 7-{3-[({[4-fluoro-3-(trifluoromethyl)phenyl]amino}carbonyl)amino]phenyl}pyrazolo[1,5-a]pyrimidine-3-carboxylate;
N-(2-methoxyethyl)-7-(3-{[3-(trifluoromethyl)benzoyl]amino}phenyl)pyrazolo[1,5-a]pyrimidine-3-carboxamide;
N-propyl-7-(3-{[3-(trifluoromethyl)benzoyl]amino}phenyl)pyrazolo[1,5-a]pyrimidine-3-carboxamide;
N-pyridin-3-yl-7-(3-{[3-(trifluoromethyl)benzoyl]amino}phenyl)pyrazolo[1,5-a]pyrimidine-3-carboxamide;
N-(2-pyrrolidin-1-ylethyl)-7-(3-{[3-(trifluoromethyl)benzoyl]amino}phenyl)pyrazolo[1,5-a]pyrimidine-3-carboxamide;
N-[2-(dimethylamino)ethyl]-7-(3-{[3-(trifluoromethyl)benzoyl]amino}phenyl)pyrazolo[1,5-a]pyrimidine-3-carboxamide;
N-[3-(4-methylpiperazin-1-yl)propyl]-7-(3-{[3-(trifluoromethyl)benzoyl]amino}phenyl)pyrazolo[1,5-a]pyrimidine-3-carboxamide;
N-ethyl-7-(3-{[3-(trifluoromethyl)benzoyl]amino}phenyl)pyrazolo[1,5-a]pyrimidine-3-carboxamide;
N-(2-morpholin-4-ylethyl)-7-(3-{[3-(trifluoromethyl)benzoyl]amino}phenyl)pyrazolo[1,5-a]pyrimidine-3-carboxamide;
N-(3-morpholin-4-ylpropyl)-7-(3-{[3-(trifluoromethyl)benzoyl]amino}phenyl)pyrazolo[1,5-a]pyrimidine-3-carboxamide;
N-[2-(1-methylpyrrolidin-2-yl)ethyl]-7-(3-{[3-(trifluoromethyl)benzoyl]amino}phenyl)pyrazolo[1,5-a]pyrimidine-3-carboxamide;
N-[3-(1H-imidazol-1-yl)propyl]-7-(3-{[3-(trifluoromethyl)benzoyl]amino}phenyl)pyrazolo[1,5-a]pyrimidine-3-carboxamide;
N-(3-methoxypropyl)-7-(3-{[3-(trifluoromethyl)benzoyl]amino}phenyl)pyrazolo[1,5-a]pyrimidine-3-carboxamide;
N-benzyl-7-(3-{[3-(trifluoromethyl)benzoyl]amino}phenyl)pyrazolo[1,5-a]pyrimidine-3-carboxamide;
ethyl 2-methyl-7-(3-{[3-(trifluoromethyl)benzoyl]amino}phenyl)pyrazolo[1,5-a]pyrimidine-3-carboxylate;
N-[3-(3-pyridin-2-ylpyrazolo[1,5-a]pyrimidin-7-yl)phenyl]-3-(trifluoromethyl)benzamide;
N-{3-[3-(2H-tetrazol-5-yl)pyrazolo[1,5-a]pyrimidin-7-yl]phenyl}-3-(trifluoromethyl)benzamide;
7-(3-{[3-(trifluoromethyl)benzoyl]amino}phenyl)pyrazolo[1,5-a]pyrimidine-3-carboxamide;
N-[3-(3-cyano-2-piperazin-1-ylpyrazolo[1,5-a]pyrimidin-7-yl)phenyl]-3-(trifluoromethyl)benzamide;
N-[3-(3-cyanopyrazolo[1,5-a]pyrimidin-7-yl)phenyl]-3-(trifluoromethyl)benzamide;
ethyl 2-methyl-7-(3-{[4-methyl-3-(trifluoromethyl)benzoyl]amino}phenyl)pyrazolo[1,5-a]pyrimidine-3-carboxylate;
4-methyl-N-[3-(3-pyridin-2-ylpyrazolo[1,5-a]pyrimidin-7-yl)phenyl]-3-(trifluoromethyl)benzamide;
4-methyl-N-{3-[3-(2H-tetrazol-5-yl)pyrazolo[1,5-a]pyrimidin-7-yl]phenyl}-3-(trifluoromethyl)benzamide;
7-(3-{[4-methyl-3-(trifluoromethyl)benzoyl]amino}phenyl)pyrazolo[1,5-a]pyrimidine-3-carboxamide;
N-[3-(3-cyano-2-piperazin-1-ylpyrazolo[1,5-a]pyrimidin-7-yl)phenyl]-4-methyl-3-(trifluoromethyl)benzamide;
N-[3-(3-cyanopyrazolo[1,5-a]pyrimidin-7-yl)phenyl]-4-methyl-3-(trifluoromethyl)benzamide;
ethyl 7-{3-[(3-chlorobenzoyl)amino]phenyl}pyrazolo[1,5-a]pyrimidine-3-carboxylate;
ethyl 7-{3-[(3,4-dichlorobenzoyl)amino]phenyl}pyrazolo[1,5-a]pyrimidine-3-carboxylate;
ethyl 7-{3-[(3,5-dichlorobenzoyl)amino]phenyl}pyrazolo[1,5-a]pyrimidine-3-carboxylate;
ethyl 7-{3-[(3-chloro-4-methoxybenzoyl)amino]phenyl}pyrazolo[1,5-a]pyrimidine-3-carboxylate;
ethyl 7-{3-[(5-chloro-2-methylbenzoyl)amino]phenyl}pyrazolo[1,5-a]pyrimidine-3-carboxylate;
ethyl 7-(3-{[3-fluoro-5-(trifluoromethyl)benzoyl]amino}phenyl)pyrazolo[1,5-a]pyrimidine-3-carboxylate;
ethyl 7-(3-{[4-fluoro-2-(trifluoromethyl)benzoyl]amino}phenyl)pyrazolo[1,5-a]pyrimidine-3-carboxylate;
ethyl 7-{3-[({[3-methoxy-5-(trifluoromethyl)phenyl]amino}carbonyl)amino]phenyl}pyrazolo[1,5-a]pyrimidine-3-carboxylate;
ethyl 7-{3-[({[4-cyano-3-(trifluoromethyl)phenyl]amino}carbonyl)amino]phenyl}pyrazolo[1,5-a]pyrimidine-3-carboxylate;
ethyl 7-{3-[({[4-methyl-3-(trifluoromethyl)phenyl]amino}carbonyl)amino]phenyl}pyrazolo[1,5-a]pyrimidine-3-carboxylate;
ethyl 7-[3-({[(3-chlorophenyl)amino]carbonyl}amino)phenyl]pyrazolo[1,5-a]pyrimidine-3-carboxylate;
ethyl 7-[3-({[(3-chloro-4-methoxyphenyl)amino]carbonyl}amino)phenyl]pyrazolo[1,5-a]pyrimidine-3-carboxylate;
ethyl 7-[3-({[(3-chloro-4-methylphenyl)amino]carbonyl}amino)phenyl]pyrazolo[1,5-a]pyrimidine-3-carboxylate;
ethyl 7-[3-({[(4-bromo-3-chlorophenyl)amino]carbonyl}amino)phenyl]pyrazolo[1,5-a]pyrimidine-3-carboxylate;
ethyl 7-[3-({[(3-chloro-4-morpholin-4-ylphenyl)amino]carbonyl}amino)phenyl]pyrazolo[1,5-a]pyrimidine-3-carboxylate;
ethyl 7-(2-nitro-5-{[3-(trifluoromethyl)benzoyl]amino}phenyl)pyrazolo[1,5-a]pyrimidine-3-carboxylate;
ethyl 7-(4-chloro-3-{[3-(trifluoromethyl)benzoyl]amino}phenyl)pyrazolo[1,5-a]pyrimidine-3-carboxylate;
ethyl 7-(4-(2,6-dimethylmorpholin-4-yl)-3-{[3-(trifluoromethyl)benzoyl]amino}phenyl)pyrazolo[1,5-a]pyrimidine-3-carboxylate;
ethyl 7-(4-methoxy-3-{[3-(trifluoromethyl)benzoyl]amino}phenyl)pyrazolo[1,5-a]pyrimidine-3-carboxylate;
ethyl 7-(4-fluoro-3-{[3-(trifluoromethyl)benzoyl]amino}phenyl)pyrazolo[1,5-a]pyrimidine-3-carboxylate;
ethyl 7-(4-(benzyloxy)-3-{[3-(trifluoromethyl)benzoyl]amino}phenyl)pyrazolo[1,5-a]pyrimidine-3-carboxylate;
ethyl 7-(2-fluoro-5-{[3-(trifluoromethyl)benzoyl]amino}phenyl)pyrazolo[1,5-a]pyrimidine-3-carboxylate;
ethyl 7-(3-{[(3-bromophenyl)acetyl]amino}phenyl)pyrazolo[1,5-a]pyrimidine-3-carboxylate;
ethyl 7-(3-{[(3-fluorophenyl)acetyl]amino}phenyl)pyrazolo[1,5-a]pyrimidine-3-carboxylate;
ethyl 7-(3-{[(3-chlorophenyl)acetyl]amino}phenyl)pyrazolo[1,5-a]pyrimidine-3-carboxylate;
ethyl 7-(3-{[(3,4-dichlorophenyl)acetyl]amino}phenyl)pyrazolo[1,5-a]pyrimidine-3-carboxylate;
ethyl 7-(3-{[(3-methoxyphenyl)acetyl]amino}phenyl)pyrazolo[1,5-a]pyrimidine-3-carboxylate;
ethyl 7-[3-({[3-(trifluoromethyl)phenyl]acetyl}amino)phenyl]pyrazolo[1,5-a]pyrimidine-3-carboxylate;
ethyl 7-(3-{[(3-methylphenyl)acetyl]amino}phenyl)pyrazolo[1,5-a]pyrimidine-3-carboxylate;
ethyl 7-[3-({[3,5-bis(trifluoromethyl)phenyl]acetyl}amino)phenyl]pyrazolo[1,5-a]pyrimidine-3-carboxylate;
ethyl 7-{3-[(1,3-benzodioxol-5-ylacetyl)amino]phenyl}pyrazolo[1,5-a]pyrimidine-3-carboxylate;
ethyl 7-(3-{[(4-methoxy-3-methylphenyl)acetyl]amino}phenyl)pyrazolo[1,5-a]pyrimidine-3-carboxylate;
ethyl 7-(3-{[(2,3,6-trifluorophenyl)acetyl]amino}phenyl)pyrazolo[1,5-a]pyrimidine-3-carboxylate;
ethyl 7-(3-{[4-chloro-3-(trifluoromethyl)benzoyl]amino}phenyl)pyrazolo[1,5-a]pyrimidine-3-carboxylate;
ethyl 7-(3-{[2-methyl-5-(trifluoromethyl)benzoyl]amino}phenyl)pyrazolo[1,5-a]pyrimidine-3-carboxylate;
ethyl 7-(5-{[4-methyl-3-(trifluoromethyl)benzoyl]amino}-2-nitrophenyl)pyrazolo[1,5-a]pyrimidine-3-carboxylate;
ethyl 7-(4-chloro-3-{[4-methyl-3-(trifluoromethyl)benzoyl]amino}phenyl)pyrazolo[1,5-a]pyrimidine-3-carboxylate;
ethyl 7-(4-methoxy-3-{[4-methyl-3-(trifluoromethyl)benzoyl]amino}phenyl)pyrazolo[1,5-a]pyrimidine-3-carboxylate;
ethyl 7-(4-(benzyloxy)-3-{[4-methyl-3-(trifluoromethyl)benzoyl]amino}phenyl)pyrazolo[1,5-a]pyrimidine-3-carboxylate;
N-[3-(2-pyridin-4-ylpyrazolo[1,5-a]pyrimidin-7-yl)phenyl]-3-(trifluoromethyl)benzamide;
N-(3-{2-[3-(dimethylamino)propyl]pyrazolo[1,5-a]pyrimidin-7-yl}phenyl)-3-(trifluoromethyl)benzamide;
N-[3-(2-pyridin-2-ylpyrazolo[1,5-a]pyrimidin-7-yl)phenyl]-3-(trifluoromethyl)benzamide;
N-[3-(2-methylpyrazolo[1,5-a]pyrimidin-7-yl)phenyl]-3-(trifluoromethyl)benzamide;
N-{3-[2-(2-furyl)pyrazolo[1,5-a]pyrimidin-7-yl]phenyl}-3-(trifluoromethyl)benzamide;
N-{3-[2-(2-thienyl)pyrazolo[1,5-a]pyrimidin-7-yl]phenyl}-3-(trifluoromethyl)benzamide;
N-{3-[2-(4-chlorophenyl)pyrazolo[1,5-a]pyrimidin-7-yl]phenyl}-3-(trifluoromethyl)benzamide;
4-methyl-N-[3-(2-methylpyrazolo[1,5-a]pyrimidin-7-yl)phenyl]-3-(trifluoromethyl)benzamide;
4-methyl-N-{3-[2-(2-thienyl)pyrazolo[1,5-a]pyrimidin-7-yl]phenyl}-3-(trifluoromethyl)benzamide;
N-{3-[2-(4-chlorophenyl)pyrazolo[1,5-a]pyrimidin-7-yl]phenyl}-4-methyl-3-(trifluoromethyl)benzamide;
4-methyl-N-[3-(2-phenylpyrazolo[1,5-a]pyrimidin-7-yl)phenyl]-3-(trifluoromethyl)benzamide;
7-{3-[({[4-chloro-3-(trifluoromethyl)phenyl]amino}carbonyl)amino]phenyl}-N-[3-(1H-imidazol-1-yl)propyl]pyrazolo[1,5-a]pyrimidine-3-carboxamide;
7-{3-[({[4-chloro-3-(trifluoromethyl)phenyl]amino}carbonyl)amino]phenyl}-N-(3-methoxypropyl)pyrazolo[1,5-a]pyrimidine-3-carboxamide;
7-{3-[({[4-chloro-3-(trifluoromethyl)phenyl]amino}carbonyl)amino]phenyl}-N-[2-(diethylamino)ethyl]pyrazolo[1,5-a]pyrimidine-3-carboxamide;
7-{3-[({[4-chloro-3-(trifluoromethyl)phenyl]amino}carbonyl)amino]phenyl}-N-(2-morpholin-4-ylethyl)pyrazolo[1,5-a]pyrimidine-3-carboxamide;
methyl 7-(3-{[3-(trifluoromethyl)benzoyl]amino}phenyl)pyrazolo[1,5-a]pyrimidine-3-carboxylate;
2,2,2-trifluoroethyl 7-(3-{[3-(trifluoromethyl)benzoyl]amino}phenyl)pyrazolo[1,5-a]pyrimidine-3-carboxylate;
pyridin-3-yl 7-(3-{[3-(trifluoromethyl)benzoyl]amino}phenyl)pyrazolo[1,5-a]pyrimidine-3-carboxylate;
2-(dimethylamino)ethyl 7-(3-{[3-(trifluoromethyl)benzoyl]amino}phenyl)pyrazolo[1,5-a]pyrimidine-3-carboxylate;
2-methoxyethyl 7-(3-{[3-(trifluoromethyl)benzoyl]amino}phenyl)pyrazolo[1,5-a]pyrimidine-3-carboxylate;
4-chloro-N-[3-(2-pyridin-4-ylpyrazolo[1,5-a]pyrimidin-7-yl)phenyl]-3-(trifluoromethyl)benzamide;
4-methoxy-N-[3-(2-pyridin-4-ylpyrazolo[1,5-a]pyrimidin-7-yl)phenyl]-3-(trifluoromethyl)benzamide;
N-[4-fluoro-3-(trifluoromethyl)phenyl]-N′-[3-(2-pyridin-4-ylpyrazolo[1,5-a]pyrimidin-7-yl)phenyl]urea;
N-[3-(2-pyridin-4-ylpyrazolo[1,5-a]pyrimidin-7-yl)phenyl]-N′-[3-(trifluoromethyl)phenyl]urea;
N-[4-chloro-3-(trifluoromethyl)phenyl]-N′-[3-(2-pyridin-4-ylpyrazolo[1,5-a]pyrimidin-7-yl)phenyl]urea;
4-methyl-N-[3-(2-pyridin-4-ylpyrazolo[1,5-a]pyrimidin-7-yl)phenyl]-3-(trifluoromethyl)benzamide;
N-(3-{2-[4-(hydroxymethyl)phenyl]pyrazolo[1,5-a]pyrimidin-7-yl}phenyl)-3-(trifluoromethyl)benzamide;
N-{3-[2-(4-methoxyphenyl)pyrazolo[1,5-a]pyrimidin-7-yl]phenyl}-3-(trifluoromethyl)benzamide;
3-(trifluoromethyl)-N-(3-{2-[3-(trifluoromethyl)phenyl]pyrazolo[1,5-a]pyrimidin-7-yl}phenyl)benzamide;
N-[3-(2-tert-butylpyrazolo[1,5-a]pyrimidin-7-yl)phenyl]-3-(trifluoromethyl)benzamide;
N-{3-[2-(4-fluorophenyl)pyrazolo[1,5-a]pyrimidin-7-yl]phenyl}-3-(trifluoromethyl)benzamide;
N-[3-(2-{4-[(ethoxymethoxy)methyl]phenyl}pyrazolo[1,5-a]pyrimidin-7-yl)phenyl]-3-(trifluoromethyl)benzamide;
methyl 3-[7-(3-{[3-(trifluoromethyl)benzoyl]amino}phenyl)pyrazolo[1,5-a]pyrimidin-2-yl]benzoate;
4-[7-(3-{[3-(trifluoromethyl)benzoyl]amino}phenyl)pyrazolo[1,5-a]pyrimidin-2-yl]benzyl acetate;
N-[3-(3-bromopyrazolo[1,5-a]pyrimidin-7-yl)phenyl]-N′-[4-chloro-3-(trifluoromethyl)phenyl]urea;
N-[3-(2-pyridin-4-ylpyrazolo[1,5-a]pyrimidin-7-yl)phenyl]benzamide;
2-chloro-N-[3-(2-pyridin-4-ylpyrazolo[1,5-a]pyrimidin-7-yl)phenyl]benzamide;
3-methoxy-N-[3-(2-pyridin-4-ylpyrazolo[1,5-a]pyrimidin-7-yl)phenyl]benzamide;
3-methyl-N-[3-(2-pyridin-4-ylpyrazolo[1,5-a]pyrimidin-7-yl)phenyl]benzamide;
4-methyl-N-[3-(2-pyridin-4-ylpyrazolo[1,5-a]pyrimidin-7-yl)phenyl]benzamide;
3,4-dichloro-N-[3-(2-pyridin-4-ylpyrazolo[1,5-a]pyrimidin-7-yl)phenyl]benzamide;
3-chloro-N-[3-(2-pyridin-4-ylpyrazolo[1,5-a]pyrimidin-7-yl)phenyl]benzamide;
4-chloro-N-[3-(2-pyridin-4-ylpyrazolo[1,5-a]pyrimidin-7-yl)phenyl]benzamide;
N-[3-(2-pyridin-3-ylpyrazolo[1,5-a]pyrimidin-7-yl)phenyl]-3-(trifluoromethyl)benzamide;
N-(3-pyrazolo[1,5-a]pyrimidin-7-ylphenyl)-3-(trifluoromethyl)benzamide;
N-[3-(3-pyridin-4-ylpyrazolo[1,5-a]pyrimidin-7-yl)phenyl]-3-(trifluoromethyl)benzamide;
N-[3-(3-pyridin-3-ylpyrazolo[1,5-a]pyrimidin-7-yl)phenyl]-3-(trifluoromethyl)benzamide;
N-{3-[3-(3-aminophenyl)pyrazolo[1,5-a]pyrimidin-7-yl]phenyl}-3-(trifluoromethyl)benzamide;
N-(3-{3-[4-(dimethylamino)phenyl]pyrazolo[1,5-a]pyrimidin-7-yl}phenyl)-3-(trifluoromethyl)benzamide;
N-{3-[3-(4-chlorophenyl)pyrazolo[1,5-a]pyrimidin-7-yl]phenyl}-3-(trifluoromethyl)benzamide;
N-{3-[3-(4-methoxyphenyl)pyrazolo[1,5-a]pyrimidin-7-yl]phenyl}-3-(trifluoromethyl)benzamide;
N-{3-[3-(4-methylphenyl)pyrazolo[1,5-a]pyrimidin-7-yl]phenyl}-3-(trifluoromethyl)benzamide;
N-[3-(3-bromopyrazolo[1,5-a]pyrimidin-7-yl)phenyl]-4-methyl-3-(trifluoromethyl)benzamide;
N-[3-(3-bromopyrazolo[1,5-a]pyrimidin-7-yl)phenyl]-4-methoxy-3-(trifluoromethyl)benzamide;
N-[3-(3-bromopyrazolo[1,5-a]pyrimidin-7-yl)phenyl]-4-fluoro-3-(trifluoromethyl)benzamide;
N-[3-(3-bromopyrazolo[1,5-a]pyrimidin-7-yl)phenyl]-4-chloro-3-(trifluoromethyl)benzamide;
N-[3-(3-bromopyrazolo[1,5-a]pyrimidin-7-yl)phenyl]-3,4-dichlorobenzamide;
N-[3-(3-bromopyrazolo[1,5-a]pyrimidin-7-yl)phenyl]-N′-[3-(trifluoromethyl)phenyl]urea;
N-[3-(3-bromopyrazolo[1,5-a]pyrimidin-7-yl)phenyl]-N′-[4-fluoro-3-(trifluoromethyl)phenyl]urea;
N-[3-(3-bromopyrazolo[1,5-a]pyrimidin-7-yl)phenyl]-N′-(3,4-dichlorophenyl)urea;
4-methyl-N-[3-(3-pyridin-3-ylpyrazolo[1,5-a]pyrimidin-7-yl)phenyl]-3-(trifluoromethyl)benzamide;
4-methoxy-N-[3-(3-pyridin-3-ylpyrazolo[1,5-a]pyrimidin-7-yl)phenyl]-3-(trifluoromethyl)benzamide;
4-fluoro-N-[3-(3-pyridin-3-ylpyrazolo[1,5-a]pyrimidin-7-yl)phenyl]-3-(trifluoromethyl)benzamide;
4-chloro-N-[3-(3-pyridin-3-ylpyrazolo[1,5-a]pyrimidin-7-yl)phenyl]-3-(trifluoromethyl)benzamide;
N-[3-(3-pyridin-3-ylpyrazolo[1,5-a]pyrimidin-7-yl)phenyl]-N′-[3-(trifluoromethyl)phenyl]urea;
N-[4-chloro-3-(trifluoromethyl)phenyl]-N′-[3-(3-pyridin-3-ylpyrazolo[1,5-a]pyrimidin-7-yl)phenyl]urea;
N-(3,4-dichlorophenyl)-N′-[3-(3-pyridin-3-ylpyrazolo[1,5-a]pyrimidin-7-yl)phenyl]urea;
ethyl 6-methyl-7-(3-{[3-(trifluoromethyl)benzoyl]amino}phenyl)pyrazolo[1,5-a]pyrimidine-3-carboxylate;
N-[3-(6-methyl-2-pyridin-4-yipyrazolo[1,5-a]pyrimidin-7-yl)phenyl]-3-(trifluoromethyl)benzamide;
N-[3-(3-bromo-6-methylpyrazolo[1,5-a]pyrimidin-7-yl)phenyl]-3-(trifluoromethyl)benzamide;
N-[3-(6-methyl-3-pyridin-3-ylpyrazolo[1,5-a]pyrimidin-7-yl)phenyl]-3-(trifluoromethyl)benzamide;
N-{3-[3-(4-aminophenyl)pyrazolo[1,5-a]pyrimidin-7-yl]phenyl}-4-methyl-3-(trifluoromethyl)benzamide;
N-{3-[3-(3-hydroxyphenyl)pyrazolo[1,5-a]pyrimidin-7-yl]phenyl}-4-methyl-3-(trifluoromethyl)benzamide;
N-{3-[3-(3-cyanophenyl)pyrazolo[1,5-a]pyrimidin-7-yl]phenyl}-4-methyl-3-(trifluoromethyl)benzamide;
N-[3-(3-{3-[(dimethylamino)carbonyl]phenyl}pyrazolo[1,5-a]pyrimidin-7-yl)phenyl]-4-methyl-3-(trifluoromethyl)benzamide;
N-(3-{3-[4-(acetylamino)phenyl]pyrazolo[1,5-a]pyrimidin-7-yl}phenyl)-4-methyl-3-(trifluoromethyl)benzamide;
ethyl 7-(3-{[3-(trifluoromethyl)benzoyl]amino}phenyl)pyrazolo[1,5-a]pyrimidine-2-carboxylate;
N-[3-(2-morpholin-4-ylpyrazolo[1,5-a]pyrimidin-7-yl)phenyl]-3-(trifluoromethyl)benzamide;
N-{3-[2-(dimethylamino)pyrazolo[1,5-a]pyrimidin-7-yl]phenyl}-3-(trifluoromethyl)benzamide;
4-methyl-N-[3-(3-pyridin-4-ylpyrazolo[1,5-a]pyrimidin-7-yl)phenyl]-3-(trifluoromethyl)benzamide;
4-methoxy-N-[3-(3-pyridin-4-ylpyrazolo[1,5-a]pyrimidin-7-yl)phenyl]-3-(trifluoromethyl)benzamide;
4-fluoro-N-[3-(3-pyridin-4-ylpyrazolo[1,5-a]pyrimidin-7-yl)phenyl]-3-(trifluoromethyl)benzamide;
4-chloro-N-[3-(3-pyridin-4-ylpyrazolo[1,5-a]pyrimidin-7-yl)phenyl]-3-(trifluoromethyl)benzamide;
N-[3-(3-pyridin-4-ylpyrazolo[1,5-a]pyrimidin-7-yl)phenyl]-N′-[3-(trifluoromethyl)phenyl]urea;
N-[4-fluoro-3-(trifluoromethyl)phenyl]-N′-[3-(3-pyridin-4-ylpyrazolo[1,5-a]pyrimidin-7-yl)phenyl]urea;
4-methyl-N-[3-(2-morpholin-4-ylpyrazolo[1,5-a]pyrimidin-7-yl)phenyl]-3-(trifluoromethyl)benzamide;
4-methoxy-N-[3-(2-morpholin-4-ylpyrazolo[1,5-a]pyrimidin-7-yl)phenyl]-3-(trifluoromethyl)benzamide;
4-fluoro-N-[3-(2-morpholin-4-ylpyrazolo[1,5-a]pyrimidin-7-yl)phenyl]-3-(trifluoromethyl)benzamide;
4-chloro-N-[3-(2-morpholin-4-ylpyrazolo[1,5-a]pyrimidin-7-yl)phenyl]-3-(trifluoromethyl)benzamide;
3,4-dichloro-N-[3-(2-morpholin-4-ylpyrazolo[1,5-a]pyrimidin-7-yl)phenyl]benzamide;
N-[3-(2-morpholin-4-ylpyrazolo[1,5-a]pyrimidin-7-yl)phenyl]-N′-[3-(trifluoromethyl)phenyl]urea;
N-[4-chloro-3-(trifluoromethyl)phenyl]-N′-[3-(2-morpholin-4-ylpyrazolo[1,5-a]pyrimidin-7-yl)phenyl]urea;
N-(3,4-dichlorophenyl)-N′-[3-(2-morpholin-4-ylpyrazolo[1,5-a]pyrimidin-7-yl)phenyl]urea;
N-[4-fluoro-3-(trifluoromethyl)phenyl]-N′-[3-(2-morpholin-4-ylpyrazolo[1,5-a]pyrimidin-7-yl)phenyl]urea;
N-{3-[2-(4-methylpiperazin-1-yl)pyrazolo[1,5-a]pyrimidin-7-yl]phenyl}-3-(trifluoromethyl)benzamide;
ethyl 7-{3-[(pyridin-3-ylcarbonyl)amino]phenyl}pyrazolo[1,5-a]pyrimidine-3-carboxylate;
N-(3-{3-[3-(dimethylamino)prop-1-yn-1-yl]pyrazolo[1,5-a]pyrimidin-7-yl}phenyl)-3-(trifluoromethyl)benzamide;
tert-butyl 4-[7-(3-{[3-(trifluoromethyl)benzoyl]amino}phenyl)pyrazolo[1,5-a]pyrimidin-2-yl]piperazine-1-carboxylate;
N-{3-[2-(4-benzylpiperazin-1-yl)pyrazolo[1,5-a]pyrimidin-7-yl]phenyl}-3-(trifluoromethyl)benzamide;
N-[3-(2-piperazin-1-ylpyrazolo[1,5-a]pyrimidin-7-yl)phenyl]-3-(trifluoromethyl)benzamide;
N-(3-{2-[3-(dimethylamino)pyrrolidin-1-yl]pyrazolo[1,5-a]pyrimidin-7-yl}phenyl)-3-(trifluoromethyl)benzamide;
N-(3-{2-[(2R)-2-(methoxymethyl)pyrrolidin-1-yl]pyrazolo[1,5-a]pyrimidin-7-yl}phenyl)-3-(trifluoromethyl)benzamide;
N-(3-{2-[(2S)-2-(pyrrolidin-1-ylmethyl)pyrrolidin-1-yl]pyrazolo[1,5-a]pyrimidin-7-yl}phenyl)-3-(trifluoromethyl)benzamide;
tert-butyl {1-[7-(3-{[3-(trifluoromethyl)benzoyl]amino}phenyl)pyrazolo[1,5-a]pyrimidin-2-yl]pyrrolidin-3-yl}carbamate;
phenyl}-3-(trifluoromethyl)benzamide;
ethyl 7-{3-[(pyrazin-2-ylcarbonyl)amino]phenyl}pyrazolo[1,5-a]pyrimidine-3-carboxylate;
ethyl 7-(3-{[(1-methyl-1H-pyrrol-2-yl)carbonyl]amino}phenyl)pyrazolo[1,5-a]pyrimidine-3-carboxylate;
ethyl 7-(3-{[(5-methylpyrazin-2-yl)carbonyl]amino}phenyl)pyrazolo[1,5-a]pyrimidine-3-carboxylate;
ethyl 7-(3-{[(4-chloropyridin-2-yl)carbonyl]amino}phenyl)pyrazolo[1,5-a]pyrimidine-3-carboxylate;
ethyl 7-{3-[(isoquinolin-1-ylcarbonyl)amino]phenyl}pyrazolo[1,5-a]pyrimidine-3-carboxylate;
ethyl 7-(3-{[(1-methyl-1H-indol-2-yl)carbonyl]amino}phenyl)pyrazolo[1,5-a]pyrimidine-3-carboxylate;
ethyl 7-(3-{[(5-methyl-2-phenyl-2H-1,2,3-triazol-4-yl)carbonyl]amino}phenyl)pyrazolo[1,5-a]pyrimidine-3-carboxylate;
ethyl 7-(3-{[(5-methyl-2-thienyl)carbonyl]amino}phenyl)pyrazolo[1,5-a]pyrimidine-3-carboxylate;
ethyl 7-(3-{[(5-chloro-2-thienyl)carbonyl]amino}phenyl)pyrazolo[1,5-a]pyrimidine-3-carboxylate;
ethyl 7-(3-{[(5-bromo-2-thienyl)carbonyl]amino}phenyl)pyrazolo[1,5-a]pyrimidine-3-carboxylate;
ethyl 7-{3-[({5-[1-methyl-3-(trifluoromethyl)-1H-pyrazol-5-yl]-2-thienyl}carbonyl)amino]phenyl}pyrazolo[1,5-a]pyrimidine-3-carboxylate;
ethyl 7-{3-[(3,3-dimethylbutanoyl)amino]phenyl}pyrazolo[1,5-a]pyrimidine-3-carboxylate;
ethyl 7-{3-[(3,5,5-trimethylhexanoyl)amino]phenyl}pyrazolo[1,5-a]pyrimidine-3-carboxylate;
ethyl 7-{3-[(3,5-di-tert-butylbenzoyl)amino]phenyl}pyrazolo[1,5-a]pyrimidine-3-carboxylate;
ethyl 7-{3-[(2-bromo-5-chlorobenzoyl)amino]phenyl}pyrazolo[1,5-a]pyrimidine-3-carboxylate;
N-[3-(3-{4-[(methoxyacetyl)amino]phenyl}pyrazolo[1,5-a]pyrimidin-7-yl)phenyl]-3-(trifluoromethyl)benzamide;
N-[3-(3-{4-[(N,N-dimethylglycyl)amino]phenyl}pyrazolo[1,5-a]pyrimidin-7-yl)phenyl]-3-(trifluoromethyl)benzamide;
N-[3-(3-{4-[(3-methoxypropanoyl)amino]phenyl}pyrazolo[1,5-a]pyrimidin-7-yl)phenyl]-3-(trifluoromethyl)benzamide;
N-[3-(3-{4-[(1H-imidazol-4-ylacetyl)amino]phenyl}pyrazolo[1,5-a]pyrimidin-7-yl)phenyl]-3-(trifluoromethyl)benzamide;
N-[3-(3-{4-[(1H-tetrazol-5-ylacetyl)amino]phenyl}pyrazolo[1,5-a]pyrimidin-7-yl)phenyl]-3-(trifluoromethyl)benzamide;
N-{3-[3-(4-{[4-(dimethylamino)butanoyl]amino}phenyl)pyrazolo[1,5-a]pyrimidin-7-yl]phenyl}-3-(trifluoromethyl)benzamide;
N-{3-[3-(4-{[(2-methoxyethoxy)acetyl]amino}phenyl)pyrazolo[1,5-a]pyrimidin-7-yl]phenyl}-3-(trifluoromethyl)benzamide;
1-methyl-N-[3-(2-pyridin-4-ylpyrazolo[1,5-a]pyrimidin-7-yl)phenyl]-1H-pyrrole-2-carboxamide;
N-[3-(2-pyridin-4-ylpyrazolo[1,5-a]pyrimidin-7-yl)phenyl]isoquinoline-1-carboxamide;
1-methyl-N-[3-(2-pyridin-4-ylpyrazolo[1,5-a]pyrimidin-7-yl)phenyl]-1H-indole-2-carboxamide;
5-bromo-N-[3-(2-pyridin-4-ylpyrazolo[1,5-a]pyrimidin-7-yl)phenyl]thiophene-2-carboxamide;
3,3-dimethyl-N-[3-(2-pyridin-4-ylpyrazolo[1,5-a]pyrimidin-7-yl)phenyl]butanamide;
2-bromo-5-chloro-N-[3-(2-pyridin-4-ylpyrazolo[1,5-a]pyrimidin-7-yl)phenyl]benzamide;
ethyl 7-{3-[(3-methylbenzoyl)amino]phenyl}pyrazolo[1,5-a]pyrimidine-3-carboxylate;
ethyl 7-(3-{[(3-tert-butyl-1-methyl-1H-pyrazol-5-yl)carbonyl]amino}phenyl)pyrazolo[1,5-a]pyrimidine-3-carboxylate;
ethyl 7-[3-({[(6-methoxy-1,3-benzothiazol-2-yl)amino]carbonyl}amino)phenyl]pyrazolo[1,5-a]pyrimidine-3-carboxylate;
ethyl 7-(3-{[(1,3-benzodioxol-5-ylamino)carbonyl]amino}phenyl)pyrazolo[1,5-a]pyrimidine-3-carboxylate;
ethyl 7-[3-({[(6-chloro-1,3-benzothiazol-2-yl)amino]carbonyl}amino)phenyl]pyrazolo[1,5-a]pyrimidine-3-carboxylate;
ethyl 7-[3-({[(3-methylisoxazol-5-yl)amino]carbonyl}amino)phenyl]pyrazolo[1,5-a]pyrimidine-3-carboxylate;
ethyl 7-[3-({[(5-methylisoxazol-3-yl)amino]carbonyl}amino)phenyl]pyrazolo[1,5-a]pyrimidine-3-carboxylate;
N-{3-[2-(3-oxopiperazin-1-yl)pyrazolo[1,5-a]pyrimidin-7-yl]phenyl}-3-(trifluoromethyl)benzamide;
N-[3-(2-hydroxypyrazolo[1,5-a]pyrimidin-7-yl)phenyl]-3-(trifluoromethyl)benzamide;
N-{3-[2-(4-oxopiperidin-1-yl)pyrazolo[1,5-a]pyrimidin-7-yl]phenyl}-3-(trifluoromethyl)benzamide;
N-[3-(3-{3-[(methoxyacetyl)amino]phenyl}pyrazolo[1,5-a]pyrimidin-7-yl)phenyl]-3-(trifluoromethyl)benzamide;
N-[3-(3-{3-[(N,N-dimethylglycyl)amino]phenyl}pyrazolo[1,5-a]pyrimidin-7-yl)phenyl]-3-(trifluoromethyl)benzamide;
N-[3-(3-{3-[(3-methoxypropanoyl)amino]phenyl}pyrazolo[1,5-a]pyrimidin-7-yl)phenyl]-3-(trifluoromethyl)benzamide;
N-[3-(3-{3-[(N-acetylglycyl)amino]phenyl}pyrazolo[1,5-a]pyrimidin-7-yl)phenyl]-3-(trifluoromethyl)benzamide;
N-[3-(3-{3-[(1H-tetrazol-5-ylacetyl)amino]phenyl}pyrazolo[1,5-a]pyrimidin-7-yl)phenyl]-3-(trifluoromethyl)benzamide;
N-{3-[2-(2-{[3-(dimethylamino)propyl]amino}pyridin-4-yl)pyrazolo[1,5-a]pyrimidin-7-yl]phenyl}-3-(trifluoromethyl)benzamide;
N-[3-(2-{2-[(3-morpholin-4-ylpropyl)amino]pyridin-4-yl}pyrazolo[1,5-a]pyrimidin-7-yl)phenyl]-3-(trifluoromethyl)benzamide;
N-[3-(2-{2-[(3-piperidin-1-ylpropyl)amino]pyridin-4-yl}pyrazolo[1,5-a]pyrimidin-7-yl)phenyl]-3-(trifluoromethyl)benzamide;
N-[3-(2-{2-[(2-morpholin-4-ylethyl)amino]pyridin-4-yl}pyrazolo[1,5-a]pyrimidin-7-yl)phenyl]-3-(trifluoromethyl)benzamide;
N-{3-[2-(2-{[3-(2-oxopyrrolidin-1-yl)propyl]amino}pyridin-4-yl)pyrazolo[1,5-a]pyrimidin-7-yl]phenyl}-3-(trifluoromethyl)benzamide;
N-{3-[2-(2-{[3-(1H-imidazol-1-yl)propyl]amino}pyridin-4-yl)pyrazolo[1,5-a]pyrimidin-7-yl]phenyl}-3-(trifluoromethyl)benzamide;
N-{3-[2-(2-{[2-(4-hydroxypiperidin-1-yl)ethyl]amino}pyridin-4-yl)pyrazolo[1,5-a]pyrimidin-7-yl]phenyl}-3-(trifluoromethyl)benzamide;
N-[3-(2-{2-[(2-piperidin-1-ylethyl)amino]pyridin-4-yl}pyrazolo[1,5-a]pyrimidin-7-yl)phenyl]-3-(trifluoromethyl)benzamide;
N-[3-(2-{2-[(2-pyrrolidin-1-ylethyl)amino]pyridin-4-yl}pyrazolo[1,5-a]pyrimidin-7-yl)phenyl]-3-(trifluoromethyl)benzamide;
N-{3-[2-(2-{[2-(dimethylamino)ethyl]amino}pyridin-4-yl)pyrazolo[1,5-a]pyrimidin-7-yl]phenyl}-3-(trifluoromethyl)benzamide;
N-[3-(2-{2-[(3-pyrrolidin-1-ylpropyl)amino]pyridin-4-yl}pyrazolo[1,5-a]pyrimidin-7-yl)phenyl]-3-(trifluoromethyl)benzamide;
N-{3-[2-(2-{[2-(2-oxoimidazolidin-1-yl)ethyl]amino}pyridin-4-yl)pyrazolo[1,5-a]pyrimidin-7-yl]phenyl}-3-(trifluoromethyl)benzamide;
N-[3-(2-{2-[(3-aminopropyl)(methyl)amino]pyridin-4-yl}pyrazolo[1,5-a]pyrimidin-7-yl)phenyl]-3-(trifluoromethyl)benzamide;
N-[3-(2-{2-[(2-aminoethyl)(methyl)amino]pyridin-4-yl}pyrazolo[1,5-a]pyrimidin-7-yl)phenyl]-3-(trifluoromethyl)benzamide;
N-(3-{3-[3-(aminocarbonyl)phenyl]pyrazolo[1,5-a]pyrimidin-7-yl}phenyl)-3-(trifluoromethyl)benzamide;
N-[3-(3-{2-[(dimethylamino)methyl]phenyl}pyrazolo[1,5-a]pyrimidin-7-yl)phenyl]-3-(trifluoromethyl)benzamide;
N-[3-(3-{3-[(dimethylamino)methyl]phenyl}pyrazolo[1,5-a]pyrimidin-7-yl)phenyl]-3-(trifluoromethyl)benzamide;
N-[3-(3-{4-[(dimethylamino)methyl]phenyl}pyrazolo[1,5-a]pyrimidin-7-yl)phenyl]-3-(trifluoromethyl)benzamide;
N-{3-[3-(1H-pyrazol-4-yl)pyrazolo[1,5-a]pyrimidin-7-yl]phenyl}-3-(trifluoromethyl)benzamide;
tert-butyl 4-[7-(3-{[3-(trifluoromethyl)benzoyl]amino}phenyl)pyrazolo[1,5-a]pyrimidin-3-yl]-1H-pyrazole-1-carboxylate;
N-{3-[3-(3-furyl)pyrazolo[1,5-a]pyrimidin-7-yl]phenyl}-3-(trifluoromethyl)benzamide;
N-{3-[3-(6-aminopyridin-3-yl)pyrazolo[1,5-a]pyrimidin-7-yl]phenyl}-3-(trifluoromethyl)benzamide;
N-(3-{3-[5-(4-methylpiperazin-1-yl)pent-1-yn-1-yl]pyrazolo[1,5-a]pyrimidin-7-yl}phenyl)-3-(trifluoromethyl)benzamide;
N-[3-(3-{2-[2-(dimethylamino)ethyl]phenyl}pyrazolo[1,5-a]pyrimidin-7-yl)phenyl]-3-(trifluoromethyl)benzamide;
N-[3-(3-{3-[2-(dimethylamino)ethyl]phenyl}pyrazolo[1,5-a]pyrimidin-7-yl)phenyl]-3-(trifluoromethyl)benzamide;
N-{3-[3-(5-morpholin-4-ylpent-1-yn-1-yl)pyrazolo[1,5-a]pyrimidin-7-yl]phenyl}-3-(trifluoromethyl)benzamide;
N-{3-[3-(6-{[2-(dimethylamino)ethyl]amino}pyridin-3-yl)pyrazolo[1,5-a]pyrimidin-7-yl]phenyl}-3-(trifluoromethyl)benzamide;
N-(3-{3-[6-(methylamino)pyridin-3-yl]pyrazolo[1,5-a]pyrimidin-7-yl}phenyl)-3-(trifluoromethyl)benzamide;
N-(3-{3-[4-(hydroxymethyl)phenyl]pyrazolo[1,5-a]pyrimidin-7-yl}phenyl)-3-(trifluoromethyl)benzamide;
N-(3-{3-[3-(hydroxymethyl)phenyl]pyrazolo[1,5-a]pyrimidin-7-yl}phenyl)-3-(trifluoromethyl)benzamide;
N-{3-[2-(4-bromophenyl)pyrazolo[1,5-a]pyrimidin-7-yl]phenyl}-3-(trifluoromethyl)benzamide;
N-[3-(3-{4-[(dimethylamino)sulfonyl]phenyl}pyrazolo[1,5-a]pyrimidin-7-yl)phenyl]-3-(trifluoromethyl)benzamide;
N-[3-(3-{4-[2-(dimethylamino)ethyl]phenyl}pyrazolo[1,5-a]pyrimidin-7-yl)phenyl]-3-(trifluoromethyl)benzamide;
3-(trifluoromethyl)-N-(3-{3-[2-(trifluoromethyl)phenyl]pyrazolo[1,5-a]pyrimidin-7-yl}phenyl)benzamide;
3-(trifluoromethyl)-N-(3-{3-[3-(trifluoromethyl)phenyl]pyrazolo[1,5-a]pyrimidin-7-yl}phenyl)benzamide;
3-(trifluoromethyl)-N-(3-{3-[4-(trifluoromethyl)phenyl]pyrazolo[1,5-a]pyrimidin-7-yl}phenyl)benzamide;
N-{3-[3-(2-cyanophenyl)pyrazolo[1,5-a]pyrimidin-7-yl]phenyl}-3-(trifluoromethyl)benzamide;
N-{3-[3-(3-cyanophenyl)pyrazolo[1,5-a]pyrimidin-7-yl]phenyl}-3-(trifluoromethyl)benzamide;
N-{3-[3-(4-cyanophenyl)pyrazolo[1,5-a]pyrimidin-7-yl]phenyl}-3-(trifluoromethyl)benzamide;
methyl 3-[7-(3-{[3-(trifluoromethyl)benzoyl]amino}phenyl)pyrazolo[1,5-a]pyrimidin-3-yl]benzoate;
methyl 4-[7-(3-{[3-(trifluoromethyl)benzoyl]amino}phenyl)pyrazolo[1,5-a]pyrimidin-3-yl]benzoate;
N-{3-[3-(2-acetylphenyl)pyrazolo[1,5-a]pyrimidin-7-yl]phenyl}-3-(trifluoromethyl)benzamide;
N-{3-[3-(3-acetylphenyl)pyrazolo[1,5-a]pyrimidin-7-yl]phenyl}-3-(trifluoromethyl)benzamide;
N-{3-[3-(4-acetylphenyl)pyrazolo[1,5-a]pyrimidin-7-yl]phenyl}-3-(trifluoromethyl)benzamide;
N-{3-[3-(2-chloropyridin-4-yl)pyrazolo[1,5-a]pyrimidin-7-yl]phenyl}-3-(trifluoromethyl)benzamide;
N-[3-(5-methyl-2-pyridin-4-ylpyrazolo[1,5-a]pyrimidin-7-yl)phenyl]-3-(trifluoromethyl)benzamide;
N-(3-{3-[2-(1-hydroxyethyl)phenyl]pyrazolo[1,5-a]pyrimidin-7-yl}phenyl)-3-(trifluoromethyl)benzamide;
N-(3-{3-[3-(1-hydroxyethyl)phenyl]pyrazolo[1,5-a]pyrimidin-7-yl}phenyl)-3-(trifluoromethyl)benzamide;
N-{3-[3-(2-methylphenyl)pyrazolo[1,5-a]pyrimidin-7-yl]phenyl}-3-(trifluoromethyl)benzamide;
N-{3-[3-(1-methyl-1,2,3,6-tetrahydropyridin-4-yl)pyrazolo[1,5-a]pyrimidin-7-yl]phenyl}-3-(trifluoromethyl)benzamide;
N-(3-{3-[1-(2-pyrrolidin-1-ylethyl)-1H-pyrazol-4-yl]pyrazolo[1,5-a]pyrimidin-7-yl}phenyl)-3-(trifluoromethyl)benzamide;
N-{3-[3-(1-methyl-1H-pyrazol-4-yl)pyrazolo[1,5-a]pyrimidin-7-yl]phenyl}-3-(trifluoromethyl)benzamide;
N-{3-[3-(1-methylpiperidin-4-yl)pyrazolo[1,5-a]pyrimidin-7-yl]phenyl}-3-(trifluoromethyl)benzamide;
N-{3-[3-(3,5-diformylphenyl)pyrazolo[1,5-a]pyrimidin-7-yl]phenyl}-3-(trifluoromethyl)benzamide;
N-{3-[3-(6-fluoropyridin-3-yl)pyrazolo[1,5-a]pyrimidin-7-yl]phenyl}-3-(trifluoromethyl)benzamide;
N-{3-[3-(6-methoxypyridin-3-yl)pyrazolo[1,5-a]pyrimidin-7-yl]phenyl}-3-(trifluoromethyl)benzamide;
N-{3-[3-(5-formyl-2-furyl)pyrazolo[1,5-a]pyrimidin-7-yl]phenyl}-3-(trifluoromethyl)benzamide;
3-[7-(3-{[3-(trifluoromethyl)benzoyl]amino}phenyl)pyrazolo[1,5-a]pyrimidin-3-yl]benzoic acid;
N-(3-{3-[4-(pyrrolidin-1-ylmethyl)phenyl]pyrazolo[1,5-a]pyrimidin-7-yl}phenyl)-3-(trifluoromethyl)benzamide;
N-(3-{3-[5-(pyrrolidin-1-ylmethyl)-2-furyl]pyrazolo[1,5-a]pyrimidin-7-yl}phenyl)-3-(trifluoromethyl)benzamide;
N-[4-fluoro-3-(2-pyridin-4-ylpyrazolo[1,5-a]pyrimidin-7-yl)phenyl]-3-(trifluoromethyl)benzamide;
N-(4-fluoro-3-pyrazolo[1,5-a]pyrimidin-7-ylphenyl)-3-(trifluoromethyl)benzamide;
N-(3-{3-[5-(pyrrolidin-1-ylmethyl)-3-furyl]pyrazolo[1,5-a]pyrimidin-7-yl}phenyl)-3-(trifluoromethyl)benzamide.
N-[3-(3-iodopyrazolo[1,5-a]pyrimidin-7-yl)phenyl]-3-(trifluoromethyl)benzamide;
N-{3-[3-(5-formyl-3-furyl)pyrazolo[1,5-a]pyrimidin-7-yl]phenyl}-3-(trifluoromethyl)benzamide;
N-[3-(3-{5-[(4-ethylpiperazin-1-yl)methyl]-3-furyl}pyrazolo[1,5-a]pyrimidin-7-yl)phenyl]-3-(trifluoromethyl)benzamide;
3-(trifluoromethyl)-N-(3-{3-[(trimethylsilyl)ethynyl]pyrazolo[1,5-a]pyrimidin-7-yl}phenyl)benzamide;
N-{3-[2-(imidazol-5-yl)pyrazolo[1,5-a]pyrimidin-7-yl]phenyl}-3-(trifluoromethyl)benzamide-3,5-difluoro-N-[3-(2-pyridin-4-ylpyrazolo[1,5-a]pyrimidin-7-yl)phenyl]benzamide;
ethyl 7-(2-methoxy-5-{[3-(trifluoromethyl)benzoyl]amino}phenyl)pyrazolo[1,5-a]pyrimidine-3-carboxylate;
N-(4-methoxy-3-pyrazolo[1,5-a]pyrimidin-7-ylphenyl)-3-(trifluoromethyl)benzamide;
N-[3-(3-ethynylpyrazolo[1,5-a]pyrimidin-7-yl)phenyl]-3-(trifluoromethyl)benzamide;
N-[3-(3-{4-[(4-methylpiperazin-1-yl)methyl]phenyl}pyrazolo[1,5-a]pyrimidin-7-yl)phenyl]-3-(trifluoromethyl)benzamide;
N-{3-[3-(2-methoxypyrimidin-5-yl)pyrazolo[1,5-a]pyrimidin-7-yl]phenyl}-3-(trifluoromethyl)benzamide;
N-[3-(3-{3,5-bis[(dimethylamino)methyl]phenyl}pyrazolo[1,5-a]pyrimidin-7-yl)phenyl]-3-(trifluoromethyl)benzamide;
N-[3-(3-iodopyrazolo[1,5-a]pyrimidin-7-yl)-4-methoxyphenyl]-3-(trifluoromethyl)benzamide;
N-[4-fluoro-3-(3-iodopyrazolo[1,5-a]pyrimidin-7-yl)phenyl]-3-(trifluoromethyl)benzamide;
3-(difluoromethyl)-N-[3-(2-pyridin-4-ylpyrazolo[1,5-a]pyrimidin-7-yl)phenyl]benzamide;
N-(3-{3-[6-(4-methylpiperazin-1-yl)pyridin-3-yl]pyrazolo[1,5-a]pyrimidin-7-yl}phenyl)-3-(trifluoromethyl)benzamide;
N-{3-[3-(3,5-dimethylisoxazol-4-yl)pyrazolo[1,5-a]pyrimidin-7-yl]phenyl}-3-(trifluoromethyl)benzamide;
N-[3-(3-pyrimidin-5-ylpyrazolo[1,5-a]pyrimidin-7-yl)phenyl]-3-(trifluoromethyl)benzamide;
N-(3-{3-[2-(dimethylamino)pyrimidin-5-yl]pyrazolo[1,5-a]pyrimidin-7-yl}phenyl)-3-(trifluoromethyl)benzamide;
N-{3-[3-(2-morpholin-4-ylpyrimidin-5-yl)pyrazolo[1,5-a]pyrimidin-7-yl]phenyl}-3-(trifluoromethyl)benzamide;
7-[3-(3-Trifluoromethyl-benzoylamino)-phenyl]-pyrazolo[1,5-a]pyrimidine-2-carboxylic acid;
N-{4-fluoro-3-[3-(1H-pyrazol-4-yl)pyrazolo[1,5-a]pyrimidin-7-yl]phenyl}-3-(trifluoromethyl)benzamide;
tert-butyl 4-[7-(2-fluoro-5-{[3-(trifluoromethyl)benzoyl]amino}phenyl)pyrazolo[1,5-a]pyrimidin-3-yl]-1H-pyrazole-1-carboxylate;
N-(3-{3-[2-(4-methylpiperazin-1-yl)pyridin-4-yl]pyrazolo[1,5-a]pyrimidin-7-yl}phenyl)-3-(trifluoromethyl)benzamide;
N-(4-fluoro-3-{3-[6-(4-methylpiperazin-1-yl)pyridin-3-yl]pyrazolo[1,5-a]pyrimidin-7-yl}phenyl)-3-(trifluoromethyl)benzamide;
N-[4-chloro-3-(2-pyridin-4-ylpyrazolo[1,5-a]pyrimidin-7-yl)phenyl]-3-(trifluoromethyl)benzamide;
ethyl 7-(2-chloro-5-{[3-(trifluoromethyl)benzoyl]amino}phenyl)pyrazolo[1,5-a]pyrimidine-3-carboxylate;
N-(4-chloro-3-pyrazolo[1,5-a]pyrimidin-7-ylphenyl)-3-(trifluoromethyl)benzamide;
N-{3-[3-(4-piperazin-1-ylphenyl)pyrazolo[1,5-a]pyrimidin-7-yl]phenyl}-3-(trifluoromethyl)benzamide;
N-{3-[2-(1-Methyl-1H-imidazol-5-yl)pyrazolo[1,5-a]pyrimidin-7-yl]phenyl}-3-(trifluoromethyl)benzamide;
tert-Butyl 2-[7-(3-{[3-(trifluoromethyl)benzoyl]amino}phenyl)pyrazolo[1,5-a]pyrimidin-3-yl]-1H-pyrrole-1-carboxylate;
N-[4-chloro-3-(3-iodopyrazolo[1,5-a]pyrimidin-7-yl)phenyl]-3-(trifluoromethyl)benzamide;
Methyl 7-(2-fluoro-5-{[3-(trifluoromethyl)benzoyl]amino}phenyl)pyrazolo[1,5-a]pyrimidine-3-carboxylate;
N-(3-{3-[4-(4-methylpiperazin-1-yl)phenyl]pyrazolo[1,5-a]pyrimidin-7-yl}phenyl)-3-(trifluoromethyl)benzamide;
N-{3-[3-(6-acetamidopyridin-3-yl)pyrazolo[1,5-a]pyrimidin-7-yl]phenyl}-3-(trifluoromethyl)benzamide;
N-[3-(3-bromopyrazolo[1,5-a]pyrimidin-7-yl)-4-chlorophenyl]-3-(trifluoromethyl)benzamide;
Ethyl 7-(3-{[4-{[(tert-butoxycarbonyl)(methyl)amino]methyl}-3-(trifluoromethyl)benzoyl]amino}phenyl)pyrazolo[1,5-a]pyrimidine-3-carboxylate;
Ethyl 7-[3-({4-[(methylamino)methyl]-3-(trifluoromethyl)benzoyl}amino)phenyl]pyrazolo[1,5-a]pyrimidine-3-carboxylate;
N-[4-chloro-3-(3-chloropyrazolo[1,5-a]pyrimidin-7-yl)phenyl]-3-(trifluoromethyl)benzamide;
Ethyl 7-(3-{[3-nitro-5-(trifluoromethyl)benzoyl]amino}phenyl)pyrazolo[1,5-a]pyrimidine-3-carboxylate;
N-[3-(2-pyridin-4-ylpyrazolo[1,5-a]pyrimidin-7-yl)phenyl]-2-[3-(trifluoromethyl)phenyl]acetamide;
Methyl 7-(2-chloro-5-{[3-(trifluoromethyl)benzoyl]amino}phenyl)pyrazolo[1,5-a]pyrimidine-3-carboxylate;
Ethyl 7-(3-{[3-amino-5-(trifluoromethyl)benzoyl]amino}phenyl)pyrazolo[1,5-a]pyrimidine-3-carboxylate;
N-{4-methoxy-3-[3-(1H-pyrazol-4-yl)pyrazolo[1,5-a]pyrimidin-7-yl]phenyl}-3-(trifluoromethyl)benzamide;
tert-butyl 4-[7-(2-methoxy-5-{[3-(trifluoromethyl)benzoyl]amino}phenyl)pyrazolo[1,5-a]pyrimidin-3-yl]-1H-pyrazole-1-carboxylate;
3-bromo-N-[3-(2-pyridin-4-ylpyrazolo[1,5-a]pyrimidin-7-yl)phenyl]benzamide, trifluoroacetic acid salt;
3-fluoro-N-[3-(2-pyridin-4-ylpyrazolo[1,5-a]pyrimidin-7-yl)phenyl]benzamide, trifluoroacetic acid salt;
3-nitro-N-[3-(2-pyridin-4-ylpyrazolo[1,5-a]pyrimidin-7-yl)phenyl]benzamide, trifluoroacetic acid salt;
3-cyano-N-[3-(2-pyridin-4-ylpyrazolo[1,5-a]pyrimidin-7-yl)phenyl]benzamide, trifluoroacetic acid salt;
N-[3-(2-pyridin-4-ylpyrazolo[1,5-a]pyrimidin-7-yl)phenyl]-3-(trifluoro-methoxy)benzamide, trifluoroacetic acid salt;
3-(dimethylamino)-N-[3-(2-pyridin-4-ylpyrazolo[1,5-a]pyrimidin-7-yl)phenyl]-benzamide, trifluoroacetic acid salt;
3,4-difluoro-N-[3-(2-pyridin-4-ylpyrazolo[1,5-a]pyrimidin-7-yl)phenyl]benzamide, trifluoroacetic acid salt;
N-[3-(2-pyridin-4-ylpyrazolo[1,5-a]pyrimidin-7-yl)phenyl]-1,3-benzodioxole-5-carboxamide, trifluoroacetic acid salt;
4-fluoro-N-[3-(2-pyridin-4-ylpyrazolo[1,5-a]pyrimidin-7-yl)phenyl]-3-(trifluoromethyl)benzamide, trifluoroacetic acid salt;
4-bromo-3-methyl-N-[3-(2-pyridin-4-ylpyrazolo[1,5-a]pyrimidin-7-yl)phenyl]-benzamide, trifluoroacetic acid salt;
3-fluoro-N-[3-(2-pyridin-4-ylpyrazolo[1,5-a]pyrimidin-7-yl)phenyl]-4-(trifluoromethyl)benzamide;
Ethyl 7-[3-({3-[(chloroacetyl)amino]-5-(trifluoromethyl)benzoyl}amino)phenyl]pyrazolo[1,5-a]pyrimidine-3-carboxylate;
1-(3-{[3-(2-Pyridin-4-ylpyrazolo[1,5-a]pyrimidin-7-yl)phenyl]carbamoyl}-benzyl)pyridinium chloride;
N-{3-[3-(3,5-dimethyl-1H-pyrazol-4-yl)pyrazolo[1,5-a]pyrimidin-7-yl]phenyl}-3-(trifluoromethyl)benzamide;
N-{3-[3-(5-Oxo-4,5-dihydro-[1,3,4]oxadiazol-2-yl)-pyrazolo[1,5-a]pyrimidin-7-yl]-phenyl}-3-trifluoromethyl-benzamide;
N-{4-chloro-3-[3-(1H-pyrazol-4-yl)pyrazolo[1,5-a]pyrimidin-7-yl]phenyl}-3-(trifluoromethyl)benzamide;
N-{3-[3-(3,5-dimethyl-1H-pyrazol-4-yl)pyrazolo[1,5-a]pyrimidin-7-yl]-4-fluorophenyl}-3-(trifluoromethyl)benzamide;
4-[(Methylamino)methyl]-N-[3-(2-pyridin-4-ylpyrazolo[1,5-a]pyrimidin-7-yl)phenyl]-3-(trifluoromethyl)benzamide;
4-{[(2-Methoxyethyl)amino]methyl}-N-[3-(2-pyridin-4-ylpyrazolo[1,5-a]pyrimidin-7-yl)phenyl]-3-(trifluoromethyl)benzamide;
3-(trifluoromethyl)-N-(3-{3-[1-(triisopropylsilyl)-1H-pyrrol-3-yl]pyrazolo[1,5-a]pyrimidin-7-yl}phenyl)benzamide;
Ethyl 7-(3-{[3-{[(4-methylpiperazin-1-yl)acetyl]amino}-5-(trifluoromethyl)benzoyl]amino}phenyl)pyrazolo[1,5-a]pyrimidine-3-carboxylate;
Ethyl 7-[3-({3-[(pyrrolidin-1-ylacetyl)amino]-5-(trifluoromethyl)benzoyl}amino)phenyl]pyrazolo[1,5-a]pyrimidine-3-carboxylate;
Ethyl 7-[3-({3-[(morpholin-4-ylacetyl)amino]-5-(trifluoromethyl)benzoyl}amino)phenyl]pyrazolo[1,5-a]pyrimidine-3-carboxylate;
N-{3-[3-(1H-pyrrol-3-yl)pyrazolo[1,5-a]pyrimidin-7-yl]phenyl}-3-(trifluoromethyl)benzamide;
N-(3-{2-[2-(2,2-Dimethyl-propionylamino)-pyridin-4-yl]-pyrazolo[1,5-a]pyrimidin-7-yl}-phenyl)-3-trifluoro methyl-benzamide;
3-Nitro-N-[3-(2-pyridin-4-ylpyrazolo[1,5-a]pyrimidin-7-yl)phenyl]-5-(trifluoromethyl)benzamide;
3-Amino-N-[3-(2-pyridin-4-ylpyrazolo[1,5-a]pyrimidin-7-yl)phenyl]-5-(trifluoromethyl)benzamide;
3-[(Chloroacetyl)amino]-N-[3-(2-pyridin-4-ylpyrazolo[1,5-a]pyrimidin-7-yl)phenyl]-5-(trifluoromethyl)benzamide;
N-[3-(2-Pyridin-4-ylpyrazolo[1,5-a]pyrimidin-7-yl)phenyl]-3-[(pyrrolidin-1-ylacetyl)amino]-5-(trifluoromethyl)benzamide;
N-(4-chloro-3-pyrazolo[1,5-a]pyrimidin-7-ylphenyl)-4-(morpholin-4-ylmethyl)-3-(trifluoromethyl)benzamide; and
4-(morpholin-4-ylmethyl)-N-[3-(2-pyridin-4-ylpyrazolo[1,5-a]pyrimidin-7-yl)phenyl]-3-(trifluoromethyl)benzamide.
